(19) United States
(12) Patent Application Publication
(10) Pub. No.: US 2023/0293409 A1
(43) Pub. Date: Sep. 21, 2023

(54) Compositions Comprising Antimicrobials and (Bio)-Alkanediols for Skin Protection

(71) Applicant: Symrise AG, Holzminden (DE)

(72) Inventors: Christin Koch, Detmold (DE); Sabine Lange, Holzminden (DE); Nikolas Bugdahn, Holzminden (DE)

(21) Appl. No.: 18/009,312

(22) PCT Filed: Dec. 9, 2021
(86) PCT No.: PCT/EP2021/084957

§ 371 Date: Dec. 8, 2022

(30) Foreign Application Priority Data
Dec. 9, 2020 (WO) PCT/EP2020/085173

(51) Int. Cl. A61K 8/34; A61Q 19/00; A61K 45/06; A61Q 17/00

(52) U.S. Cl. CPC A61K 8/345; A61Q 19/008; A61K 45/06; A61Q 17/005

(57) ABSTRACT

The present invention relates to a cosmetic or pharmaceutical, preferably dermatological, composition comprising or consisting of a specific antimicrobial component and an effective amount of 1,2-nonanediol and/or 2,3-nonanediol or of a specific alkanediol or a mixture of two or more different specific alkanediols and the use of said compositions as a cosmetic, for personal care, as a pharmaceutical or for animal care. Additionally, the present invention relates to the use of 1,2-nonanediol and/or 2,3-nonanediol or of a specific alkanediol or a mixture of two or more different specific alkanediols for reducing or inhibiting antimicrobial activity on skin or scalp, and/or for balancing sebum production or reducing sebum overproduction on skin or scalp and/or for suppressing dandruff on skin, scalp or nails and/or as antimicrobial active component replacer. Finally, the present invention relates to specific formulations such as personal hygiene product, deodorant, anti-perspirant, anti-acne product, or anti-dandruff product comprising a specific 1,2-alkanediol or a 2,3-alkanediol or a mixture of a specific 1,2-alkanediol and a specific 2,3-alkanediol as well as a method for reducing the sebum overproduction on skin or scalp and/or for suppressing dandruff on skin, scalp or nails.

### TECHNICAL FIELD

The present invention relates to a cosmetic or pharmaceutical, preferably dermatological, composition comprising or consisting of a synergistic combination of a specific antimicrobial component and an effective amount of 1,2-nonanediol and/or 2,3-nonanediol or of a specific alkanediol or a mixture of two or more different specific alkanediols and the use of said compositions as a cosmetic, for personal care, as a pharmaceutical or for animal care. Additionally, the present invention relates to the use of 1,2-nonanediol and/or 2,3-nonanediol or of a specific alkanediol or a mixture of two or more different specific alkanediols for reducing or inhibiting antimicrobial activity on skin or scalp, and/or for balancing sebum production or reducing sebum overproduction on skin or scalp and/or for suppressing dandruff on skin, scalp or nails and/or as antimicrobial active component replacer. Finally, the present invention relates to specific formulations such as personal hygiene product, deodorant, anti-perspirant, anti-acne product, or anti-dandruff product comprising a specific 1,2-alkanediol or a 2,3-alkanediol or a mixture of a specific 1,2-alkanediol and a specific 2,3-alkanediol as well as a method for reducing the sebum overproduction on skin or scalp and/or for suppressing dandruff on skin, scalp or nails.

### BACKGROUND ART

The skin is a human body's largest organ. It is colonized by diverse microorganisms, most of which are harmless or even beneficial to their host. Colonization is driven by the ecology of the skin surface, which is highly variable depending on topographical location, endogenous host factors and exogenous environmental factors. For instance, the armpits present a rather humid and nutrient-rich habitat that provides favorable conditions for growth of many microorganisms. Other habitats include sebum-rich habitats like the face and rather dry and nutrient-poor habitats like most other body regions.

Many common skin pathogens, such as Staphylococcus aureus and Streptococcus pyogenes, are inhibited by an acidic pH, thus the growth of coagulase-negative Staphylococci and Corynebacteria is favored. However, skin occlusion results in an elevated pH, which favors the growth of S. aureus and S. pyogenes.

Areas with a high density of sebaceous glands, such as the face, chest and back, encourage the growth of lipophilic microorganisms such as Corynebacterium spp, Propionibacterium spp and Malassezia spp.

Sebaceous glands are microscopic exocrine glands found throughout all areas of the skin. They secret a natural oil, called sebum, which participates with the sweat to compose the hydrolipidic film that covers the skin. Human sebum is a complex mixture of triglycerides, fatty acids, wax esters, sterol esters, cholesterol, cholesterol esters and squalene. Sebum is involved in epidermal development and barrier maintenance, transporting antioxidants, contributing to mechanical protection, body odour, and generation of pheromones. Sebum is directly involved in hormonal signalling, epidermal differentiation, and protection from ultraviolet (UV) radiation. It cooperates to reduce skin water loss and modulates composition and proliferation of the natural micro-flora of the skin.

There are two types of sebaceous glands, those connected to hair follicles, in pilosebaceous units, and those that exist independently. When they are associated to the hair follicles, one or more glands may surround each hair follicle, and the glands themselves are surrounded by arrector pili muscles. Sebaceous glands are also found in hairless areas (glabrous skin) of the eyelids (called meibomian glands), nose, labia minora, the inner mucosa of the cheek (Fordyce spots), etc.

Undesirable hyperactivity of sebaceous glands (seborrhoea) can also occur in other parts of the body, especially on the face. Here the overproduction of sebum gives the skin a shiny and aesthetically undesirable appearance (oily skin) and can promote other slight blemishes, such as comedones. In some cases, more serious disorders can occur in the presence of excessive sebum, such as acne, a skin disease characterized by an inflammatory process of the hair follicle and annexed sebaceous gland. Propionibacterium acnes (P. acnes; recently reclassified/renamed as: Cutibacterium acnes) is considered to be the infectious agent in acne.

The overproduction of sebum by sebaceous glands of the scalp is the cause of greasy hair, which is considered a significant aesthetic problem. The seborrhoea is involved in the occurrence of dandruff, a disorder of the scalp characterized by patches of abundant and loosely adherent flakes, usually accompanied by itching. This accentuated desquamation of the scalp can evolve into seborrheic dermatitis, which is a severe form of dandruff accompanied by inflammation and erythema. The etiology of dandruff and seborrheic dermatitis appears to be dependent upon three factors: sebaceous gland secretions, micro-flora metabolism, and individual susceptibility which are complemented by environmental conditions and personal lifestyle.

P. acnes bacteria are aerotolerant anaerobic bacteria that live deep within follicles and pores, using sebum, cellular debris and metabolic by-products from the surrounding skin tissue as their primary sources of energy and nutrients. Elevated production of sebum by hyperactive sebaceous glands or blockage of the follicle can favour P. acnes bacteria proliferation, causing the inflamed pustules (pimples) characteristic of acne.

In cases of acne, which usually occurs in puberty, the anaerobic microorganism P. acnes is involved decisively in the development of the condition and decomposes the sebum to glycerin and fatty acids, as a result of which the sebaceous glands are in turn stimulated to produce an increased amount of sebum, these degradation products attacking or destroying the follicle walls. This regularly causes inflammations in the skin (pimples, pustules, nodules, cysts), which often heal only with scarring, as a result of which the visual appearance of the person suffering from impure skin is permanently damaged.

Finally, female external genitals have many sebaceous glands and their sebum secretion interacts with the bacterial microflora, regulating the pH of the genital area. The fresh sebum does not contain significant quantities of free fatty acids, but these are released as an effect of the lipases produced by bacteria, inducing the acidification of the genital environment. The regulation of sebum can therefore represent an important condition for preventing alterations of the genital microflora, irritations, itching, etc.

Other skin disorders and infections can be caused by bacteria and fungi: Chronic wounds, affecting diabetic, elderly, and immobile individuals, are an example where commensal skin organisms invade and become pathogenic upon breach of the skin barrier. Although bacteria do not cause the initial wounding event, they are thought to contribute to the lack of healing and persistent inflammation that is associated with chronic wounds. Burn wounds commonly become infected with S. pyogenes, Enterococcus spp. or Pseudomonas aeruginosa.

A further common skin commensal is Staphylococcus epidermidis. It is also the most frequent cause of hospital-acquired infection on in-dwelling medical devices such as catheters or heart valves.

Hence, there is a need for products capable of controlling the skin microbiota, and in particular of inhibiting the growth of skin pathogens.

Additionally, the regulation of sebum production is therefore a pivotal issue for the prevention of dandruff and seborrheic dermatitis, and thereby controlling the colonization of the skin with specific microbial species which are lipid dependent and are feed on human serum.

As a consequence, the cosmetics industry is strongly interested in acquiring compounds or compositions suitable to inhibit sebum production, especially if this activity is combined with anti-inflammatory properties.

WO 2008/046791 discloses the use of 1,2-decanediol for sebum reduction of the skin and a cosmetic or dermatological formulation for topical application comprising 1,2-decanediol. In addition to 1,2-decanediol, the formulations include optionally cleansing substances and antimicrobials.

Meanwhile, 1,2-decanediol is an established antimicrobial used in deodorant, anti-perspirant, anti-dandruff, anti-acne and other hygiene applications for oral or personal care.

However, the formulation and in particular 1,2-decanediol has the disadvantage of an unpleasant inherent smell.

When searching for agents having an antimicrobial or sebum reduction action it must be taken into account that the substances used in the cosmetic or pharmaceutical sector must be toxicologically acceptable, well tolerated by the skin, stable, especially in the customary cosmetic and/or pharmaceutical formulations, substantially and preferably odourless and able to be prepared inexpensively, i.e. using standard methods and/or starting from standard precursors.

However, there is no clear dependency between the chemical structure of a substance, on the one hand, and its biological activity towards specific microorganisms or sebum production, on the other hand, which makes the search for suitable substances more difficult. Furthermore, there is no predictable relationship between antimicrobial action, toxicological acceptability, tolerability and the stability of a substance.

Thus, there is still an ongoing need to provide further substances or combinations which have a suppressive leverage on the microorganisms' growth on a mammal's skin or mucosa, and/or which have a suppressive leverage on sebum production of the sebaceous glands of the skin or scalp.

It is therefore an object of the present invention to provide a cosmetic or pharmaceutical, in particular dermatological, composition which has similar or even better antimicrobial properties than the cosmetic or pharmaceutical composition according to the prior art.

A further object of the present invention is to provide a cosmetic or pharmaceutical composition, which concomitantly reduces or minimizes the sebum overproduction on skin or scalp and/or and/or reduces dandruff on skin, scalp or nails.

A further object of the present invention is to provide a cosmetic or pharmaceutical composition, which has no unpleasant smell.

It is another object of the present invention to provide a substance, which boosts the effect of an antimicrobial component in a cosmetic or pharmaceutical composition, either by achieving a higher performance of an antimicrobial component with the same amounts, or vice versa, by achieving the same performance with lower amounts of an antimicrobial component, so that the overall content of antimicrobial component can be reduced in the ready-for-use formulation; and/or which reduces or minimizes or inhibits the sebum overproduction on skin or scalp and/or reduces dandruff on skin, scalp or nails upon application of the cosmetic or pharmaceutical composition.

Furthermore, it is an object of the present invention to provide a substance by which the amount of applied antimicrobial component in a cosmetic or pharmaceutical composition can be reduced, i.e., a substance which is suited as an antimicrobial replacer.

It is a further object of the present invention to provide a substance which does not have an unpleasant inherent smell.

Surprisingly it has been found that the addition of an effective amount of 1,2-nonanediol or 2,3-nonanediol or a mixture comprising 1,2-nonanediol and 2,-nonanediol or a specific alkanediol or a mixture of two or more different specific alkanediols as defined herein to a topical composition comprising an antimicrobial component significantly boosts the antimicrobial effect of said antimicrobial component, i.e. leads to a synergistic increase in antimicrobial activity of said antimicrobial component, against a plurality of microorganism of the mammalian natural microbiota on the skin or scalp or mucosa.

Concurrently, it has been found that the use of an effective amount of 1,2-nonanediol or 2,3-nonanediol or a mixture comprising 1,2-nonanediol and 2,3-nonanediol or a specific alkanediol or a mixture of two or more different specific alkanediols as defined herein in a topical composition has an advantageously, i.e., reducing, effect on sebum overproduction and/or on dandruff.

Surprisingly, it has been further observed that the overall amount of antimicrobial component in a topical composition can be significantly reduced by the addition of an effective amount of 1,2-nonanediol or 2,3-nonanediol or a mixture comprising 1,2-nonanediol and 2,3-nonanediol or a specific alkanediol or a mixture of two or more different specific alkanediols as defined herein.

### SUMMARY OF THE INVENTION

In order to accomplish the above problem, the present invention provides in a first aspect a cosmetic or pharmaceutical composition, comprising or consisting of
(a1) 1,2-nonanediol;
(b1) at least one antimicrobial component; and
(c1) optionally at least one cosmetically or pharmaceutically active substance and/or additive;
or
(a2) 2,3-nonanediol;
(b2) at least one antimicrobial component; and
(c2) optionally at least one cosmetically or pharmaceutically active substance and/or additive;
or
(a3) a mixture comprising 1,2-nonanediol and 2,3-nonanediol;
(b3) at least one antimicrobial component; and
(c3) optionally at least one cosmetically or pharmaceutically active substance and/or additive.

In a second aspect, the present invention provides a cosmetic or pharmaceutical composition, comprising or consisting of
(a) at least one linear alkanediol having a carbon chain of 5 to 14 carbon atoms or a mixture comprising at least one first linear alkanediol having a carbon chain of 5 to 14 carbon atoms and one or more second linear alkanediols having a carbon chain of 5 to 14 carbon atoms which is different from the first linear alkanediol;
(b) at least one antimicrobial component; and
(c) optionally at least one cosmetically or pharmaceutically active substance and/or additive.

In a further aspect, the present invention provides for the cosmetic or non-therapeutic use of the composition according to the present invention for personal care, in skin protection, skin care, scalp protection, scalp care, hair care, nail care, or in the prevention and/or treatment of skin conditions, intolerant or sensitive skin, skin irritation, skin reddening, pruritus (itching), loss of skin volume, loss of skin elasticity, skin dryness, flaking, greasiness; or for animal care.

In as still further aspect, the present invention provides for the use of the composition according to the present invention as a medicament.

In a still further aspect, the present invention provides for the use of 1,2-nonanediol or 2,3-nonanediol or a mixture comprising 1,2,-nonanediol and 2,3-nonanediol or at least one linear alkanediol or a mixture comprising at least one first linear alkanediol and one or more second linear alkanediols as defined therein as cosmetic or pharmaceutical, especially for reducing or inhibiting the microbial activity on skin or scalp, in particular the activity of a microorganism selected from the group consisting of microorganism from the genus Staphylococcus, Corynebacterium, Anaerococcus, Finegoldia, Moraxella, Porphyromonas, Fusobacterium, Aspergillus, Candida, Malassezia, Escherichia, Peptoniphilus, Streptococcus, Lactobacillus, Gardnerella, Fannyhessea, Epidermophyton, Trichophyton, Cutibacterium and combinations thereof, and/or for balancing sebum production or reducing sebum overproduction on skin or scalp and/or for suppressing dandruff on skin, scalp or nails and/or as antimicrobial component replacer in a cosmetic or pharmaceutical composition.

Finally, the present invention provides for personal hygiene products, a deodorant, an antiperspirant, an antiacne product, or an antidandruff product comprising a linear 1,2-alkanediol selected from the group consisting of 1,2-heptanediol, 1,2-nonanediol, 1,2-undecanediol, and 1,2-tridecanediol or comprising a linear 2,3-alkanediol selected from the group consisting of 2,3-pentanediol, 2,3-hexanediol, 2,3-heptanediol, 2,3-octanediol, 2,3-nonanediol, 2,3-decanediol, 2,3-undecanediol, 2,3-dodecanediol and 2,3-tridecanediol or comprising a mixture of a linear 1,2-alkanediol with a linear 2,3-alkanediol selected from the group consisting of
a mixture comprising 1,2-pentanediol and 2,3-pentanediol;
a mixture comprising 1,2-hexanediol and 2,3-hexanediol;
a mixture comprising 1,2-heptanediol and 2,3-heptanediol;
a mixture comprising 1,2-octanediol and 2,3-octanediol;
a mixture comprising 1,2-nonanediol and 2,3-nonanediol;
a mixture comprising 1,2-undecanediol and 2,3-undecanediol; and
a mixture comprising 1,2-tridecanediol and 2,3-tridecanediol.

### DESCRIPTION OF FIGURES

FIG. 1 is a diagram showing the inherent smell of different 1,2-alkanediols.

FIG. 2 is a diagram showing the suppressive leverage of different 1,2-alkanediols and 2,3-alkanediols on the sebum production of sebaceous glands.

FIG. 3 is a diagram showing the suppressive leverage of 1,2-nonanediol at different concentrations on the sebum production of sebaceous glands.

### DETAILED DESCRIPTION OF THE INVENTION

The present invention is specified in the appended claims. The invention itself, and its preferred variants, other objects and advantages, are however also apparent from the following detailed description in conjunction with the accompanying examples and figures.

In a first aspect, the present invention relates to a cosmetic or pharmaceutical composition, comprising or consisting of:
(a1) 1,2-nonanediol;
(b1) at least one antimicrobial component; and
(c1) optionally at least one cosmetically or pharmaceutically active substance and/or additive.

Alternatively, the present invention relates in a first aspect to a cosmetic or pharmaceutical composition, comprising or consisting of:
(a2) 2,3-nonanediol;
(b2) at least one antimicrobial component; and
(c2) optionally at least one cosmetically or pharmaceutically active substance and/or additive.

In a further alternative, the present invention relates in a first aspect to a cosmetic or pharmaceutical composition, comprising or consisting of:
(a3) a mixture comprising 1,2-nonanediol and 2,3-nonanediol;
(b3) at least one antimicrobial component; and
(c3) optionally at least one cosmetically or pharmaceutically active substance and/or additive.

In a second aspect, the present invention relates to a cosmetic or pharmaceutical composition, comprising or consisting of:
(a) at least one linear alkanediol having a carbon chain of 5 to 14 carbon atoms or a mixture comprising at least one first linear alkanediol having a carbon chain of 5 to 14 carbon atoms with one or more second linear alkanediols having a carbon chain of 5 to 14 carbon atoms which is different from the first linear alkanediol;
(b) at least one antimicrobial component; and
(c) optionally at least one cosmetically or pharmaceutically active substance and/or additive.

In a more preferred variant of the second aspect, the present invention relates to a cosmetic or pharmaceutical composition, comprising or consisting of:
(a′) a mixture comprising at least one first linear alkanediol having a carbon chain of 5 to 14 carbon atoms and one or more second linear alkanediols having a carbon chain of 5 to 14 carbon atoms, wherein the number of the carbon atoms of the first and the second alkanediol is either same or different;
(b′) at least one antimicrobial component; and
(c′) optionally at least one cosmetically or pharmaceutically active substance and/or additive.

In a preferred variant of the second aspect of the present invention, the component (b) is different from the component (a) or the component (b′) is different form the component (a′).

The term “comprising” means that the named components are essential, but other components may be added and is still embraced by the present invention.

The term “consisting of” as used according to the present invention means that the total amount of components (a) to (c) adds up to 100% by weight, based on the total weight of the cosmetic or pharmaceutical composition, and signifies that the subject matter is closed-ended and can only include the limitations that are expressly recited.

Whenever reference is made to “comprising” it is intended to cover both meanings as alternatives, that is the meaning can be either “comprising” or “consisting of” unless the context dictates otherwise.

The term “at least one . . . ” means that the cosmetic or pharmaceutical composition according to the present invention can comprise either one or a mixture of two, three, four, five, six or even more different of the respective components following the term.

The term “optionally” means that the subsequently described compound may but need not to be present in the composition, and that the description includes variants, where the compound is included or variants, where the compound is absent.

Alkanediols are glycols, i.e. any of a class of organic compounds belonging to the alcohol family; in the molecule of a glycol, two hydroxyl (—OH) groups are attached to different carbon atoms of a carbon chain.

The component (a) in the cosmetic or pharmaceutical composition according to the first aspect of the present invention is either 1,2-nonanediol (a1) or 2,3-nonanediol (a2), or a mixture of comprising both nonanediols, i.e. 1,2-nonanediol plus 2,3-nonanediol (a3).

1,2-nonanediol belongs to the category of alkanediols and is a straight chain alkanediol with the general formula:

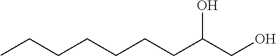

It has a carbon chain with 9 carbon atoms and the two functional OH groups of the alkanediol are in alpha,beta position and chemically bonded to the C1 and C2 carbon atoms in the alkanediol chain two OH groups of the alkanediol are chemically bonded to the C1 and C2 carbon atoms in the alkanediol chain.

2,3-nonanediol belongs to the category of alkanediols and is a straight chain alkanediol with the general formula:

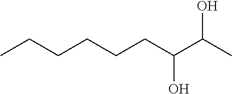

It has a carbon chain with 9 carbon atoms and the two functional OH groups of the alkanediol are in beta,gamma position and chemically bonded to the C2 and C3 carbon atoms in the alkanediol chain.

Straight chain 1,2-alkanediols have been used for more than 15 years as multifunctional actives. Short chain 1,2-alkanediols are amphiphilic compounds and thus, like 1,2-pentanediol and 1,2-hexanediol, are soluble both in water and cosmetic oils. In contrast, 1,2-octanediol is a solid and tends to precipitate or recrystallize in oily solutions. On the other hand, 1,2-decanediol is a solid and soluble only in cosmetic oils. Apart from moisturizing, some 1,2-alkanediols are used as viscosity modifiers.

The component (a) in the cosmetic or pharmaceutical composition according to the second aspect of the present invention is in a first alternative at least one linear alkanediol having a carbon chain of 5 to 14 carbon atoms.

Moreover, in a second alternative of this second aspect, the component (a) may include a first linear alkanediol in combination with a second linear alkanediol, preferably at least one first linear alkanediol having a carbon chain of 5 to 14 carbon atoms and at least one second linear alkanediol having a carbon chain of 5 to 14 carbon atoms. Therefore, the invention also entails a mixture of a first and a second alkanediol as described herein.

Since the first linear alkanediol of the invention can be selected from the same lists and types of compounds as the linear alkanediol according to the first alternative of the second aspect, in the following, reference is made to the linear alkanediol in general, which can also be the first linear alkanediol. In mixtures of first and second linear alkanediols, where these are specifically different, the specific terms “first” and “second” will be used to distinguish the two alkanediol components.

As used in this document, the phrase “at least one linear alkanediol” or “at least one first linear alkanediol” means that the composition can comprise one linear alkanediol having a carbon chain of 5 to 14 carbon atoms or can comprise one first linear alkanediol having a carbon chain of 5 to 14 carbon atoms or can comprise more than one linear alkanediol or can comprise more than one first linear alkanediol having a carbon chain of 5 to 14 carbon atoms, i.e. two, three, four or more different linear alkanediols or first linear alkanediols having a carbon chain of 5 to 14 carbon atoms.

The linear alkanediol or the first linear alkanediol consist of a chain of 5 to 14 carbon atoms joined to each other by single covalent bonds with two OH functional groups attached to two different carbon atoms in the chain.

The at least one linear alkanediol or the at least one first linear alkanediol having a carbon chain of 5 to 14 carbon atoms in the cosmetic or pharmaceutical composition according to the second aspect of the present invention is preferably selected from the group consisting of pentanediol, hexanediol, heptanediol, octanediol, nonanediol, decanediol, undecanediol, dodecanediol, tridecanediol and tetradecanediol.

In a preferred variant, the at least one linear alkanediol or the at least one first linear alkanediol is selected from the group consisting of pentanediol, hexanediol, heptanediol, octanediol, nonanediol, decanediol, undecanediol, dodecanediol and tridecanediol.

In a more preferred variant, the at least one linear alkanediol or the at least one first linear alkanediol is selected from the group consisting of alkanediols having an uneven number of carbon atoms of 5 to 13, i.e., pentanediol, heptanediol, nonanediol, undecanediol, tridecanediol, and mixtures thereof.

In a still more preferred variant, the at least one linear alkanediol or the at least one first linear alkanediol is selected from the group consisting of alkanediols having an uneven number of carbon atoms of 7, 9, 11 and 13, i.e. heptanediol, nonanediol, undecanediol and tridecanediol, or is selected from the group consisting of alkanediols having an uneven number of carbon atoms of 9 and 11, i.e. nonanediol and undecanediol, or is selected from the group consisting of alkanediols having an uneven number of carbon atoms of 9 and 13, i.e. nonanediol and tridecanediol, or is selected from the group consisting of alkanediols having an uneven number of carbon atoms of 11 and 13, i.e. undecanediol and tridecanediol.

In a still more preferred variant, the at least one linear alkanediol or the at least one first linear alkanediol is selected from the group consisting of nonanediol, undecanediol, tridecanediol.

In a particularly preferred variant, the at least one linear alkanediol or the at least one first linear alkanediol is either nonanediol or undecanediol.

In a most preferred variant, the at least one linear alkanediol or the at least first linear alkanediol is nonanediol.

In an alternative, the cosmetic or pharmaceutical composition according to the second aspect of the present invention comprises a mixture comprising at least one first linear alkanediol having a carbon chain of 5 to 14 carbon atoms with one or more second linear alkanediols having a carbon chain of 5 to 14 carbon atoms which is different from the first linear alkanediol.

This means that the alkanediol mixture is a mixture comprising at least one first linear alkanediol and one or more, i.e., two, three, four or more second linear alkanediols having a carbon chain of 5 to 14 carbon atoms. In this alternative, the first linear alkanediol can be any of the alkanediols as described herein for the linear alkanediol in general and in particular for the first alternative of the second aspect.

For example, the mixture can include one first linear alkanediol and one, two, three or more second linear alkanediols; or the mixture can include two first linear alkanediols with one, two, three or more second linear alkanediols, etc. with the proviso, that in each mixture, the first linear alkanediol and the second linear alkanediol are different from each other.

The phrase “different from each other means” that the first linear alkanediol and the second linear alkanediol in the mixture are either different with regard to the length of their carbon chain, i.e., number of the carbon atoms, and/or with regard to their constitutional isomerism or with regard to their stereoisomerism.

Likewise, the number of the carbon atoms of the first linear alkanediol and the second linear alkanediol in the mixture can also be same. For example, the first linear alkanediol and the second linear alkanediol have a carbon chain of 7 carbon atoms, but the first linear alkanediol and the second linear alkanediol are different with regard to their constitutional isomerism or with regard to their stereoisomerism.

The second linear alkanediol preferably consists of a chain of 5 to 14 carbon atoms joined to each other by single covalent bonds with two OH functional groups attached to two different carbon atoms in the chain.

The at least one second linear alkanediol having a carbon chain of 5 to 14 carbon atoms in the cosmetic or pharmaceutical composition according to the second aspect of the present invention is selected from the group consisting of pentanediol, hexanediol, heptanediol, octanediol, nonanediol, decanediol, undecanediol, dodecanediol, tridecanediol and tetradecanediol.

In a preferred variant, the at least one second linear alkanediol is selected from the group consisting of pentanediol, hexanediol, heptanediol, octanediol, nonanediol, decanediol, undecanediol, dodecanediol and tridecanediol.

If both the first and the second alkanediol is for example heptanediol, then the second linear alkanediol heptanediol is a different constitutional isomer or stereoisomer from the first linear alkanediol.

In a more preferred variant, the at least one second linear alkanediol is selected from the group consisting of pentanediol, hexanediol, heptanediol, octanediol, nonanediol and decanediol, most preferably octanediol and nonanediol.

The term “alkanediol” within the context of the present invention also includes its constitutional isomers or position isomers. Constitutional isomers are compounds that have the same molecular formula and different connectivity. Position isomers, a particular form of constitutional isomerism, are structural isomers that can be viewed as differing only on the position of a functional group on a parent structure, which in this case is the position of the two alcohol functions.

Depending on the number of the carbon atoms of the carbon chain of the alkanediol, there are various position isomers of the alkanediol: (x,x+1) constitutional isomers; (x,x+2) constitutional isomers; (x,x+3) constitutional isomers; etc. and alpha,omega constitutional isomers when the alcohol functions are at the terminal ends of the carbon chain, wherein x stands for the number of the carbon atom in the alkanediol chain, to which the OH groups of the alkanediol are chemically bonded. For example: if x is 1, the two OH groups of the alkanediol are chemically bonded to the C1 and C2 carbon atoms in the alkanediol chain; if x is 2, the two OH groups of the alkanediol are chemically bonded to the C2 and C3 carbon atoms in the alkanediol chain, etc.

In the (x,x+1) constitutional isomers, the two OH functional groups are vicinal attached to two different adjacent carbon atoms in the chain. In the (x,x+2) constitutional isomers, the two OH functional groups are attached to two different carbon atoms in the chain where the two carbon atoms are separated by one C atom.

In the (x,x+3) constitutional isomers, the two OH functional groups are attached to two different carbon atoms in the chain where the two carbon atoms are separated by two C atoms. In the alpha,omega constitutional isomers, the two functional groups are attached to the first C atom and to the terminal C atom.

In a preferred variant of the cosmetic or pharmaceutical composition according to the second aspect of the present invention, the linear alkanediol having a carbon chain of 5 to 14 carbon atoms, is preferably a vicinal (x,x+1) diol, selected from the group consisting of a 1,2-diol, 2,3-diol, 3,4-diol, 4,5-diol, further (x,x+1) diols, and mixtures thereof, preferably an alpha,beta 1,2 constitutional isomer.

In a preferred variant of the cosmetic or pharmaceutical composition according to the second aspect of the present invention, the first linear alkanediol and/or the second linear alkanediol having a carbon chain of 5 to 14 carbon atoms is a vicinal (x,x+1) diol, selected from the group consisting of a 1,2-diol, 2,3-diol, 3,4-diol, 4,5-diol, further (x,x+1) diols, and mixtures thereof, preferably an alpha,beta 1,2 constitutional isomer.

In a further preferred variant of the cosmetic or pharmaceutical composition according to the second aspect of the present invention, the linear alkanediol having a carbon chain of 5 to 14 carbon atoms, is preferably a non-vicinal (x,x+2) diol, selected from the group consisting of a 1,3-diol, 2,4-diol, 3,5-diol, further (x,x+2) diols, and mixtures thereof, preferably an alpha,gamma 1,3 constitutional isomer.

In a preferred variant of the cosmetic or pharmaceutical composition according to the second aspect of the present invention, the first linear alkanediol and/or the second linear alkanediol having a carbon chain of 5 to 14 carbon atoms is a non-vicinal (x,x+2) diol, selected from the group consisting of a 1,3-diol, 2,4-diol, 3,5-diol, 4,6-diol, further (x,x+2) diols, and mixtures thereof, preferably an alpha,gamma 1,3-constitutional isomer.

In a further preferred variant of the cosmetic or pharmaceutical composition according to the second aspect of the present invention, the linear alkanediol having a carbon chain of 5 to 14 carbon atoms, is preferably a non-vicinal (x,x+3) diol, selected from the group consisting of a 1,4 diol, 2,5 diol, further (x,x+3) diols, and mixtures thereof, preferably an alpha,delta 1,4 constitutional isomer.

In a preferred variant of the cosmetic or pharmaceutical composition according to the second aspect of the present invention, the first linear alkanediol and/or the second linear alkanediol having a carbon chain of 5 to 14 carbon atoms is a non-vicinal (x,x+3) diol, selected from the group consisting of a 1,4 diol, 2,5 diol, further (x,x+3) diols, or mixtures thereof, preferably an alpha,delta 1,4 constitutional isomer.

In a preferred variant of the cosmetic or pharmaceutical composition according to the second aspect of the present invention, the linear alkanediol or the first linear alkanediol and/or the second linear alkanediol is preferably an alpha,omega alkanediol, more preferably, 1,8-octanediol or 1,9-nonanediol.

According to the present invention, vicinal (x,x+1) diols are most preferred, such as alpha,beta or beta,gamma or gamma,delta etc.

In a still preferred variant of the cosmetic or pharmaceutical composition according to the second aspect of the present invention, the linear alkanediol and in particular the first linear alkanediol and/or the second linear alkanediol, is a 1,2-alkanediol, a 2,3-alkanediol, a 3,4-alkanediol, or mixtures thereof, more preferred 2,3-alkanediol.

The 1,2-alkanediols of the linear alkanediol, in particular the first linear alkanediol having a carbon chain of 5 to 14 carbon atoms and/or the second linear alkanediol having a carbon chain of 5 to 14 carbon atoms, can preferably be those as represented by the following formulae:

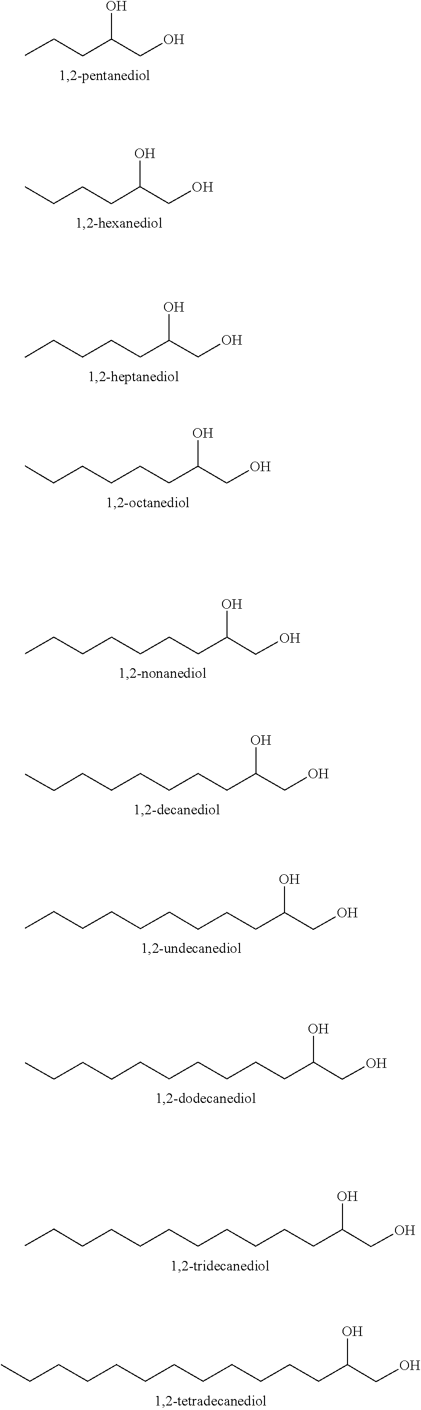

The 2,3-alkanediols of the linear alkanediol of the invention, in particular the first linear alkanediol having a carbon chain of 5 to 14 carbon atoms and/or the second linear alkanediol having a carbon chain of 5 to 14 carbon atoms, can preferably be those as represented by the following formulae:

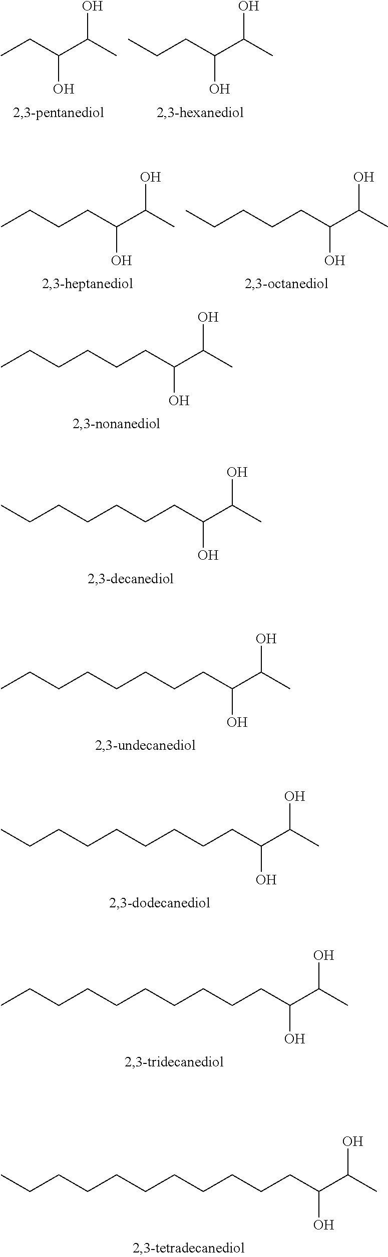

The 3,4-alkanediols of the linear alkanediol, preferably the first linear alkanediol having a carbon chain of 5 to 14 carbon atoms and/or the second linear alkanediol having a carbon chain of 5 to 14 carbon atoms, can preferably be those as represented by the following formulae:

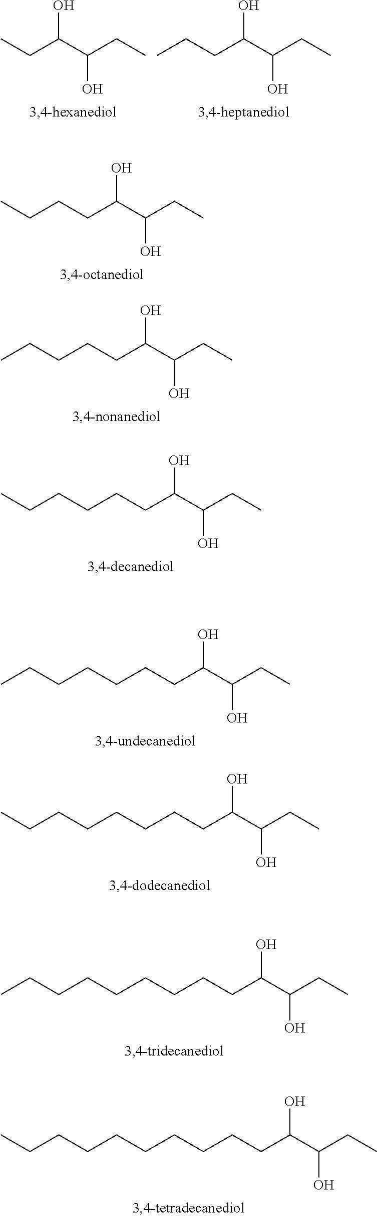

In a preferred variant, the cosmetic or pharmaceutical composition according to the second aspect of the present invention comprises a mixture comprising at least one first linear alkanediol having a carbon chain of 5 to 14 carbon atoms and one or more second linear alkanediols having a carbon chain of 5 to 14 carbon atoms wherein the number of the carbon atoms of the first and the second alkanediol is either same or different.

If both, the first and the second alkanediol have the same number of carbon atoms, such an alkanediol combination is herein also referred to as “homo alkanediol mixture” or “homo combination”. For example, the first linear alkanediol and the second linear alkanediol have a carbon chain of 7 carbon atoms, but the first linear alkanediol and the second linear alkanediol are different with regard to their constitutional isomerism or with regard to their stereoisomerism.

If the first and the second alkanediol have a different number of carbon atoms, such an alkanediol combination is herein also referred to as “hetero alkanediol mixture” or “hetero combination”. For example, the first linear alkanediol has a carbon chain of 7 carbon atoms and the second linear alkanediol has a carbon chain of 8 carbon atoms. However, beyond that, the first linear alkanediol and the second linear alkanediol can be different with regard to their constitutional isomerism or with regard to their stereoisomerism.

In a preferred variant of the cosmetic or pharmaceutical composition according to the second aspect of the present invention, the linear alkanediol or the first linear alkanediol is selected from the group consisting of:
1,2-pentanediol, 2,3-pentanediol, 3,4-pentanediol,
1,2-hexanediol, 2,3-hexanediol, 3,4-hexanediol,
1,2-heptanediol, 2,3-heptanediol, 3,4-heptanediol,
1,2-octanediol, 2,3-octanediol, 3,4-octanediol,
1,2-nonanediol, 2,3-nonanediol, 3,4-nonanediol,
1,2-decanediol, 2,3-decanediol, 3,4-decanediol,
1,2-undecanediol, 2,3-undecanediol, 3,4-undecanediol,
1,2-dodecanediol, 2,3-dodecanediol, 3,4-dodecanediol,
1,2-tridecanediol, 2,3-tridecanediol, 3,4-tridecanediol, and mixtures thereof.

Of the aforesaid linear alkanediols or the first linear alkanediols, the alpha, beta constitutional isomers are preferred: 1,2-pentanediol, 2,3-pentanediol, 1,2-hexanediol, 2,3-hexanediol, 1,2-heptanediol, 2,3-heptanediol, 2,3-octanediol, 2,3-nonanediol, 2,3-decanediol, 2,3-undecanediol, 2,3-dodecanediol, 2,3-tridecanediol, or mixtures thereof. Said alkanediols are liquid at a purity of 90 to 99%.

Of the aforesaid liquid alkanediols 1,2-pentanediol, 2,3-pentanediol, 1,2-hexanediol, 2,3-hexanediol, 1,2-heptanediol, 2,3-heptanediol, 2,3-octanediol, 2,3-nonanediol, 2,3-decanediol, 2,3-undecanediol or mixtures of said liquid alkanediols are particularly preferred. Said alkanediols can be easier incorporated into semi-finished products or final products.

In a more preferred variant, the at least one linear alkanediol or the at least one first linear alkanediol is selected from the group consisting of 1,2-alkanediols having an uneven number of carbon atoms of 5 to 13, i.e. 1,2-pentanediol, 1,2-heptanediol, 1,2-nonanediol, 1,2-undecanediol, 1,2-tridecanediol, and mixtures thereof.

In a more preferred variant, the linear alkanediol or the first linear alkanediol is selected from the group consisting of 1,2-alkanediols having an uneven number of carbon atoms of 7, 9, 11 and 13, i.e. 1,2-heptanediol, 1,2-nonanediol, 1,2-undecanediol or 1,2-tridecanediol, or is selected from the group consisting of 1,2-alkanediols having an uneven number of carbon atoms of 9 and 11, i.e. 1,2-nonanediol and 1,2-undecanediol, or is selected from the group consisting of 1,2-alkanediols having an uneven number of carbon atoms of 9 and 13, i.e. 1,2-nonanediol and 1,2-tridecanediol, or is selected from the group consisting of 1,2-alkanediols having an uneven number of carbon atoms of 11 and 13, i.e. 1,2-undecanediol and 1,2-tridecanediol.

In a more preferred variant, the linear alkanediol or the first linear alkanediol is selected from the group consisting of 1,2-alkanediols having an uneven number of carbon atoms of 9, 11 and 13, i.e., 1,2-nonanediol, 1,2-undecanediol or 1,2-tridecanediol.

In a more preferred variant, the at least one linear alkanediol or the at least one first linear alkanediol is selected from the group consisting of 2,3-alkanediols having an uneven number of carbon atoms of 5 to 13, i.e. 2,3-pentanediol, 2,3-heptanediol, 2,3-nonanediol, 2,3-undecanediol, 2,3-tridecanediol, and mixtures thereof.

In a still more preferred variant, the linear alkanediol or the first linear alkanediol is selected from the group consisting of 2,3-alkanediols having an uneven number of carbon atoms of 7, 9, 11 and 13, i.e. 2,3-heptanediol, 2,3-nonanediol, 2,3-undecanediol or 2,3-tridecanediol, or is selected from the group consisting of 2,3-alkanediols having an uneven number of carbon atoms of 9 and 11, i.e. 2,3-nonanediol and 2,3-undecanediol, or is selected from the group consisting of 2,3-alkanediols having an uneven number of carbon atoms of 9 and 13, i.e. 2,3-nonanediol and 2,3-tridecanediol, or is selected from the group consisting of 2,3-alkanediols having an uneven number of carbon atoms of 11 and 13, i.e. 2,3-undecanediol and 2,3-tridecanediol.

In a still more preferred variant, the linear alkanediol or the first linear alkanediol is selected from the group consisting of 2,3-alkanediols having an uneven number of carbon atoms of 9, 11 and 13, i.e., 2,3-nonanediol, 2,3-undecanediol or 2,3-tridecanediol.

In a further preferred variant of the cosmetic or pharmaceutical composition according to the second aspect of the present invention, the linear alkanediol or the first linear alkanediol is selected from the group consisting of:
1,2-nonanediol, 2,3-nonanediol, 3,4-nonanediol,
1,2-undecanediol, 2,3-undecanediol, 3,4-undecanediol,
1,2-tridecanediol, 2,3-tridecanediol, 3,4-tridecanediol,
1,2-octanediol, 2,3-octanediol, 3,4-octanediol, and mixtures thereof.

More preferred, in the cosmetic or pharmaceutical composition according to the second aspect of the present invention, the linear alkanediol or the first linear alkanediol is selected from the group consisting of 1,2-nonanediol, 2,3-nonanediol, 3,4-nonanediol, and mixtures thereof. However, the linear alkanediol or the first linear alkanediol can also preferably be selected from the group consisting of 1,2-octanediol, 2,3-octanediol, 3,4-octanediol, and mixtures thereof.

Even more preferred, in the cosmetic or pharmaceutical composition according to the second aspect of the present invention, the linear alkanediol or the first linear alkanediol is an alpha,beta or a beta,gamma diol as either 1,2-nonanediol, 2,3-nonanediol, or a mixture thereof. However, the linear alkanediol or the first linear alkanediol can also preferably be 1,2-octanediol, 2,3-octanediol, or a mixture thereof. A mixture of 1,2-nonanediol and 2,3-octanediol or a mixture of 1,2-octanediol and 2,3-nonanediol is also possible.

Most preferred, the linear alkanediol or the first linear alkanediol is 1,2-octanediol or 1,2-nonanediol.

Likewise, in a further preferred variant of the cosmetic or pharmaceutical composition according to the second aspect of the present invention, the linear alkanediol or the first linear alkanediol is selected from the group consisting of: 1,2-nonanediol, 2,3-nonanediol, 3,4-nonanediol, 1,2-undecanediol, 2,3-undecanediol, 3,4-undecanediol, 1,2-tridecanediol, 2,3-tridecanediol, 3,4-tridecanediol, and mixtures thereof.

More preferred, in the cosmetic or pharmaceutical composition according to the second aspect of the present invention, the linear alkanediol or the first linear alkanediol is selected from the group consisting of 1,2-nonanediol, 2,3-nonanediol, 3,4-nonanediol, and mixtures thereof, or can also be preferably selected from the group consisting of 1,2-undecanediol, 2,3-undecanediol, 3,4-undecanediol, and mixtures thereof or can also be preferably selected from the group consisting of 1,2-tridecanediol, 2,3-tridecanediol, 3,4-tridecanediol, and mixtures thereof.

Even more preferred, in the cosmetic or pharmaceutical composition according to the second aspect of the present invention, the linear alkanediol or the first linear alkanediol is an alpha,beta or a beta,gamma diol as either 1,2-nonanediol, 2,3-nonanediol, or a mixture thereof.

Additionally, the linear alkanediol or the first linear alkanediol can also preferably be 1,2-undecanediol, 2,3-undecanediol, or a mixture thereof. A mixture of 1,2-nonanediol and/or 2,3-undecanediol or a mixture of 2,3-nonanediol and/or 2,3-undecanediol or a mixture of 1,2-undecanediol and/or 2,3-nonanediol is also possible.

In a still more preferred variant, the at least one linear alkanediol or the at least one first linear alkanediol is selected from the group consisting of 1,2-nonanediol and 1,2-undecanediol. Both 1,2-alkanediols are characterized by a pronounced antimicrobial activity, have a distinguished sebum reducing effect and are odorless compared to the 1,2-decanediol, which has an unpleasant inherent smell.

In a most preferred variant, the at least one linear alkanediol or the at least one first linear alkanediol is 1,2-nonanediol.

Preferably, the at least one linear alkanediol is selected from the group consisting of 2,3-alkanediols such as 2,3-pentanediol, 2,3-hexanediol, 2,3-heptanediol, 2,3-octanediol, 2,3-nonanediol, 2,3-decanediol, 2,3-undecanediol, 2,3-dodecanediol and 2,3-tridecanediol. More preferred that at least one linear alkanediol is selected from the group consisting of 2,3-heptanediol, 2,3-nonanediol and 2,3-undecanediol.

The 2,3-alkanediols have the benefit that they have an antimicrobial efficacy on the one hand and an enhanced sebum reducing effect on the other hand compared to the respective 1,2-alkanediols. Additionally, the 2,3-alkanediols have the advantage that they are liquid, and, thus, can be easier incorporated into semi-finished products or final products. Hence, a good compromise can be achieved when the alkanediol is combined in such a way as to maintain the antimicrobial and sebum reducing effect, while improving processability in formulations.

In a preferred variant of the cosmetic or pharmaceutical composition according to the second aspect of the present invention, the second linear alkanediol is selected from the group consisting of:
1,2-pentanediol, 2,3-pentanediol, 3,4-pentanediol,
1,2-hexanediol, 2,3-hexanediol, 3,4-hexanediol,
1,2-heptanediol, 2,3-heptanediol, 3,4-heptanediol,
1,2-octanediol, 2,3-octanediol, 3,4-octanediol,
1,2-nonanediol, 2,3-nonanediol, 3,4-nonanediol,
1,2-decanediol, 2,3-decanediol, 3,4-decanediol,
1,2-undecanediol, 2,3-undecanediol, 3,4-undecanediol,
1,2-dodecanediol, 2,3-dodecanediol, 3,4-dodecanediol,
1,2-tridecanediol, 2,3-tridecanediol, 3,4-tridecanediol, and mixtures thereof.

Of the aforesaid second linear alkanediols, the alpha,beta constitutional isomers are particularly preferred: 1,2-pentanediol, 2,3-pentanediol, 1,2-hexanediol, 2,3-hexanediol, 1,2-heptanediol, 2,3-heptanediol, 2,3-octanediol, 2,3-nonanediol, 2,3-decanediol, 2,3-undecanediol, 2,3-dodecanediol, 2,3-tridecanediol, or mixtures thereof. Said alkanediols are liquid at a purity of 90 to 99%.

Of the aforesaid liquid alkanediols 1,2-pentanediol, 2,3-pentanediol, 1,2-hexanediol, 2,3-hexanediol, 1,2-heptanediol, 2,3-heptanediol, 2,3-octanediol, 2,3-nonanediol, 2,3-decanediol, 2,3-undecanediol or mixtures of said liquid alkanediols are particularly preferred. Said alkanediols can be easier incorporated into semi-finished products or final products.

In a more preferred variant, the second linear alkanediol is selected from the group consisting of 1,2-alkanediols having an uneven number of carbon atoms of 5 to 13, i.e. 1,2-pentanediol, 1,2-heptanediol, 1,2-nonanediol, 1,2-undecanediol, 1,2-tridecanediol, and mixtures thereof.

In a more preferred variant, the second linear alkanediol is selected from the group consisting of 1,2-alkanediols having an uneven number of carbon atoms of 7, 9, 11 and 13, i.e. 1,2-heptanediol, 1,2-nonanediol, 1,2-undecanediol and 1,2-tridecanediol, or is selected from the group consisting of 1,2-alkanediols having an uneven number of carbon atoms of 9 and 11, i.e. 1,2-nonanediol and 1,2-undecanediol, or is selected from the group consisting of 1,2-alkanediols having an uneven number of carbon atoms of 9 and 13, i.e. 1,2-nonanediol and 1,2-tridecanediol, or is selected from the group consisting of 1,2-alkanediols having an uneven number of carbon atoms of 11 and 13, i.e. 1,2-undecanediol and 1,2-tridecanediol.

In a more preferred variant, the second linear alkanediol is selected from the group consisting of 1,2-alkanediols having an uneven number of carbon atoms of 9, 11 and 13, i.e., 1,2-nonanediol, 1,2-undecanediol or 1,2-tridecanediol.

In a more preferred variant, the second linear alkanediol is selected from the group consisting of 2,3-alkanediols having an uneven number of carbon atoms of 5 to 13, i.e. 2,3-pentanediol, 2,3-heptanediol, 2,3-nonanediol, 2,3-undecanediol, 2,3-tridecanediol, and mixtures thereof.

In a still more preferred variant, the second linear alkanediol is selected from the group consisting of 2,3-alkanediols having an uneven number of carbon atoms of 7, 9, 11 and 13, i.e. 2,3-heptanediol, 2,3-nonanediol, 2,3-undecanediol and 2,3-tridecanediol, or is selected from the group consisting of 2,3-alkanediols having an uneven number of carbon atoms of 9 and 11, i.e. 2,3-nonanediol and 2,3-undecanediol, or is selected from the group consisting of 2,3-alkanediols having an uneven number of carbon atoms of 9 and 13, i.e. 2,3-nonanediol and 2,3-tridecanediol, or is selected from the group consisting of 2,3-alkanediols having an uneven number of carbon atoms of 11 and 13, i.e. 2,3-undecanediol and 2,3-tridecanediol.

In a still more preferred variant, the second linear alkanediol is selected from the group consisting of 2,3-alkanediols having an uneven number of carbon atoms of 9, 11 and 13, i.e., 2,3-nonanediol, 2,3-undecanediol or 2,3-tridecanediol.

In a more preferred variant of the cosmetic or pharmaceutical composition according to the second aspect of the present invention, the second linear alkanediol is selected from the group consisting of: 1,2-pentanediol, 2,3-pentanediol, 3,4-pentanediol, 1,2-hexanediol, 2,3-hexanediol, 3,4-hexanediol, and mixtures thereof.

More preferred, in the cosmetic or pharmaceutical composition according to the second aspect of the present invention, the second linear alkanediol is selected from the group consisting of 1,2-pentanediol, 2,3-pentanediol, 3,4-pentanediol, and mixtures thereof. However, the second linear alkanediol can also preferably be selected from the group consisting of 1,2-hexanediol, 2,3-hexanediol, 3,4-hexanediol, and mixtures thereof.

Even more preferred, in the cosmetic or pharmaceutical composition according to the second aspect of the present invention, the second linear alkanediol is an alpha,beta or a beta,gamma diol as either 1,2-pentanediol, 2,3-pentanediol or a mixture thereof. However, the second linear alkanediol can also preferably be 1,2-hexanediol or 2,3-hexanediol or a mixture thereof. A mixture of 1,2-pentanediol and 2,3-hexanediol or of 2,3-pentanediol and 1,2-hexanediol is also possible.

Most preferred, the second linear alkanediol is 1,2-pentanediol or 2,3-pentanediol or 1,2-hexanediol or 2,3-hexanediol or 1,2-heptanediol or 2,3-heptanediol or 2,3-octanediol or 2,3-nonanediol or 2,3-undecanediol.

In a particularly preferred variant, the cosmetic or pharmaceutical composition according to the second aspect of the present invention which comprises a mixture or a combination comprising at least one first linear alkanediol and one or more second linear alkanediol includes any one of the following mixtures/combinations:
a mixture comprising 1,2-pentanediol and 2,3-pentanediol;
a mixture comprising 1,2-hexanediol and 2,3-hexanediol;
a mixture comprising 1,2-heptanediol and 2,3-heptanediol;
a mixture comprising 1,2-octanediol and 2,3-octanediol;
a mixture comprising 1,2-nonanediol and 2,3-nonanediol;
a mixture comprising 1,2-decanediol and 2,3-decanediol;
a mixture comprising 1,2-undecanediol and 2,3-undecanediol;
a mixture comprising 1,2-dodecanediol and 2,3-dodecanediol; or
a mixture comprising 1,2-tridecanediol and 2,3-tridecanediol.

In said homo alkanediol mixtures the first linear alkanediol and the second alkanediol have the same number of carbon atoms.

Particularly favorable is a combination including 1,2-nonanediol in combination with 2,3-nonanediol and/or 3,4-nonanediol, or a combination including 1,2-undecanediol in combination with 2,3-undecanediol and/or 3,4-undecanediol, or a combination including 1,2-octanediol in combination with 2,3-octanediol and/or 3,4-octanediol.

Due to their outstanding antimicrobial and sebum reducing effect an alkanediol mixture comprising 1,2-nonanediol and 2,3-nonanediol or an alkanediol mixture comprising 1,2-undecanediol and 2,3-undecanediol are most preferred.

The above specified homo alkanediol mixtures according to the second aspect of the present invention, comprising a 1,2-alkanediol and a 2,3-alkanediol either having the same number of carbon atoms, are characterized by a synergistically antimicrobial efficacy, i.e. antimicrobial effect inhibiting microorganisms' growth when used in combination with an antimicrobial component, compared to their respective single 1,2-alkanediol or 2,3-alkanediol substances as it is demonstrated in the following examples. Additionally, the application of a mixture including a 1,2-alkanediol and 2,3-alkanediol has a favourable, i.e. suppressing, effect on the sebum production of sebaceous glands by suppressing, and, thus, reducing sebum production.

Said synergistic antimicrobial and sebum reducing effect or action is found for the homo mixture including 1,2-pentanediol and 2,3-pentanediol in combination with an antimicrobial component.

Said synergistic antimicrobial and sebum reducing effect is also observed for the homo mixture including 1,2-hexanediol and 2,3-hexanediol in combination with an antimicrobial component.

The homo mixture including 1,2-heptanediol and 2,3-heptanediol in combination with an antimicrobial component is particularly beneficial since it has a particular pronounced synergistic antimicrobial and sebum reducing effect.

The alkanediol mixture comprising 1,2-octanediol and 2,3-octanediol in combination with an antimicrobial component or an alkanediol mixture comprising 1,2-nonanediol and 2,3-nonanediol in combination with an antimicrobial component are particularly advantageous; both mixtures result in a low MIC value against the tested microorganisms and are effective in reducing sebum production.

Also, the alkanediol mixture comprising 1,2-decanediol and 2,3-decanediol or the alkanediol mixture comprising 1,2-undecanediol and 2,3-undecanediol synergistically reduces the microbial load when used in combination with different antimicrobials and reduces sebum production as well.

A synergistic effect is also true for an alkanediol mixture including 1,2-dodecanediol and 2,3-dodecanediol or an alkanediol mixture including 1,2-tridecanediol and 2,3-tridecanediol in combination with an antimicrobial component. The antimicrobial efficacy is found with SI values below 1. With said mixtures, also a sebum secretion can be reduced.

The afore specified homo alkanediol mixtures comprising a 1,2-alkanediol and the corresponding 2,3-alkanediol when used in combination with an antimicrobial component act synergistically against microorganisms of the natural microbiota of mammalian skin.

The afore-mentioned synergistically intensified antimicrobial effect is particularly pronounced for the homo mixture including 1,2-nonanediol and 2,3-nonanediol or the homo mixture including 1,2-undecanediol and 2,3-undecanediol, compared to the corresponding individual 1,2-alkanediol or 2,3-alkanediol substances while simultaneously reducing sebum production.

With the above-described antimicrobial efficacy and sebum reduction of the mixtures including a 1,2-alkanediol and the corresponding 2,3-alkanediol, microorganisms on the skin can be reduced, thus resulting in dandruff or acne reduction.

Additionally, with the admixture of the corresponding liquid 2,3-alkanediol to a solid 1,2-alkanediol, the solid 1,2-alkanediols, such as 1,2-octanediol, 1,2-nonanediol etc., can be solved, resulting in a liquid alkanediol mixture. The 2,3-alkanediol serves as solvent for the solid 1,2-alkanediols. Such liquid mixtures have the benefit that firstly the availability of the solid 1,2-alkanediol in the mixture is improved and secondly the incorporation of the solid 1,2-alkanediol in semi-finished products or final products is facilitated. This effect is particularly favorably for emulsions, in which lipophilic 1,2-alkanediols having a carbon chain of 8 or more carbon atoms, when used alone, tend to migrate into the oil phase. With the admixture of the corresponding liquid 2,3-alkanediol the 1,2-alkanediol remains in the water phase where usually microbial contamination takes place. This is in particular beneficial for the development of the antimicrobial effect of the 1,2-alkanediol in the water phase.

With the solution of the solid 1,2-nonanediol in the liquid 2,3-nonanediol or of the solid 1,2-undecanediol in the liquid 2,3-undecanediol, the availability of the strong antimicrobial 1,2-nonanediol or 1,2-undecanediol substance can be likewise improved in the end use.

In addition, the above alkanediol mixtures comprising a 1,2-alkanediol and the corresponding 2,3-alkanediol render possible the cold preparation of cosmetic or pharmaceutical formulations, i.e. no heating during preparation is necessary.

Thus, with the afore-mentioned properties of the specified alkanediol mixtures including a 1,2-alkanediol and the respective 2,3-alkanediol the processability in formulations can be improved.

Hence, a good compromise can be achieved when the active antimicrobial mixture is combined in such a way as to maintain the antimicrobial and sebum reducing effect, while improving processability in formulations. The above specified 1,2-alkanediol and corresponding 2,3-alkanediol combinations solve this balancing problem over the 1,2-alkanediol substances or 2,3-alkanediol substances alone. This is well achieved by combinations such as comprising 1,2-pentanediol and 2,3-pentanediol but also for 1,2-hexanediol and 2,3-hexanediol. Also combinations such as comprising 1,2-heptanediol and 2,3-heptanediol, but also 1,2-octanediol and 2,3-octanediol show this effect. The same also counts especially for 1,2-nonanediol in combination with 2,3-nonanediol, but also for 1,2-undecanediol in combination with 2,3-undecanediol.

The afore specified homo alkanediol mixtures including a 1,2-alkanediol and the corresponding 2,3-alkanediol in combination with an antimicrobial component display a remarkably synergistic activity and are clearly superior to the individually corresponding 1,2-alkanediols or 2,3-alkanediols and having the same concentration.

In a particularly preferred variant, the cosmetic or pharmaceutical composition according to the second aspect of the present invention which comprises a mixture or a combination comprising at least one first linear alkanediol and one or more second linear alkanediol thus includes any one of the following mixtures/combinations:
1,2-pentanediol in combination with one of 1,2-hexanediol, 1,2-heptanediol, 1,2-octanediol, 1,2-nonanediol, 1,2-decanediol, 1,2-undecanediol, 1,2-doedecanediol, or 1,2-tridecanediol;
1,2-hexanediol in combination with one of 1,2-pentanediol, 1,2-heptanediol, 1,2-octanediol, 1,2-nonanediol, 1,2-decanediol, 1,2-undecanediol, 1,2-doedecanediol, or 1,2-tridecanediol;
1,2-heptanediol in combination with one of 1,2-pentanediol, 1,2-hexanediol, 1,2-octanediol, 1,2-nonanediol, 1,2-decanediol, 1,2-undecanediol, 1,2-doedecanediol, or 1,2-tridecanediol;
1,2-octanediol in combination with one of 1,2-pentanediol, 1,2-hexanediol, 1,2-heptanediol, 1,2-nonanediol, 1,2-decanediol, 1,2-undecanediol, 1,2-doedecanediol, or 1,2-tridecanediol;
1,2-nonanediol in combination with one of 1,2-pentanediol, 1,2-hexanediol, 1,2-heptanediol, 1,2-octanediol, 1,2-decanediol, 1,2-undecanediol, 1,2-doedecanediol, or 1,2-tridecanediol;
1,2-decanediol in combination with one of 1,2-pentanediol, 1,2-hexanediol, 1,2-heptanediol, 1,2-octanediol, 1,2-nonanediol, 1,2-undecanediol, 1,2-doedecanediol, or 1,2-tridecanediol;
1,2-undecanediol in combination with one of 1,2-pentanediol, 1,2-hexanediol, 1,2-heptanediol, 1,2-octanediol, 1,2-nonanediol, 1,2-decanediol, 1,2-dodecanediol, or 1,2-tridecanediol;
1,2-dodecanediol in combination with one of 1,2-pentanediol, 1,2-hexanediol, 1,2-heptanediol, 1,2-octanediol, 1,2-nonanediol, 1,2-decanediol, 1,2-undecanediol, or 1,2-tridecanediol;
1,2-tridecanediol in combination with one of 1,2-pentanediol, 1,2-hexanediol, 1,2-heptanediol, 1,2-octanediol, 1,2-nonanediol, 1,2-decanediol, 1,2-undecanediol, or 1,2-doedecanediol;
2,3-pentanediol in combination with one of 1,2-hexanediol, 1,2-heptanediol, 1,2-octanediol, 1,2-nonanediol, 1,2-decanediol, 1,2-undecanediol, 1,2-doedecanediol, or 1,2-tridecanediol;
2,3-hexanediol in combination with one of 1,2-pentanediol, 1,2-heptanediol, 1,2-octanediol, 1,2-nonanediol, 1,2-decanediol, 1,2-undecanediol, 1,2-doedecanediol, or 1,2-tridecanediol;
2,3-heptanediol in combination with one of 1,2-pentanediol, 1,2-hexanediol, 1,2-octanediol, 1,2-nonanediol, 1,2-decanediol, 1,2-undecanediol, 1,2-doedecanediol, or 1,2-tridecanediol;
2,3-octanediol in combination with one of 1,2-pentanediol, 1,2-hexanediol, 1,2-heptanediol, 1,2-nonanediol, 1,2-decanediol, 1,2-undecanediol, 1,2-doedecanediol, or 1,2-tridecanediol;
2,3-nonanediol in combination with one of 1,2-pentanediol, 1,2-hexanediol, 1,2-heptanediol, 1,2-octanediol, 1,2-decanediol, 1,2-undecanediol, 1,2-doedecanediol, or 1,2-tridecanediol;
2,3-decanediol in combination with one of 1,2-pentanediol, 1,2-hexanediol, 1,2-heptanediol, 1,2-octanediol, 1,2-nonanediol, 1,2-undecanediol, 1,2-doedecanediol, or 1,2-tridecanediol;
2,3-undecanediol in combination with one of 1,2-pentanediol, 1,2-hexanediol, 1,2-heptanediol, 1,2-octanediol, 1,2-nonanediol, 1,2-decanediol, 1,2-dodecanediol, or 1,2-tridecanediol;
2,3-dodecanediol in combination with one of 1,2-pentanediol, 1,2-hexanediol, 1,2-heptanediol, 1,2-octanediol, 1,2-nonanediol, 1,2-decanediol, 1,2-undecanediol, or 1,2-tridecanediol; or
2,3-tridecanediol in combination with one of 1,2-pentanediol, 1,2-hexanediol, 1,2-heptanediol, 1,2-octanediol, 1,2-nonanediol, 1,2-decanediol, 1,2-undecanediol, or 1,2-dodecanediol;
2,3-pentanediol in combination with one of 2,3-hexanediol, 2,3-heptanediol, 2,3-octanediol, 2,3-nonanediol, 2,3-decanediol, 2,3-undecanediol, 2,3-doedecanediol, or 2,3-tridecanediol;
2,3-hexanediol in combination with one of 2,3-pentanediol, 2,3-heptanediol, 2,3-octanediol, 2,3-nonanediol, 2,3-decanediol, 2,3-undecanediol, 2,3-dodecanediol, or 2,3-tridecanediol;
2,3-heptanediol in combination with one of 2,3-pentanediol, 2,3-hexanediol, 2,3-octanediol, 2,3-nonanediol, 2,3-decanediol, 2,3-undecanediol, 2,3-dodecanediol, or 2,3-tridecanediol;
2,3-octanediol in combination with one of 2,3-pentanediol, 2,3-hexanediol, 2,3-heptanediol, 2,3-nonanediol, 2,3-decanediol, 2,3-undecanediol, 2,3-dodecanediol, or 2,3-tridecanediol;
2,3-nonanediol in combination with one of 2,3-pentanediol, 2,3-hexanediol, 2,3-heptanediol, 2,3-octanediol, 2,3-decanediol, 2,3-undecanediol, 2,3-dodecanediol, or 2,3-tridecanediol;
2,3-decanediol in combination with one of 2,3-pentanediol, 2,3-hexanediol, 2,3-heptanediol, 2,3-octanediol, 2,3-nonanediol, 2,3-undecanediol, 2,3-dodecanediol, or 2,3-tridecanediol;
2,3-undecanediol in combination with one of 2,3-pentanediol, 2,3-hexanediol, 2,3-heptanediol, 2,3-octanediol, 2,3-nonanediol, 2,3-decanediol, 2,3-dodecanediol, or 2,3-tridecanediol;
2,3-dodecanediol in combination with one of 2,3-pentanediol, 2,3-hexanediol, 2,3-heptanediol, 2,3-octanediol, 2,3-nonanediol, 2,3-decanediol, 2,3-undecanediol, or 2,3-tridecanediol; or
2,3-tridecanediol in combination with one of 2,3-pentanediol, 2,3-hexanediol, 2,3-heptanediol, 2,3-octanediol, 2,3-nonanediol, 2,3-decanediol, 2,3-undecanediol, or 2,3-dodecanediol.

In a particular preferred variant, the cosmetic or pharmaceutical composition according to the second aspect of the present invention which comprises a mixture or a combination comprising at least one first linear alkanediol and one or more second linear alkanediol thus may include any of the following mixtures/combinations:
1,2-heptanediol and 1,2-pentanediol; or
1,2-heptanediol and 1,2-hexanediol; or
1,2-heptanediol and 1,2-octanediol; or
1,2-heptanediol and 1,2-nonanediol; or
1,2-heptanediol and 1,2-decanediol; or
1,2-heptanediol and 1,2-undecanediol; or
1,2-heptanediol and 1,2-dodecanediol; or
1,2-heptanediol and 1,2-tridecanediol; or
1,2-nonanediol and 1,2-pentanediol; or
1,2-nonanediol and 1,2-hexanediol; or
1,2-nonanediol and 1,2-octanediol; or
1,2-nonanediol and 1,2-decanediol; or
1,2-nonanediol and 1,2-undecanediol; or
1,2-nonanediol and 1,2-dodecanediol; or
1,2-nonanediol and 1,2-tridecanediol; or
1,2-undecanediol and 1,2-pentanediol; or
1,2-undecanediol and 1,2-hexanediol; or
1,2-undecanediol and 1,2-octanediol; or
1,2-undecanediol and 1,2-nonanediol; or
1,2-undecanediol and 1,2-decanediol; or
1,2-undecanediol and 1,2-dodecanediol; or
1,2-undecanediol and 1,2-tridecanediol.

In a particular preferred variant, the cosmetic or pharmaceutical composition according to the second aspect of the present invention which comprises a mixture or a combination comprising at least one first linear alkanediol and one or more second linear alkanediol thus may include any of the following mixtures/combinations:
1,2-pentanediol and 2,3-pentanediol;
1,2-pentanediol and 2,3-hexanediol;
1,2-pentanediol and 2,3-heptanediol;
1,2-pentanediol and 2,3-octanediol;
1,2-pentanediol and 2,3-nonanediol;
1,2-pentanediol and 2,3-decanediol;
1,2-pentanediol and 2,3-undecanediol;
1,2-pentanediol and 2,3-dodecanediol;
1,2-pentanediol and 2,3-tridecanediol;
1,2-hexanediol and 2,3-pentanediol;
1,2-hexanediol and 2,3-hexanediol;
1,2-hexanediol and 2,3-heptanediol;
1,2-hexanediol and 2,3-octanediol;
1,2-hexanediol and 2,3-nonanediol;
1,2-hexanediol and 2,3-decanediol;
1,2-hexanediol and 2,3-undecanediol;
1,2-hexanediol and 2,3-dodecanediol;
1,2-hexanediol and 2,3-tridecanediol;
1,2-heptanediol and 2,3-pentanediol;
1,2-heptanediol and 2,3-hexanediol;
1,2-heptanediol and 2,3-heptanediol;
1,2-heptanediol and 2,3-octanediol;
1,2-heptanediol and 2,3-nonanediol;
1,2-heptanediol and 2,3-decanediol;
1,2-heptanediol and 2,3-undecanediol;
1,2-heptanediol and 2,3-dodecanediol;
1,2-heptanediol and 2,3-tridecanediol;
1,2-octanediol and 2,3-pentanediol;
1,2-octanediol and 2,3-hexanediol;
1,2-octanediol and 2,3-heptanediol;
1,2-octanediol and 2,3-octanediol;
1,2-octanediol and 2,3-nonanediol;
1,2-octanediol and 2,3-decanediol;
1,2-octanediol and 2,3-undecanediol;
1,2-octanediol and 2,3-dodecanediol;
1,2-octanediol and 2,3-tridecanediol;
1,2-nonanediol and 2,3-pentanediol;
1,2-nonanediol and 2,3-hexanediol;
1,2-nonanediol and 2,3-heptanediol;
1,2-nonanediol and 2,3-octanediol;
1,2-nonanediol and 2,3-nonanediol;
1,2-nonanediol and 2,3-decanediol;
1,2-nonanediol and 2,3-undecanediol;
1,2-nonanediol and 2,3-dodecanediol;
1,2-nonanediol and 2,3-tridecanediol;
1,2-decanediol and 2,3-pentanediol;
1,2-decanediol and 2,3-hexanediol;
1,2-decanediol and 2,3-heptanediol;
1,2-decanediol and 2,3-octanediol;
1,2-decanediol and 2,3-nonanediol;
1,2-decanediol and 2,3-decanediol;
1,2-decanediol and 2,3-undecanediol;
1,2-decanediol and 2,3-dodecanediol;
1,2-decanediol and 2,3-tridecanediol;
1,2-undecanediol and 2,3-pentanediol;
1,2-undecanediol and 2,3-hexanediol;
1,2-undecanediol and 2,3-heptanediol;
1,2-undecanediol and 2,3-octanediol;
1,2-undecanediol and 2,3-nonanediol;
1,2-undecanediol and 2,3-decanediol;
1,2-undecanediol and 2,3-undecanediol;
1,2-undecanediol and 2,3-dodecanediol;
1,2-undecanediol and 2,3-tridecanediol; or
1,2-dodecanediol and 2,3-pentanediol;
1,2-dodecanediol and 2,3-hexanediol;
1,2-dodecanediol and 2,3-heptanediol;
1,2-dodecanediol and 2,3-octanediol;
1,2-dodecanediol and 2,3-nonanediol;
1,2-dodecanediol and 2,3-decanediol;
1,2-dodecanediol and 2,3-undecanediol;
1,2-dodecanediol and 2,3-dodecanediol;
1,2-dodecanediol and 2,3-tridecanediol.

In a particular preferred variant, the cosmetic or pharmaceutical composition according to the second aspect of the present invention which comprises a mixture or a combination comprising at least one first linear alkanediol and one or more second linear alkanediol thus may include any of the following combinations:
1,2-heptanediol and 2,3-heptanediol; or
1,2-heptanediol and 2,3-pentanediol; or
1,2-heptanediol and 2,3-hexanediol; or
1,2-heptanediol and 2,3-nonanediol; or
1,2-heptanediol and 2,3-decanediol; or
1,2-heptanediol and 2,3-undecanediol; or
1,2-heptanediol and 2,3-dodecanediol; or
1,2-heptanediol and 2,3-tridecanediol; or
1,2-nonanediol and 2,3-pentanediol; or
1,2-nonanediol and 2,3-hexanediol; or
1,2-nonanediol and 2,3-octanediol; or
1,2-nonanediol and 2,3-decanediol; or
1,2-nonanediol and 2,3-undecanediol; or
1,2-nonanediol and 2,3-dodecanediol; or
1,2-nonanediol and 2,3-tridecanediol; or
1,2-undecanediol and 2,3-pentanediol; or
1,2-undecanediol and 2,3-hexanediol; or
1,2-undecanediol and 2,3-octanediol; or
1,2-undecanediol and 2,3-nonanediol; or
1,2-undecanediol and 2,3-decanediol; or
1,2-undecanediol and 2,3-dodecanediol; or
1,2-undecanediol and 2,3-tridecanediol.

In a particular preferred variant, the cosmetic or pharmaceutical composition according to the second aspect of the present invention which comprises a mixture or a combination comprising at least one first linear alkanediol and one or more second linear alkanediol thus may include any of the following mixtures/combinations:
2,3-pentanediol and 2,3-hexanediol;
2,3-pentanediol and 2,3-heptanediol;
2,3-pentanediol and 2,3-octanediol;
2,3-pentanediol and 2,3-nonanediol;
2,3-pentanediol and 2,3-decanediol;
2,3-pentanediol and 2,3-undecanediol;
2,3-pentanediol and 2,3-dodecanediol;
2,3-pentanediol and 2,3-tridecanediol;
2,3-hexanediol and 2,3-pentanediol;
2,3-hexanediol and 2,3-heptanediol;
2,3-hexanediol and 2,3-octanediol;
2,3-hexanediol and 2,3-nonanediol;
2,3-hexanediol and 2,3-decanediol;
2,3-hexanediol and 2,3-undecanediol;
2,3-hexanediol and 2,3-dodecanediol;
2,3-hexanediol and 2,3-tridecanediol;
2,3-heptanediol and 2,3-pentanediol;
2,3-heptanediol and 2,3-hexanediol;
2,3-heptanediol and 2,3-octanediol;
2,3-heptanediol and 2,3-nonanediol;
2,3-heptanediol and 2,3-decanediol;
2,3-heptanediol and 2,3-undecanediol;
2,3-heptanediol and 2,3-dodecanediol;
2,3-heptanediol and 2,3-tridecanediol;
2,3-octanediol and 2,3-pentanediol;
2,3-octanediol and 2,3-hexanediol;
2,3-octanediol and 2,3-heptanediol;
2,3-octanediol and 2,3-nonanediol;
2,3-octanediol and 2,3-decanediol;
2,3-octanediol and 2,3-undecanediol;
2,3-octanediol and 2,3-dodecanediol;
2,3-octanediol and 2,3-tridecanediol;
2,3-nonanediol and 2,3-pentanediol;
2,3-nonanediol and 2,3-hexanediol;
2,3-nonanediol and 2,3-heptanediol;
2,3-nonanediol and 2,3-octanediol;
2,3-nonanediol and 2,3-decanediol;
2,3-nonanediol and 2,3-undecanediol;
2,3-nonanediol and 2,3-dodecanediol;
2,3-nonanediol and 2,3-tridecanediol;
2,3-decanediol and 2,3-pentanediol;
2,3-decanediol and 2,3-hexanediol;
2,3-decanediol and 2,3-heptanediol;
2,3-decanediol and 2,3-octanediol;
2,3-decanediol and 2,3-nonanediol;
2,3-decanediol and 2,3-undecanediol;
2,3-decanediol and 2,3-dodecanediol;
2,3-decanediol and 2,3-tridecanediol;
2,3-undecanediol and 2,3-pentanediol;
2,3-undecanediol and 2,3-hexanediol;
2,3-undecanediol and 2,3-heptanediol;
2,3-undecanediol and 2,3-octanediol;
2,3-undecanediol and 2,3-nonanediol;
2,3-undecanediol and 2,3-decanediol;
2,3-undecanediol and 2,3-dodecanediol;
2,3-undecanediol and 2,3-tridecanediol; or
2,3-dodecanediol and 2,3-tridecanediol.

Particularly favorable is a mixture including 2,3-hexanediol and 2,3-heptanediol or 2,3-hexanediol and 2,3-octanediol or 2,3-heptanediol and 2,3-octanediol.

In the hetero alkanediol mixtures described before the first linear alkanediol and the second linear alkanediol have a different number of carbon atoms.

The above specified hetero alkanediol mixtures according to the second aspect of the present invention, comprising at least one first linear alkanediol and one or more second linear alkanediols when used in combination with an antimicrobial component are characterized by a synergistically antimicrobial efficacy, i.e. antimicrobial effect by inhibiting microorganisms' growth when used in combination with an antimicrobial component, compared to their respective single alkanediol substances as it is demonstrated in the following examples. Additionally, the application of said alkanediol mixtures has a favourable, i.e. suppressing, effect on the sebum production of sebaceous glands by suppressing, and, thus, reducing sebum production.

Of the above specified alkanediol mixtures particularly favourable is a hetero alkanediol mixture comprising 1,2-hexanediol and 2,3-octanediol or a hetero alkanediol mixture comprising 1,2-octanediol and 2,3-hexanediol or a hetero alkanediol mixture comprising 1,2-octanediol and 2,3-heptanediol showing a synergistic antimicrobial and sebum reducing effect compared to their corresponding single 1,2-alkanediol or 2,3-alkanediol substances, namely 1,2-hexanediol or 1,2-octanediol or 2,3-octanediol or 2,3-hexanediol or 2,3-heptanediol.

Said synergistic antimicrobial and sebum reducing effect or action is found for the hetero mixture including 1,2-hexanediol and 2,3-octanediol when used in combination with an antimicrobial substance.

Said synergistic antimicrobial and sebum reducing effect is also observed for the hetero mixture including 1,2-octanediol and 2,3-hexanediol in combination with an antimicrobial substance.

The hetero mixture including 1,2-octanediol and 2,3-heptanediol combined with an antimicrobial is particularly beneficial since it has a particular pronounced synergistic antimicrobial and sebum reducing effect.

Furthermore, a distinct antimicrobial effect and sebum reduction can also be observed for the following specified alkanediol mixtures, namely a mixture of alkanediols including 1,2-pentanediol and 2,3-hexanediol; or a mixture including 1,2-pentanediol and 1,2-heptanediol; or a mixture including 1,2-pentanediol and 2,3-heptanediol; or a mixture including 1,2-pentanediol and 2,3-octanediol; or a mixture including 1,2-pentanediol and 1,2-nonanediol; or a mixture including 1,2-pentanediol and 2,3-nonanediol when used in combination with an antimicrobial component.

The afore specified hetero alkanediol mixtures including at least one first linear alkanediol and one or more second alkanediols display a synergistic activity by inhibiting microorganisms' growth when used in combination with an antimicrobial component and suppressing sebum production of sebaceous glands.

The hetero mixture including 1,2-pentanediol and 2,3-hexanediol is particularly beneficial since it has a particular pronounced synergistic antimicrobial and sebum reducing effect when used in combination with an antimicrobial component.

The alkanediol mixture comprising 1,2-pentanediol and 1,2-heptanediol or an alkanediol mixture comprising 1,2-pentanediol and 2,3-heptanediol are particularly advantageous, since the mixtures result in a low MIC value against the tested microorganisms and are effective in reducing sebum production.

Likewise, a synergistic antimicrobial and sebum reducing effect or action is found for the hetero mixture including 1,2-pentanediol and 2,3-octanediol when used in combination with an antimicrobial component.

Also, the alkanediol mixture comprising 1,2-pentanediol and 1,2-nonanediol or the alkanediol mixture comprising 1,2-pentanediol and 2,3-nonanediol synergistically reduces the microbial load when used in combination with an antimicrobial and reduces sebum production as well.

Furthermore, a distinct antimicrobial effect and sebum reduction can also be observed for the following specified alkanediol mixtures when used in combination with an antimicrobial component, namely a mixture of alkanediols including 1,2-heptanediol and 1,2-octanediol or a mixture of alkanediols including 1,2-heptanediol and 2,3-octanediol or a mixture of alkanediols including 1,2-heptanediol and 1,2-nonanediol or a mixture or a combination of alkanediols including 1,2-heptanediol and 2,3-nonanediol.

The alkanediol mixture comprising 1,2-heptanediol and 1,2-octanediol or an alkanediol mixture comprising 1,2-heptanediol and 2,3-octanediol when used in combination with an antimicrobial component are particularly advantageous, since the mixtures result in a low MIC value against the tested microorganisms and are effective in reducing sebum production.

Also, the alkanediol mixture comprising 1,2-heptanediol and 1,2-nonanediol or the alkanediol mixture comprising 1,2-heptanediol and 2,3-nonanediol synergistically reduces the microbial load when used in combination with different antimicrobials and reduces sebum production as well.

The use of the alkanediol mixtures as specified before in combination with an antimicrobial component result in an enhanced, i.e. synergistically, antimicrobial effect by inhibiting microorganisms' growth when used with an antimicrobial component, compared to the use of the corresponding single alkanediol substances. Additionally, the application of these alkanediol mixtures has a favourable, i.e. suppressing, effect on the sebum production of sebaceous glands, and, thus, reducing sebum production.

With the above-described antimicrobial efficacy and sebum reduction of the mixtures including a first linear alkanediol and a second linear alkanediol, microorganisms on the skin can be reduced, thus resulting for example in dandruff or acne reduction.

In a preferred variant according to the second aspect of the present invention in the cosmetic or pharmaceutical composition the linear alkanediol is a 2,3-alkanediol. Preferably it is selected from the group consisting of 2,3-pentanediol, 2,3-hexanediol, 2,3-heptanediol, 2,3-octanediol, 2,3-nonanediol, 2,3-decanediol, 2,3-undecanediol, 2,3-dodecanediol, 2,3-tridecanediol, and mixtures thereof.

In a more preferred variant of the cosmetic or pharmaceutical composition according to the second aspect of the present invention, the linear alkanediol is selected from the group consisting of 1,2-alkanediols having an uneven number of carbon atoms of 7 and 9, i.e. 1,2-heptanediol and 1,2-undecanediol.

In a more preferred variant of the cosmetic or pharmaceutical composition according to the second aspect of the present invention, the linear alkanediol is selected from the group consisting of 2,3-alkanediols having an uneven number of carbon atoms of 7 to 11, i.e. 2,3-heptanediol, 2,3-nonanediol, 2,3-undecanediol or mixtures thereof.

In a still more preferred variant of the cosmetic or pharmaceutical composition according to the second aspect of the present invention, the linear alkanediol is selected from the group consisting of 2,3-nonanediol, 2,3-undecanediol, and mixtures thereof.

Surprisingly, the use of 2,3-pentanediol, 2,3-hexanediol, 2,3-heptanediol, 2,3-octanediol, 2,3-nonanediol, 2,3-decanediol, 2,3-undecanediol, 2,3-dodecanediol and 2,3-tridecanediol in combination with an antimicrobial component results in an enhanced, i.e. synergistically, antimicrobial effect by inhibiting microorganisms' growth on a mammal's skin or mucosa. Additionally, the application of 2,3-pentanediol, 2,3-hexanediol, 2,3-heptanediol, 2,3-octanediol, 2,3-nonanediol, 2,3-decanediol, 2,3-undecanediol, 2,3-dodecanediol or 2,3-tridecanediol shows a pronounced sebum reducing effect. This effect is even enhanced in comparison to the corresponding single 1,2-alkanediol substances. With the suppression of the sebum production of sebaceous glands microorganisms on the skin can be reduced, which indirectly supports e.g. dandruff or acne reduction. This sebum reducing effect is demonstrated in the following examples.

In addition, the 2,3-alkanediols as specified above have the benefit to be liquids, whereby their incorporation in semi-finished products or final products can be facilitated. Additionally, due to its better solubility, the 2,3-alkanediols can be furnished in a higher concentration in the final formulation.

Hence, a good compromise can be achieved when the active antimicrobial mixture is combined in such a way as to maintain the antimicrobial and sebum reducing effect, while improving processability in formulations. The above specified 2,3-alkanediols solve this balancing problem over the solid 1,2-alkanediol substances.

In a particularly advantageous variant according to the second aspect of the present invention, the linear alkanediol or the first alkanediol or the second alkanediol is a liquid alkanediol.

The term “liquid alkanediol” within the context of the present invention means an alkanediol component which is liquid at room or ambient temperature and under normal pressure, i.e., standard RTP conditions.

In a very preferred variant according to the second aspect of the present invention, the linear alkanediol or the first alkanediol or the second alkanediol is a liquid alkanediol selected from the group consisting of 1,2-pentanediol, 2,3-pentanediol, 1,2-hexanediol, 2,3-hexanediol, 1,2-heptanediol, 2,3-heptanediol, 2,3-octanediol, 2,3-nonanediol, 2,3-decanediol, 2,3-undecanediol, and mixtures thereof.

Of the aforesaid alkanediols, 1,2-pentanediol, 2,3-pentanediol, 1,2-hexanediol, 2,3-hexanediol, 1,2-heptanediol, 2,3-heptanediol, 2,3-octanediol, 2,3-nonanediol, or mixtures thereof are most preferred.

In a most particularly advantageous variant according to the second aspect of the present invention, the linear alkanediol or the first alkanediol or the second alkanediol are characterized by a maximum water solubility of less than or equal to 10% by weight. In a preferred variant, the maximum water solubility of the linear alkanediol or the first alkanediol or the second alkanediol is less than or equal to 10% by weight and more than or equal to 1.2% by weight. More preferred, the maximum water solubility of the linear alkanediol or the first linear alkanediol or the second linear alkanediol is less than or equal to 5% by weight and more than or equal to 1.4% by weight.

Preferably, the alkanediol with said water solubility is selected from the group consisting of 1,2-heptanediol, 2,3-heptanediol, 2,3-octanediol, and mixtures thereof.

The water solubility of a substance is the saturation mass concentration of the substance in water at a given temperature. The determination of the solubility in water relates to essentially pure substances which are stable in water and not volatile. The determination of the water solubility of the alkanediols is further described in the following Example 5.

The term “alkanediol” within the context of the present invention also includes its stereoisomers. Stereoisomers are molecules that have the same molecular formula and differ only in how their atoms are arranged in three-dimensional space. In accordance with said definition, the linear alkanediol of the invention and the first alkanediol having a carbon chain of 5 to 14 carbon atoms or the second linear alkanediol having a carbon chain of 5 to 14 carbon atoms as described above in detail encompass the following stereoisomers:

1,2-alkanediol stereoisomers:
(2S)-pentane-1,2-diol,
(2S)-hexane-1,2-diol,
(2S)-heptane-1,2-diol,
(2S)-octane-1,2-diol,
(2S)-nonane-1,2-diol,
(2S)-decane-1,2-diol,
(2S)-undecane-1,2-diol
(2S)-dodecane-1,2-diol,
(2S)-tridecane-1,2-diol,
(2S)-tetradecane-1,2-diol,
(2R)-pentane-1,2-diol,
(2R)-hexane-1,2-diol,
(2R)-heptane-1,2-diol,
(2R)-octane-1,2-diol,
(2R)-nonane-1,2-diol,
(2R)-decane-1,2-diol,
(2R)-undecane-1,2-diol,
(2R)-dodecane-1,2-diol,
(2R)-tridecane-1,2-diol,
(2R)-tetradecane-1,2-diol,

2,3-alkanediol stereoisomers:
(2S,3S)-pentane-2,3-diol,
(2S,3S)-hexane-2,3-diol,
(2S,3S)-heptane-2,3-diol,
(2S,3S)-octane-2,3-diol,
(2S,3S)-nonane-2,3-diol,
(2S,3S)-decane-2,3-diol,
(2S,3S)-undecane-2,3-diol
(2S,3S)-dodecane-2,3-diol,
(2S,3S)-tridecane-2,3-diol,
(2S,3S)-tetradecane-2,3-diol,
(2R,3R)-pentane-2,3-diol,
(2R,3R)-hexane-2,3-diol,
(2R,3R)-heptane-2,3-diol,
(2R,3R)-octane-2,3-diol,
(2R,3R)-nonane-2,3-diol,
(2R,3R)-decane-2,3-diol,
(2R,3R)-undecane-2,3-diol,
(2R,3R)-dodecane-2,3-diol,
(2R,3R)-tridecane-2,3-diol,
(2R,3R)-tetradecane-2,3-diol,
(2S,3R)-pentane-2,3-diol,
(2S,3R)-hexane-2,3-diol,
(2S,3R)-heptane-2,3-diol,
(2S,3R)-octane-2,3-diol,
(2S,3R)-nonane-2,3-diol,
(2S,3R)-decane-2,3-diol,
(2S,3R)-undecane-2,3-diol,
(2S,3R)-dodecane-2,3-diol,
(2S,3R)-tridecane-2,3-diol,
(2S,3R)-tetradecane-2,3-diol,
(2R,3S)-pentane-2,3-diol,
(2R,3S)-hexane-2,3-diol,
(2R,3S)-heptane-2,3-diol,
(2R,3S)-octane-2,3-diol,
(2R,3S)-nonane-2,3-diol,
(2R,3S)-decane-2,3-diol,
(2R,3S)-undecane-2,3-diol,
(2R,3S)-dodecane-2,3-diol,
(2R,3S)-tridecane-2,3-diol, and
(2R,3S)-tetradecane-2,3-diol.

3,4-alkanediol stereoisomers:
(3R,4R)-hexane-3,4-diol,
(3R,4R)-heptane-3,4-diol,
(3R,4R)-octane-3,4-diol,
(3R,4R)-nonane-3,4-diol,
(3R,4R)-decane-3,4-diol,
(3R,4R)-undecane-3,4-diol,
(3R,4R)-dodecane-3,4-diol,
(3R,4R)-tridecane-3,4-diol,
(3R,4R)-tetradecane-3,4-diol,
(3S,4S)-hexane-3,4-diol,
(3S,4S)-heptane-3,4-diol,
(3S,4S)-octane-3,4-diol,
(3S,4S)-nonane-3,4-diol,
(3S,4S)-decane-3,4-diol,
(3S,4S)-undecane-3,4-diol,
(3S,4S)-dodecane-3,4-diol,
(3S,4S)-tridecane-3,4-diol,
(3S,4S)-tetradecane-3,4-diol,
(3R,4S)-hexane-3,4-diol,
(3R,4S)-heptane-3,4-diol,
(3R,4S)-octane-3,4-diol,
(3R,4S)-nonane-3,4-diol,
(3R,4S)-decane-3,4-diol,
(3R,4S)-undecane-3,4-diol,
(3R,4S)-dodecane-3,4-diol,
(3R,4S)-tridecane-3,4-diol,
(3R,4S)-tetradecane-3,4-diol,
(3S,4R)-hexane-3,4-diol,
(3S,4R)-heptane-3,4-diol,
(3S,4R)-octane-3,4-diol,
(3S,4R)-nonane-3,4-diol,
(3S,4R)-decane-3,4-diol,
(3S,4R)-undecane-3,4-diol,
(3S,4R)-dodecane-3,4-diol,
(3S,4R)-tridecane-3,4-diol, and
(3S,4R)-tetradecane-3,4-diol.

In a preferred variant, the cosmetic or pharmaceutical composition according to the present invention does not contain 1,2-decanediol.

In the context of the present text, the terms “1,2-diol”, “2,3-diol” or “3,4-diol” includes both the corresponding S-configured enantiomers and also the R-enantiomer as well as arbitrary mixtures of these S- and R-configured enantiomers, i.e. mixtures of racemates of the respective diols.

The alkanediol mixture of the cosmetic or pharmaceutical composition according to the first aspect of the present invention comprises the 1,2-nonanediol and the 2,3-nonanediol in a ratio in a range of 50:50 to 99.9:0.1, preferably in a ratio in a range of 75:25 to 99:1, more preferred in a ratio in a range of 80:20 to 98:2 or still more preferred in a ratio in a range of 90:10 to 95:5.

In the cosmetic or pharmaceutical composition according to the first aspect of the present invention, 1,2-nonanediol and 2,3-nonanediol are comprised in the mixture preferably in a ratio in a range of 98:2 to 99.9:0.1.

Preferably, 1,2-nonanediol and 2,3-nonanediol are comprised in the mixture of the cosmetic or pharmaceutical composition according to the first aspect of the present invention in a ratio of ≥95:≤5, more preferred in a ratio of ≥96:≤4, still more preferred in a ratio of ≥97:≤3, and most preferred in a ratio of ≥98:≤2.

Most of all the mixture of the cosmetic or pharmaceutical composition according to the first aspect of the present invention comprises 1,2-nonanediol and 2,3-nonanediol in a ratio in a range of ≥95:≤5, including the ratios ≥95.5:≤4.5; ≥96:≤4; ≥96.5:≤3.5; ≥97:≤3; ≥97.5:2,5 and ≥98.0:≤2.0.

Even more preferred the alkanediol mixture of the cosmetic or pharmaceutical composition according to the first aspect of the present invention comprises 1,2-nonanediol and 2,3-nonanediol in a ratio in a range of ≥98:≤≤2, including the ratios of ≥98.1≤1.9; ≥98.2:≤1.8; ≥98.3:≤1.7; ≥98.4≤1.6; ≥98.5≤:1.5; ≥98.6≤1.4; ≥98.7≤1.3; ≥98.81:≤1.2; ≥98.9:≤1.1; ≥99:≤1.0; ≥99.1:≤0.9; ≥99.2:≤0.8; ≥99.3≤0.7; ≥99.4:≤0.6; ≥99.5:≤0.5; ≥99.6≤0.4; ≥99.7:≤0.3; ≥99.8:≤0.2 and ≥99.9:≤0.1.

The first linear alkanediol and the second linear alkanediol as defined in detail above are present in the alkanediol mixture according to the second aspect of the present invention in a ratio in a range of 50:50 to 99.9:0.1, preferably in a ratio in a range of 75:25 to 99:1, more preferred in a ratio in a range of 80:20 to 98:2, still more preferred in a ratio in a range of 90:10 to 95:5.

In the cosmetic or pharmaceutical preparation according to the second aspect of the present invention, the first linear alkanediol and the second linear alkanediol are comprised in the mixture preferably in a ratio in a range of 98:2 to 99.9:0.1

Preferably, the first linear alkanediol and the second alkanediol are comprised in the mixture of the cosmetic or pharmaceutical composition according to the second aspect of the present invention in a ratio in a range of ≥95:≤5, more preferred in a ratio of ≥96:≤4; still more preferred in a ratio of ≥97:≤3, and most preferred in a ratio of ≥98:≤2.

Most of all the mixture of the cosmetic or pharmaceutical composition according to the second aspect of the present invention comprises the first linear alkanediol and the second linear alkanediol in a ratio of ≥95:≤5, including the ratios ≥95.5:≤4.5; ≥96:≤4; ≥96.5:≤3.5; ≥97:≤3; ≥97.5:2,5 and ≥98.0:≤2.0.

Even more preferred the alkanediol mixture of the cosmetic or pharmaceutical composition according to the second aspect of the present invention comprises the first linear alkanediol and the second alkanediol in a ratio of ≥98:≤2, including the ratios of ≥98.1:≤1.9; ≥98.2:≤0.8; ≥98.3:≤1.7; ≥98.4≤1.6; ≥98.5:≤1.5; ≥98.6:≤1.4; ≥98.7:≤1.3; ≥98.8:≤1.2; ≥98.9≤1.1; ≥99:≤1.0; ≥99.1:≤0.9; ≥99.2: ≤0.8; ≥99.3≤0.7; ≥99.4:≤0.6; ≥99.5:≤0.5; ≥99.6:≤0.4; ≥99.7≤0.3; ≥99.8: ≤0.2 and ≥99.9:≤0.1.

Alternatively, said ratio ranges for the first and second alkanediol are switched, such that the second alkanediol is the main component and the first alkanediol is the secondary component.

In an advantageous variant according to the second aspect of the present invention, the mixture comprises as first linear alkanediol an 1,2-alkanediol, such as 1,2-pentanediol, 1,2-hexanediol, 1,2-heptanediol, 1,2-octanediol, 1,2-nonanediol, 1,2-decanediol, 1,2-undecanediol, 1,2-dodecanediol, or 1,2-tridecanediol and as second linear alkanediol the corresponding 2,3-alkanediols, such as 2,3-pentanediol, 2,3-hexanediol, 2,3-heptanediol, 2,3-octanediol, 2,3-nonanediol, 2,3-decanediol, 2,3-undecanediol, 2,3-dodecanediol, or 2,3-tridecanediol in a ratio in a range of 50:50 to 99.9:0.1, preferably in a ratio in a range of 75:25 to 99:1, more preferred in a ratio in a range of 80:20 to 98:2, still more preferred in a ratio in a range of 90:10 to 95:5.

Likewise, for said mixtures, the ratios of first linear alkanediol:second alkanediol or ranges of as described above are also applicable.

In a still further variant according to the second aspect of the present invention the mixture comprises as first linear alkanediol a 2,3-alkanediol, such as 2,3-pentanediol, 2,3-hexanediol, 2,3-heptanediol, 2,3-octanediol, 2,3-nonanediol, 2,3-decanediol, 2,3-undecanediol, 2,3-dodecanediol, or 2,3-tridecanediol and as second linear alkanediol the corresponding 1,2-alkanediols, such as 1,2-pentanediol, 1,2-hexanediol, 1,2-heptanediol, 1,2-octanediol, 1,2-nonanediol, 1,2-decanediol, 1,2-undecanediol, 1,2-dodecanediol, or 1,2-tridecanediol in a ratio in a range of 50:50 to 99.9:0.1, preferably in a ratio in a range of 75:25 to 99:1, more preferred in a ratio in a range of 80:20 to 98:2, still more preferred in a ratio in a range of 90:10 to 95:5.

Likewise, for said mixtures, the ratios of first linear alkanediol:second alkanediol or ranges of ratios as described above are also applicable.

In a more advantageous variant according to the second aspect of the present invention, in the mixtures comprising a combination of a 1,2-alkanediol as first linear alkanediol and the corresponding 2,3-alkanediol as second linear alkanediol, such as
1,2-pentanediol and 2,3-pentanediol, or
1,2-hexanediol and 2,3-hexanediol, or
1,2-heptanediol and 2,3-heptanediol, or
1,2-octanediol and 2,3-octanediol, or
1,2-nonanediol and 2,3-nonanediol, or
1,2-decanediol and 2,2-decanediol, or
1,2-undecanediol and 2,3-undecenaediol, or
1,2-dodecanediol and 2,3-dodecanediol, or
1,2-tridecanediol and 2,3-tridecanediol,
the first linear alkanediol and the second linear alkanediol are present in a ratio in a range of 50:50 to 99.9:0.1, preferably in a ratio in a range of 75:25 to 99:1, more preferred in a ratio in a range of 80:20 to 98:2, still more preferred in ratio in a range of 90:10 to 95:5.

Likewise, for said mixtures, the ratios of first linear alkanediol:second alkanediol or ranges of ratios as described above are also applicable.

By these mixing ratios the afore mentioned mixtures show a synergistically antimicrobial and sebum reducing effect compared to the corresponding individual substances as it is described and demonstrated in the following experimental part.

In a more advantageous variant according to the second aspect of the present invention, in the mixtures comprising as first linear alkanediol a 2,3-alkanediol and as second linear alkanediol the corresponding 1,2-alkanediols, such as mixtures including
2,3-pentanediol and 1,2-pentanediol, or
2,3-hexanediol and 1,2-hexanediol, or
2,3-heptanediol and 1,2-heptanediol, or
2,3-octanediol and 1,2-octanediol, or
2,3-nonanediol and 1,2-nonanediol, or
2,3-decanediol and 1,2-decanediol, or
2,3-undecanediol and 1,2-undecenaediol, or
2,3-dodecanediol and 1,2-dodecanediol, or
2,3-tridecanediol and 1,2-tridecanediol,
the first linear alkanediol and the second linear alkanediol are present in a ratio in a range of 50:50 to 99.9:0.1, preferably in a ratio in a range of 75:25 to 99:1, more preferred in a ratio in a range of 80:20 to 98:2 or still more preferred in a ratio in a range of 90:10 to 95:5.

Likewise, for said mixtures, the ratios of first linear alkanediol:second alkanediol or ranges of ratios as described above are also applicable.

By these mixing ratios the afore mentioned mixtures show a synergistically antimicrobial and sebum reducing effect compared to the corresponding individual substances as it is described and demonstrated in the following experimental part.

In a most preferred variant according to the second aspect of the present invention, the mixture comprises as first linear alkanediol 1,2-nonanediol or 1,2-undecanediol or 1,2-tridecanediol and as second linear alkanediol 2,3-nonanediol or 2,3-undecanediol or 2,3-tridecanediol in a ratio in a range of 50:50 to 99.9:0.1, preferably in a ratio in a range of 75:25 to 99:1, more preferred in a ratio in a range of 80:20 to 98:2, still more preferred in a ratio in a range of 90:10 to 95:5.

Likewise, for said mixtures, the ratios of first linear alkanediol:second alkanediol or ranges of ratios as described above are also applicable.

In a very most preferred variant according to the second aspect of the present invention, the mixture comprises as first linear alkanediol 2,3-nonanediol or 2,3-undecanediol or 2,3-tridecanediol and as second linear alkanediol 1,2-nonanediol or 1,2-undecanediol or 1,2-tridecanediol in a ratio in a range of 50:50 to 99.9:0.1, preferably in a ratio in a range of 75:25 to 99:1, more preferred in a ratio in a range of 80:20 to 98:2, still more preferred in a ratio in a range of 90:10 to 95:5.

Likewise, for said mixtures, the ratios of first linear alkanediol:second alkanediol or ranges of ratios as described above are also applicable.

A preferred variant according to the second aspect the present invention also encompasses a mixture including as first linear alkanediol 1,2-hexanediol and as second linear alkanediol 2,3-octanediol or a mixture including as first linear alkanediol 1,2-octanediol and as second linear alkanediol 2,3-hexanediol or a mixture including as first linear alkanediol 1,2-octanediol and and as second linear alkanediol 2,3-heptanediol either in a ratio in a range of 50:50 to 99.9:0.1, preferably in a ratio in a range of 75:25 to 99:1, more preferred in a ratio in a range of 80:20 to 98:2 or still more preferred in a ratio in a range of 90:10 to 95:5.

Likewise, for said mixtures, the ratios of first linear alkanediol:second alkanediol or ranges of ratios as described above are also applicable.

By these mixing ratios the afore mentioned mixtures show a synergistically antimicrobial and sebum reducing effect compared to the corresponding individual substances as it is described and demonstrated in the following experimental part.

In a further beneficial variant according to the second aspect of the present invention, the mixtures comprise a first linear alkanediol and a second linear alkanediol, such as mixtures including
1,2-pentanediol and 2,3-hexanediol; or
1,2-pentanediol and 1,2-heptanediol; or
1,2-pentanediol and 2,3-heptanediol; or
1,2-pentanediol and 2,3-octanediol; or
1,2-pentanediol and 1,2-nonanediol; or
1,2-pentanediol and 2,3-nonanediol,
wherein the first linear alkanediol and the second linear alkanediol are present in a ratio in a range of 50:50 to 99.9:0.1, preferably in a ratio in a range of 75:25 to 99:1, more preferred in a ratio in a range of 80:20 to 98:2 or still more preferred in a ratio in a range of 90:10 to 95:5.

Likewise, for said mixtures, the ratios of first linear alkanediol:second alkanediol or ranges of ratios as described above are also applicable.

By these mixing ratios the afore mentioned mixtures show a synergistically antimicrobial and sebum reducing effect compared to the corresponding individual substances as it is described and demonstrated in the following experimental part.

A particularly preferred variant according to the second aspect the present invention also encompasses a mixture including as first linear alkanediol 1,2-heptanediol and as second linear alkanediol 1,2-octanediol, or a mixture including as first Hear alkanediol 1,2-heptanediol and as second linear alkanediol 2,3-octanediol or a mixture including as first linear alkanediol 1,2-heptanediol and as second linear alkanediol 1,2-nonanediol or a mixture including as first linear alkanediol 1,2-heptanediol and as second linear alkanediol 2,3-nonanediol wherein the first linear alkanediol and the second linear alkanediol are present in a ratio in a range of 50:50 to 99.9:0.1, preferably in a ratio in a range of 75:25 to 99:1, more preferred in a ratio in a range of 80:20 to 98:2 or still more preferred in a ratio in a range of 90:10 to 95:5.

Likewise, for said mixtures, the ratios of first linear alkanediol:second alkanediol or ranges of ratios as described above are also applicable.

By these mixing ratios the afore mentioned mixtures show a synergistically antimicrobial and sebum reducing effect compared to the corresponding individual substances as it is described and demonstrated in the following experimental part.

The alkanediols are obtained either by synthesis from petrochemical or other fossil fuel sources by known methods such as olefin bishydroxylation, hydrolysis from epoxide or various chemical transformations or from bioderived feedstock by fermentation or from bio-based natural and renewable feedstock such as biomass by catalytic synthesis as it is described in US 2019/0241491 A1 and US 2020/0189995 A1. The alkanediols used according to the present invention comprise either petrochemically derived and biobased natural and renewable feedstock derived alkanediols. Preferably, the alkanediols are from bio-based sources and are thus bio-alkanediols.

The component (b) of the cosmetic or pharmaceutical composition according to the first or second aspect of the present invention relates to an antimicrobial component, which is used in many cosmetic compositions, in particular skincare, haircare or body care, or for pharmaceutical compositions.

The term “antimicrobial” or “antimicrobial component” is a general term that refers to a group of agents or substances that kills microorganisms or stops their growth. Antimicrobial agents or substances can be grouped according to the microorganisms they act primarily against. For example, antibiotics are used against bacteria, antifungals are used against fungi, antiprotozoals are used against protozoans, and antivirals are used against virus. They can also be classified according to their function. Agents that kill microbes are classified as biocidal or microbicidal, while those that merely inhibit their growth are called biostatic.

The antimicrobial component according to the present invention act primarily against microorganisms, in particular bacteria and/or fungi, of the natural microbiota of mammalian skin or mucosa. Preferably, the microorganisms are selected from the group consisting of but are not limited to microorganisms from the genus Staphylococcus, Corynebacterium, Anaerococcus, Finegoldia, Moraxella, Porphyromonas, Fusobacterium, Aspergillus, Candida, Malassezia, Escherichia, Peptoniphilus, Streptococcus, Lactobacillus, Gardnerella, Fannyhessea, Epidermophyton, Trichophyton, Cutibacterium, and combinations thereof.

More preferably, the microorganisms are selected from the group consisting of Staphylococcus epidermidis, Staphylococcus hominis, Corynebacterium xerosis, Corynebacterium jeikeium, Anaerococcus Octavius, Finegoldia magna, Moraxella osloensis, Moraxella atlantae, Porphyromonas gingivalis, Aspergillus brasiliensis, Candida albicans, Escherichia coli, Staphylococcus aureus, Peptoniphilus lacrimalis, Streptococcus agalactiae, Lactobacillus acidophilus, Gardnerella vaginalis, Fannyhessea vaginae (Atopobium vaginae), Epidermophyton floccosum, Trichophyton rubrum, Streptococcus mutans, Fusobacterium nucleatum, and combinations thereof.

The cosmetic or pharmaceutical composition according to the first or second aspect of the present invention encompasses at least one antimicrobial component selected from the group consisting of Phenoxyethanol, Farnesol, Bisabolol, Ethylhexylglycerin, Triethyl Citrate, Hydroxyacetophenone, Benzyl Alcohol, 2-Methyl 5-cyclohexylpentanol, Benzoic Acid, Sorbic Acid, Dehydroacetic Acid, Sorbitan Caprylate, Caprylhydroxamic Acid, Dimethyl phenylbutanol, Dimethyl phenylpropanol, Levulinic Acid and esters and/or ketals thereof, Anisic Acid, 2-Benzylheptanol, Polyglyceryl-3 Caprylate, Polyglyceryl-10 Laurate, Glyceryl Caprate, Polyglyceryl-2 Caprate, Zinc Ricinoleate, Glyceryl Laurate, Glyceryl Caprylate, Xylityl Sesquicaprylate, Phenylpropanol, Lauryl Alcohol, o-Cymen-5-ol, Octenidine HCl, Undecylenoyl Glycine, Sodium Caproyl/Lauroyl Lactylate, Ethyl Lauroyl Arginate Laurate, PCA Ethyl Cocoyl Arginate, Salvia Officinalis (Sage) Oil, Tropolone, Oiroctone Olamine, Triclosan, Triclocarban, Chloroxylenol, Chlorocresol, Benzalkonium Chloride, Benzalkonium Bromide, Benzalkonium Saccharinate, Benzethonium Chloride, Chlorphenesin, Alkyl (C12-22) Trimethyl Ammonium Bromide and Chloride, Zinc Pyrithione, Climbazole, Chlorhexidine, Chlorhexidine Diacetate, Chlorhexidine Digluconate, Chlorhexidine Dihydrochloride, Hexamidine, Hexamidine Diisethionate, Silver Chloride, Silver Citrate, Hexylglycerin, Cyclohexylglycerin, Glycerin, Tetraselmis suecica extract, Mannitol, Sorbitol, Lactic Acid, Sodium Lauroyl Lactylate, Sodium Caproyl Lactylate, Salicylic Acid and its salts, Capryloyl Glycine, Ethyl Lauroyl Arginate HCl, Thymol, Jasmol, Zinc Citrate, Cetylpyridinium Chloride, Menthol, Eucalyptol, Methyl Salicylate, Ethanol, Camphor, Polyarginine, Polylysine, Gluconic Acid and salts thereof, Ascorbic Acid and salts thereof, Benzyl Salicylate, Disodium EDTA, Methylparaben and its salts, Sodium Benzoate, Potassium Sorbate, Propylparaben, Methylchloroisothiazolinone, Methylisothiazolinone, Butylparaben, Ethylparaben, Sodium Propylparaben, DMDM Hydantoin, Hydroxyethoxyphenyl Butanone, Hydroxyethoxyphenyl Butanol, Itaconic Acid and salts thereof, Propanediol Caprylate, 4-Hydroxyacetophenone, Iodopropynyl Butylcarbamate, Isobutylparaben, Sodium Ethylparaben, Diazolidinyl Urea, Glyceryl Caprylate, Sodium Phytate or Phytic acid, Sodium Levulinate or Levulinic Acid and esters and/or ketals thereof, TEA-Salicylate, Phenethyl Alcohol, Tetrasodium Glutamate Diacetate, Trisodium Ethylenediamine Disuccinate, Sodium Lactate, 4-Hydroxybenzoic acid and its salts and esters, thyme oil, eugenol, oil of cloves, mint oil, DMC, antibacterial fragrances, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more the aforesaid antimicrobial components.

In a preferred variant, the cosmetic or pharmaceutical composition according to the first or second aspect of the present invention encompasses at least one antimicrobial component selected from the group consisting of Phenoxyethanol, Farnesol, Ethylhexylglycerin, Triethyl Citrate, Hydroxyacetophenone, Benzyl Alcohol, 2-Methyl 5-cyclohexylpentanol, Benzoic Acid, Sorbic Acid, Dehydroacetic Acid, Sorbitan Caprylate, Caprylhydroxamic Acid, Dimethyl phenylbutanol, Dimethyl phenylpropanol, Levulinic Acid and esters and/or ketals thereof, Anisic Acid, 2-Benzylheptanol, Polyglyceryl-3 Caprylate, Polyglyceryl-10 Laurate, Glyceryl Caprate, Polyglyceryl-2 Caprate, Zinc Ricinoleate, Glyceryl Laurate, Glyceryl Caprylate, Xylityl Sesquicaprylate, Phenylpropanol, Lauryl Alcohol, o-Cymen-5-ol, Octenidine HCl, Undecylenoyl Glycine, Sodium Caproyl/Lauroyl Lactylate, Ethyl Lauroyl Arginate Laurate, PCA Ethyl Cocoyl Arginate, Salvia Officinalis (Sage) Oil, Tropolone, Oiroctone Olamine, Triclosan, Triclocarban, Chloroxylenol, Chlorocresol, Benzalkonium Chloride, Benzalkonium Bromide, Benzalkonium Saccharinate, Benzethonium Chloride, Chlorphenesin, Alkyl (C12-22) Trimethyl Ammonium Bromide and Chloride, Zinc Pyrithione, Climbazole, Chlorhexidine, Chlorhexidine Diacetate, Chlorhexidine Digluconate, Chlorhexidine Dihydrochloride, Hexamidine, Hexamidine Diisethionate, Silver Chloride, Silver Citrate, Hexylglycerin, Cyclohexylglycerin, Glycerin, Tetraselmis suecica extract, Mannitol, Sorbitol, Sodium Lauroyl Lactylate, Sodium Caproyl Lactylate, Salicylic Acid and its salts, Capryloyl Glycine, Ethyl Lauroyl Arginate HCl, Thymol, Jasmol, Zinc Citrate, Cetylpyridinium Chloride, Menthol, Eucalyptol, Methyl Salicylate, Ethanol, Camphor, Polyarginine, Polylysine, Gluconic Acid and salts thereof, Ascorbic Acid and salts thereof, Benzyl Salicylate, Disodium EDTA, Methylparaben and its salts, Sodium Benzoate, Potassium Sorbate, Propylparaben, Methylchloroisothiazolinone, Methylisothiazolinone, Butylparaben, Ethylparaben, Sodium Propylparaben, DMDM Hydantoin, Hydroxyethoxyphenyl Butanone, Hydroxyethoxyphenyl Butanol, Itaconic Acid and salts thereof, Propanediol Caprylate, 4-Hydroxyacetophenone, Iodopropynyl Butylcarbamate, Isobutylparaben, Sodium Ethylparaben, Diazolidinyl Urea, Glyceryl Caprylate, Sodium Phytate or Phytic acid, Sodium Levulinate or Levulinic Acid and esters and/or ketals thereof, TEA-Salicylate, Phenethyl Alcohol, Tetrasodium Glutamate Diacetate, Trisodium Ethylenediamine Disuccinate, Sodium Lactate, 4-Hydroxybenzoic acid and its salts and esters, thyme oil, eugenol, oil of cloves, mint oil, DMC, antibacterial fragrances, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more the aforesaid antimicrobial components.

In a preferred variant, the antimicrobial component of the cosmetic or pharmaceutical composition of the present invention is selected from the group consisting of Phenoxyethanol, 2-Methyl 5-cyclohexylpentanol, Dimethyl phenylbutanol, Dimethyl Phenylpropanol, 2-Benzylheptanol, Ethylhexylglycerin, Hydroxyacetophenone, Triethyl Citrate, Polyglyceryl-3 Caprylate, Polyglyceryl-10 Laurate, Glyceryl Caprylate, Benzyl Alcohol, Benzoic Acid, Sorbic Acid, Levulinic Acid and esters and/or ketals thereof, Anisic Acid and salts thereof, Polyglyceryl-2 Caprate, Caprylhydroxamic Acid, Zinc Ricinoleate, Farnesol, Bisabolol, Glyceryl Laurate, Glyceryl Caprylate, Xylityl Sesquicaprylate, Phenylpropanol, Lauryl Alcohol, o-Cymen-5-ol, Tropolone, Climbazole, Piroctone Olamine, Octenidine HCl, Undecylenoyl Glycine, Sodium Caproyl/Lauroyl Lactylate, Ethyl Lauroyl Arginate Laurate, PCA Ethyl Cocoyl Arginate, Salvia Officinalis (Sage) Oil, Triclosan, Tetraselmis suecica extract, Mannitol, Sorbitol, Lactic Acid, Sodium Lauroyl Lactylate, Sodium Caproyl Lactylate, Salicylic Acid and its salts, Capryloyl Glycine, Ethyl Lauroyl Arginate HCl, Thymol, Jasmol, Gluconic Acid and salts thereof, Benzyl Salicylate, Propanediol Caprylate, 4-Hydroxyacetophenone, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more the aforesaid antimicrobial components.

In a preferred variant, the antimicrobial component of the cosmetic or pharmaceutical composition of the present invention is selected from the group consisting of Phenoxyethanol, 2-Methyl 5-cyclohexylpentanol, Dimethyl phenylbutanol, Dimethyl Phenylpropanol, 2-Benzylheptanol, Ethylhexylglycerin, Hydroxyacetophenone, Triethyl Citrate, Polyglyceryl-3 Caprylate, Polyglyceryl-10 Laurate, Glyceryl Caprylate, Benzyl Alcohol, Benzoic Acid, Sorbic Acid, Levulinic Acid and esters and/or ketals thereof, Anisic Acid and salts thereof, Polyglyceryl-2 Caprate, Caprylhydroxamic Acid, Zinc Ricinoleate, Farnesol, Glyceryl Laurate, Glyceryl Caprylate, Xylityl Sesquicaprylate, Phenylpropanol, Lauryl Alcohol, o-Cymen-5-ol, Tropolone, Climbazole, Piroctone Olamine, Octenidine HCl, Undecylenoyl Glycine, Sodium Caproyl/Lauroyl Lactylate, Ethyl Lauroyl Arginate Laurate, PCA Ethyl Cocoyl Arginate, Salvia Officinalis (Sage) Oil, Triclosan, Tetraselmis suecica extract, Mannitol, Sorbitol, Sodium Lauroyl Lactylate, Sodium Caproyl Lactylate, Salicylic Acid and its salts, Capryloyl Glycine, Ethyl Lauroyl Arginate HCl, Thymol, Jasmol, Gluconic Acid and salts thereof, Benzyl Salicylate, Propanediol Caprylate, 4-Hydroxyacetophenone, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more the aforesaid antimicrobial components.

In a particularly preferred variant, the antimicrobial component of the cosmetic or pharmaceutical composition of the present invention is selected from the group consisting of Ethylhexylglycerin, Glyceryl Caprylate, Phenoxyethanol, Caprylhydroxamic Acid, Farnesol, Climbazole, Triethyl Citrate, Benzyl Alcohol, o-Cymen-5-ol, Salicylic Acid and its salts, Benzyl Salicylate, Propanediol Caprylate, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more of the aforesaid antimicrobial components.

In a still more preferred variant, the antimicrobial component in the cosmetic or pharmaceutical composition according to the first or second aspect of the present invention is selected from the group consisting of 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more the aforesaid antimicrobial components. If the antimicrobial component (b) of the cosmetic or pharmaceutical component according to the first and second aspect of the present invention is an alkanediol, then the antimicrobial alkanediol component (b) is different from the alkanediol component (a).

The aforesaid antimicrobial component can be used either as a single component or in combination with one or more further antimicrobial components as specified above. In order to enhance the antimicrobial effect and to cover a broader range of antimicrobial effects, a combination of two or more antimicrobial components is preferably used.

In a preferred advantageously variant, the cosmetic or pharmaceutical composition according to the first or second aspect of the present invention comprises one of the following combinations of components (a) and (b):
1,2-pentanediol plus one or more antimicrobial component(s) selected from the group consisting of Phenoxyethanol, 2-Methyl 5-cyclohexylpentanol, Dimethyl phenylbutanol, Dimethyl Phenylpropanol, 2-Benzylheptanol, Ethylhexylglycerin, Hydroxyacetophenone, Triethyl Citrate, Polyglyceryl-3 Caprylate, Polyglyceryl-10 Laurate, Glyceryl Caprylate, Benzyl Alcohol, Benzoic Acid, Sorbic Acid, Levulinic Acid and esters and/or ketals thereof, Anisic Acid and salts thereof, Polyglyceryl-2 Caprate, Caprylhydroxamic Acid, Zinc Ricinoleate, Farnesol, Bisabolol, Glyceryl Laurate, Glyceryl Caprylate, Xylityl Sesquicaprylate, Phenylpropanol, Lauryl Alcohol, o-Cymen-5-ol, Tropolone, Climbazole, Piroctone Olamine, Octenidine HCl, Undecylenoyl Glycine, Sodium Caproyl/Lauroyl Lactylate, Ethyl Lauroyl Arginate Laurate, PCA Ethyl Cocoyl Arginate, Salvia Officinalis (Sage) Oil, Triclosan, Tetraselmis suecica extract, Mannitol, Sorbitol, Lactic Acid, Sodium Lauroyl Lactylate, Sodium Caproyl Lactylate, Salicylic Acid and its salts, Capryloyl Glycine, Ethyl Lauroyl Arginate HCl, Thymol, Jasmol, Gluconic Acid and salts thereof, Benzyl Salicylate, Propanediol Caprylate, 4-Hydroxyacetophenone, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more the aforesaid antimicrobial components; or
1,2-hexanediol plus one or more antimicrobial component(s) selected from the group consisting of Phenoxyethanol, 2-Methyl 5-cyclohexylpentanol, Dimethyl phenylbutanol, Dimethyl Phenylpropanol, 2-Benzylheptanol, Ethylhexylglycerin, Hydroxyacetophenone, Triethyl Citrate, Polyglyceryl-3 Caprylate, Polyglyceryl-10 Laurate, Glyceryl Caprylate, Benzyl Alcohol, Benzoic Acid, Sorbic Acid, Levulinic Acid and esters and/or ketals thereof, Anisic Acid and salts thereof, Polyglyceryl-2 Caprate, Caprylhydroxamic Acid, Zinc Ricinoleate, Farnesol, Bisabolol, Glyceryl Laurate, Glyceryl Caprylate, Xylityl Sesquicaprylate, Phenylpropanol, Lauryl Alcohol, o-Cymen-5-ol, Tropolone, Climbazole, Piroctone Olamine, Octenidine HCl, Undecylenoyl Glycine, Sodium Caproyl/Lauroyl Lactylate, Ethyl Lauroyl Arginate Laurate, PCA Ethyl Cocoyl Arginate, Salvia Officinalis (Sage) Oil, Triclosan, Tetraselmis suecica extract, Mannitol, Sorbitol, Lactic Acid, Sodium Lauroyl Lactylate, Sodium Caproyl Lactylate, Salicylic Acid and its salts, Capryloyl Glycine, Ethyl Lauroyl Arginate HCl, Thymol, Jasmol, Gluconic Acid and salts thereof, Benzyl Salicylate, Propanediol Caprylate, 4-Hydroxyacetophenone, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more the aforesaid antimicrobial components; or
1,2-heptanediol plus one or more antimicrobial component(s) selected from the group consisting of Phenoxyethanol, 2-Methyl 5-cyclohexylpentanol, Dimethyl phenylbutanol, Dimethyl Phenylpropanol, 2-Benzylheptanol, Ethylhexylglycerin, Hydroxyacetophenone, Triethyl Citrate, Polyglyceryl-3 Caprylate, Polyglyceryl-10 Laurate, Glyceryl Caprylate, Benzyl Alcohol, Benzoic Acid, Sorbic Acid, Levulinic Acid and esters and/or ketals thereof, Anisic Acid and salts thereof, Polyglyceryl-2 Caprate, Caprylhydroxamic Acid, Zinc Ricinoleate, Farnesol, Bisabolol, Glyceryl Laurate, Glyceryl Caprylate, Xylityl Sesquicaprylate, Phenylpropanol, Lauryl Alcohol, o-Cymen-5-ol, Tropolone, Climbazole, Piroctone Olamine, Octenidine HCl, Undecylenoyl Glycine, Sodium Caproyl/Lauroyl Lactylate, Ethyl Lauroyl Arginate Laurate, PCA Ethyl Cocoyl Arginate, Salvia Officinalis (Sage) Oil, Triclosan, Tetraselmis suecica extract, Mannitol, Sorbitol, Lactic Acid, Sodium Lauroyl Lactylate, Sodium Caproyl Lactylate, Salicylic Acid and its salts, Capryloyl Glycine, Ethyl Lauroyl Arginate HCl, Thymol, Jasmol, Gluconic Acid and salts thereof, Benzyl Salicylate, Propanediol Caprylate, 4-Hydroxyacetophenone, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more the aforesaid antimicrobial components; or
1,2-octanediol plus one or more antimicrobial component(s) selected from the group consisting of Phenoxyethanol, 2-Methyl 5-cyclohexylpentanol, Dimethyl phenylbutanol, Dimethyl Phenylpropanol, 2-Benzylheptanol, Ethylhexylglycerin, Hydroxyacetophenone, Triethyl Citrate, Polyglyceryl-3 Caprylate, Polyglyceryl-10 Laurate, Glyceryl Caprylate, Benzyl Alcohol, Benzoic Acid, Sorbic Acid, Levulinic Acid and esters and/or ketals thereof, Anisic Acid and salts thereof, Polyglyceryl-2 Caprate, Caprylhydroxamic Acid, Zinc Ricinoleate, Farnesol, Bisabolol, Glyceryl Laurate, Glyceryl Caprylate, Xylityl Sesquicaprylate, Phenylpropanol, Lauryl Alcohol, o-Cymen-5-ol, Tropolone, Climbazole, Piroctone Olamine, Octenidine HCl, Undecylenoyl Glycine, Sodium Caproyl/Lauroyl Lactylate, Ethyl Lauroyl Arginate Laurate, PCA Ethyl Cocoyl Arginate, Salvia Officinalis (Sage) Oil, Triclosan, Tetraselmis suecica extract, Mannitol, Sorbitol, Lactic Acid, Sodium Lauroyl Lactylate, Sodium Caproyl Lactylate, Salicylic Acid and its salts, Capryloyl Glycine, Ethyl Lauroyl Arginate HCl, Thymol, Jasmol, Gluconic Acid and salts thereof, Benzyl Salicylate, Propanediol Caprylate, 4-Hydroxyacetophenone, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more the aforesaid antimicrobial components; or
1,2-nonanediol plus one or more antimicrobial component(s) selected from the group consisting of Phenoxyethanol, 2-Methyl 5-cyclohexylpentanol, Dimethyl phenylbutanol, Dimethyl Phenylpropanol, 2-Benzylheptanol, Ethylhexylglycerin, Hydroxyacetophenone, Triethyl Citrate, Polyglyceryl-3 Caprylate, Polyglyceryl-10 Laurate, Glyceryl Caprylate, Benzyl Alcohol, Benzoic Acid, Sorbic Acid, Levulinic Acid and esters and/or ketals thereof, Anisic Acid and salts thereof, Polyglyceryl-2 Caprate, Caprylhydroxamic Acid, Zinc Ricinoleate, Farnesol, Glyceryl Laurate, Glyceryl Caprylate, Xylityl Sesquicaprylate, Phenylpropanol, Lauryl Alcohol, o-Cymen-5-ol, Tropolone, Climbazole, Piroctone Olamine, Octenidine HCl, Undecylenoyl Glycine, Sodium Caproyl/Lauroyl Lactylate, Ethyl Lauroyl Arginate Laurate, PCA Ethyl Cocoyl Arginate, Salvia Officinalis (Sage) Oil, Triclosan, Tetraselmis suecica extract, Mannitol, Sorbitol, Sodium Lauroyl Lactylate, Sodium Caproyl Lactylate, Salicylic Acid and its salts, Capryloyl Glycine, Ethyl Lauroyl Arginate HCl, Thymol, Jasmol, Gluconic Acid and salts thereof, Benzyl Salicylate, Propanediol Caprylate, 4-Hydroxyacetophenone, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more the aforesaid antimicrobial components; or
1,2-decanediol plus one or more antimicrobial component(s) selected from the group consisting of Phenoxyethanol, 2-Methyl 5-cyclohexylpentanol, Dimethyl phenylbutanol, Dimethyl Phenylpropanol, 2-Benzylheptanol, Ethylhexylglycerin, Hydroxyacetophenone, Triethyl Citrate, Polyglyceryl-3 Caprylate, Polyglyceryl-10 Laurate, Glyceryl Caprylate, Benzyl Alcohol, Benzoic Acid, Sorbic Acid, Levulinic Acid and esters and/or ketals thereof, Anisic Acid and salts thereof, Polyglyceryl-2 Caprate, Caprylhydroxamic Acid, Zinc Ricinoleate, Farnesol, Bisabolol, Glyceryl Laurate, Glyceryl Caprylate, Xylityl Sesquicaprylate, Phenylpropanol, Lauryl Alcohol, o-Cymen-5-ol, Tropolone, Climbazole, Piroctone Olamine, Octenidine HCl, Undecylenoyl Glycine, Sodium Caproyl/Lauroyl Lactylate, Ethyl Lauroyl Arginate Laurate, PCA Ethyl Cocoyl Arginate, Salvia Officinalis (Sage) Oil, Triclosan, Tetraselmis suecica extract, Mannitol, Sorbitol, Lactic Acid, Sodium Lauroyl Lactylate, Sodium Caproyl Lactylate, Salicylic Acid and its salts, Capryloyl Glycine, Ethyl Lauroyl Arginate HCl, Thymol, Jasmol, Gluconic Acid and salts thereof, Benzyl Salicylate, Propanediol Caprylate, 4-Hydroxyacetophenone, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more the aforesaid antimicrobial components; or
1,2-undecanediol plus one or more antimicrobial component(s) selected from the group consisting of Phenoxyethanol, 2-Methyl 5-cyclohexylpentanol, Dimethyl phenylbutanol, Dimethyl Phenylpropanol, 2-Benzylheptanol, Ethylhexylglycerin, Hydroxyacetophenone, Triethyl Citrate, Polyglyceryl-3 Caprylate, Polyglyceryl-10 Laurate, Glyceryl Caprylate, Benzyl Alcohol, Benzoic Acid, Sorbic Acid, Levulinic Acid and esters and/or ketals thereof, Anisic Acid and salts thereof, Polyglyceryl-2 Caprate, Caprylhydroxamic Acid, Zinc Ricinoleate, Farnesol, Bisabolol, Glyceryl Laurate, Glyceryl Caprylate, Xylityl Sesquicaprylate, Phenylpropanol, Lauryl Alcohol, o-Cymen-5-ol, Tropolone, Climbazole, Piroctone Olamine, Octenidine HCl, Undecylenoyl Glycine, Sodium Caproyl/Lauroyl Lactylate, Ethyl Lauroyl Arginate Laurate, PCA Ethyl Cocoyl Arginate, Salvia Officinalis (Sage) Oil, Triclosan, Tetraselmis suecica extract, Mannitol, Sorbitol, Lactic Acid, Sodium Lauroyl Lactylate, Sodium Caproyl Lactylate, Salicylic Acid and its salts, Capryloyl Glycine, Ethyl Lauroyl Arginate HCl, Thymol, Jasmol, Gluconic Acid and salts thereof, Benzyl Salicylate, Propanediol Caprylate, 4-Hydroxyacetophenone, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more the aforesaid antimicrobial components; or
1,2-dodecanediol plus one or more antimicrobial component(s) selected from the group consisting of Phenoxyethanol, 2-Methyl 5-cyclohexylpentanol, Dimethyl phenylbutanol, Dimethyl Phenylpropanol, 2-Benzylheptanol, Ethylhexylglycerin, Hydroxyacetophenone, Triethyl Citrate, Polyglyceryl-3 Caprylate, Polyglyceryl-10 Laurate, Glyceryl Caprylate, Benzyl Alcohol, Benzoic Acid, Sorbic Acid, Levulinic Acid and esters and/or ketals thereof, Anisic Acid and salts thereof, Polyglyceryl-2 Caprate, Caprylhydroxamic Acid, Zinc Ricinoleate, Farnesol, Bisabolol, Glyceryl Laurate, Glyceryl Caprylate, Xylityl Sesquicaprylate, Phenylpropanol, Lauryl Alcohol, o-Cymen-5-ol, Tropolone, Climbazole, Piroctone Olamine, Octenidine HCl, Undecylenoyl Glycine, Sodium Caproyl/Lauroyl Lactylate, Ethyl Lauroyl Arginate Laurate, PCA Ethyl Cocoyl Arginate, Salvia Officinalis (Sage) Oil, Triclosan, Tetraselmis suecica extract, Mannitol, Sorbitol, Lactic Acid, Sodium Lauroyl Lactylate, Sodium Caproyl Lactylate, Salicylic Acid and its salts, Capryloyl Glycine, Ethyl Lauroyl Arginate HCl, Thymol, Jasmol, Gluconic Acid and salts thereof, Benzyl Salicylate, Propanediol Caprylate, 4-Hydroxyacetophenone, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more the aforesaid antimicrobial components; or
1,2-tridecanediol plus one or more antimicrobial component(s) selected from the group consisting of Phenoxyethanol, 2-Methyl 5-cyclohexylpentanol, Dimethyl phenylbutanol, Dimethyl Phenylpropanol, 2-Benzylheptanol, Ethylhexylglycerin, Hydroxyacetophenone, Triethyl Citrate, Polyglyceryl-3 Caprylate, Polyglyceryl-10 Laurate, Glyceryl Caprylate, Benzyl Alcohol, Benzoic Acid, Sorbic Acid, Levulinic Acid and esters and/or ketals thereof, Anisic Acid and salts thereof, Polyglyceryl-2 Caprate, Caprylhydroxamic Acid, Zinc Ricinoleate, Farnesol, Bisabolol, Glyceryl Laurate, Glyceryl Caprylate, Xylityl Sesquicaprylate, Phenylpropanol, Lauryl Alcohol, o-Cymen-5-ol, Tropolone, Climbazole, Piroctone Olamine, Octenidine HCl, Undecylenoyl Glycine, Sodium Caproyl/Lauroyl Lactylate, Ethyl Lauroyl Arginate Laurate, PCA Ethyl Cocoyl Arginate, Salvia Officinalis (Sage) Oil, Triclosan, Tetraselmis suecica extract, Mannitol, Sorbitol, Lactic Acid, Sodium Lauroyl Lactylate, Sodium Caproyl Lactylate, Salicylic Acid and its salts, Capryloyl Glycine, Ethyl Lauroyl Arginate HCl, Thymol, Jasmol, Gluconic Acid and salts thereof, Benzyl Salicylate, Propanediol Caprylate, 4-Hydroxyacetophenone, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more the aforesaid antimicrobial components; or
2,3-pentanediol plus one or more antimicrobial component(s) selected from the group consisting of Phenoxyethanol, 2-Methyl 5-cyclohexylpentanol, Dimethyl phenylbutanol, Dimethyl Phenylpropanol, 2-Benzylheptanol, Ethylhexylglycerin, Hydroxyacetophenone, Triethyl Citrate, Polyglyceryl-3 Caprylate, Polyglyceryl-10 Laurate, Glyceryl Caprylate, Benzyl Alcohol, Benzoic Acid, Sorbic Acid, Levulinic Acid and esters and/or ketals thereof, Anisic Acid and salts thereof, Polyglyceryl-2 Caprate, Caprylhydroxamic Acid, Zinc Ricinoleate, Farnesol, Bisabolol, Glyceryl Laurate, Glyceryl Caprylate, Xylityl Sesquicaprylate, Phenylpropanol, Lauryl Alcohol, o-Cymen-5-ol, Tropolone, Climbazole, Piroctone Olamine, Octenidine HCl, Undecylenoyl Glycine, Sodium Caproyl/Lauroyl Lactylate, Ethyl Lauroyl Arginate Laurate, PCA Ethyl Cocoyl Arginate, Salvia Officinalis (Sage) Oil, Triclosan, Tetraselmis suecica extract, Mannitol, Sorbitol, Lactic Acid, Sodium Lauroyl Lactylate, Sodium Caproyl Lactylate, Salicylic Acid and its salts, Capryloyl Glycine, Ethyl Lauroyl Arginate HCl, Thymol, Jasmol, Gluconic Acid and salts thereof, Benzyl Salicylate, Propanediol Caprylate, 4-Hydroxyacetophenone, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more the aforesaid antimicrobial components; or
2,3-hexanediol plus one or more antimicrobial component(s) selected from the group consisting of Phenoxyethanol, 2-Methyl 5-cyclohexylpentanol, Dimethyl phenylbutanol, Dimethyl Phenylpropanol, 2-Benzylheptanol, Ethylhexylglycerin, Hydroxyacetophenone, Triethyl Citrate, Polyglyceryl-3 Caprylate, Polyglyceryl-10 Laurate, Glyceryl Caprylate, Benzyl Alcohol, Benzoic Acid, Sorbic Acid, Levulinic Acid and esters and/or ketals thereof, Anisic Acid and salts thereof, Polyglyceryl-2 Caprate, Caprylhydroxamic Acid, Zinc Ricinoleate, Farnesol, Bisabolol, Glyceryl Laurate, Glyceryl Caprylate, Xylityl Sesquicaprylate, Phenylpropanol, Lauryl Alcohol, o-Cymen-5-ol, Tropolone, Climbazole, Piroctone Olamine, Octenidine HCl, Undecylenoyl Glycine, Sodium Caproyl/Lauroyl Lactylate, Ethyl Lauroyl Arginate Laurate, PCA Ethyl Cocoyl Arginate, Salvia Officinalis (Sage) Oil, Triclosan, Tetraselmis suecica extract, Mannitol, Sorbitol, Lactic Acid, Sodium Lauroyl Lactylate, Sodium Caproyl Lactylate, Salicylic Acid and its salts, Capryloyl Glycine, Ethyl Lauroyl Arginate HCl, Thymol, Jasmol, Gluconic Acid and salts thereof, Benzyl Salicylate, Propanediol Caprylate, 4-Hydroxyacetophenone, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more the aforesaid antimicrobial components; or
2,3-heptanediol plus one or more antimicrobial component(s) selected from the group consisting of Phenoxyethanol, 2-Methyl 5-cyclohexylpentanol, Dimethyl phenylbutanol, Dimethyl Phenylpropanol, 2-Benzylheptanol, Ethylhexylglycerin, Hydroxyacetophenone, Triethyl Citrate, Polyglyceryl-3 Caprylate, Polyglyceryl-10 Laurate, Glyceryl Caprylate, Benzyl Alcohol, Benzoic Acid, Sorbic Acid, Levulinic Acid and esters and/or ketals thereof, Anisic Acid and salts thereof, Polyglyceryl-2 Caprate, Caprylhydroxamic Acid, Zinc Ricinoleate, Farnesol, Bisabolol, Glyceryl Laurate, Glyceryl Caprylate, Xylityl Sesquicaprylate, Phenylpropanol, Lauryl Alcohol, o-Cymen-5-ol, Tropolone, Climbazole, Piroctone Olamine, Octenidine HCl, Undecylenoyl Glycine, Sodium Caproyl/Lauroyl Lactylate, Ethyl Lauroyl Arginate Laurate, PCA Ethyl Cocoyl Arginate, Salvia Officinalis (Sage) Oil, Triclosan, Tetraselmis suecica extract, Mannitol, Sorbitol, Lactic Acid, Sodium Lauroyl Lactylate, Sodium Caproyl Lactylate, Salicylic Acid and its salts, Capryloyl Glycine, Ethyl Lauroyl Arginate HCl, Thymol, Jasmol, Gluconic Acid and salts thereof, Benzyl Salicylate, Propanediol Caprylate, 4-Hydroxyacetophenone, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more the aforesaid antimicrobial components; or
2,3-octanediol plus one or more antimicrobial component(s) selected from the group consisting of Phenoxyethanol, 2-Methyl 5-cyclohexylpentanol, Dimethyl phenylbutanol, Dimethyl Phenylpropanol, 2-Benzylheptanol, Ethylhexylglycerin, Hydroxyacetophenone, Triethyl Citrate, Polyglyceryl-3 Caprylate, Polyglyceryl-10 Laurate, Glyceryl Caprylate, Benzyl Alcohol, Benzoic Acid, Sorbic Acid, Levulinic Acid and esters and/or ketals thereof, Anisic Acid and salts thereof, Polyglyceryl-2 Caprate, Caprylhydroxamic Acid, Zinc Ricinoleate, Farnesol, Bisabolol, Glyceryl Laurate, Glyceryl Caprylate, Xylityl Sesquicaprylate, Phenylpropanol, Lauryl Alcohol, o-Cymen-5-ol, Tropolone, Climbazole, Piroctone Olamine, Octenidine HCl, Undecylenoyl Glycine, Sodium Caproyl/Lauroyl Lactylate, Ethyl Lauroyl Arginate Laurate, PCA Ethyl Cocoyl Arginate, Salvia Officinalis (Sage) Oil, Triclosan, Tetraselmis suecica extract, Mannitol, Sorbitol, Lactic Acid, Sodium Lauroyl Lactylate, Sodium Caproyl Lactylate, Salicylic Acid and its salts, Capryloyl Glycine, Ethyl Lauroyl Arginate HCl, Thymol, Jasmol, Gluconic Acid and salts thereof, Benzyl Salicylate, Propanediol Caprylate, 4-Hydroxyacetophenone, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more the aforesaid antimicrobial components; or
2,3-nonanediol plus one or more antimicrobial component(s) selected from the group consisting of Phenoxyethanol, 2-Methyl 5-cyclohexylpentanol, Dimethyl phenylbutanol, Dimethyl Phenylpropanol, 2-Benzylheptanol, Ethylhexylglycerin, Hydroxyacetophenone, Triethyl Citrate, Polyglyceryl-3 Caprylate, Polyglyceryl-10 Laurate, Glyceryl Caprylate, Benzyl Alcohol, Benzoic Acid, Sorbic Acid, Levulinic Acid and esters and/or ketals thereof, Anisic Acid and salts thereof, Polyglyceryl-2 Caprate, Caprylhydroxamic Acid, Zinc Ricinoleate, Farnesol, Bisabolol, Glyceryl Laurate, Glyceryl Caprylate, Xylityl Sesquicaprylate, Phenylpropanol, Lauryl Alcohol, o-Cymen-5-ol, Tropolone, Climbazole, Piroctone Olamine, Octenidine HCl, Undecylenoyl Glycine, Sodium Caproyl/Lauroyl Lactylate, Ethyl Lauroyl Arginate Laurate, PCA Ethyl Cocoyl Arginate, Salvia Officinalis (Sage) Oil, Triclosan, Tetraselmis suecica extract, Mannitol, Sorbitol, Lactic Acid, Sodium Lauroyl Lactylate, Sodium Caproyl Lactylate, Salicylic Acid and its salts, Capryloyl Glycine, Ethyl Lauroyl Arginate HCl, Thymol, Jasmol, Gluconic Acid and salts thereof, Benzyl Salicylate, Propanediol Caprylate, 4-Hydroxyacetophenone, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more the aforesaid antimicrobial components; or
2,3-decanediol plus one or more antimicrobial component(s) selected from the group consisting of Phenoxyethanol, 2-Methyl 5-cyclohexylpentanol, Dimethyl phenylbutanol, Dimethyl Phenylpropanol, 2-Benzylheptanol, Ethylhexylglycerin, Hydroxyacetophenone, Triethyl Citrate, Polyglyceryl-3 Caprylate, Polyglyceryl-10 Laurate, Glyceryl Caprylate, Benzyl Alcohol, Benzoic Acid, Sorbic Acid, Levulinic Acid and esters and/or ketals thereof, Anisic Acid and salts thereof, Polyglyceryl-2 Caprate, Caprylhydroxamic Acid, Zinc Ricinoleate, Farnesol, Bisabolol, Glyceryl Laurate, Glyceryl Caprylate, Xylityl Sesquicaprylate, Phenylpropanol, Lauryl Alcohol, o-Cymen-5-ol, Tropolone, Climbazole, Piroctone Olamine, Octenidine HCl, Undecylenoyl Glycine, Sodium Caproyl/Lauroyl Lactylate, Ethyl Lauroyl Arginate Laurate, PCA Ethyl Cocoyl Arginate, Salvia Officinalis (Sage) Oil, Triclosan, Tetraselmis suecica extract, Mannitol, Sorbitol, Lactic Acid, Sodium Lauroyl Lactylate, Sodium Caproyl Lactylate, Salicylic Acid and its salts, Capryloyl Glycine, Ethyl Lauroyl Arginate HCl, Thymol, Jasmol, Gluconic Acid and salts thereof, Benzyl Salicylate, Propanediol Caprylate, 4-Hydroxyacetophenone, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more the aforesaid antimicrobial components; or
2,3-undecanediol plus one or more antimicrobial component(s) selected from the group consisting of Phenoxyethanol, 2-Methyl 5-cyclohexylpentanol, Dimethyl phenylbutanol, Dimethyl Phenylpropanol, 2-Benzylheptanol, Ethylhexylglycerin, Hydroxyacetophenone, Triethyl Citrate, Polyglyceryl-3 Caprylate, Polyglyceryl-10 Laurate, Glyceryl Caprylate, Benzyl Alcohol, Benzoic Acid, Sorbic Acid, Levulinic Acid and esters and/or ketals thereof, Anisic Acid and salts thereof, Polyglyceryl-2 Caprate, Caprylhydroxamic Acid, Zinc Ricinoleate, Farnesol, Bisabolol, Glyceryl Laurate, Glyceryl Caprylate, Xylityl Sesquicaprylate, Phenylpropanol, Lauryl Alcohol, o-Cymen-5-ol, Tropolone, Climbazole, Piroctone Olamine, Octenidine HCl, Undecylenoyl Glycine, Sodium Caproyl/Lauroyl Lactylate, Ethyl Lauroyl Arginate Laurate, PCA Ethyl Cocoyl Arginate, Salvia Officinalis (Sage) Oil, Triclosan, Tetraselmis suecica extract, Mannitol, Sorbitol, Lactic Acid, Sodium Lauroyl Lactylate, Sodium Caproyl Lactylate, Salicylic Acid and its salts, Capryloyl Glycine, Ethyl Lauroyl Arginate HCl, Thymol, Jasmol, Gluconic Acid and salts thereof, Benzyl Salicylate, Propanediol Caprylate, 4-Hydroxyacetophenone, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more the aforesaid antimicrobial components; or
2,3-dodecanediol plus one or more antimicrobial component(s) selected from the group consisting of Phenoxyethanol, 2-Methyl 5-cyclohexylpentanol, Dimethyl phenylbutanol, Dimethyl Phenylpropanol, 2-Benzylheptanol, Ethylhexylglycerin, Hydroxyacetophenone, Triethyl Citrate, Polyglyceryl-3 Caprylate, Polyglyceryl-10 Laurate, Glyceryl Caprylate, Benzyl Alcohol, Benzoic Acid, Sorbic Acid, Levulinic Acid and esters and/or ketals thereof, Anisic Acid and salts thereof, Polyglyceryl-2 Caprate, Caprylhydroxamic Acid, Zinc Ricinoleate, Farnesol, Bisabolol, Glyceryl Laurate, Glyceryl Caprylate, Xylityl Sesquicaprylate, Phenylpropanol, Lauryl Alcohol, o-Cymen-5-ol, Tropolone, Climbazole, Piroctone Olamine, Octenidine HCl, Undecylenoyl Glycine, Sodium Caproyl/Lauroyl Lactylate, Ethyl Lauroyl Arginate Laurate, PCA Ethyl Cocoyl Arginate, Salvia Officinalis (Sage) Oil, Triclosan, Tetraselmis suecica extract, Mannitol, Sorbitol, Lactic Acid, Sodium Lauroyl Lactylate, Sodium Caproyl Lactylate, Salicylic Acid and its salts, Capryloyl Glycine, Ethyl Lauroyl Arginate HCl, Thymol, Jasmol, Gluconic Acid and salts thereof, Benzyl Salicylate, Propanediol Caprylate, 4-Hydroxyacetophenone, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more the aforesaid antimicrobial components; or
2,3-tridecanediol plus one or more antimicrobial component(s) selected from the group consisting of Phenoxyethanol, 2-Methyl 5-cyclohexylpentanol, Dimethyl phenylbutanol, Dimethyl Phenylpropanol, 2-Benzylheptanol, Ethylhexylglycerin, Hydroxyacetophenone, Triethyl Citrate, Polyglyceryl-3 Caprylate, Polyglyceryl-10 Laurate, Glyceryl Caprylate, Benzyl Alcohol, Benzoic Acid, Sorbic Acid, Levulinic Acid and esters and/or ketals thereof, Anisic Acid and salts thereof, Polyglyceryl-2 Caprate, Caprylhydroxamic Acid, Zinc Ricinoleate, Farnesol, Bisabolol, Glyceryl Laurate, Glyceryl Caprylate, Xylityl Sesquicaprylate, Phenylpropanol, Lauryl Alcohol, o-Cymen-5-ol, Tropolone, Climbazole, Piroctone Olamine, Octenidine HCl, Undecylenoyl Glycine, Sodium Caproyl/Lauroyl Lactylate, Ethyl Lauroyl Arginate Laurate, PCA Ethyl Cocoyl Arginate, Salvia Officinalis (Sage) Oil, Triclosan, Tetraselmis suecica extract, Mannitol, Sorbitol, Lactic Acid, Sodium Lauroyl Lactylate, Sodium Caproyl Lactylate, Salicylic Acid and its salts, Capryloyl Glycine, Ethyl Lauroyl Arginate HCl, Thymol, Jasmol, Gluconic Acid and salts thereof, Benzyl Salicylate, Propanediol Caprylate, 4-Hydroxyacetophenone, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more the aforesaid antimicrobial components; or
1,2-pentanediol in combination with 2,3-pentanediol plus one or more antimicrobial component(s) selected from the group consisting of Phenoxyethanol, 2-Methyl 5-cyclohexylpentanol, Dimethyl phenylbutanol, Dimethyl Phenylpropanol, 2-Benzylheptanol, Ethylhexylglycerin, Hydroxyacetophenone, Triethyl Citrate, Polyglyceryl-3 Caprylate, Polyglyceryl-10 Laurate, Glyceryl Caprylate, Benzyl Alcohol, Benzoic Acid, Sorbic Acid, Levulinic Acid and esters and/or ketals thereof, Anisic Acid and salts thereof, Polyglyceryl-2 Caprate, Caprylhydroxamic Acid, Zinc Ricinoleate, Farnesol, Bisabolol, Glyceryl Laurate, Glyceryl Caprylate, Xylityl Sesquicaprylate, Phenylpropanol, Lauryl Alcohol, o-Cymen-5-ol, Tropolone, Climbazole, Piroctone Olamine, Octenidine HCl, Undecylenoyl Glycine, Sodium Caproyl/Lauroyl Lactylate, Ethyl Lauroyl Arginate Laurate, PCA Ethyl Cocoyl Arginate, Salvia Officinalis (Sage) Oil, Triclosan, Tetraselmis suecica extract, Mannitol, Sorbitol, Lactic Acid, Sodium Lauroyl Lactylate, Sodium Caproyl Lactylate, Salicylic Acid and its salts, Capryloyl Glycine, Ethyl Lauroyl Arginate HCl, Thymol, Jasmol, Gluconic Acid and salts thereof, Benzyl Salicylate, Propanediol Caprylate, 4-Hydroxyacetophenone, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more the aforesaid antimicrobial components; or
1,2-hexanediol in combination with 2,3-hexanediol plus one or more antimicrobial component(s) selected from the group consisting of Phenoxyethanol, 2-Methyl 5-cyclohexylpentanol, Dimethyl phenylbutanol, Dimethyl Phenylpropanol, 2-Benzylheptanol, Ethylhexylglycerin, Hydroxyacetophenone, Triethyl Citrate, Polyglyceryl-3 Caprylate, Polyglyceryl-10 Laurate, Glyceryl Caprylate, Benzyl Alcohol, Benzoic Acid, Sorbic Acid, Levulinic Acid and esters and/or ketals thereof, Anisic Acid and salts thereof, Polyglyceryl-2 Caprate, Caprylhydroxamic Acid, Zinc Ricinoleate, Farnesol, Bisabolol, Glyceryl Laurate, Glyceryl Caprylate, Xylityl Sesquicaprylate, Phenylpropanol, Lauryl Alcohol, o-Cymen-5-ol, Tropolone, Climbazole, Piroctone Olamine, Octenidine HCl, Undecylenoyl Glycine, Sodium Caproyl/Lauroyl Lactylate, Ethyl Lauroyl Arginate Laurate, PCA Ethyl Cocoyl Arginate, Salvia Officinalis (Sage) Oil, Triclosan, Tetraselmis suecica extract, Mannitol, Sorbitol, Lactic Acid, Sodium Lauroyl Lactylate, Sodium Caproyl Lactylate, Salicylic Acid and its salts, Capryloyl Glycine, Ethyl Lauroyl Arginate HCl, Thymol, Jasmol, Gluconic Acid and salts thereof, Benzyl Salicylate, Propanediol Caprylate, 4-Hydroxyacetophenone, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more the aforesaid antimicrobial components; or
1,2-heptanediol in combination with 2,3-heptanediol plus one or more antimicrobial component(s) selected from the group consisting of Phenoxyethanol, 2-Methyl 5-cyclohexylpentanol, Dimethyl phenylbutanol, Dimethyl Phenylpropanol, 2-Benzylheptanol, Ethylhexylglycerin, Hydroxyacetophenone, Triethyl Citrate, Polyglyceryl-3 Caprylate, Polyglyceryl-10 Laurate, Glyceryl Caprylate, Benzyl Alcohol, Benzoic Acid, Sorbic Acid, Levulinic Acid and esters and/or ketals thereof, Anisic Acid and salts thereof, Polyglyceryl-2 Caprate, Caprylhydroxamic Acid, Zinc Ricinoleate, Farnesol, Bisabolol, Glyceryl Laurate, Glyceryl Caprylate, Xylityl Sesquicaprylate, Phenylpropanol, Lauryl Alcohol, o-Cymen-5-ol, Tropolone, Climbazole, Piroctone Olamine, Octenidine HCl, Undecylenoyl Glycine, Sodium Caproyl/Lauroyl Lactylate, Ethyl Lauroyl Arginate Laurate, PCA Ethyl Cocoyl Arginate, Salvia Officinalis (Sage) Oil, Triclosan, Tetraselmis suecica extract, Mannitol, Sorbitol, Lactic Acid, Sodium Lauroyl Lactylate, Sodium Caproyl Lactylate, Salicylic Acid and its salts, Capryloyl Glycine, Ethyl Lauroyl Arginate HCl, Thymol, Jasmol, Gluconic Acid and salts thereof, Benzyl Salicylate, Propanediol Caprylate, 4-Hydroxyacetophenone, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more the aforesaid antimicrobial components; or
1,2-octanediol in combination with 2,3-octanediol plus one or more antimicrobial component(s) selected from the group consisting of Phenoxyethanol, 2-Methyl 5-cyclohexylpentanol, Dimethyl phenylbutanol, Dimethyl Phenylpropanol, 2-Benzylheptanol, Ethylhexylglycerin, Hydroxyacetophenone, Triethyl Citrate, Polyglyceryl-3 Caprylate, Polyglyceryl-10 Laurate, Glyceryl Caprylate, Benzyl Alcohol, Benzoic Acid, Sorbic Acid, Levulinic Acid and esters and/or ketals thereof, Anisic Acid and salts thereof, Polyglyceryl-2 Caprate, Caprylhydroxamic Acid, Zinc Ricinoleate, Farnesol, Bisabolol, Glyceryl Laurate, Glyceryl Caprylate, Xylityl Sesquicaprylate, Phenylpropanol, Lauryl Alcohol, o-Cymen-5-ol, Tropolone, Climbazole, Piroctone Olamine, Octenidine HCl, Undecylenoyl Glycine, Sodium Caproyl/Lauroyl Lactylate, Ethyl Lauroyl Arginate Laurate, PCA Ethyl Cocoyl Arginate, Salvia Officinalis (Sage) Oil, Triclosan, Tetraselmis suecica extract, Mannitol, Sorbitol, Lactic Acid, Sodium Lauroyl Lactylate, Sodium Caproyl Lactylate, Salicylic Acid and its salts, Capryloyl Glycine, Ethyl Lauroyl Arginate HCl, Thymol, Jasmol, Gluconic Acid and salts thereof, Benzyl Salicylate, Propanediol Caprylate, 4-Hydroxyacetophenone, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more the aforesaid antimicrobial components; or
1,2-nonanediol in combination with 2,3-nonanediol plus one or more antimicrobial component(s) selected from the group consisting of Phenoxyethanol, 2-Methyl 5-cyclohexylpentanol, Dimethyl phenylbutanol, Dimethyl Phenylpropanol, 2-Benzylheptanol, Ethylhexylglycerin, Hydroxyacetophenone, Triethyl Citrate, Polyglyceryl-3 Caprylate, Polyglyceryl-10 Laurate, Glyceryl Caprylate, Benzyl Alcohol, Benzoic Acid, Sorbic Acid, Levulinic Acid and esters and/or ketals thereof, Anisic Acid and salts thereof, Polyglyceryl-2 Caprate, Caprylhydroxamic Acid, Zinc Ricinoleate, Farnesol, Bisabolol, Glyceryl Laurate, Glyceryl Caprylate, Xylityl Sesquicaprylate, Phenylpropanol, Lauryl Alcohol, o-Cymen-5-ol, Tropolone, Climbazole, Piroctone Olamine, Octenidine HCl, Undecylenoyl Glycine, Sodium Caproyl/Lauroyl Lactylate, Ethyl Lauroyl Arginate Laurate, PCA Ethyl Cocoyl Arginate, Salvia Officinalis (Sage) Oil, Triclosan, Tetraselmis suecica extract, Mannitol, Sorbitol, Lactic Acid, Sodium Lauroyl Lactylate, Sodium Caproyl Lactylate, Salicylic Acid and its salts, Capryloyl Glycine, Ethyl Lauroyl Arginate HCl, Thymol, Jasmol, Gluconic Acid and salts thereof, Benzyl Salicylate, Propanediol Caprylate, 4-Hydroxyacetophenone, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more the aforesaid antimicrobial components; or
1,2-decanediol in combination with 2,3-decanediol plus one or more antimicrobial component(s) selected from the group consisting of Phenoxyethanol, 2-Methyl 5-cyclohexylpentanol, Dimethyl phenylbutanol, Dimethyl Phenylpropanol, 2-Benzylheptanol, Ethylhexylglycerin, Hydroxyacetophenone, Triethyl Citrate, Polyglyceryl-3 Caprylate, Polyglyceryl-10 Laurate, Glyceryl Caprylate, Benzyl Alcohol, Benzoic Acid, Sorbic Acid, Levulinic Acid and esters and/or ketals thereof, Anisic Acid and salts thereof, Polyglyceryl-2 Caprate, Caprylhydroxamic Acid, Zinc Ricinoleate, Farnesol, Bisabolol, Glyceryl Laurate, Glyceryl Caprylate, Xylityl Sesquicaprylate, Phenylpropanol, Lauryl Alcohol, o-Cymen-5-ol, Tropolone, Climbazole, Piroctone Olamine, Octenidine HCl, Undecylenoyl Glycine, Sodium Caproyl/Lauroyl Lactylate, Ethyl Lauroyl Arginate Laurate, PCA Ethyl Cocoyl Arginate, Salvia Officinalis (Sage) Oil, Triclosan, Tetraselmis suecica extract, Mannitol, Sorbitol, Lactic Acid, Sodium Lauroyl Lactylate, Sodium Caproyl Lactylate, Salicylic Acid and its salts, Capryloyl Glycine, Ethyl Lauroyl Arginate HCl, Thymol, Jasmol, Gluconic Acid and salts thereof, Benzyl Salicylate, Propanediol Caprylate, 4-Hydroxyacetophenone, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more the aforesaid antimicrobial components; or
1,2-undecanediol in combination with 2,3-undecanediol plus one or more antimicrobial component(s) selected from the group consisting of Phenoxyethanol, 2-Methyl 5-cyclohexylpentanol, Dimethyl phenylbutanol, Dimethyl Phenylpropanol, 2-Benzylheptanol, Ethylhexylglycerin, Hydroxyacetophenone, Triethyl Citrate, Polyglyceryl-3 Caprylate, Polyglyceryl-10 Laurate, Glyceryl Caprylate, Benzyl Alcohol, Benzoic Acid, Sorbic Acid, Levulinic Acid and esters and/or ketals thereof, Anisic Acid and salts thereof, Polyglyceryl-2 Caprate, Caprylhydroxamic Acid, Zinc Ricinoleate, Farnesol, Bisabolol, Glyceryl Laurate, Glyceryl Caprylate, Xylityl Sesquicaprylate, Phenylpropanol, Lauryl Alcohol, o-Cymen-5-ol, Tropolone, Climbazole, Piroctone Olamine, Octenidine HCl, Undecylenoyl Glycine, Sodium Caproyl/Lauroyl Lactylate, Ethyl Lauroyl Arginate Laurate, PCA Ethyl Cocoyl Arginate, Salvia Officinalis (Sage) Oil, Triclosan, Tetraselmis suecica extract, Mannitol, Sorbitol, Lactic Acid, Sodium Lauroyl Lactylate, Sodium Caproyl Lactylate, Salicylic Acid and its salts, Capryloyl Glycine, Ethyl Lauroyl Arginate HCl, Thymol, Jasmol, Gluconic Acid and salts thereof, Benzyl Salicylate, Propanediol Caprylate, 4-Hydroxyacetophenone, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more the aforesaid antimicrobial components; or
1,2-dodecanediol in combination with 2,3-dodecanediol plus one or more antimicrobial component(s) selected from the group consisting of Phenoxyethanol, 2-Methyl 5-cyclohexylpentanol, Dimethyl phenylbutanol, Dimethyl Phenylpropanol, 2-Benzylheptanol, Ethylhexylglycerin, Hydroxyacetophenone, Triethyl Citrate, Polyglyceryl-3 Caprylate, Polyglyceryl-10 Laurate, Glyceryl Caprylate, Benzyl Alcohol, Benzoic Acid, Sorbic Acid, Levulinic Acid and esters and/or ketals thereof, Anisic Acid and salts thereof, Polyglyceryl-2 Caprate, Caprylhydroxamic Acid, Zinc Ricinoleate, Farnesol, Bisabolol, Glyceryl Laurate, Glyceryl Caprylate, Xylityl Sesquicaprylate, Phenylpropanol, Lauryl Alcohol, o-Cymen-5-ol, Tropolone, Climbazole, Piroctone Olamine, Octenidine HCl, Undecylenoyl Glycine, Sodium Caproyl/Lauroyl Lactylate, Ethyl Lauroyl Arginate Laurate, PCA Ethyl Cocoyl Arginate, Salvia Officinalis (Sage) Oil, Triclosan, Tetraselmis suecica extract, Mannitol, Sorbitol, Lactic Acid, Sodium Lauroyl Lactylate, Sodium Caproyl Lactylate, Salicylic Acid and its salts, Capryloyl Glycine, Ethyl Lauroyl Arginate HCl, Thymol, Jasmol, Gluconic Acid and salts thereof, Benzyl Salicylate, Propanediol Caprylate, 4-Hydroxyacetophenone, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more the aforesaid antimicrobial components; or
1,2-tridecanediol in combination with 2,3-tridecanediol plus one or more antimicrobial component(s) selected from the group consisting of Phenoxyethanol, 2-Methyl 5-cyclohexylpentanol, Dimethyl phenylbutanol, Dimethyl Phenylpropanol, 2-Benzylheptanol, Ethylhexylglycerin, Hydroxyacetophenone, Triethyl Citrate, Polyglyceryl-3 Caprylate, Polyglyceryl-10 Laurate, Glyceryl Caprylate, Benzyl Alcohol, Benzoic Acid, Sorbic Acid, Levulinic Acid and esters and/or ketals thereof, Anisic Acid and salts thereof, Polyglyceryl-2 Caprate, Caprylhydroxamic Acid, Zinc Ricinoleate, Farnesol, Bisabolol, Glyceryl Laurate, Glyceryl Caprylate, Xylityl Sesquicaprylate, Phenylpropanol, Lauryl Alcohol, o-Cymen-5-ol, Tropolone, Climbazole, Piroctone Olamine, Octenidine HCl, Undecylenoyl Glycine, Sodium Caproyl/Lauroyl Lactylate, Ethyl Lauroyl Arginate Laurate, PCA Ethyl Cocoyl Arginate, Salvia Officinalis (Sage) Oil, Triclosan, Tetraselmis suecica extract, Mannitol, Sorbitol, Lactic Acid, Sodium Lauroyl Lactylate, Sodium Caproyl Lactylate, Salicylic Acid and its salts, Capryloyl Glycine, Ethyl Lauroyl Arginate HCl, Thymol, Jasmol, Gluconic Acid and salts thereof, Benzyl Salicylate, Propanediol Caprylate, 4-Hydroxyacetophenone, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more the aforesaid antimicrobial components; or
1,2-heptanediol in combination with one or more of 1,2-pentanediol, 1,2-hexanediol, 1,2-octanediol, 1,2-nonanediol, 1,2-decanediol, 1,2-undecanediol, 1,2-dodecanediol or 1,2-tridecanediol, plus one or more antimicrobial component(s) selected from the group consisting of Phenoxyethanol, 2-Methyl 5-cyclohexylpentanol, Dimethyl phenylbutanol, Dimethyl Phenylpropanol, 2-Benzylheptanol, Ethylhexylglycerin, Hydroxyacetophenone, Triethyl Citrate, Polyglyceryl-3 Caprylate, Polyglyceryl-10 Laurate, Glyceryl Caprylate, Benzyl Alcohol, Benzoic Acid, Sorbic Acid, Levulinic Acid and esters and/or ketals thereof, Anisic Acid and salts thereof, Polyglyceryl-2 Caprate, Caprylhydroxamic Acid, Zinc Ricinoleate, Farnesol, Bisabolol, Glyceryl Laurate, Glyceryl Caprylate, Xylityl Sesquicaprylate, Phenylpropanol, Lauryl Alcohol, o-Cymen-5-ol, Tropolone, Climbazole, Piroctone Olamine, Octenidine HCl, Undecylenoyl Glycine, Sodium Caproyl/Lauroyl Lactylate, Ethyl Lauroyl Arginate Laurate, PCA Ethyl Cocoyl Arginate, Salvia Officinalis (Sage) Oil, Triclosan, Tetraselmis suecica extract, Mannitol, Sorbitol, Lactic Acid, Sodium Lauroyl Lactylate, Sodium Caproyl Lactylate, Salicylic Acid and its salts, Capryloyl Glycine, Ethyl Lauroyl Arginate HCl, Thymol, Jasmol, Gluconic Acid and salts thereof, Benzyl Salicylate, Propanediol Caprylate, 4-Hydroxyacetophenone, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more the aforesaid antimicrobial components; or
1,2-heptanediol in combination with one or more of 2,3-pentanediol, 2,3-hexanediol, 2,3-heptanediol, 2,3-octanediol, 2,3-nonanediol, 2,3-decanediol, 2,3-undecanediol, 2,3-dodecanediol, or 2,3-tridecanediol plus one or more antimicrobial component(s) selected from the group consisting of Phenoxyethanol, 2-Methyl 5-cyclohexylpentanol, Dimethyl phenylbutanol, Dimethyl Phenylpropanol, 2-Benzylheptanol, Ethylhexylglycerin, Hydroxyacetophenone, Triethyl Citrate, Polyglyceryl-3 Caprylate, Polyglyceryl-10 Laurate, Glyceryl Caprylate, Benzyl Alcohol, Benzoic Acid, Sorbic Acid, Levulinic Acid and esters and/or ketals thereof, Anisic Acid and salts thereof, Polyglyceryl-2 Caprate, Caprylhydroxamic Acid, Zinc Ricinoleate, Farnesol, Bisabolol, Glyceryl Laurate, Glyceryl Caprylate, Xylityl Sesquicaprylate, Phenylpropanol, Lauryl Alcohol, o-Cymen-5-ol, Tropolone, Climbazole, Piroctone Olamine, Octenidine HCl, Undecylenoyl Glycine, Sodium Caproyl/Lauroyl Lactylate, Ethyl Lauroyl Arginate Laurate, PCA Ethyl Cocoyl Arginate, Salvia Officinalis (Sage) Oil, Triclosan, Tetraselmis suecica extract, Mannitol, Sorbitol, Lactic Acid, Sodium Lauroyl Lactylate, Sodium Caproyl Lactylate, Salicylic Acid and its salts, Capryloyl Glycine, Ethyl Lauroyl Arginate HCl, Thymol, Jasmol, Gluconic Acid and salts thereof, Benzyl Salicylate, Propanediol Caprylate, 4-Hydroxyacetophenone, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more the aforesaid antimicrobial components; or
1,2-nonanediol in combination with one or more of 1,2-pentanediol, 1,2-hexanediol, 1,2-heptanediol, 1,2-octanediol, 1,2-decanediol, 1,2-undecanediol, 1,2-dodecanediol or 1,2-tridecanediol, plus one or more antimicrobial component(s) selected from the group consisting of Phenoxyethanol, 2-Methyl 5-cyclohexylpentanol, Dimethyl phenylbutanol, Dimethyl Phenylpropanol, 2-Benzylheptanol, Ethylhexylglycerin, Hydroxyacetophenone, Triethyl Citrate, Polyglyceryl-3 Caprylate, Polyglyceryl-10 Laurate, Glyceryl Caprylate, Benzyl Alcohol, Benzoic Acid, Sorbic Acid, Levulinic Acid and esters and/or ketals thereof, Anisic Acid and salts thereof, Polyglyceryl-2 Caprate, Caprylhydroxamic Acid, Zinc Ricinoleate, Farnesol, Bisabolol, Glyceryl Laurate, Glyceryl Caprylate, Xylityl Sesquicaprylate, Phenylpropanol, Lauryl Alcohol, o-Cymen-5-ol, Tropolone, Climbazole, Piroctone Olamine, Octenidine HCl, Undecylenoyl Glycine, Sodium Caproyl/Lauroyl Lactylate, Ethyl Lauroyl Arginate Laurate, PCA Ethyl Cocoyl Arginate, Salvia Officinalis (Sage) Oil, Triclosan, Tetraselmis suecica extract, Mannitol, Sorbitol, Lactic Acid, Sodium Lauroyl Lactylate, Sodium Caproyl Lactylate, Salicylic Acid and its salts, Capryloyl Glycine, Ethyl Lauroyl Arginate HCl, Thymol, Jasmol, Gluconic Acid and salts thereof, Benzyl Salicylate, Propanediol Caprylate, 4-Hydroxyacetophenone, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more the aforesaid antimicrobial components; or
1,2-nonanediol in combination with one or more of 2,3-pentanediol, 2,3-hexanediol, 2,3-heptanediol, 2,3-octanediol, 2,3-nonanediol, 2,3-decanediol, 2,3-undecanediol, 2,3-dodecanediol, or 2,3-tridecanediol plus one or more antimicrobial component(s) selected from the group consisting of Phenoxyethanol, 2-Methyl 5-cyclohexylpentanol, Dimethyl phenylbutanol, Dimethyl Phenylpropanol, 2-Benzylheptanol, Ethylhexylglycerin, Hydroxyacetophenone, Triethyl Citrate, Polyglyceryl-3 Caprylate, Polyglyceryl-10 Laurate, Glyceryl Caprylate, Benzyl Alcohol, Benzoic Acid, Sorbic Acid, Levulinic Acid and esters and/or ketals thereof, Anisic Acid and salts thereof, Polyglyceryl-2 Caprate, Caprylhydroxamic Acid, Zinc Ricinoleate, Farnesol, Bisabolol, Glyceryl Laurate, Glyceryl Caprylate, Xylityl Sesquicaprylate, Phenylpropanol, Lauryl Alcohol, o-Cymen-5-ol, Tropolone, Climbazole, Piroctone Olamine, Octenidine HCl, Undecylenoyl Glycine, Sodium Caproyl/Lauroyl Lactylate, Ethyl Lauroyl Arginate Laurate, PCA Ethyl Cocoyl Arginate, Salvia Officinalis (Sage) Oil, Triclosan, Tetraselmis suecica extract, Mannitol, Sorbitol, Lactic Acid, Sodium Lauroyl Lactylate, Sodium Caproyl Lactylate, Salicylic Acid and its salts, Capryloyl Glycine, Ethyl Lauroyl Arginate HCl, Thymol, Jasmol, Gluconic Acid and salts thereof, Benzyl Salicylate, Propanediol Caprylate, 4-Hydroxyacetophenone, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more the aforesaid antimicrobial components; or
1,2-undecanediol in combination with one or more of 1,2-pentanediol, 1,2-hexanediol, 1,2-heptanediol, 1,2-octanediol, 1,2-nonanediol, 1,2-decanediol, 1,2-dodecanediol or 1,2-tridecanediol, plus one or more antimicrobial component(s) selected from the group consisting of Phenoxyethanol, 2-Methyl 5-cyclohexylpentanol, Dimethyl phenylbutanol, Dimethyl Phenylpropanol, 2-Benzylheptanol, Ethylhexylglycerin, Hydroxyacetophenone, Triethyl Citrate, Polyglyceryl-3 Caprylate, Polyglyceryl-10 Laurate, Glyceryl Caprylate, Benzyl Alcohol, Benzoic Acid, Sorbic Acid, Levulinic Acid and esters and/or ketals thereof, Anisic Acid and salts thereof, Polyglyceryl-2 Caprate, Caprylhydroxamic Acid, Zinc Ricinoleate, Farnesol, Bisabolol, Glyceryl Laurate, Glyceryl Caprylate, Xylityl Sesquicaprylate, Phenylpropanol, Lauryl Alcohol, o-Cymen-5-ol, Tropolone, Climbazole, Piroctone Olamine, Octenidine HCl, Undecylenoyl Glycine, Sodium Caproyl/Lauroyl Lactylate, Ethyl Lauroyl Arginate Laurate, PCA Ethyl Cocoyl Arginate, Salvia Officinalis (Sage) Oil, Triclosan, Tetraselmis suecica extract, Mannitol, Sorbitol, Lactic Acid, Sodium Lauroyl Lactylate, Sodium Caproyl Lactylate, Salicylic Acid and its salts, Capryloyl Glycine, Ethyl Lauroyl Arginate HCl, Thymol, Jasmol, Gluconic Acid and salts thereof, Benzyl Salicylate, Propanediol Caprylate, 4-Hydroxyacetophenone, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more the aforesaid antimicrobial components; or
1,2-undecanediol in combination with one or more of 2,3-pentanediol, 2,3-hexanediol, 2,3-heptanediol, 2,3-octanediol, 2,3-nonanediol, 2,3-decanediol, 2,3-undecanediol, 2,3-dodecanediol, or 2,3-tridecanediol plus one or more antimicrobial component(s) selected from the group consisting of Phenoxyethanol, 2-Methyl 5-cyclohexylpentanol, Dimethyl phenylbutanol, Dimethyl Phenylpropanol, 2-Benzylheptanol, Ethylhexylglycerin, Hydroxyacetophenone, Triethyl Citrate, Polyglyceryl-3 Caprylate, Polyglyceryl-10 Laurate, Glyceryl Caprylate, Benzyl Alcohol, Benzoic Acid, Sorbic Acid, Levulinic Acid and esters and/or ketals thereof, Anisic Acid and salts thereof, Polyglyceryl-2 Caprate, Caprylhydroxamic Acid, Zinc Ricinoleate, Farnesol, Bisabolol, Glyceryl Laurate, Glyceryl Caprylate, Xylityl Sesquicaprylate, Phenylpropanol, Lauryl Alcohol, o-Cymen-5-ol, Tropolone, Climbazole, Piroctone Olamine, Octenidine HCl, Undecylenoyl Glycine, Sodium Caproyl/Lauroyl Lactylate, Ethyl Lauroyl Arginate Laurate, PCA Ethyl Cocoyl Arginate, Salvia Officinalis (Sage) Oil, Triclosan, Tetraselmis suecica extract, Mannitol, Sorbitol, Lactic Acid, Sodium Lauroyl Lactylate, Sodium Caproyl Lactylate, Salicylic Acid and its salts, Capryloyl Glycine, Ethyl Lauroyl Arginate HCl, Thymol, Jasmol, Gluconic Acid and salts thereof, Benzyl Salicylate, Propanediol Caprylate, 4-Hydroxyacetophenone, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more the aforesaid antimicrobial components; or
1,2-tridecanediol in combination with one or more of 1,2-pentanediol, 1,2-hexanediol, 1,2-heptanediol, 1,2-octanediol, 1,2-nonanediol, 1,2-decanediol, 1,2-undecanediol, or 1,2-dodecanediol, plus one or more antimicrobial component(s) selected from the group consisting of Phenoxyethanol, 2-Methyl 5-cyclohexylpentanol, Dimethyl phenylbutanol, Dimethyl Phenylpropanol, 2-Benzylheptanol, Ethylhexylglycerin, Hydroxyacetophenone, Triethyl Citrate, Polyglyceryl-3 Caprylate, Polyglyceryl-10 Laurate, Glyceryl Caprylate, Benzyl Alcohol, Benzoic Acid, Sorbic Acid, Levulinic Acid and esters and/or ketals thereof, Anisic Acid and salts thereof, Polyglyceryl-2 Caprate, Caprylhydroxamic Acid, Zinc Ricinoleate, Farnesol, Bisabolol, Glyceryl Laurate, Glyceryl Caprylate, Xylityl Sesquicaprylate, Phenylpropanol, Lauryl Alcohol, o-Cymen-5-ol, Tropolone, Climbazole, Piroctone Olamine, Octenidine HCl, Undecylenoyl Glycine, Sodium Caproyl/Lauroyl Lactylate, Ethyl Lauroyl Arginate Laurate, PCA Ethyl Cocoyl Arginate, Salvia Officinalis (Sage) Oil, Triclosan, Tetraselmis suecica extract, Mannitol, Sorbitol, Lactic Acid, Sodium Lauroyl Lactylate, Sodium Caproyl Lactylate, Salicylic Acid and its salts, Capryloyl Glycine, Ethyl Lauroyl Arginate HCl, Thymol, Jasmol, Gluconic Acid and salts thereof, Benzyl Salicylate, Propanediol Caprylate, 4-Hydroxyacetophenone, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more the aforesaid antimicrobial components; or
1,2-tridecanediol in combination with one or more of 2,3-pentanediol, 2,3-hexanediol, 2,3-heptanediol, 2,3-octanediol, 2,3-nonanediol, 2,3-decanediol, 2,3-undecanediol, 2,3-dodecanediol, or 2,3-tridecanediol plus one or more antimicrobial component(s) selected from the group consisting of Phenoxyethanol, 2-Methyl 5-cyclohexylpentanol, Dimethyl phenylbutanol, Dimethyl Phenylpropanol, 2-Benzylheptanol, Ethylhexylglycerin, Hydroxyacetophenone, Triethyl Citrate, Polyglyceryl-3 Caprylate, Polyglyceryl-10 Laurate, Glyceryl Caprylate, Benzyl Alcohol, Benzoic Acid, Sorbic Acid, Levulinic Acid and esters and/or ketals thereof, Anisic Acid and salts thereof, Polyglyceryl-2 Caprate, Caprylhydroxamic Acid, Zinc Ricinoleate, Farnesol, Bisabolol, Glyceryl Laurate, Glyceryl Caprylate, Xylityl Sesquicaprylate, Phenylpropanol, Lauryl Alcohol, o-Cymen-5-ol, Tropolone, Climbazole, Piroctone Olamine, Octenidine HCl, Undecylenoyl Glycine, Sodium Caproyl/Lauroyl Lactylate, Ethyl Lauroyl Arginate Laurate, PCA Ethyl Cocoyl Arginate, Salvia Officinalis (Sage) Oil, Triclosan, Tetraselmis suecica extract, Mannitol, Sorbitol, Lactic Acid, Sodium Lauroyl Lactylate, Sodium Caproyl Lactylate, Salicylic Acid and its salts, Capryloyl Glycine, Ethyl Lauroyl Arginate HCl, Thymol, Jasmol, Gluconic Acid and salts thereof, Benzyl Salicylate, Propanediol Caprylate, 4-Hydroxyacetophenone, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more the aforesaid antimicrobial components; or
1,2-hexanediol in combination with 2,3-octanediol plus one or more antimicrobial component(s) selected from the group consisting of Phenoxyethanol, 2-Methyl 5-cyclohexylpentanol, Dimethyl phenylbutanol, Dimethyl Phenylpropanol, 2-Benzylheptanol, Ethylhexylglycerin, Hydroxyacetophenone, Triethyl Citrate, Polyglyceryl-3 Caprylate, Polyglyceryl-10 Laurate, Glyceryl Caprylate, Benzyl Alcohol, Benzoic Acid, Sorbic Acid, Levulinic Acid and esters and/or ketals thereof, Anisic Acid and salts thereof, Polyglyceryl-2 Caprate, Caprylhydroxamic Acid, Zinc Ricinoleate, Farnesol, Bisabolol, Glyceryl Laurate, Glyceryl Caprylate, Xylityl Sesquicaprylate, Phenylpropanol, Lauryl Alcohol, o-Cymen-5-ol, Tropolone, Climbazole, Piroctone Olamine, Octenidine HCl, Undecylenoyl Glycine, Sodium Caproyl/Lauroyl Lactylate, Ethyl Lauroyl Arginate Laurate, PCA Ethyl Cocoyl Arginate, Salvia Officinalis (Sage) Oil, Triclosan, Tetraselmis suecica extract, Mannitol, Sorbitol, Lactic Acid, Sodium Lauroyl Lactylate, Sodium Caproyl Lactylate, Salicylic Acid and its salts, Capryloyl Glycine, Ethyl Lauroyl Arginate HCl, Thymol, Jasmol, Gluconic Acid and salts thereof, Benzyl Salicylate, Propanediol Caprylate, 4-Hydroxyacetophenone, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more the aforesaid antimicrobial components; or
1,2-octanediol in combination with 2,3-hexanediol plus one or more antimicrobial component(s) selected from the group consisting of Phenoxyethanol, 2-Methyl 5-cyclohexylpentanol, Dimethyl phenylbutanol, Dimethyl Phenylpropanol, 2-Benzylheptanol, Ethylhexylglycerin, Hydroxyacetophenone, Triethyl Citrate, Polyglyceryl-3 Caprylate, Polyglyceryl-10 Laurate, Glyceryl Caprylate, Benzyl Alcohol, Benzoic Acid, Sorbic Acid, Levulinic Acid and esters and/or ketals thereof, Anisic Acid and salts thereof, Polyglyceryl-2 Caprate, Caprylhydroxamic Acid, Zinc Ricinoleate, Farnesol, Bisabolol, Glyceryl Laurate, Glyceryl Caprylate, Xylityl Sesquicaprylate, Phenylpropanol, Lauryl Alcohol, o-Cymen-5-ol, Tropolone, Climbazole, Piroctone Olamine, Octenidine HCl, Undecylenoyl Glycine, Sodium Caproyl/Lauroyl Lactylate, Ethyl Lauroyl Arginate Laurate, PCA Ethyl Cocoyl Arginate, Salvia Officinalis (Sage) Oil, Triclosan, Tetraselmis suecica extract, Mannitol, Sorbitol, Lactic Acid, Sodium Lauroyl Lactylate, Sodium Caproyl Lactylate, Salicylic Acid and its salts, Capryloyl Glycine, Ethyl Lauroyl Arginate HCl, Thymol, Jasmol, Gluconic Acid and salts thereof, Benzyl Salicylate, Propanediol Caprylate, 4-Hydroxyacetophenone, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more the aforesaid antimicrobial components; or
1,2-octanediol in combination with 2,3-heptanediol plus one or more antimicrobial component(s) selected from the group consisting of Phenoxyethanol, 2-Methyl 5-cyclohexylpentanol, Dimethyl phenylbutanol, Dimethyl Phenylpropanol, 2-Benzylheptanol, Ethylhexylglycerin, Hydroxyacetophenone, Triethyl Citrate, Polyglyceryl-3 Caprylate, Polyglyceryl-10 Laurate, Glyceryl Caprylate, Benzyl Alcohol, Benzoic Acid, Sorbic Acid, Levulinic Acid and esters and/or ketals thereof, Anisic Acid and salts thereof, Polyglyceryl-2 Caprate, Caprylhydroxamic Acid, Zinc Ricinoleate, Farnesol, Bisabolol, Glyceryl Laurate, Glyceryl Caprylate, Xylityl Sesquicaprylate, Phenylpropanol, Lauryl Alcohol, o-Cymen-5-ol, Tropolone, Climbazole, Piroctone Olamine, Octenidine HCl, Undecylenoyl Glycine, Sodium Caproyl/Lauroyl Lactylate, Ethyl Lauroyl Arginate Laurate, PCA Ethyl Cocoyl Arginate, Salvia Officinalis (Sage) Oil, Triclosan, Tetraselmis suecica extract, Mannitol, Sorbitol, Lactic Acid, Sodium Lauroyl Lactylate, Sodium Caproyl Lactylate, Salicylic Acid and its salts, Capryloyl Glycine, Ethyl Lauroyl Arginate HCl, Thymol, Jasmol, Gluconic Acid and salts thereof, Benzyl Salicylate, Propanediol Caprylate, 4-Hydroxyacetophenone, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more the aforesaid antimicrobial components; or
1,2-pentanediol in combination with 2,3-hexanediol plus one or more antimicrobial component(s) selected from the group consisting of Phenoxyethanol, 2-Methyl 5-cyclohexylpentanol, Dimethyl phenylbutanol, Dimethyl Phenylpropanol, 2-Benzylheptanol, Ethylhexylglycerin, Hydroxyacetophenone, Triethyl Citrate, Polyglyceryl-3 Caprylate, Polyglyceryl-10 Laurate, Glyceryl Caprylate, Benzyl Alcohol, Benzoic Acid, Sorbic Acid, Levulinic Acid and esters and/or ketals thereof, Anisic Acid and salts thereof, Polyglyceryl-2 Caprate, Caprylhydroxamic Acid, Zinc Ricinoleate, Farnesol, Bisabolol, Glyceryl Laurate, Glyceryl Caprylate, Xylityl Sesquicaprylate, Phenylpropanol, Lauryl Alcohol, o-Cymen-5-ol, Tropolone, Climbazole, Piroctone Olamine, Octenidine HCl, Undecylenoyl Glycine, Sodium Caproyl/Lauroyl Lactylate, Ethyl Lauroyl Arginate Laurate, PCA Ethyl Cocoyl Arginate, Salvia Officinalis (Sage) Oil, Triclosan, Tetraselmis suecica extract, Mannitol, Sorbitol, Lactic Acid, Sodium Lauroyl Lactylate, Sodium Caproyl Lactylate, Salicylic Acid and its salts, Capryloyl Glycine, Ethyl Lauroyl Arginate HCl, Thymol, Jasmol, Gluconic Acid and salts thereof, Benzyl Salicylate, Propanediol Caprylate, 4-Hydroxyacetophenone, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more the aforesaid antimicrobial components; or
1,2-pentanediol in combination with 1,2-heptanediol plus one or more antimicrobial component(s) selected from the group consisting of Phenoxyethanol, 2-Methyl 5-cyclohexylpentanol, Dimethyl phenylbutanol, Dimethyl Phenylpropanol, 2-Benzylheptanol, Ethylhexylglycerin, Hydroxyacetophenone, Triethyl Citrate, Polyglyceryl-3 Caprylate, Polyglyceryl-10 Laurate, Glyceryl Caprylate, Benzyl Alcohol, Benzoic Acid, Sorbic Acid, Levulinic Acid and esters and/or ketals thereof, Anisic Acid and salts thereof, Polyglyceryl-2 Caprate, Caprylhydroxamic Acid, Zinc Ricinoleate, Farnesol, Bisabolol, Glyceryl Laurate, Glyceryl Caprylate, Xylityl Sesquicaprylate, Phenylpropanol, Lauryl Alcohol, o-Cymen-5-ol, Tropolone, Climbazole, Piroctone Olamine, Octenidine HCl, Undecylenoyl Glycine, Sodium Caproyl/Lauroyl Lactylate, Ethyl Lauroyl Arginate Laurate, PCA Ethyl Cocoyl Arginate, Salvia Officinalis (Sage) Oil, Triclosan, Tetraselmis suecica extract, Mannitol, Sorbitol, Lactic Acid, Sodium Lauroyl Lactylate, Sodium Caproyl Lactylate, Salicylic Acid and its salts, Capryloyl Glycine, Ethyl Lauroyl Arginate HCl, Thymol, Jasmol, Gluconic Acid and salts thereof, Benzyl Salicylate, Propanediol Caprylate, 4-Hydroxyacetophenone, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more the aforesaid antimicrobial components; or
1,2-pentanediol in combination with 2,3-heptanediol plus one or more antimicrobial component(s) selected from the group consisting of Phenoxyethanol, 2-Methyl 5-cyclohexylpentanol, Dimethyl phenylbutanol, Dimethyl Phenylpropanol, 2-Benzylheptanol, Ethylhexylglycerin, Hydroxyacetophenone, Triethyl Citrate, Polyglyceryl-3 Caprylate, Polyglyceryl-10 Laurate, Glyceryl Caprylate, Benzyl Alcohol, Benzoic Acid, Sorbic Acid, Levulinic Acid and esters and/or ketals thereof, Anisic Acid and salts thereof, Polyglyceryl-2 Caprate, Caprylhydroxamic Acid, Zinc Ricinoleate, Farnesol, Bisabolol, Glyceryl Laurate, Glyceryl Caprylate, Xylityl Sesquicaprylate, Phenylpropanol, Lauryl Alcohol, o-Cymen-5-ol, Tropolone, Climbazole, Piroctone Olamine, Octenidine HCl, Undecylenoyl Glycine, Sodium Caproyl/Lauroyl Lactylate, Ethyl Lauroyl Arginate Laurate, PCA Ethyl Cocoyl Arginate, Salvia Officinalis (Sage) Oil, Triclosan, Tetraselmis suecica extract, Mannitol, Sorbitol, Lactic Acid, Sodium Lauroyl Lactylate, Sodium Caproyl Lactylate, Salicylic Acid and its salts, Capryloyl Glycine, Ethyl Lauroyl Arginate HCl, Thymol, Jasmol, Gluconic Acid and salts thereof, Benzyl Salicylate, Propanediol Caprylate, 4-Hydroxyacetophenone, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more the aforesaid antimicrobial components; or
1,2-pentanediol in combination with 2,3-octanediol plus one or more antimicrobial component(s) selected from the group consisting of Phenoxyethanol, 2-Methyl 5-cyclohexylpentanol, Dimethyl phenylbutanol, Dimethyl Phenylpropanol, 2-Benzylheptanol, Ethylhexylglycerin, Hydroxyacetophenone, Triethyl Citrate, Polyglyceryl-3 Caprylate, Polyglyceryl-10 Laurate, Glyceryl Caprylate, Benzyl Alcohol, Benzoic Acid, Sorbic Acid, Levulinic Acid and esters and/or ketals thereof, Anisic Acid and salts thereof, Polyglyceryl-2 Caprate, Caprylhydroxamic Acid, Zinc Ricinoleate, Farnesol, Bisabolol, Glyceryl Laurate, Glyceryl Caprylate, Xylityl Sesquicaprylate, Phenylpropanol, Lauryl Alcohol, o-Cymen-5-ol, Tropolone, Climbazole, Piroctone Olamine, Octenidine HCl, Undecylenoyl Glycine, Sodium Caproyl/Lauroyl Lactylate, Ethyl Lauroyl Arginate Laurate, PCA Ethyl Cocoyl Arginate, Salvia Officinalis (Sage) Oil, Triclosan, Tetraselmis suecica extract, Mannitol, Sorbitol, Lactic Acid, Sodium Lauroyl Lactylate, Sodium Caproyl Lactylate, Salicylic Acid and its salts, Capryloyl Glycine, Ethyl Lauroyl Arginate HCl, Thymol, Jasmol, Gluconic Acid and salts thereof, Benzyl Salicylate, Propanediol Caprylate, 4-Hydroxyacetophenone, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more the aforesaid antimicrobial components; or
1,2-pentanediol in combination with 1,2-nonanediol plus one or more antimicrobial component(s) selected from the group consisting of Phenoxyethanol, 2-Methyl 5-cyclohexylpentanol, Dimethyl phenylbutanol, Dimethyl Phenylpropanol, 2-Benzylheptanol, Ethylhexylglycerin, Hydroxyacetophenone, Triethyl Citrate, Polyglyceryl-3 Caprylate, Polyglyceryl-10 Laurate, Glyceryl Caprylate, Benzyl Alcohol, Benzoic Acid, Sorbic Acid, Levulinic Acid and esters and/or ketals thereof, Anisic Acid and salts thereof, Polyglyceryl-2 Caprate, Caprylhydroxamic Acid, Zinc Ricinoleate, Farnesol, Bisabolol, Glyceryl Laurate, Glyceryl Caprylate, Xylityl Sesquicaprylate, Phenylpropanol, Lauryl Alcohol, o-Cymen-5-ol, Tropolone, Climbazole, Piroctone Olamine, Octenidine HCl, Undecylenoyl Glycine, Sodium Caproyl/Lauroyl Lactylate, Ethyl Lauroyl Arginate Laurate, PCA Ethyl Cocoyl Arginate, Salvia Officinalis (Sage) Oil, Triclosan, Tetraselmis suecica extract, Mannitol, Sorbitol, Lactic Acid, Sodium Lauroyl Lactylate, Sodium Caproyl Lactylate, Salicylic Acid and its salts, Capryloyl Glycine, Ethyl Lauroyl Arginate HCl, Thymol, Jasmol, Gluconic Acid and salts thereof, Benzyl Salicylate, Propanediol Caprylate, 4-Hydroxyacetophenone, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more the aforesaid antimicrobial components; or
1,2-pentanediol in combination with 2,3-nonanediol plus one or more antimicrobial component(s) selected from the group consisting of Phenoxyethanol, 2-Methyl 5-cyclohexylpentanol, Dimethyl phenylbutanol, Dimethyl Phenylpropanol, 2-Benzylheptanol, Ethylhexylglycerin, Hydroxyacetophenone, Triethyl Citrate, Polyglyceryl-3 Caprylate, Polyglyceryl-10 Laurate, Glyceryl Caprylate, Benzyl Alcohol, Benzoic Acid, Sorbic Acid, Levulinic Acid and esters and/or ketals thereof, Anisic Acid and salts thereof, Polyglyceryl-2 Caprate, Caprylhydroxamic Acid, Zinc Ricinoleate, Farnesol, Bisabolol, Glyceryl Laurate, Glyceryl Caprylate, Xylityl Sesquicaprylate, Phenylpropanol, Lauryl Alcohol, o-Cymen-5-ol, Tropolone, Climbazole, Piroctone Olamine, Octenidine HCl, Undecylenoyl Glycine, Sodium Caproyl/Lauroyl Lactylate, Ethyl Lauroyl Arginate Laurate, PCA Ethyl Cocoyl Arginate, Salvia Officinalis (Sage) Oil, Triclosan, Tetraselmis suecica extract, Mannitol, Sorbitol, Lactic Acid, Sodium Lauroyl Lactylate, Sodium Caproyl Lactylate, Salicylic Acid and its salts, Capryloyl Glycine, Ethyl Lauroyl Arginate HCl, Thymol, Jasmol, Gluconic Acid and salts thereof, Benzyl Salicylate, Propanediol Caprylate, 4-Hydroxyacetophenone, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more the aforesaid antimicrobial components; or
1,2-heptanediol in combination with 1,2-octanediol plus one or more antimicrobial component(s) selected from the group consisting of Phenoxyethanol, 2-Methyl 5-cyclohexylpentanol, Dimethyl phenylbutanol, Dimethyl Phenylpropanol, 2-Benzylheptanol, Ethylhexylglycerin, Hydroxyacetophenone, Triethyl Citrate, Polyglyceryl-3 Caprylate, Polyglyceryl-10 Laurate, Glyceryl Caprylate, Benzyl Alcohol, Benzoic Acid, Sorbic Acid, Levulinic Acid and esters and/or ketals thereof, Anisic Acid and salts thereof, Polyglyceryl-2 Caprate, Caprylhydroxamic Acid, Zinc Ricinoleate, Farnesol, Bisabolol, Glyceryl Laurate, Glyceryl Caprylate, Xylityl Sesquicaprylate, Phenylpropanol, Lauryl Alcohol, o-Cymen-5-ol, Tropolone, Climbazole, Piroctone Olamine, Octenidine HCl, Undecylenoyl Glycine, Sodium Caproyl/Lauroyl Lactylate, Ethyl Lauroyl Arginate Laurate, PCA Ethyl Cocoyl Arginate, Salvia Officinalis (Sage) Oil, Triclosan, Tetraselmis suecica extract, Mannitol, Sorbitol, Lactic Acid, Sodium Lauroyl Lactylate, Sodium Caproyl Lactylate, Salicylic Acid and its salts, Capryloyl Glycine, Ethyl Lauroyl Arginate HCl, Thymol, Jasmol, Gluconic Acid and salts thereof, Benzyl Salicylate, Propanediol Caprylate, 4-Hydroxyacetophenone, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more the aforesaid antimicrobial components; or
1,2-heptanediol in combination with 2,3-octanediol plus one or more antimicrobial component(s) selected from the group consisting of Phenoxyethanol, 2-Methyl 5-cyclohexylpentanol, Dimethyl phenylbutanol, Dimethyl Phenylpropanol, 2-Benzylheptanol, Ethylhexylglycerin, Hydroxyacetophenone, Triethyl Citrate, Polyglyceryl-3 Caprylate, Polyglyceryl-10 Laurate, Glyceryl Caprylate, Benzyl Alcohol, Benzoic Acid, Sorbic Acid, Levulinic Acid and esters and/or ketals thereof, Anisic Acid and salts thereof, Polyglyceryl-2 Caprate, Caprylhydroxamic Acid, Zinc Ricinoleate, Farnesol, Bisabolol, Glyceryl Laurate, Glyceryl Caprylate, Xylityl Sesquicaprylate, Phenylpropanol, Lauryl Alcohol, o-Cymen-5-ol, Tropolone, Climbazole, Piroctone Olamine, Octenidine HCl, Undecylenoyl Glycine, Sodium Caproyl/Lauroyl Lactylate, Ethyl Lauroyl Arginate Laurate, PCA Ethyl Cocoyl Arginate, Salvia Officinalis (Sage) Oil, Triclosan, Tetraselmis suecica extract, Mannitol, Sorbitol, Lactic Acid, Sodium Lauroyl Lactylate, Sodium Caproyl Lactylate, Salicylic Acid and its salts, Capryloyl Glycine, Ethyl Lauroyl Arginate HCl, Thymol, Jasmol, Gluconic Acid and salts thereof, Benzyl Salicylate, Propanediol Caprylate, 4-Hydroxyacetophenone, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more the aforesaid antimicrobial components; or
1,2-heptanediol in combination with 1,2-nonanediol plus one or more antimicrobial component(s) selected from the group consisting of Phenoxyethanol, 2-Methyl 5-cyclohexylpentanol, Dimethyl phenylbutanol, Dimethyl Phenylpropanol, 2-Benzylheptanol, Ethylhexylglycerin, Hydroxyacetophenone, Triethyl Citrate, Polyglyceryl-3 Caprylate, Polyglyceryl-10 Laurate, Glyceryl Caprylate, Benzyl Alcohol, Benzoic Acid, Sorbic Acid, Levulinic Acid and esters and/or ketals thereof, Anisic Acid and salts thereof, Polyglyceryl-2 Caprate, Caprylhydroxamic Acid, Zinc Ricinoleate, Farnesol, Bisabolol, Glyceryl Laurate, Glyceryl Caprylate, Xylityl Sesquicaprylate, Phenylpropanol, Lauryl Alcohol, o-Cymen-5-ol, Tropolone, Climbazole, Piroctone Olamine, Octenidine HCl, Undecylenoyl Glycine, Sodium Caproyl/Lauroyl Lactylate, Ethyl Lauroyl Arginate Laurate, PCA Ethyl Cocoyl Arginate, Salvia Officinalis (Sage) Oil, Triclosan, Tetraselmis suecica extract, Mannitol, Sorbitol, Lactic Acid, Sodium Lauroyl Lactylate, Sodium Caproyl Lactylate, Salicylic Acid and its salts, Capryloyl Glycine, Ethyl Lauroyl Arginate HCl, Thymol, Jasmol, Gluconic Acid and salts thereof, Benzyl Salicylate, Propanediol Caprylate, 4-Hydroxyacetophenone, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more the aforesaid antimicrobial components; or
1,2-heptanediol in combination with 2,3-nonanediol plus one or more antimicrobial component(s) selected from the group consisting of Phenoxyethanol, 2-Methyl 5-cyclohexylpentanol, Dimethyl phenylbutanol, Dimethyl Phenylpropanol, 2-Benzylheptanol, Ethylhexylglycerin, Hydroxyacetophenone, Triethyl Citrate, Polyglyceryl-3 Caprylate, Polyglyceryl-10 Laurate, Glyceryl Caprylate, Benzyl Alcohol, Benzoic Acid, Sorbic Acid, Levulinic Acid and esters and/or ketals thereof, Anisic Acid and salts thereof, Polyglyceryl-2 Caprate, Caprylhydroxamic Acid, Zinc Ricinoleate, Farnesol, Bisabolol, Glyceryl Laurate, Glyceryl Caprylate, Xylityl Sesquicaprylate, Phenylpropanol, Lauryl Alcohol, o-Cymen-5-ol, Tropolone, Climbazole, Piroctone Olamine, Octenidine HCl, Undecylenoyl Glycine, Sodium Caproyl/Lauroyl Lactylate, Ethyl Lauroyl Arginate Laurate, PCA Ethyl Cocoyl Arginate, Salvia Officinalis (Sage) Oil, Triclosan, Tetraselmis suecica extract, Mannitol, Sorbitol, Lactic Acid, Sodium Lauroyl Lactylate, Sodium Caproyl Lactylate, Salicylic Acid and its salts, Capryloyl Glycine, Ethyl Lauroyl Arginate HCl, Thymol, Jasmol, Gluconic Acid and salts thereof, Benzyl Salicylate, Propanediol Caprylate, 4-Hydroxyacetophenone, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more the aforesaid antimicrobial components.

More preferred combinations of components (a) and (b) in the cosmetic or pharmaceutical composition according to the first or second aspect of the present invention are:
1,2-pentanediol plus one or more antimicrobial component(s) selected from the group consisting of Ethylhexylglycerin, Glyceryl Caprylate, Phenoxyethanol, Caprylhydroxamic Acid, Farnesol, Climbazole, Triethyl Citrate, Benzyl Alcohol, o-Cymen-5-ol, Salicylic Acid and its salts, Benzyl Salicylate, Propanediol Caprylate, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more of the aforesaid antimicrobial components; or
1,2-hexanediol plus one or more antimicrobial component(s) selected from the group consisting of Ethylhexylglycerin, Glyceryl Caprylate, Phenoxyethanol, Caprylhydroxamic Acid, Farnesol, Climbazole, Triethyl Citrate, Benzyl Alcohol, o-Cymen-5-ol, Salicylic Acid and its salts, Benzyl Salicylate, Propanediol Caprylate, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more of the aforesaid antimicrobial components; or
1,2-heptanediol plus one or more antimicrobial component(s) selected from the group consisting of Ethylhexylglycerin, Glyceryl Caprylate, Phenoxyethanol, Caprylhydroxamic Acid, Farnesol, Climbazole, Triethyl Citrate, Benzyl Alcohol, o-Cymen-5-ol, Salicylic Acid and its salts, Benzyl Salicylate, Propanediol Caprylate, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more of the aforesaid antimicrobial components; or
1,2-octanediol plus one or more antimicrobial component(s) selected from the group consisting of Ethylhexylglycerin, Glyceryl Caprylate, Phenoxyethanol, Caprylhydroxamic Acid, Farnesol, Climbazole, Triethyl Citrate, Benzyl Alcohol, o-Cymen-5-ol, Salicylic Acid and its salts, Benzyl Salicylate, Propanediol Caprylate, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more of the aforesaid antimicrobial components; or
1,2-nonanediol plus one or more antimicrobial component(s) selected from the group consisting of Ethylhexylglycerin, Glyceryl Caprylate, Phenoxyethanol, Caprylhydroxamic Acid, Farnesol, Climbazole, Triethyl Citrate, Benzyl Alcohol, o-Cymen-5-ol, Salicylic Acid and its salts, Benzyl Salicylate, Propanediol Caprylate, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more of the aforesaid antimicrobial components; or
1,2-decanediol plus one or more antimicrobial component(s) selected from the group consisting of Ethylhexylglycerin, Glyceryl Caprylate, Phenoxyethanol, Caprylhydroxamic Acid, Farnesol, Climbazole, Triethyl Citrate, Benzyl Alcohol, o-Cymen-5-ol, Salicylic Acid and its salts, Benzyl Salicylate, Propanediol Caprylate, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more of the aforesaid antimicrobial components; or
1,2-undecanediol plus one or more antimicrobial component(s) selected from the group consisting of Ethylhexylglycerin, Glyceryl Caprylate, Phenoxyethanol, Caprylhydroxamic Acid, Farnesol, Climbazole, Triethyl Citrate, Benzyl Alcohol, o-Cymen-5-ol, Salicylic Acid and its salts, Benzyl Salicylate, Propanediol Caprylate, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more of the aforesaid antimicrobial components; or
1,2-dodecanediol plus one or more antimicrobial component(s) selected from the group consisting of Ethylhexylglycerin, Glyceryl Caprylate, Phenoxyethanol, Caprylhydroxamic Acid, Farnesol, Climbazole, Triethyl Citrate, Benzyl Alcohol, o-Cymen-5-ol, Salicylic Acid and its salts, Benzyl Salicylate, Propanediol Caprylate, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more of the aforesaid antimicrobial components; or
1,2-tridecanediol plus one or more antimicrobial component(s) selected from the group consisting of Ethylhexylglycerin, Glyceryl Caprylate, Phenoxyethanol, Caprylhydroxamic Acid, Farnesol, Climbazole, Triethyl Citrate, Benzyl Alcohol, o-Cymen-5-ol, Salicylic Acid and its salts, Benzyl Salicylate, Propanediol Caprylate, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more of the aforesaid antimicrobial components; or
2,3-pentanediol plus one or more antimicrobial component(s) selected from the group consisting of Ethylhexylglycerin, Glyceryl Caprylate, Phenoxyethanol, Caprylhydroxamic Acid, Farnesol, Climbazole, Triethyl Citrate, Benzyl Alcohol, o-Cymen-5-ol, Salicylic Acid and its salts, Benzyl Salicylate, Propanediol Caprylate, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more of the aforesaid antimicrobial components; or
2,3-hexanediol plus one or more antimicrobial component(s) selected from the group consisting of Ethylhexylglycerin, Glyceryl Caprylate, Phenoxyethanol, Caprylhydroxamic Acid, Farnesol, Climbazole, Triethyl Citrate, Benzyl Alcohol, o-Cymen-5-ol, Salicylic Acid and its salts, Benzyl Salicylate, Propanediol Caprylate, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more of the aforesaid antimicrobial components; or
2,3-heptanediol plus one or more antimicrobial component(s) selected from the group consisting of Ethylhexylglycerin, Glyceryl Caprylate, Phenoxyethanol, Caprylhydroxamic Acid, Farnesol, Climbazole, Triethyl Citrate, Benzyl Alcohol, o-Cymen-5-ol, Salicylic Acid and its salts, Benzyl Salicylate, Propanediol Caprylate, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more of the aforesaid antimicrobial components; or
2,3-octanediol plus one or more antimicrobial component(s) selected from the group consisting of Ethylhexylglycerin, Glyceryl Caprylate, Phenoxyethanol, Caprylhydroxamic Acid, Farnesol, Climbazole, Triethyl Citrate, Benzyl Alcohol, o-Cymen-5-ol, Salicylic Acid and its salts, Benzyl Salicylate, Propanediol Caprylate, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more of the aforesaid antimicrobial components; or
2,3-nonanediol plus one or more antimicrobial component(s) selected from the group consisting of Ethylhexylglycerin, Glyceryl Caprylate, Phenoxyethanol, Caprylhydroxamic Acid, Farnesol, Climbazole, Triethyl Citrate, Benzyl Alcohol, o-Cymen-5-ol, Salicylic Acid and its salts, Benzyl Salicylate, Propanediol Caprylate, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more of the aforesaid antimicrobial components; or
2,3-decanediol plus one or more antimicrobial component(s) selected from the group consisting of Ethylhexylglycerin, Glyceryl Caprylate, Phenoxyethanol, Caprylhydroxamic Acid, Farnesol, Climbazole, Triethyl Citrate, Benzyl Alcohol, o-Cymen-5-ol, Salicylic Acid and its salts, Benzyl Salicylate, Propanediol Caprylate, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more of the aforesaid antimicrobial components; or
2,3-undecanediol plus one or more antimicrobial component(s) selected from the group consisting of Ethylhexylglycerin, Glyceryl Caprylate, Phenoxyethanol, Caprylhydroxamic Acid, Farnesol, Climbazole, Triethyl Citrate, Benzyl Alcohol, o-Cymen-5-ol, Salicylic Acid and its salts, Benzyl Salicylate, Propanediol Caprylate, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more of the aforesaid antimicrobial components; or
2,3-dodecanediol plus one or more antimicrobial component(s) selected from the group consisting of Ethylhexylglycerin, Glyceryl Caprylate, Phenoxyethanol, Caprylhydroxamic Acid, Farnesol, Climbazole, Triethyl Citrate, Benzyl Alcohol, o-Cymen-5-ol, Salicylic Acid and its salts, Benzyl Salicylate, Propanediol Caprylate, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more of the aforesaid antimicrobial components; or
2,3-tridecanediol plus one or more antimicrobial component(s) selected from the group consisting of Ethylhexylglycerin, Glyceryl Caprylate, Phenoxyethanol, Caprylhydroxamic Acid, Farnesol, Climbazole, Triethyl Citrate, Benzyl Alcohol, o-Cymen-5-ol, Salicylic Acid and its salts, Benzyl Salicylate, Propanediol Caprylate, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more of the aforesaid antimicrobial components; or
1,2-pentanediol in combination with 2,3-pentanediol plus one or more antimicrobial component(s) selected from the group consisting of Ethylhexylglycerin, Glyceryl Caprylate, Phenoxyethanol, Caprylhydroxamic Acid, Farnesol, Climbazole, Triethyl Citrate, Benzyl Alcohol, o-Cymen-5-ol, Salicylic Acid and its salts, Benzyl Salicylate, Propanediol Caprylate, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more of the aforesaid antimicrobial components; or
1,2-hexanediol in combination with 2,3-hexanediol plus one or more antimicrobial component(s) selected from the group consisting of Ethylhexylglycerin, Glyceryl Caprylate, Phenoxyethanol, Caprylhydroxamic Acid, Farnesol, Climbazole, Triethyl Citrate, Benzyl Alcohol, o-Cymen-5-ol, Salicylic Acid and its salts, Benzyl Salicylate, Propanediol Caprylate, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more of the aforesaid antimicrobial components; or
1,2-heptanediol in combination with 2,3-heptanediol plus one or more antimicrobial component(s) selected from the group consisting of Ethylhexylglycerin, Glyceryl Caprylate, Phenoxyethanol, Caprylhydroxamic Acid, Farnesol, Climbazole, Triethyl Citrate, Benzyl Alcohol, o-Cymen-5-ol, Salicylic Acid and its salts, Benzyl Salicylate, Propanediol Caprylate, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more of the aforesaid antimicrobial components; or
1,2-octanediol in combination with 2,3-octanediol plus one or more antimicrobial component(s) selected from the group consisting of Ethylhexylglycerin, Glyceryl Caprylate, Phenoxyethanol, Caprylhydroxamic Acid, Farnesol, Climbazole, Triethyl Citrate, Benzyl Alcohol, o-Cymen-5-ol, Salicylic Acid and its salts, Benzyl Salicylate, Propanediol Caprylate, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more of the aforesaid antimicrobial components; or
1,2-nonanediol in combination with 2,3-nonanediol plus one or more antimicrobial component(s) selected from the group consisting of Ethylhexylglycerin, Glyceryl Caprylate, Phenoxyethanol, Caprylhydroxamic Acid, Farnesol, Climbazole, Triethyl Citrate, Benzyl Alcohol, o-Cymen-5-ol, Salicylic Acid and its salts, Benzyl Salicylate, Propanediol Caprylate, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more of the aforesaid antimicrobial components; or
1,2-decanediol in combination with 2,3-decanediol plus one or more antimicrobial component(s) selected from the group consisting of Ethylhexylglycerin, Glyceryl Caprylate, Phenoxyethanol, Caprylhydroxamic Acid, Farnesol, Climbazole, Triethyl Citrate, Benzyl Alcohol, o-Cymen-5-ol, Salicylic Acid and its salts, Benzyl Salicylate, Propanediol Caprylate, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more of the aforesaid antimicrobial components; or
1,2-undecanediol in combination with 2,3-undecanediol plus one or more antimicrobial component(s) selected from the group consisting of Ethylhexylglycerin, Glyceryl Caprylate, Phenoxyethanol, Caprylhydroxamic Acid, Farnesol, Climbazole, Triethyl Citrate, Benzyl Alcohol, o-Cymen-5-ol, Salicylic Acid and its salts, Benzyl Salicylate, Propanediol Caprylate, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more of the aforesaid antimicrobial components; or
1,2-dodecanediol in combination with 2,3-dodecanediol plus one or more antimicrobial component(s) selected from the group consisting of Ethylhexylglycerin, Glyceryl Caprylate, Phenoxyethanol, Caprylhydroxamic Acid, Farnesol, Climbazole, Triethyl Citrate, Benzyl Alcohol, o-Cymen-5-ol, Salicylic Acid and its salts, Benzyl Salicylate, Propanediol Caprylate, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more of the aforesaid antimicrobial components; or
1,2-tridecanediol in combination with 2,3-tridecanediol plus one or more antimicrobial component(s) selected from the group consisting of Ethylhexylglycerin, Glyceryl Caprylate, Phenoxyethanol, Caprylhydroxamic Acid, Farnesol, Climbazole, Triethyl Citrate, Benzyl Alcohol, o-Cymen-5-ol, Salicylic Acid and its salts, Benzyl Salicylate, Propanediol Caprylate, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more of the aforesaid antimicrobial components; or
1,2-heptanediol in combination with one or more of 1,2-pentanediol, 1,2-hexanediol, 1,2-octanediol, 1,2-nonanediol, 1,2-decanediol, 1,2-undecanediol, 1,2-dodecanediol or 1,2-tridecanediol, plus one or more antimicrobial component(s) selected from the group consisting of Ethylhexylglycerin, Glyceryl Caprylate, Phenoxyethanol, Caprylhydroxamic Acid, Farnesol, Climbazole, Triethyl Citrate, Benzyl Alcohol, o-Cymen-5-ol, Salicylic Acid and its salts, Benzyl Salicylate, Propanediol Caprylate, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more of the aforesaid antimicrobial components; or
1,2-heptanediol in combination with one or more of 2,3-pentanediol, 2,3-hexanediol, 2,3-heptanediol, 2,3-octanediol, 2,3-nonanediol, 2,3-decanediol, 2,3-undecanediol, 2,3-dodecanediol, or 2,3-tridecanediol plus one or more antimicrobial component(s) selected from the group consisting of Ethylhexylglycerin, Glyceryl Caprylate, Phenoxyethanol, Caprylhydroxamic Acid, Farnesol, Climbazole, Triethyl Citrate, Benzyl Alcohol, o-Cymen-5-ol, Salicylic Acid and its salts, Benzyl Salicylate, Propanediol Caprylate, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more of the aforesaid antimicrobial components; or
1,2-nonanediol in combination with one or more of 1,2-pentanediol, 1,2-hexanediol, 1,2-heptanediol, 1,2-octanediol, 1,2-decanediol, 1,2-undecanediol, 1,2-dodecanediol or 1,2-tridecanediol, plus one or more antimicrobial component(s) selected from the group consisting of Ethylhexylglycerin, Glyceryl Caprylate, Phenoxyethanol, Caprylhydroxamic Acid, Farnesol, Climbazole, Triethyl Citrate, Benzyl Alcohol, o-Cymen-5-ol, Salicylic Acid and its salts, Benzyl Salicylate, Propanediol Caprylate, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more of the aforesaid antimicrobial components; or
1,2-nonanediol in combination with one or more of 2,3-pentanediol, 2,3-hexanediol, 2,3-heptanediol, 2,3-octanediol, 2,3-nonanediol, 2,3-decanediol, 2,3-undecanediol, 2,3-dodecanediol, or 2,3-tridecanediol plus one or more antimicrobial component(s) selected from the group consisting of Ethylhexylglycerin, Glyceryl Caprylate, Phenoxyethanol, Caprylhydroxamic Acid, Farnesol, Climbazole, Triethyl Citrate, Benzyl Alcohol, o-Cymen-5-ol, Salicylic Acid and its salts, Benzyl Salicylate, Propanediol Caprylate, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more of the aforesaid antimicrobial components; or
1,2-undecanediol in combination with one or more of 1,2-pentanediol, 1,2-hexanediol, 1,2-heptanediol, 1,2-octanediol, 1,2-nonanediol, 1,2-decanediol, 1,2-dodecanediol or 1,2-tridecanediol, plus one or more antimicrobial component(s) selected from the group consisting of Ethylhexylglycerin, Glyceryl Caprylate, Phenoxyethanol, Caprylhydroxamic Acid, Farnesol, Climbazole, Triethyl Citrate, Benzyl Alcohol, o-Cymen-5-ol, Salicylic Acid and its salts, Benzyl Salicylate, Propanediol Caprylate, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more of the aforesaid antimicrobial components; or
1,2-undecanediol in combination with one or more of 2,3-pentanediol, 2,3-hexanediol, 2,3-heptanediol, 2,3-octanediol, 2,3-nonanediol, 2,3-decanediol, 2,3-undecanediol, 2,3-dodecanediol, or 2,3-tridecanediol plus one or more antimicrobial component(s) selected from the group consisting of Ethylhexylglycerin, Glyceryl Caprylate, Phenoxyethanol, Caprylhydroxamic Acid, Farnesol, Climbazole, Triethyl Citrate, Benzyl Alcohol, o-Cymen-5-ol, Salicylic Acid and its salts, Benzyl Salicylate, Propanediol Caprylate, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more of the aforesaid antimicrobial components; or
1,2-tridecanediol in combination with one or more of 1,2-pentanediol, 1,2-hexanediol, 1,2-heptanediol, 1,2-octanediol, 1,2-nonanediol, 1,2-decanediol, 1,2-undecanediol, or 1,2-dodecanediol, plus one or more antimicrobial component(s) selected from the group consisting of Ethylhexylglycerin, Glyceryl Caprylate, Phenoxyethanol, Caprylhydroxamic Acid, Farnesol, Climbazole, Triethyl Citrate, Benzyl Alcohol, o-Cymen-5-ol, Salicylic Acid and its salts, Benzyl Salicylate, Propanediol Caprylate, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more of the aforesaid antimicrobial components; or
1,2-tridecanediol in combination with one or more of 2,3-pentanediol, 2,3-hexanediol, 2,3-heptanediol, 2,3-octanediol, 2,3-nonanediol, 2,3-decanediol, 2,3-undecanediol, 2,3-dodecanediol, or 2,3-tridecanediol plus one or more antimicrobial component(s) selected from the group consisting of Ethylhexylglycerin, Glyceryl Caprylate, Phenoxyethanol, Caprylhydroxamic Acid, Farnesol, Climbazole, Triethyl Citrate, Benzyl Alcohol, o-Cymen-5-ol, Salicylic Acid and its salts, Benzyl Salicylate, Propanediol Caprylate, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more of the aforesaid antimicrobial components; or
1,2-hexanediol in combination with 2,3-octanediol plus one or more antimicrobial component(s) selected from the group consisting of Ethylhexylglycerin, Glyceryl Caprylate, Phenoxyethanol, Caprylhydroxamic Acid, Farnesol, Climbazole, Triethyl Citrate, Benzyl Alcohol, o-Cymen-5-ol, Salicylic Acid and its salts, Benzyl Salicylate, Propanediol Caprylate, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more of the aforesaid antimicrobial components; or
1,2-octanediol in combination with 2,3-hexanediol plus one or more antimicrobial component(s) selected from the group consisting of Ethylhexylglycerin, Glyceryl Caprylate, Phenoxyethanol, Caprylhydroxamic Acid, Farnesol, Climbazole, Triethyl Citrate, Benzyl Alcohol, o-Cymen-5-ol, Salicylic Acid and its salts, Benzyl Salicylate, Propanediol Caprylate, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more of the aforesaid antimicrobial components; or
1,2-octanediol in combination with 2,3-heptanediol plus one or more antimicrobial component(s) selected from the group consisting of Ethylhexylglycerin, Glyceryl Caprylate, Phenoxyethanol, Caprylhydroxamic Acid, Farnesol, Climbazole, Triethyl Citrate, Benzyl Alcohol, o-Cymen-5-ol, Salicylic Acid and its salts, Benzyl Salicylate, Propanediol Caprylate, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more of the aforesaid antimicrobial components; or
1,2-pentanediol in combination with 2,3-hexanediol plus one or more antimicrobial component(s) selected from the group consisting of Ethylhexylglycerin, Glyceryl Caprylate, Phenoxyethanol, Caprylhydroxamic Acid, Farnesol, Climbazole, Triethyl Citrate, Benzyl Alcohol, o-Cymen-5-ol, Salicylic Acid and its salts, Benzyl Salicylate, Propanediol Caprylate, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more of the aforesaid antimicrobial components; or
1,2-pentanediol in combination with 1,2-heptanediol plus one or more antimicrobial component(s) selected from the group consisting of Ethylhexylglycerin, Glyceryl Caprylate, Phenoxyethanol, Caprylhydroxamic Acid, Farnesol, Climbazole, Triethyl Citrate, Benzyl Alcohol, o-Cymen-5-ol, Salicylic Acid and its salts, Benzyl Salicylate, Propanediol Caprylate, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more of the aforesaid antimicrobial components; or
1,2-pentanediol in combination with 2,3-heptanediol plus one or more antimicrobial component(s) selected from the group consisting of Ethylhexylglycerin, Glyceryl Caprylate, Phenoxyethanol, Caprylhydroxamic Acid, Farnesol, Climbazole, Triethyl Citrate, Benzyl Alcohol, o-Cymen-5-ol, Salicylic Acid and its salts, Benzyl Salicylate, Propanediol Caprylate, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more of the aforesaid antimicrobial components; or
1,2-pentanediol in combination with 2,3-octanediol plus one or more antimicrobial component(s) selected from the group consisting of Ethylhexylglycerin, Glyceryl Caprylate, Phenoxyethanol, Caprylhydroxamic Acid, Farnesol, Climbazole, Triethyl Citrate, Benzyl Alcohol, o-Cymen-5-ol, Salicylic Acid and its salts, Benzyl Salicylate, Propanediol Caprylate, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more of the aforesaid antimicrobial components; or
1,2-pentanediol in combination with 1,2-nonanediol plus one or more antimicrobial component(s) selected from the group consisting of Ethylhexylglycerin, Glyceryl Caprylate, Phenoxyethanol, Caprylhydroxamic Acid, Farnesol, Climbazole, Triethyl Citrate, Benzyl Alcohol, o-Cymen-5-ol, Salicylic Acid and its salts, Benzyl Salicylate, Propanediol Caprylate, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more of the aforesaid antimicrobial components; or
1,2-pentanediol in combination with 2,3-nonanediol plus one or more antimicrobial component(s) selected from the group consisting of Ethylhexylglycerin, Glyceryl Caprylate, Phenoxyethanol, Caprylhydroxamic Acid, Farnesol, Climbazole, Triethyl Citrate, Benzyl Alcohol, o-Cymen-5-ol, Salicylic Acid and its salts, Benzyl Salicylate, Propanediol Caprylate, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more of the aforesaid antimicrobial components; or
1,2-heptanediol in combination with 1,2-octanediol plus one or more antimicrobial component(s) selected from the group consisting of Ethylhexylglycerin, Glyceryl Caprylate, Phenoxyethanol, Caprylhydroxamic Acid, Farnesol, Climbazole, Triethyl Citrate, Benzyl Alcohol, o-Cymen-5-ol, Salicylic Acid and its salts, Benzyl Salicylate, Propanediol Caprylate, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more of the aforesaid antimicrobial components; or
1,2-heptanediol in combination with 2,3-octanediol plus one or more antimicrobial component(s) selected from the group consisting of Ethylhexylglycerin, Glyceryl Caprylate, Phenoxyethanol, Caprylhydroxamic Acid, Farnesol, Climbazole, Triethyl Citrate, Benzyl Alcohol, o-Cymen-5-ol, Salicylic Acid and its salts, Benzyl Salicylate, Propanediol Caprylate, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more of the aforesaid antimicrobial components; or
1,2-heptanediol in combination with 1,2-nonanediol plus one or more antimicrobial component(s) selected from the group consisting of Ethylhexylglycerin, Glyceryl Caprylate, Phenoxyethanol, Caprylhydroxamic Acid, Farnesol, Climbazole, Triethyl Citrate, Benzyl Alcohol, o-Cymen-5-ol, Salicylic Acid and its salts, Benzyl Salicylate, Propanediol Caprylate, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more of the aforesaid antimicrobial components; or
1,2-heptanediol in combination with 2,3-nonanediol plus one or more antimicrobial component(s) selected from the group consisting of Ethylhexylglycerin, Glyceryl Caprylate, Phenoxyethanol, Caprylhydroxamic Acid, Farnesol, Climbazole, Triethyl Citrate, Benzyl Alcohol, o-Cymen-5-ol, Salicylic Acid and its salts, Benzyl Salicylate, Propanediol Caprylate, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more of the aforesaid antimicrobial components.

Most preferred combinations of components (a) and (b) in the cosmetic or pharmaceutical composition according to the first or second aspect of the present invention are:
1,2-nonanediol plus Ethylhexylglycerin; or
1,2-nonanediol plus 4-Hydroxyacetophenone; or
1,2-nonanediol plus Glyceryl Caprylate; or
1,2-nonanediol plus Phenoxyethanol; or
1,2-nonanediol plus Caprylhydroxamic Acid; or
1,2-nonanediol plus Farnesol; or
1,2-nonanediol plus Climbazole; or
1,2-nonanediol plus Triethyl Citrate; or
1,2-nonanediol plus Benzyl Alcohol; or
1,2-nonanediol plus o-Cymen-5-ol; or
1,2-nonanediol plus Salicylic Acid and its salts; or
1,2-nonanediol plus Benzyl Salicylate; or
1,2-nonanediol plus Propanediol Caprylate.

Of the above combinations, the combinations including 1,2-heptanediol, 1,2-octanediol, 1,2-nonanediol, 1,2-undecanediol, or 1,2-tridecanediol or combinations including 2,3-alkanediols, in particular 2,3-pentanediol, 2,3-hexanediol, 2,3-heptanediole, 2,3-octanediol, 2,3-nonanediol, 2,3-decanediol, 2,3-undecanediol, 2,3-dodencanediol or 2,3-tridecanediol, result in distinguished antimicrobial properties, i.e., the antimicrobial effect of the antimicrobial component in the ready-to-use composition on the skin or scalp can be boosted. In particular, with the concomitantly use of one or more of the above alkanediols, the antimicrobial effect of the antimicrobial component on skin is even synergistically enhanced. Concomitantly, the use of one or more of the above alkanediols reduces or inhibits sebum overproduction of the sebaceous glands of the skin or scalp and/or reduces or inhibits for example dandruff on skin, scalp or nails. This sebum reducing effect is particularly distinctive for the 2,3-alkanediols as specified before. This effect is demonstrated by the following examples.

In a preferred advantageously variant, the cosmetic or pharmaceutical composition according to the first or second aspect of the present invention comprises one of the following combinations of components (a) and (b), in which the antimicrobial component (b) is a 2,3-alkanediol:
1,2-pentanediol plus 2,3-pentanediol; or
1,2-hexanediol plus 2,3-hexanediol; or
1,2-heptanediol plus 2,3-heptanediol; or
1,2-octanediol plus 2,3-octanediol; or
1,2-nonanediol plus 2,3-nonanediol; or
1,2-decanediol plus 2,3-decanediol; or
1,2-undecanediol plus 2,3-undecanediol; or
1,2-dodecanediol plus 2,3-dodecanediol.

Such combinations have a particularly synergistic antimicrobial effect on the one hand and an enhanced sebum secretion inhibiting effect, as it is demonstrated in the following examples.

Since dermatological conditions or diseases are usually associated with dry skin, scratched skin, skin lesions or even inflammation, it is also possible and in some cases advantageous, to combine the cosmetic or pharmaceutical, in particular dermatological, composition according to the first or second aspect of the present invention with one or more other active agents. These agents can in some instances provide one or more than one additional benefit or effect or operate via more than one mode of action.

These active agents are preferably selected from the group of agents consisting of sebum reducing agents, anti-acne agents, anti-dandruff agents, anti-inflammatory and/or redness and/or itch ameliorating agents, odour absorbers and/or antiperspirant active agents, lenitive substances, moisturising and/or moisture-retaining substances, physiological cooling agents, Tetraselmis suecica extract, or mixtures of two or more of aforementioned agents, as further described below.

Sebum reducing and/or anti-acne agents: The cosmetic or pharmaceutical composition according to the present invention preferably further contains sebum reducing and/or anti-acne agents. The sebum reducing and/or anti-acne agents are preferably selected from the group consisting of capryloyl glycine, propanediol caprylate, undecylenoyl glycine, farnesol, retinoids like 13-cis retinoic acid (isotretinoin), all-trans-retinoic acid, adapalene, their salts or derivatives, androgen inhibitors like spironolactone and cyproterone, antibiotics, preferably clindamycin, erythromycin and tetracycline, zinc or zinc salts, and antiandrogens, 5-alpha-reductase inhibitors, D-panthenol, alpha-hydroxy acids, such as e.g. salicylic acid and lactic acid, pyruvic (alfa-keto acid) acids, aliphatic dicarboxylic acids, such as e.g. azelaic acid, L-carnitine, bakuchiol, 1,2-decanediol, senkyunolide-A and senkyunolide-A containing Apium graveolens seed oil, Tetraselmis extract, Quillaja saponaria extract, Enantia chlorantha bark extract, Spiraea ulmaria extract, butyl avocate, vitamin B6 (also known as pyridoxine) or its salts or derivatives, vitamin B3 (also known as niacin or nicotinic acid) or its salts or derivatives, benzoylperoxide, phloretin, Camellia sinensis extract and contained polyphenols such as e.g. epigallocatechin-3-gallate, red clover (Trifolium pretense) extract, soybean (Glycine Soja) seed extract, isoflavonoids or isoflavonoid containing extracts, preferably biochanin A, genistein, daidzein, genistin, daizin, and mixture of two or more of the aforesaid agents.

Anti-dandruff substances: In addition, the cosmetic or pharmaceutical composition according to the present invention can also advantageously be used in combination with one or more anti-dandruff substances, including triclosan, climbazole, octoxyglycerin, Octopirox® (1-hydroxy-4-methyl-6-(2,4,4-trimethylpentyl)-2(1H)-pyridone 2-aminoethanol salt), chitosan, farnesol, glycerol monolaurate, Propanediol Monocaprylate or combinations of said substances, which are used inter alia against dandruff.

Further suitable anti-dandruff substances are, Baypival® (Climbazole), Ketoconazol® (4-acetyl-1-{4-[2-(2,4-dichlorophenyl) r-2-(1H-imidazol-1-ylmethyl)-1,3-dioxylan-c-4-ylmethoxyphenyl}-piperazine, ketoconazole, elubiol, selenium disulfide, colloidal sulfur, sulfur polyethylene glycol sorbitan monooleate, sulfur ricinol polyethoxylate, sulfur tar distillate, salicylic acid (or in combination with hexachlorophene), undecylenic acid, monoethanolamide sulfosuccinate Na salt, Lamepon® UD (protein/undecylenic acid condensate), zinc pyrithione, aluminium pyrithione, magnesium pyrithione/dipyrithione magnesium sulfate, or mixtures of two or more of the aforesaid substances.

Anti-inflammatory and/or redness and/or itch ameliorating agents: The cosmetic or pharmaceutical composition according to the present invention preferably also contains anti-inflammatory and/or redness and/or itch ameliorating ingredients, in particular steroidal substances of the corticosteroid type, selected from the group consisting of hydrocortisone, dexamethasone, dexamethasone phosphate, methyl prednisolone or cortisone, are advantageously used as anti-inflammatory active ingredients or active ingredients to relieve reddening and itching, the list of which can be extended by the addition of other steroidal anti-inflammatories. Non-steroidal anti-inflammatories can also be used. More particularly:
(i) steroidal anti-inflammatory substances of the corticosteroid type, in particular hydrocortisone, hydrocortisone derivatives such as hydrocortisone 17-butyrate, dexamethasone, dexamethasone phosphate, methylprednisolone or cortisone;
(ii) non-steroidal anti-inflammatory substances, in particular oxicams such as piroxicam or tenoxicam, salicylates such as aspirin, disalcid, solprin or fendosal, acetic acid derivatives such as diclofenac, fenclofenac, indomethacin, sulindac, tolmetin or clindanac, fenamates such as mefenamic, meclofenamic, flufenamic or niflumic, propionic acid derivatives such as ibuprofen, naproxen or benoxaprofen, pyrazoles such as phenylbutazone, oxyphenylbutazone, febrazone or azapropazone, anthranilic acid derivatives, in particular avenanthramides;
(iii) natural or naturally occurring anti-inflammatory substances or substances that alleviate reddening and/or itching, in particular extracts or fractions from camomile, Aloe vera, Commiphora species, Rubia species, willow, willow-herb, oats, calendula, arnica, St John's wort, honeysuckle, rosemary, Passiflora incarnata, witch hazel, ginger or Echinacea, or single active compounds thereof as for example natural alpha-bisabolol, synthetic bisabolol, apigenin, apigenin-7-glucoside, gingerols, shogaols, gingerdiols, dehydrogingerdiones, paradols, especially natural or synthetic 6-paradol, naturally occuring avenanthramides, preferably avenanthramide A, avenanthramide B, avenanthramide C, avenanthramide D, avenanthramide E, non-natural or non-naturally occuring avenanthramides, preferably dihydroavenanthramide D, dihydroavenanthramide E, tranilast, boswellic acid, phytosterols, glycyrrhizin, glabridin, sclareolide and licochalcone A; preferably selected from the group consisting of natural alpha-bisabolol, synthetic bisabolol, natural avenanthramides, non-natural avenanthramides, preferably dihydroavenanthramide D, ginger extract, gingerols, shogaols, gingerdiols, dehydrogingerdiones, paradols, especially natural or synthetic 6-paradol, boswellic acid, phytosterols, glycyrrhizin, and licochalcone A, and/or allantoin, sclareolide, panthenol, (pseudo-)ceramides [preferably Ceramide 2, hydroxypropyl bispalmitamide M EA, cetyloxypropyl glyceryl methoxypropyl myristamide, N-(I-hexadecanoyl)-4-hydroxy-L-proline (1-hexadecyl) ester, hydroxyethyl palmityl oxyhydroxypropyl palmitamide], phytosterols, chitosan, and β-glucans, in particular 1,3-1,4-glucan from oats; or
(iv) histamine receptor antagonists, serine protease inhibitors (e.g. of Soy extracts), TRPV1 antagonists (e.g. 4-t-Butylcyclohexanol), NK1 antagonists (e.g. Aprepitant, Hydroxyphenyl Propamidobenzoic Acid), cannabinoid receptor agonists (e.g. Palmitoyl Ethanolamine) and TRPV3 antagonists, or mixtures of two or more of the abovementioned agents.

Examples which can be cited here are oxicams such as piroxicam or tenoxicam; salicylates such as aspirin, disalcid, solprin or fendosal; acetic acid derivatives such as diclofenac, fenclofenac, indomethacin, sulindac, tolmetin or clindanac; fenamates such as mefenamic, meclofenamic, flufenamic or niflumic; propionic acid derivatives such as ibuprofen, naproxen, benoxaprofen or pyrazoles such as phenylbutazone, oxyphenylbutazone, febrazone or azapropazone. Anthranilic acid derivatives are preferred anti-itch ingredients in a composition according to the present invention.

Also useful are natural or naturally occurring anti-inflammatory mixtures of substances or mixtures of substances that alleviate reddening and/or itching, in particular extracts or fractions from camomile, Aloe vera, Commiphora species, Rubia species, willow, willow-herb, oats, calendula, arnica, St John's wort, honeysuckle, rosemary, Passiflora incarnata, witch hazel, ginger or Echinacea; preferably selected from the group consisting of extracts or fractions from camomile, Aloe vera, oats, calendula, arnica, honeysuckle, rosemary, witch hazel, ginger or Echinacea, and/or pure substances, preferably alpha-bisabolol, apigenin, apigenin-7-glucoside, gingerols, shogaols, gingerdiols, dehydrogingerdiones, paradols, natural or naturally occuring avenanthramides, preferably tranilast, avenanthramide A, avenanthramide B, avenanthramide C, non-natural or non-naturally occuring avenanthramides, preferably dihydroavenanthramide D, dihydroavenanthramide E, avenanthramide D, avenanthramide E, avenanthramide F, boswellic acid, phytosterols, glycyrrhizin, glabridin and licochalcone A; preferably selected from the group consisting of alpha-bisabolol, natural avenanthramides, non-natural avenanthramides, preferably dihydroavenanthramide D (as described in WO 2004 047833 A1), boswellic acid, phytosterols, glycyrrhizin, and licochalcone A, and/or allantoin, panthenol, lanolin, (pseudo-)ceramides [preferably Ceramide 2, hydroxypropyl bispalmitamide MEA, cetyloxypropyl glyceryl methoxypropyl myristamide, N-(1-hexadecanoyl)-4-hydroxy-L-proline (1-hexadecyl) ester, hydroxyethyl palmityl oxyhydroxypropyl palmitamide], glycosphingolipids, phytosterols, chitosan, mannose, lactose and β-glucans, in particular 1,3->1,4-β-glucan from oats.

When bisabolol is used in the context of the present invention it can be of natural or synthetic origin, and is preferably “alpha-bisabolol”. Preferably, the bisabolol used is synthetically prepared or natural (−)-alpha-bisabolol and/or synthetic mixed-isomer alpha-bisabolol. If natural (−)-alpha-bisabolol is used, this can also be employed as a constituent of an essential oil or of a plant extract or of a fraction thereof, for example as a constituent of (fractions of) oil or extracts of camomile or of Vanillosmopsis (in particular Vanillosmopsis erythropappa or Vanillosmopsis arborea). Synthetic alpha-bisabolol is obtainable, for example, under the name “Dragosantol” from Symrise.

In case ginger extract is used in the context of the present invention, preferably extracts of the fresh or dried ginger root are used which are prepared by extraction with methanol, ethanol, iso-propanol, acetone, ethyl acetate, carbon dioxide (CO₂), hexane, methylene chloride, chloroform or other solvents or solvent mixtures of comparable polarity. The extracts are characterized by the presence of active skin irritation-reducing amounts of constituents such as e.g., gingerols, shogaols, gingerdiols, dehydrogingerdiones and/or paradols.

Odour absorbers and/or antiperspirant active agents: The cosmetic or pharmaceutical composition according to the present invention may include one or more odour absorbers and/or antiperspirant active agents (antiperspirants). Suitable odour absorbers are substances which are able to absorb and largely retain odour-forming compounds. They lower the partial pressure of the individual components, thus also reducing their rate of diffusion. It is important that perfumes must remain unimpaired in this process. Odour absorbers are not effective against bacteria. They comprise, for example, as main constituent, a complex zinc salt of ricinoleic acid or specific, largely odour-neutral fragrances which are known to the person skilled in the art as “fixatives”, such as, for example, extracts of labdanum or styrax or certain abietic acid derivatives. The odour masking agents are fragrances or perfume oils, which, in addition to their function as odour masking agents, give the deodorants their respective fragrance note. Perfume oils which may be mentioned are, for example, mixtures of natural and synthetic fragrances. Natural fragrances are extracts from flowers, stems and leaves, fruits, fruit peels, roots, woods, herbs and grasses, needles and branches, and resins and balsams. Also suitable are animal products, such as, for example, civet and castoreum. Typical synthetic fragrance compounds are products of the ester, ether, aldehyde, ketone, alcohol, and hydrocarbon type. Fragrance compounds of the ester type are, for example, benzyl acetate, p-tert-butylcyclohexyl acetate, linalyl acetate, phenylethyl acetate, linalyl benzoate, benzyl formate, allyl cyclohexylpropionate, styrallyl propionate and benzyl salicylate. The ethers include, for example, benzyl ethyl ether, and the aldehydes include, for example, the linear alkanals having 8 to 18 carbon atoms, citral, citronellal, citronellyloxyacetaldehyde, cyclamen aldehyde, hydroxycitronellal, lilial and bourgeonal, the ketones include, for example, the ionones and methyl cedryl ketone, the alcohols include anethole, citronellol, eugenol, isoeugenol, geraniol, linalool, phenylethyl alcohol and terpineol, and the hydrocarbons include mainly the terpenes and balsams. Preference is, however, given to using mixtures of different fragrances which together produce a pleasing fragrance note. Essential oils of relatively low volatility, which are mostly used as aroma components, are also suitable as perfume oils, e.g. sage oil, camomile oil, oil of cloves, melissa oil, mint oil, cinnamon leaf oil, linden flower oil, juniperberry oil, vetiver oil, olibanum oil, galbanum oil, labdanum oil and lavandin oil. Preference is given to using bergamot oil, dihydromyrcenol, lilial, lyral, citronellol, phenylethyl alcohol, α-hexylcinnamaldehyde, geraniol, benzylacetone, cyclamen aldehyde, linalool, boisambrene forte, ambroxan, indole, hedione, sandelice, lemon oil, mandarin oil, orange oil, allyl amyl glycolate, cyclovertal, lavandin oil, clary sage oil, 3-damascone, geranium oil bourbon, cyclohexyl salicylate, Vertofix coeur, iso-E-super, Fixolide NP, evernyl, iraldein gamma, phenylacetic acid, geranyl acetate, benzyl acetate, rose oxide, romilat, irotyl and floramat alone or in mixtures.

Suitable astringent antiperspirant active ingredients are primarily salts of aluminium, zirconium or of zinc. Such suitable antihydrotic active ingredients are, for example, aluminium chloride, aluminium chlorohydrate, aluminium dichlorohydrate, aluminium sesquichlorohydrate and complex compounds thereof, e.g., with 1,2-propylene glycol, aluminium hydroxyallantoinate, aluminium chloride tartrate, aluminium zirconium trichlorohydrate, aluminium zirconium tetrachlorohydrate, aluminium zirconium pentachlorohydrate and complex compounds thereof, e.g. with amino acids, such as glycine.

Lenitive substances: The cosmetic or pharmaceutical composition according to the present invention can also contain advantageously one or more lenitive substances, wherein any lenitive substances can be used which are suitable or customary in cosmetic or pharmaceutical applications such as alpha-bisabolol, azulene, guaiazulene, 18-beta-glycyrrhetinic acid, allantoin, Aloe vera juice or gel, extracts of Hamamelis virginiana (witch hazel), Echinacea species, Centella asiatica, chamomile, Arnica monatana, Glycyrrhiza species, algae, seaweed and Calendula officinalis, and vegetable oils such as sweet almond oil, baobab oil, olive oil and panthenol, Laureth-9, Trideceth-9 and 4-t-butylcyclohexanol.

Moisturising and/or moisture-retaining substances: Itching occurs with particular intensity when the skin is dry. The use of skin-moisturising and/or moisture-retaining substances can significantly alleviate itching. The cosmetic or pharmaceutical composition according to the present invention can therefore advantageously also contain one or more of the following moisturising and/or moisture-retaining substances: sodium lactate, urea, urea derivatives, alcohols, glycerol, diols such as propylene glycol, hexylene glycol, collagen, elastin or hyaluronic acid, diacyl adipates, petrolatum, urocanic acid, lecithin, panthenol, phytantriol, lycopene, (pseudo-)ceramides, glycosphingolipids, cholesterol, phytosterols, chitosan, chondroitin sulphate, lanolin, lanolin esters, amino acids, alpha-hydroxy acids (such as citric acid, lactic acid, malic acid) and their derivatives, mono-, di- and oligosaccharides such as glucose, galactose, fructose, mannose, fructose and lactose, polysugars such as R-glucans, in particular 1,3-1,4-β-glucan from oats, alpha-hydroxy fatty acids, triterpene acids such as betulinic acid or ursolic acid, and algae extracts. Preferred skin moisturizing agents are selected from the group consisting of alkane diols or alkane triols comprising 3 to 12 carbon atoms, preferably C₃-C₁₀-alkane diols and C₃-C₁₀-alkane triols. More preferably the skin moisturizing agents are selected from the group consisting of glycerol, 1,2-propylene glycol, 1,2-butylene glycol, and 1,3-butylene glycol.

Physiological cooling agents: The cosmetic or pharmaceutical composition according to the present invention can be particularly advantageously combined with one or more physiological cooling agent(s). The use of cooling agents can alleviate itching. Preferred individual cooling agents for use within the framework of the present invention are listed below. The person skilled in the art can add many other cooling agents to this list; the cooling agents listed can also be used in combination with one another: which are preferably selected here from the following list: menthol and menthol derivatives (for example L-menthol, D-menthol, racemic menthol, isomenthol, neoisomenthol, neomenthol) menthylethers (for example (1-menthoxy)-1,2-propanediol, (1-menthoxy)-2-methyl-1,2-propanediol, 1-menthyl-methylether), menthone glyceryl acetal, menthone glyceryl ketal or mixtures of both, menthylesters (for example menthylformiate, menthylacetate, menthylisobutyrate, menthyhydroxyisobutyrat, menthyllactates, L-menthyl-L-lactate, L-menthyl-D-lactate, menthyl-(2-methoxy)acetate, menthyl-(2-methoxyethoxy)acetate, menthylpyro-glutamate), menthylcarbonates (for example menthylpropyleneglycolcarbonate, menthylethyleneglycolcarbonate, menthylglycerolcarbonate or mixtures thereof), the semi-esters of menthols with a dicarboxylic acid or derivatives thereof (for example mono-menthylsuccinate, mono-menthylglutarate, mono-menthylmalonate, O-menthyl succinic acid ester-N,N-(dimethyl)amide, O-menthyl succinic acid ester amide), menthanecarboxylic acid amides (in this case preferably menthanecarboxylic acid-N-ethylamide [WS3] or N^{α}-(menthanecarbonyl)glycinethylester [WS5], menthanecarboxylic acid-N-(4-cyanophenyl)amide or menthanecarboxylic acid-N-(4-cyanomethylphenyl)amide, menthanecarboxylic acid-N-(alkoxyalkyl)amides), menthone and menthone derivatives (for example L-menthone glycerol ketal), 2,3-dimethyl-2-(2-propyl)-butyric acid derivatives (for example 2,3-dimethyl-2-(2-propyl)-butyric acid-N-methylamide [WS23]), isopulegol or its esters (I-(−)-isopulegol, I-(−)-isopulegolacetate), menthane derivatives (for example p-menthane-3,8-diol), cubebol or synthetic or natural mixtures, containing cubebol, pyrrolidone derivatives of cycloalkyldione derivatives (for example 3-methyl-2(1-pyrrolidinyl)-2-cyclopentene-1-one) or tetrahydropyrimidine-2-one (for example iciline or related compounds, as described in WO 2004/026840), further carboxamides (for example N-(2-(pyridin-2-yl)ethyl)-3-p-menthanecarboxamide or related compounds), (1R,2S,5R)—N-(4-Methoxyphenyl)-5-methyl-2-(1-isopropyl)cyclohexane-carboxamide [WS12], oxamates and [(1R,2S,5R)-2-isopropyl-5-methyl-cyclohexyl] 2-(ethylamino)-2-oxo-acetate (X Cool). Cooling agents which are preferred due to their particular synergistic effect are I-menthol, d-menthol, racemic menthol, menthone glycerol acetal (trade name: Frescolat® MGA), menthyl lactate (preferably I-menthyl lactate, in particular I-menthyl I-lactate (trade name: Frescolat® ML)), substituted menthyl-3-carboxamides (such as menthyl-3-carboxylic acid N-ethyl amide), 2-isopropyl-N-2,3-trimethyl butanamide, substituted cyclohexane carboxamides, 3-menthoxypropane-1,2-diol, 2-hydroxyethyl menthyl carbonate, 2-hydroxypropyl menthyl carbonate and isopulegol. Particularly preferred cooling agents are I-menthol, racemic menthol, menthone glycerol acetal (trade name: Frescolat® MGA), menthyl lactate (preferably I-menthyl lactate, in particular I-menthyl I-lactate (trade name: Frescolat® ML)), 3-menthoxypropane-1,2-diol, 2-hydroxyethyl menthyl carbonate and 2-hydroxypropyl menthyl carbonate. Very particularly preferred cooling agents are I-menthol, menthone glycerol acetal (trade name: Frescolat® MGA) and menthyl lactate (preferably I-menthyl lactate, in particular I-menthyl I-lactate (trade name: Frescolat® ML), 3-((−)-Menthoxy)propane-1,2-diol, N-Ethyl-p-menthane-3-carboxamide, Menthyl-ethylamidooxalat, and mixtures oft wo or more of the aforesaid cooling agents.

Tetraselmis Suecica extract: A Tetraselmis Suecica extract can also particularly advantageously be used in the cosmetic or pharmaceutical composition according to the present invention. Tetraselmis suecica is a marine green microalga belonging to the class Chlorophyceae, widely used in aquaculture for the feeding of mollusks and crustacean larvae and as a probiotic in fish. T. suecica is rich in vitamin E, carotenoids, chlorophyll, and tocopherols and has been suggested as a food supplement in human and animal diets. The ethanol/water extract from T. suecica is known for its ability to enhance dermal pigmentation and reduce psoriasis lesions.

The cosmetic or pharmaceutical, in particular dermatological, composition as defined herein, are preferably based on a carrier, which comprises at least one oil phase. Accordingly, oils, oil-in-water and water-in-oil emulsions, etc. are suitable. However, preparations solely based on water or water/alcohol or water/glycol are likewise possible or even preferred.

The oil phase or oil component in the cosmetic or pharmaceutical composition according to the present invention which may be suitable are for example plant oils, hydrocarbon, fatty alcohols, fatty acid esters, liquid UV filters, or mixtures of two or more of the aforesaid liquid oil components.

The oil phase or oil component in the cosmetic or pharmaceutical composition according to the present invention is preferably a plant oil and even more preferably a liquid plant oil. It can also advantageously be a mixture of two or more plant oils components, especially liquid plant oil mixtures.

Plant oils or vegetable oils are oils extracted from seeds, or less often, from other parts of fruits. Like animal fats, plant oils are mixtures of triglycerides. Soybean oil, rapeseed oil and cocoa butter are examples of plant oils from seeds. Olive oil, palm oil and rice bran oil are examples of oils from other parts of fruits. In common usage, plant oil or vegetable oil may refer exclusively to vegetable fats which are liquid at room temperature or at 35 to 37° C. skin temperature. Vegetable oils are usually edible.

The term “plant oils” also includes unsaturated plant oils. Unsaturated oils or vegetable oils can be transformed through partial or complete “hydrogenation” into oils of higher melting point. The hydrogenation process involves “sparging” the oil at high temperature and pressure with hydrogen in the presence of a catalyst, typically a powdered nickel compound. As each carbon-carbon double-bond is chemically reduced to a single bond, two hydrogen atoms each form single bonds with the two carbon atoms. The elimination of double bonds by adding hydrogen atoms is called saturation; as the degree of saturation increases, the oil progresses toward being fully hydrogenated. An oil may be hydrogenated to increase resistance to rancidity (oxidation) or to change its physical characteristics. As the degree of saturation increases, the oil's viscosity and melting point increase.

In a preferred variant, the plant oil is selected from the group consisting of Persea Gratissima (Avocado Oil), Abies Alba Seed Oil, Acacia Victoriae Seed Oil, Actinidia Chinensis (Kiwi) Seed Oil, Amaranthus Hypochondriacus Seed Oil, Arachis Hypogaea (Peanut) Oil, Astrocaryum Murumuru Seed Butter, Astrocaryum Tucuma Seed Butter, Astrocaryum Tucuma Seed Oil, Astrocaryum Vulgare Fruit Oil, Astrocaryum Vulgare Kernel Oil, Avena Sativa (Oat) Kernel Oil, Brassica Alba Seed Oil, Brassica Campestris (Rapeseed) Seed Oil, Butyrospermum Parkii (Shea) Butter, Butyrospermum Parkii (Shea) Oil, Calendula Officinalis Seed Oil, Calophyllum Inophyllum Seed Oil, Calophyllum Tacamahaca Seed Oil, Camellia Oleifera Seed Oil, Camellia Reticulata Seed Oil, Camellia Sinensis Seed Oil, Cannabis Sativa Seed Oil, Cannabis Sativa Seed/Stem Oil, Canola Oil, Carthamus Tinctorius (Safflower) Seed Oil, Chlorella Vulgaris Oil, Citrullus Lanatus (Watermelon) Seed Oil, Citrus Aurantifolia (Lime) Seed Oil, Citrus Aurantium Dulcis (Orange) Seed Oil, Citrus Grandis (Grapefruit) Seed Oil, Cocos Nucifera (Coconut) Oil, Cocos Nucifera (Coconut) Seed Butter, Coffea Arabica (Coffee) Seed Oil, Chlorella Oil (biotech), Corylus Americana (Hazelnut) Seed Oil, Corylus Avellana (Hazelnut) Seed Oil, Cucumis Melo (Melon) Seed Oil, Cucumis Sativus (Cucumber) Seed Oil, Cucurbita Pepo (Pumpkin) Seed Oil, Elaeis Guineensis (Palm) Oil, Elaeis (Palm) Fruit Oil, Glycine Soja (Soybean) Oil, Gossypium Herbaceum (Cotton) Seed Oil, Gossypium Hirsutum (Cotton) Seed Oil, Helianthus Annuus (Sunflower) Seed Oil, Macadamia Integrifolia Seed Oil, Macadamia Ternifolia Seed Oil, Mangifera Indica (Mango) Seed Butter, Mangifera Indica (Mango) Seed Oil, Melissa Officinalis Seed Oil, Microalgae Oil, Moringa Oleifera Seed Oil, Moringa Peregrina Seed Oil, Oenothera Biennis (Evening Primrose) Oil, Olea Europaea (Olive) Fruit Oil, Olus Oil, Orbignya Oleifera Seed Oil, Orbignya Speciosa Kernel Oil, Oryza Sativa (Rice) Bran/Germ Oil, Oryza Sativa (Rice) Bran Oil, Oryza Sativa (Rice) Germ Oil, Oryza Sativa (Rice) Lipids, Oryza Sativa (Rice) Seed Oil, Papaver Somniferum Seed Oil, Passiflora Edulis Seed Oil, Persea Gratissima (Avocado) Butter, Persea Gratissima (Avocado) Oil, Prunus Amygdalus Dulcis (Sweet Almond) Oil, Prunus Armeniaca (Apricot) Kernel Oil, Prunus Persica (Peach) Kernel Oil, Punica Granatum Seed Oil, Pyrus Malus (Apple) Seed Oil, Ricinoleic/Caproic/Caprylic/Capric Triglyceride(s), Ricinus Communis (Castor) Seed Oil, Rosa Canina Fruit Oil, Rosa Moschata Seed Oil, Rubus Idaeus (Raspberry) Seed Oil, Sesamum Indicum (Sesame) Seed Butter, Soybean Glycerides, Theobroma Cacao (Cocoa) Seed Butter, Theobroma Grandiflorum Seed Butter, Triticum Vulgare (Wheat) Bran Lipids, Triticum Vulgare (Wheat) Germ Oil, Vitis Vinifera (Grape) Seed Oil, Zea Mays (Corn) Germ Oil, and Zea Mays (Corn) Oil.

In a particular preferred variant, the cosmetic or pharmaceutical composition according to the first or second aspect of the present invention comprises as oil component a plant oil, selected from the group consisting of Caprylic Capric Triglycerides, Helianthus Annuus (Sunflower) Seed Oil, Simmondsia Chinensis ((Jojoba) Seed Oil, Olea Europaea (Olive) Fruit Oil, Argania spinosa kernel oil (Argan oil), Prunus Amygdalus Dulcis (Sweet Almond) Oil, Persea Gratissima (Avocado Oil), Butyrospermum Parkii (Shea) Butter, Cocos Nucifera (Coconut) Oil, Theobroma Cacao (Cocoa) Seed Butter, and mixtures of two or more of the aforesaid plant oils.

The plant oil can be used either as a single component or in mixture with one or more further different plant oil(s) as specified above.

Hydrocarbons (mineral oils) are in general organic compounds consisting entirely of hydrogen and carbon. As defined by IUPAC nomenclature or organic chemistry, the classifications for hydrocarbons are:
1. Saturated hydrocarbons are the simplest of the hydrocarbon species. They are composed entirely of single bonds and are saturated with hydrogen. The formula for acyclic saturated hydrocarbons (i.e., alkanes) is CₙH₂ₙ₊₂. The most general form of saturated hydrocarbons is CₙH₂ₙ₊₂₍₁₋ᵣ₎, where r is the number of rings. Those with exactly one ring are the cycloalkanes. Saturated hydrocarbons are the basis of petroleum fuels and are found as either linear or branched species.
2. Unsaturated hydrocarbons have one or more double or triple bonds between carbon atoms. Those with double bond are called alkenes. Those with one double bond have the formula CₙH₂ₙ (assuming non-cyclic structures). Those containing triple bonds are called alkynes. Those with one triple bond have the formula CₙH₂ₙ₋₂.
3. Aromatic hydrocarbons, also known as arenes, are hydrocarbons that have at least one aromatic ring.

Hydrocarbons can be inter alia liquids (e.g. hexane and benzene), waxes or low melting solids (e.g. paraffin wax and naphthalene). The term ‘aliphatic’ refers to non-aromatic hydrocarbons. Saturated aliphatic hydrocarbons are sometimes referred to as “paraffins”. Mineral oils and waxes are mixtures of predominantly saturated hydrocarbons consisting of straight-chain, branched and ring structures with carbon chain lengths greater than C14. Mineral oils and waxes are chemical substances prepared from naturally occurring crude petroleum oil. They mainly consist of mineral oil saturated hydrocarbons (MOSH) and mineral oil aromatic hydrocarbons (MOAH). Hydrocarbons have been used for many decades in skin and lip care cosmetic products due to their excellent skin tolerance as well as their high protecting and cleansing performance and broad viscosity options. In contrast to vegetable oils, mineral oils are non-allergenic since they are highly stable and not susceptible to oxidation or rancidity.

In a preferred variant, the hydrocarbon as the oil component of the cosmetic or pharmaceutical composition of the present invention is selected from the group consisting of undecane, tridecane, mineral oil, petrolatum, squalane, isohexadecane, C7-C8 isoparaffin, C8-C9 isoparaffin, C9-C11 isoparaffin, C9-C12 isoparaffin, C9-C13 isoparaffin, C9-C14 isoparaffin, C9-C16 isoparaffin, C10-C11 isoparaffin, C10-C12 isoparaffin, C10-C13 isoparaffin, C11-C12 isoparaffin, C11-C13 isoparaffin, C11-C14 isoparaffin, C12-C14 isoparaffin, C12-C15 isoparaffin, C12-C20 isoparaffin, C13-C14 isoparaffin, C13-C16 isoparaffin, C14-C16 isoparaffin, C15-C19 isoparaffin, and C18-C70 isoparaffin.

In a particular preferred variant, the oil component of the cosmetic or pharmaceutical composition of the present invention is a hydrocarbon selected from the group consisting of hydrocarbons, petrolatum, squalane, isohexadecane, C13-C14 isoparaffin, and mixtures of two or more of the aforesaid hydrocarbons.

The hydrocarbon can be used either as a single component or in mixture with one or more further different hydrocarbon(s) as specified above.

A fatty alcohol (or long-chain alcohol) is usually a high-molecular-weight, straight-chain primary alcohol, but can also range from as few as 4 to 6 carbons to as many as 22 to 26, derived from natural fats and oils. The precise chain length varies with the source. Some commercially important fatty alcohols are lauryl, stearyl and oleyl alcohols. They are colourless oily liquids (for smaller carbon numbers) or waxy solids, although impure samples may appear yellow. Fatty alcohols usually have an even number of carbon atoms and a single alcohol group (—OH) attached to the terminal carbon. Some are unsaturated and some are branched. Most fatty alcohols in nature are found as waxes which are esters with fatty acids and fatty alcohols. The traditional sources of fatty alcohols have largely been various vegetable oils and these remain a large-scale feedstock. The alcohols are obtained from the triglycerides (fatty acid triesters), which form the bulk of the oil. The process involves the transesterification of the triglycerides to give methyl esters which are then hydrogenated to give the fatty alcohols. Fatty alcohols are also prepared from petrochemical sources. In the Ziegler process, ethylene is oligomerized using triethylaluminium followed by air oxidation. Alternatively, ethylene can be oligomerized to give mixtures of alkenes, which are subjected to hydroformylation, this process affording odd-numbered aldehyde, which is subsequently hydrogenated. Fatty alcohols are mainly used in the production of detergents and surfactants. They are components also of cosmetic solvents. They find use as co-emulsifiers, emollients and thickeners in cosmetics.

In a preferred variant, the fatty alcohol as the oil component of the cosmetic or pharmaceutical composition of the present invention is selected from the group consisting of phenyl propanol, dimethyl phenylbutanol, hexyldecanol, octyldodecanol, octyldecanol, tridecylalcohol, isostearyl alcohol, phenylisohexanol, phenylpropanol, trimethylbenzenepropanol, isoamylalcohol, isostearyl alcohol, and isotridecyl alcohol.

The fatty alcohol can be used either as a single component or in mixture with one or more further different fatty alcohol(s) as specified above.

A fatty acid ester is a type of ester that results from the combination of a fatty acid with an alcohol. When the alcohol component is glycerol, the fatty acid esters produced can be monoglycerides, diglycerides or triglycerides. Fatty acid esters have a conditioning effect of softening the skin to create a smoothing sensation. They are also added to cosmetics to dissolve high-polarity active ingredients and UV absorbers. Esters of straight-chain fatty acids and lower alcohols are effective for dissolving slightly soluble ingredients for oils with a light touch during application. Isostearic acids and other liquid oils with branched fatty acids and unsaturated fatty acids are commonly used as emollients. Higher fatty acid esters and esters of higher alcohols with relatively high melting points are added to skin creams to adjust the application touch.

In a preferred variant, the fatty acid ester as the oil component of the cosmetic or pharmaceutical composition of the present invention is selected from the group consisting of C12-C15 Alkyl Benzoate, Capric/Lauric/Myristic/Oleic Triglyceride, Caprylic/Capric Triglyceride, Caprylic/Capric/Lauric Triglyceride, Caprylic/Capric/Linoleic Triglyceride, Caprylic/Capric/Myristic/Stearic Triglyceride, Caprylic/Capric/Palmitic/Stearic Triglyceride, Caprylic/Capric/Stearic Triglyceride, Caprylic/Capric/Succinic Triglyceride, Caprylyl Caprylate, Cetearyl Ethylhexanoate, Cetearyl Isononanoate, Cetearyl Nonanoate, Coco-Caprylate, Decyl Cocoate, Decyl Oleate, Dicaprylyl Carbonate, Diethyl Succinate, Diethylhexyl 2,6-Naphthalate, Diethylhexyl Carbonate, Dibutyl Adipate, Diisopropyl Adipate, Dipropylheptyl Carbonate, Ethyl Laurate, Ethylhexyl Isononanoate, Ethylhexyl Palmitate, Ethylhexyl Stearate, Glyceryl Caprylate Caprate, Glyceryl Caprylate, Glyceryl Laurate, Glyceryl Triacetyl Hydroxystearate, Glyceryl Triacetyl Ricinoleate, Hexyl Laurate, Isoamyl acetate, Isoamyl Cocoate, Isopropyl Palmitate, Isosorbide Dicaprylate, Isopropyl Myristate, Myristyl Myristate, Oleic/Linoleic Triglyceride, Oleic/Palmitic Triglyceride, Oleic/Palmitic/Lauric/Myristic/Linoleic Triglyceride, Propanediol Caprylate, Ricinoleic/Caproic/Caprylic/Capric Triglyceride, Oleostearine, Oleyl Erucate, Palmitic/Stearic Triglyceride, Propanediol Dicaprylate Caprate, Propylheptyl Caprylate, Stearyl Heptanoate/Stearyl Caprylate, Triheptanoin, Trihydroxystearin, Triisononanoin, Triisopalmitin, Triisostearin, Trilaurin, Trilinolein, Trilinolenin, Trimyristin, Triolein, Tripalmitin, Tripalmitolein, Tripelargonin, Triricinolein, and Tristearin.

In a particular preferred variant, the oil component of the cosmetic or pharmaceutical composition of the present invention is a fatty acid ester selected from the group consisting of C12-C15 Alkyl Benzoate, Caprylic/Capric Triglyceride, Caprylyl Caprylate, Cetearyl Ethylhexanoate, Cetearyl Isononanoate, Cetearyl Nonanoate, Coco-Caprylate, Decyl Cocoate, Decyl Oleate, Dicaprylyl Carbonate, Diethylhexyl 2,6-Naphthalate, Dibutyl Adipate, Diisopropyl Adipate, Ethyl Laurate, Ethylhexyl Isononanoate, Ethylhexyl Palmitate, Ethylhexyl Stearate, Glyceryl Caprylate, Glyceryl Laurate, Hexyl Laurate, Isoamyl Cocoate, Isopropyl Palmitate, Isopropyl Myristate, Myristyl Myristate, Propanediol Caprylate, Oleyl Erucate, Palmitic/Stearic Triglyceride, Propanediol Dicaprylate Caprate, Trihydroxystearin, Triisostearin, Triisononanoin, Tristearin, and mixtures of two or more of the aforesaid fatty acid esters.

The fatty acid ester can be used either as a single component or in mixture with one or more further different fatty acid ester(s) as specified above.

An UV filter is a compound or a mixture of compounds that block or absorb ultraviolet (UV) light. Since excessive UV radiation can cause sunburn, photoaging, and skin cancer, care products such as sunscreen usually include a classification for the specific wavelengths they filter. UV classifications include UVA (320-400 nm), UVB (290-320 nm) and UVC (200-280 nm). UV-absorbing compounds are used not only in sunscreen, but also in other personal care products, such as lipstick, shampoo, hair spray, body wash, toilet soap, and insect repellent. Chemical filters protect against UV radiation by absorbing, reflecting, or scattering. Reflection and scattering are accomplished by inorganic physical UV filters, such as titanium dioxide (TiO₂) and zinc oxide (ZnO). Absorption, mainly of UVB, is done by organic UV filters, which are known as chemical UV filters.

In a preferred variant, the liquid UV filter as the oil component of the cosmetic or pharmaceutical composition of the present invention is selected from the group consisting of Octocrylene, Ethylhexyl Salicylate, Homosalate, Ethylhexyl Methoxycinnamate, Isoamyl p-Methoxycinnamate, Camphor Benzalkonium Methosulfate, Polyarylamidomethyl Benzylidene Camphor, Isooctyl Methoxycionnamate, Ethylhexyl Triazone, Drometrizole Trisiloxane, Diethylhexyl Butamido Triazone, 3-Benzylidene Camphor, Octyl Salicylate, Ethylhexyl Dimethyl PABA, Bis-Ethylhexyloxyphenol Methoxyphenyl Triazine, Polysilicone-15, Diethylamino Hydroxybenzoyl Hexyl benzoate, N,N,N-Trimethyl-4-(2-oxoborn-3-ylidenemethyl) anilinium methyl sulfate, Benzoic acid-2-hydroxy-3,3,5-trimethylcyclohexyl ester/Homosalate, 2-Cyano-3,3-diphenyl acrylic acid, 2-Ethylhexyl ester/Octocrilene, 2-Ethylhexyl-4-methoxycinnamate/Octinoxate, Ethoxylated Ethyl-4-aminobenzoate, 2-Ethylhexyl salicylate/Octisalate, 2-Ethylhexyl-4-(dimethylamino)benzoate/Padimate O (USAN:BAN), 2,2′-Methylene-bis(6-(2H-benzotriazol-2-yl)-4-(1,1,3,3-tetramethyl-butyl)phenol)/Bisoctrizole, and Dimethicodiethylbenzalmalonate.

In a particular preferred variant the oil component of the cosmetic or pharmaceutical composition of the present invention is a liquid UV filter selected from the group consisting of Octocrylene, Ethylhexyl Salicylate, Homosalate, Ethylhexyl Methoxycinnamate, Isoamyl p-Methoxycinnamate, N,N,N-Trimethyl-4-(2-oxoborn-3-ylidenemethyl) anilinium methyl sulfate, Benzoic acid-2-hydroxy-3,3,5-trimethylcyclohexyl ester/Homosalate, 2-Cyano-3,3-diphenyl acrylic acid, 2-Ethylhexyl ester/Octocrilene, 2-Ethylhexyl-4-methoxycinnamate/Octinoxate, Ethoxylated Ethyl-4-aminobenzoate, 2-Ethylhexyl salicylate/Octisalate, 2-Ethylhexyl-4-(dimethylamino)benzoate/Padimate O (USAN:BAN), 2,2′-Methylene-bis(6-(2H-benzotriazol-2-yl)-4-(1,1,3,3-tetramethyl-butyl)phenol)/Bisoctrizole, Dimethicodi-ethylbenzalmalonate, and mixtures of two or more of the aforesaid UV filters.

The UV filter can be used either as a single component or in mixture with one or more further different UV filter (s) as specified above.

In addition to oil component(s) as defined before, the cosmetic or pharmaceutical composition according to the present invention preferably includes one or more further oil bodies. Suitable oil bodies, which form constituents of the O/W emulsions, are, for example, Guerbet alcohols based on fatty alcohols having 6 to 18, preferably 8 to 10, carbon atoms, esters of linear C5-C22 fatty acids with linear or branched C5-C22 fatty alcohols or esters of branched C1-C13 carboxylic acids with linear or branched C6-C22 fatty alcohols, such as, for example, myristyl myristate, myristyl palmitate, myristyl stearate, myristyl isostearate, myristyl oleate, myristyl behenate, myristyl erucate, cetyl myristate, cetyl palmitate, cetyl stearate, cetyl isostearate, cetyl oleate, cetyl behenate, cetyl erucate, stearyl myristate, stearyl palmitate, stearyl stearate, stearyl isostearate, stearyl oleate, stearyl behenate, stearyl erucate, isostearyl myristate, isostearyl palmitate, isostearyl stearate, isostearyl isostearate, isostearyl oleate, isostearyl behenate, isostearyl oleate, oleyl myristate, oleyl palmitate, oleyl stearate, oleyl isostearate, oleyl oleate, oleyl behenate, oleyl erucate, behenyl myristate, behenyl palmitate, behenyl stearate, behenyl isostearate, behenyl oleate, behenyl behenate, behenyl erucate, erucyl myristate, erucyl palmitate, erucyl stearate, erucyl isostearate, erucyl oleate, erucyl behenate and erucyl erucate. Also suitable are esters of linear C6-C22 fatty acids with branched alcohols, in particular 2-ethylhexanol, esters of C18-C38 alkylhydroxy carboxylic acids with linear or branched C6-C22 fatty alcohols, in particular Dioctyl Malate, esters of linear and/or branched fatty acids with polyhydric alcohols (such as, for example, propylene glycol, dimerdiol or trimertriol) and/or Guerbet alcohols, triglycerides based on C6-C10 fatty acids, liquid mono-/di-/triglyceride mixtures based on C6-C18 fatty acids, esters of C6-C22 fatty alcohols and/or Guerbet alcohols with aromatic carboxylic acids, in particular benzoic acid, esters of C2-C12 dicarboxylic acids with linear or branched alcohols having 1 to 22 carbon atoms or polyols having 2 to 10 carbon atoms and 2 to 6 hydroxyl groups, vegetable oils, branched primary alcohols, substituted cyclohexanes, linear and branched CC-C22 fatty alcohol carbonates, such as, for example, Dicaprylyl Carbonate (Cetiol® CC), Guerbet carbonates, based on fatty alcohols having 6 to 18, preferably 8 to 10, carbon atoms, esters of benzoic acid with linear and/or branched C6-C22-alcohols (e.g. Finsolv® TN), linear or branched, symmetrical or asymmetrical dialkyl ethers having 6 to 22 carbon atoms per alkyl group, such as, for example, dicaprylyl ether (Cetiol® OE), ring-opening products of epoxidized fatty acid esters with polyols, silicone oils (cyclomethicones, silicone methicone grades, etc.) and/or aliphatic or naphthenic hydrocarbons, such as, for example, squalane, squalene or dialkylcyclohexanes.

Within the context of the present invention, it is also possible and in some cases advantageous to combine the cosmetic or pharmaceutical, in particular dermatological, composition according to the first or second aspect of the present invention with other active substances, adjuvants, additives or auxiliaries which are different from the antimicrobial component (b) of the cosmetic or pharmaceutical composition according to the present invention and the further active agents which may incorporated into the cosmetic or pharmaceutical composition and already described above, such as sebum reducing agents, anti-acne agents, anti-dandruff agents, anti-inflammatory and/or redness and/or itch ameliorating agents, odour absorbers and/or antiperspirant active agents, lenitive substances, moisturising and/or moisture-retaining substances, physiological cooling agents, or Tetraselmis suecica extract, as described before.

Thus, optionally, other conventional cosmetically and/or pharmaceutically active substances, adjuvants, additives or auxiliaries, as further described below, may be added as component (c), i.e., in order to obtain a ready-for-use composition or formulation.

The cosmetic or pharmaceutical composition according to the present invention can advantageously be combined with other cosmetically or pharmaceutically active substances, adjuvants, additives or auxiliaries, such as are customarily used in such compositions, such as for example abrasives, agents against ageing of the skin, anti-cellulitis agents, antioxidants, astringents, odor absorbers, perspiration-inhibiting agents, anti-statics, binders, buffers, carrier materials, chelating agents, cell stimulants, depilatory agents, surface-active substances, deodorizing agents, antiperspirants, softeners, emulsifiers, enzymes, enzyme inhibitors, essential oils, fibers, film-forming agents, fixatives, foam-forming agents, foam stabilizers, substances for preventing foaming, foam boosters, gelling agents, gel-forming agents, hair care agents, hair-setting agents, hair-straightening agents, bleaching agents, strengthening agents, stain-removing agents, optically brightening agents, impregnating agents, dirt-repellent agents, dyes, friction-reducing agents, lubricants, opacifying agents, plasticizing agents, covering agents, polish, preservatives, gloss agents, green and synthetic polymers, powders, proteins, re-oiling agents, abrading agents, silicones, skin-soothing agents, skin care agents, skin-healing agents, skin-lightening agents, skin-protecting agents, skin-softening agents, hair promotion agents, warming agents, skin-warming agents, stabilizers, surfactants, UV-absorbing agents, UV filters, primary sun protection factors, secondary sun protection factors, detergents, fabric conditioning agents, suspending agents, skin-tanning agents, actives modulating skin or hair pigmentation, matrix-metalloproteinase inhibitors, glycosaminoglycan stimulators, TRPV1 antagonists, desquamating agents, anti-cellulite agents or fat enhancing agents, hair growth activators or inhibitors, thickeners, rheology additives, vitamins, oils, waxes, pearlizing waxes, fats, phospholipids, saturated fatty acids, mono- or polyunsaturated fatty acids, α-hydroxy acids, polyhydroxyfatty acids, liquefiers, dyestuffs, colour-protecting agents, pigments, anti-corrosives, fragrances or perfume oils, aromas, flavouring substances, odoriferous substances, polyols, electrolytes, organic solvents, and mixtures of two or more of the aforementioned substances, as further described below.

Of the above cosmetically or pharmaceutically active substances, adjuvants, additives or auxiliary agents against ageing of the skin, antioxidants, chelating agents, emulsifiers, surfactants, preservatives, green and synthetic polymers, rheology additives, oils, fragrances or perfume oils, polyols, and mixtures of two or more of the aforementioned agents are particularly preferred in the preparation of cosmetic and pharmaceutical composition.

Anti-ageing actives: The cosmetic or pharmaceutical composition according to the present invention preferably contains one or more anti-ageing actives. In the context of the invention, anti-ageing or biogenic agents are, for example antioxidants, matrix-metalloproteinase inhibitors (MMPI), skin moisturizing agents, glycosaminglycan stimulators, anti-inflammatory agents, TRPV1 antagonists and plant extracts.

Antioxidants: A preferred cosmetic or pharmaceutical composition according to the present invention comprises one or more antioxidants. Suitable antioxidants encompass amino acids (preferably glycine, histidine, tyrosine, tryptophan) and derivatives thereof, imidazoles (preferably urocanic acid) and derivatives thereof, peptides, preferably D,L-carnosine, D-carnosine, L-carnosine and derivatives thereof (preferably anserine), carnitine, creatine, matrikine peptides (preferably lysyl-threonyl-threonyl-lysyl-serine) and palmitoylated pentapeptides, carotenoids, carotenes (preferably alpha-carotene, beta-carotene, lycopene) and derivatives thereof, lipoic acid and derivatives thereof (preferably dihydrolipoic acid), aurothioglucose, propyl thiouracil and other thiols (preferably thioredoxine, glutathione, cysteine, cystine, cystamine and glycosyl, N-acetyl, methyl, ethyl, propyl, amyl, butyl and lauryl, palmitoyl, oleyl, gamma-linoleyl, cholesteryl, glyceryl and oligoglyceryl esters thereof) and salts thereof, dilauryl thiodipropionate, distearyl thiodipropionate, thiodipropionic acid and derivatives thereof (preferably esters, ethers, peptides, lipids, nucleotides, nucleosides and salts) and sulfoximine compounds (preferably buthionine sulfoximines, homocysteine sulfoximine, buthionine sulfones, penta-, hexa-, heptathionine sulfoximine) in very small tolerated doses (e.g. pmol to μmol/kg), also (metal) chelators (preferably alpha-hydroxy fatty acids, palmitic acid, phytic acid, lactoferrin, alpha-hydroxy acids (preferably citric acid, lactic acid, malic acid), humic acid, bile acid, bile extracts, tannins, bilirubin, biliverdin, EDTA, EGTA and derivatives thereof), unsaturated fatty acids and derivatives thereof (preferably gamma-linolenic acid, linoleic acid, oleic acid), folic acid and derivatives thereof, ubiquinone and derivatives thereof, ubiquinol and derivatives thereof, vitamin C and derivatives (preferably ascorbyl palmitate, Mg ascorbyl phosphate, ascorbyl acetate, ascorbyl glucoside), tocopherols and derivatives (preferably vitamin E acetate), vitamin A and derivatives (vitamin A palmitate) and coniferyl benzoate of benzoic resin, rutinic acid and derivatives thereof, flavonoids and glycosylated precursors thereof, in particular quercetin and derivatives thereof, preferably alpha-glucosyl rutin, rosmarinic acid, carnosol, carnosolic acid, resveratrol, caffeic acid and derivatives thereof, sinapic acid and derivatives thereof, ferulic acid and derivatives thereof, curcuminoids, chlorogenic acid and derivatives thereof, retinoids, preferably retinyl palmitate, retinol or tretinoin, ursolic acid, Levulinic Acid and esters and/or ketals thereof, butyl hydroxytoluene, butyl hydroxyanisole, nordihydroguaiac acid, nordihydroguaiaretic acid, trihydroxybutyrophenone, uric acid and derivatives thereof, mannose and derivatives thereof, zinc and derivatives thereof (preferably ZnO, ZnSO₄), selenium and derivatives thereof (preferably selenium methionine), superoxide dismutase, stilbenes and derivatives thereof (preferably stilbene oxide, trans-stilbene oxide) and the derivatives (salts, esters, ethers, sugars, nucleotides, nucleosides, peptides and lipids) of these cited active ingredients which are suitable according to the invention or extracts or fractions of plants having an antioxidant effect, preferably green tea, rooibos, honeybush, grape, rosemary, sage, melissa, thyme, lavender, olive, oats, cocoa, ginkgo, ginseng, liquorice, honeysuckle, sophora, pueraria, pinus, citrus, Phyllanthus emblica or St. John's wort, grape seeds, wheat germ, Phyllanthus emblica, coenzymes, preferably coenzyme Q10, plastoquinone and menaquinone. Preferred antioxidants are selected from the group consisting of vitamin A and derivatives, vitamin C and derivatives, tocopherol and derivatives, preferably tocopheryl acetate, and ubiquinone. If vitamin E and/or derivatives thereof are used as the antioxidant(s), it is advantageous to choose their concentrations from the range from about 0.001 to about 10% by weight, based on the total weight of the composition. If vitamin A or vitamin A derivatives or carotenes or derivatives thereof are used as the antioxidant(s), it is advantageous to choose their concentrations from the range from about 0.001 to about 10% by weight based on the total weight of the composition.

Matrix-Metalloproteinase inhibitors (MMPI): A preferred cosmetic or pharmaceutical composition according to the present invention comprises one or more matrix-metalloproteinase inhibitors, especially those inhibiting matrix-metalloproteinases enzymatically cleaving collagen, selected from the group consisting of ursolic acid, retinyl palmitate, propyl gallate, precocenes, 6-hydroxy-7-methoxy-2,2-dimethyl-1(2H)-benzopyran, 3,4-dihydro-6-hydroxy-7-methoxy-2,2-dimethyl-1(2H)-benzopyran, benzamidine hydrochloride, the cysteine proteinase inhibitors N-ethylmalemide and epsilon-amino-n-caproic acid of the serinprotease inhibitors: phenylmethylsufonylfluoride, collhibin (company Pentapharm; INCI: hydrolysed rice protein), oenotherol (company Soliance; INCI: propylene glycol, aqua, Oenothera biennis root extract, ellagic acid and ellagitannins, for example from pomegranate), phosphoramidone hinokitiol, EDTA, galardin, EquiStat (company Collaborative Group; apple fruit extract, soya seed extract, ursolic acid, soya isoflavones and soya proteins), sage extracts, MDI (company Atrium; INCI: glycosaminoglycans), fermiskin (company Silab/Mawi; INCI: water and Lentinus edodes extract), actimp 1.9.3 (company Expanscience/Rahn; INCI: hydrolysed lupine protein), lipobelle soyaglycone (company Mibelle; INCI: alcohol, polysorbate 80, lecithin and soy isoflavones), extracts from green and black tea and further plant extracts, proteins or glycoproteins from soya, hydrolysed proteins from rice, pea or lupine, plant extracts which inhibit MMPs, preferably extracts from shitake mushrooms, extracts from the leaves of the Rosaceae family, sub-family Rosoideae, quite particularly extracts of blackberry leaf, as e.g. SymMatrix (company Symrise, INCI: Maltodextrin, Rubus Fruticosus (Blackberry) Leaf Extract). Preferred actives of are selected from the group consisting of retinyl palmitate, ursolic acid, extracts from the leaves of the Rosaceae family, sub-family Rosoideae, genistein and daidzein.

Glycosaminoglycan stimulators: A preferred cosmetic or pharmaceutical composition according to the present invention comprises one or more substances stimulating the synthesis of glycosaminoglycans which are selected from the group consisting of hyaluronic acid and derivatives or salts, Subliskin (Sederma, INCI: Sinorhizobium Meliloti Ferment Filtrate, Cetyl Hydroxyethylcellulose, Lecithin), Hyalufix (BASF, INCI: Water, Butylene Glycol, Alpinia galanga leaf extract, Xanthan Gum, Caprylic/Capric Triglyceride), Stimulhyal (Soliance, INCI: Calcium ketogluconate), Syn-Glycan (DSM, INCI: Tetradecyl Aminobutyroylvalylaminobutyric Urea Trifluoroacetate, Glycerin, Magnesium chloride), Kalpariane (Biotech Marine), DC Upregulex (Distinctive Cosmetic Ingredients, INCI: Water, Butylene Glycol, Phospholipids, Hydrolyzed Sericin), glucosamine, N-acetyl glucosamine, retinoids, preferably retinol and vitamin A, Arctium lappa fruit extract, Eriobotrya japonica extract, Genkwanin, N-Methyl-L-serine, (−)-alpha-bisabolol or synthetic alpha-bisabolol such as e.g. Dragosantol and Dragosantol 100 from Symrise, oat glucan, Echinacea purpurea extract and soy protein hydrolysate. Preferred actives are selected from the group consisting of hyaluronic acid and derivatives or salts, retinol and derivatives, (−)-alpha-bisabolol or synthetic alpha-bisabolol such as e.g. Dragosantol and Dragosantol 100 from Symrise, oat glucan, Echinacea purpurea extract, Sinorhizobium Meliloti Ferment Filtrate, Calcium ketogluconate, Alpinia galanga leaf extract and tetradecyl aminobutyroylvalylaminobutyric urea trifluoroacetate.

TRPV1 antagonists: A preferred cosmetic or pharmaceutical composition according to the present invention comprises one or more TRPV1 antagonsits. Suitable compounds which reduce the hypersensitivity of skin nerves based on their action as TRPV1 antagonists, encompass e.g., trans-4-tert-butyl cyclohexanol, or indirect modulators of TRPV1 by an activation of the p-receptor, e.g., acetyl tetrapeptide-15, are preferred.

Plant extracts: A preferred cosmetic or pharmaceutical composition according to the present invention comprises one or more plant extracts. Plant extracts, special highly active plant extract fractions and also highly pure active substances isolated from plant extracts can also be used in the cosmetic or pharmaceutical composition according to the present invention. Extracts, fractions and active substances from camomile, aloe vera, Commiphora species, Rubia species, willows, willow-herb, ginger, marigold, arnica, Glycyrrhiza species, Echinacea species, Rubus species and pure substances such as inter alia bisabolol, apigenin, apigenin-7-glucoside, gingerols such as [6]-gingerol, paradols such as [6]-paradol, boswellic acid, phytosterols, glycyrrhizine, glabridin or licochalcone A are particularly preferred.

Desquamating agents: The cosmetic or pharmaceutical composition according to the present invention preferably contains one or more desquamating agents. The expression “desquamating agent” is understood to mean any compound capable of acting:
either directly on desquamation by promoting exfoliation, such as β-hydroxy acids, in particular salicylic acid and its derivatives (including 5-n-octanoylsalicylic acid); α-hydroxy acids, such as glycolic, citric, lactic, tartaric, malic or mandelic acids; urea; gentisic acid; oligofucoses; cinnamic acid; extract of Sophora japonica; resveratrol and some derivatives of jasmonic acid;
or on the enzymes involved in the desquamation or the degradation of the corneodesmosomes, glycosidases, stratum corneum chymotryptic enzyme (SCCE) or other proteases (trypsin, chymotrypsin-like). There may be mentioned agents chelating inorganic salts: EDTA; N-acyl-N,N′,N′-ethylenediaminetriacetic acid; aminosulphonic compounds and in particular (N-2-hydroxyethylpiperazine-N-2-ethane)sulphonic acid (HEPES); derivatives of 2-oxothiazolidine-4-carboxylic acid (procysteine); derivatives of alpha-amino acids of the glycine type (as described in EP-0 852 949, and sodium methylglycine diacetate marketed by BASF under the trade name TRILON M); honey; sugar derivatives such as 0-octanoyl-6-D-maltose and N-acetylglucosamine; chestnut extracts such as those marketed by the company SILAB under the name Recoverine®, prickly pear extracts such as those marketed under the name Exfolactive® by the company SILAB, or Phytosphingosine SLC® (phytosphingosine grafted with a salicylic acid) marketed by the company Degussa.

Desquamating agents suitable for the invention may be chosen in particular from the group comprising sulphonic acids, calcium chelators, α-hydroxy acids such as glycolic, citric, lactic, tartaric, malic or mandelic acids; ascorbic acid and its derivatives such as ascorbyl glucoside and magnesium ascorbyl phosphate; nicotinamide; urea; (N-2-hydroxyethylpiperazine-N-2-ethane)sulphonic acid (HEPES), β-hydroxy acids such as salicylic acid and its derivatives, retinoids such as retinol and its esters, retinal, retinoic acid and its derivatives, chestnut or prickly pear extracts, in particular marketed by SILAB; reducing compounds such as cysteine or cysteine precursors.

Desquamating agents which can be used are also nicotinic acid and its esters and nicotinamide, also called vitamin B3 or vitamin PP, and ascorbic acid and its precursors.

(Metal) chelating agents: A combination with one or more (metal) chelating agents can also be advantageous used in the cosmetic or pharmaceutical composition according to the present invention, wherein any metal chelating agents can be used which are suitable or customary in cosmetic or pharmaceutical applications. Preferred (metal) chelating agents include α-hydroxy fatty acids, phytic acid, lactoferrin, α-hydroxy acids, such as interalia gluconic acid, glyceric acid, glycolic acid, isocitric acid, citric acid, lactic acid, malic acid, mandelic acid, tartaric acid, as well as humic acids, bile acids, bile extracts, bilirubin, biliverdin or EDTA, EGTA and their derivatives. The use of one or more chelating agent(s) improves the stability of the composition according to the present invention.

Emulsifiers: In addition, the cosmetic or pharmaceutical composition according to the present invention can also advantageously contain one or more emulsifiers, including for example:
products of the addition of 2 to 30 mol ethylene oxide and/or 0 to 5 mol propylene oxide onto linear C₈₋₂₂ fatty alcohols, onto C₁₂₋₂₂ fatty acids and onto alkyl phenols containing 8 to 15 carbon atoms in the alkyl group;
C_{12/18} fatty acid monoesters and diesters of addition products of 1 to 30 mol ethylene oxide onto glycerol;
glycerol mono- and diesters and sorbitan mono- and diesters of saturated and unsaturated fatty acids containing 6 to 22 carbon atoms and ethylene oxide addition products thereof;
addition products of 15 to 60 mol ethylene oxide onto castor oil and/or hydrogenated castor oil;
polyol esters and, in particular, polyglycerol esters such as, for example, polyglycerol polyricinoleate, polyglycerol poly-12-hydroxystearate or polyglycerol dimerate isostearate. Mixtures of compounds from several of these classes are also suitable;
addition products of 2 to 15 mol ethylene oxide onto castor oil and/or hydrogenated castor oil;
partial esters based on linear, branched, unsaturated or saturated C_{6/22} fatty acids, ricinoleic acid and 12-hydroxystearic acid and glycerol, polyglycerol, pentaerythritol, dipentaerythritol, sugar alcohols (for example sorbitol), alkyl glucosides (for example methyl glucoside, butyl glucoside, lauryl glucoside) and polyglucosides (for example cellulose);
mono-, di and trialkyl phosphates and mono-, di- and/or tri-PEG-alkyl phosphates and salts thereof;
wool wax alcohols;
polysiloxane/polyalkyl polyether copolymers and corresponding derivatives;
mixed esters of pentaerythritol, fatty acids, citric acid and fatty alcohol and/or mixed esters of C₆₋₂₂ fatty acids, methyl glucose and polyols, preferably glycerol or polyglycerol,
polyalkylene glycols and
glycerol carbonate.

The addition products of ethylene oxide and/or propylene oxide onto fatty alcohols, fatty acids, alkylphenols, glycerol mono- and diesters and sorbitan mono- and diesters of fatty acids or onto castor oil are known commercially available products. They are homologue mixtures of which the average degree of alkoxylation corresponds to the ratio between the quantities of ethylene oxide and/or propylene oxide and substrate with which the addition reaction is carried out. C_{12/18} fatty acid monoesters and diesters of addition products of ethylene oxide onto glycerol are known as lipid layer enhancers for cosmetic formulations. The preferred emulsifiers are described in more detail as follows:

Partial glycerides: Typical examples of suitable partial glycerides are hydroxystearic acid monoglyceride, hydroxystearic acid diglyceride, isostearic acid monoglyceride, isostearic acid diglyceride, oleic acid monoglyceride, oleic acid diglyceride, ricinoleic acid monoglyceride, ricinoleic acid diglyceride, linoleic acid monoglyceride, linoleic acid diglyceride, linolenic acid monoglyceride, linolenic acid diglyceride, erucic acid monoglyceride, erucic acid diglyceride, tartaric acid monoglyceride, tartaric acid diglyceride, citric acid monoglyceride, citric acid diglyceride, malic acid monoglyceride, malic acid diglyceride and technical mixtures thereof which may still contain small quantities of triglyceride from the production process. Addition products of 1 to 30 and preferably 5 to 10 mol ethylene oxide onto the partial glycerides mentioned are also suitable.

Sorbitan esters: Suitable sorbitan esters are sorbitan monoisostearate, sorbitan sesquiisostearate, sorbitan diisostearate, sorbitan triisostearate, sorbitan monooleate, sorbitan sesquioleate, sorbitan dioleate, sorbitan trioleate, sorbitan monoerucate, sorbitan sesquierucate, sorbitan dierucate, sorbitan trierucate, sorbitan monoricinoleate, sorbitan sesquiricinoleate, sorbitan diricinoleate, sorbitan triricinoleate, sorbitan monohydroxystearate, sorbitan sesquihydroxystearate, sorbitan dihydroxystearate, sorbitan trihydroxystearate, sorbitan monotartrate, sorbitan sesquitartrate, sorbitan ditartrate, sorbitan tritartrate, sorbitan monocitrate, sorbitan sesquicitrate, sorbitan dicitrate, sorbitan tricitrate, sorbitan monomaleate, sorbitan sesquimaleate, sorbitan dimaleate, sorbitan trimaleate and technical mixtures thereof. Addition products of 1 to 30 and preferably 5 to 10 mol ethylene oxide onto the sorbitan esters mentioned are also suitable.

Polyglycerol esters: Typical examples of suitable polyglycerol esters are Polyglyceryl-2 Dipolyhydroxystearate (Dehymuls® PGPH), Polyglycerin-3-Diisostearate (Lameform® TGI), Polyglyceryl-4 Isostearate (Isolan® GI 34), Polyglyceryl-3 Oleate, Diisostearoyl Polyglyceryl-3 Diisostearate (Isolan® PDI), Poly-glyceryl-3 Methylglucose Distearate (Tego Care® 450), Polyglyceryl-3 Beeswax (Cera Bellina®), Polyglyceryl-4 Caprate (Polyglycerol Caprate T2010/90), Polyglyceryl-3 Cetyl Ether (Chimexane® NL), Polyglyceryl-3 Distearate (Cremophor® GS 32), Polyglyceryl Polyricinoleate (Admul® WOL 1403), Polyglyceryl Dimerate Isostearate, and mixtures thereof. Examples of other suitable polyolesters are the mono-, di- and triesters of trimethylol propane or pentaerythritol with lauric acid, cocofatty acid, tallow fatty acid, palmitic acid, stearic acid, oleic acid, behenic acid and the like optionally reacted with 1 to 30 mol ethylene oxide.

Anionic emulsifiers: Typical anionic emulsifiers are aliphatic C12 to C 22 fatty acids, such as palmitic acid, stearic acid or behenic acid for example, and C12 to C22 dicarboxylic acids, such as azelaic acid or sebacic acid for example.

Amphoteric emulsifiers: Other suitable emulsifiers are amphoteric or zwitterionic surfactants. Zwitterionic surfactants are surface-active compounds which contain at least one quaternary ammonium group and at least one carboxylate and one sulfonate group in the molecule. Particularly suitable zwitterionic surfactants are the so-called betaines, such as the N-alkyl-N,N-dimethyl ammonium glycinates, for example cocoalkyl dimethyl ammonium glycinate, N-acylaminopropyl-N,N-dimethyl ammonium glycinates, for example cocoacylaminopropyl dimethyl ammonium glycinate, and 2-alkyl-3-carboxymethyl-3-hydroxyethyl imidazolines containing 8 to 18 carbon atoms in the alkyl or acyl group and cocoacylaminoethyl hydroxyethyl carboxymethyl glycinate. The fatty acid amide derivative known under the CTFA name of Cocamidopropyl Betaine is particularly preferred. Ampholytic surfactants are also suitable emulsifiers. Ampholytic surfactants are surface-active compounds which, in addition to a C_{8/18} alkyl or acyl group, contain at least one free amino group and at least one —COOH— or —SO₃H— group in the molecule and which are capable of forming inner salts. Examples of suitable ampholytic surfactants are N-alkyl glycines, N-alkyl propionic acids, N-alkylaminobutyric acids, N-alkyliminodipropionic acids, N-hydroxyethyl-N-alkylamidopropyl glycines, N-alkyl taurines, N-alkyl sarcosines, 2-alkylaminopropionic acids and alkylaminoacetic acids containing around 8 to 18 carbon atoms in the alkyl group. Particularly preferred ampholytic surfactants are N-coco-alkylaminopropionate, cocoacylaminoethyl aminopropionate and C12/18 acyl sarcosine.

Surfactants: The cosmetic or pharmaceutical composition according to the present invention preferably includes one or more anionic and/or amphoteric or zwitterionic surfactants. Typical examples encompass: Almondamidopropylamine Oxide, Almondamidopropyl Betaine, Aminopropyl Laurylglutamine, Ammonium C12-15 Alkyl Sulfate, Ammonium C12-16 Alkyl Sulfate, Ammonium Capryleth Sulfate, Ammonium Cocomonoglyceride Sulfate, Ammonium Coco-Sulfate, Ammonium Cocoyl Isethionate, Ammonium Cocoyl Sarcosinate, Ammonium C12-15 Pareth Sulfate, Ammonium C9-10 Perfluoroalkylsulfonate, Ammonium Dinonyl Sulfosuccinate, Ammonium Dodecylbenzenesulfonate, Ammonium Isostearate, Ammonium Laureth-6 Carboxylate, Ammonium Laureth-8 Carboxylate, Ammonium Laureth Sulfate, Ammonium Laureth-5 Sulfate, Ammonium Laureth-7 Sulfate, Ammonium Laureth-9 Sulfate, Ammonium Laureth-12 Sulfate, Ammonium Lauroyl Sarcosinate, Ammonium Lauryl Sulfate, Ammonium Lauryl Sulfosuccinate, Ammonium Myreth Sulfate, Ammonium Myristyl Sulfate, Ammonium Nonoxynol-4 Sulfate, Ammonium Nonoxynol-30 Sulfate, Ammonium Oleate, Ammonium Palm Kernel Sulfate, Ammonium Stearate, Ammonium Tallate, AMPD-Isostearoyl Hydrolyzed Collagen, AMPD-Rosin Hydrolyzed Collagen, AMP-Isostearoyl Hydrolyzed Collagen, AMP-Isostearoyl Hydrolyzed Keratin, AMP-Isostearoyl Hydrolyzed Soy Protein, AMP-Isostearoyl Hydrolyzed Wheat Protein, Apricotamidopropyl Betaine, Arachidic Acid, Arginine Hexyldecyl Phosphate, Avocadamidopropyl Betaine, Avocado Oil Glycereth-8 Esters, Babassu Acid, Babassuamidopropylamine Oxide, Babassuamidopropyl Betaine, Beeswax Acid, Behenamidopropyl Betaine, Behenamine Oxide, Beheneth-25, Beheneth-30, Behenic Acid, Behenyl Betaine, Bis-Butyldimethicone Polyglyceryl-3, Butoxynol-5 Carboxylic Acid, Butoxynol-19 Carboxylic Acid, Butyldimoniumhydroxypropyl Butylglucosides Chloride, Butyldimoniumhydroxypropyl Laurylglucosides Chloride, Butyl Glucoside, Butylglucoside Caprate, Butylglucosides Hydroxypropyltrimonium Chloride, Butyloctanoic Acid, C18-36 Acid, C20-40 Acid, C30-50 Acid, C16-22 Acid Amide MEA, Calcium Dodecylbenzenesulfonate, Calcium Lauroyl Taurate, C9-16 Alkane/Cycloalkane, C10-14 Alkyl Benzenesulfonic Acid, C12-14 Alkyl Diaminoethylglycine HCL, C9-15 Alkyl Phosphate, Candida Bombicola/Glucose/Methyl Rapeseedate Ferment, Canolamidopropyl Betaine, Capric Acid, Caproic Acid, Caproyl Ethyl Glucoside, Capryl/Capramidopropyl Betaine, Capryleth-4 Carboxylic Acid, Capryleth-6 Carboxylic Acid, Capryleth-9 Carboxylic Acid, Caprylic Acid, Capryloyl Collagen Amino Acids, Capryloyl Glycine, Capryloyl Hydrolyzed Collagen, Capryloyl Hydrolyzed Keratin, Capryloyl Keratin Amino Acids, Capryloyl Silk Amino Acids, Caprylyl/Capryl Glucoside, Caprylyl/Capryl Wheat Bran/Straw Glycosides, Caprylyl Glucoside, Caprylyl Glyceryl Ether, Caprylyl Pyrrolidone, Carnitine, Ceteareth-20, Ceteareth-23, Ceteareth-24, Ceteareth-25, Ceteareth-27, Ceteareth-28, Ceteareth-29, Ceteareth-30, Ceteareth-33, Ceteareth-34, Ceteareth-40, Ceteareth-50, Ceteareth-55, Ceteareth-60, Ceteareth-80, Ceteareth-100, Ceteareth-25 Carboxylic Acid, Ceteareth-2 Phosphate, Ceteareth-4 Phosphate, Ceteareth-5 Phosphate, Ceteareth-10 Phosphate, Ceteth-20, Ceteth-23, Ceteth-24, Ceteth-25, Ceteth-30, Ceteth-40, Ceteth-45, Ceteth-150, Ceteth-8 Phosphate, Ceteth-10 Phosphate, Ceteth-20 Phosphate, Cetoleth-22, Cetoleth-24, Cetoleth-25, Cetoleth-30, Cetyl Betaine, Chrysanthemum Sinense Flower Extract, C12-14 Hydroxyalkyl Hydroxyethyl Beta-Alanine, C12-14 Hydroxyalkyl Hydroxyethyl Sarcosine, Cocamidoethyl Betaine, Cocamidopropylamine Oxide, Cocamidopropyl Betainamide MEA Chloride, Cocamidopropyl Betaine, Cocamidopropyl Hydroxysultaine, Cocamine Oxide, Cocaminobutyric Acid, Cocaminopropionic Acid, Coceth-7 Carboxylic Acid, Coceth-4 Glucoside, Cocoamphodipropionic Acid, Cocobetainamido Amphopropionate, Coco-Betaine, Cocodimonium Hydroxypropyl Hydrolyzed Rice Protein, Cocodimonium Hydroxypropyl Hydrolyzed Soy Protein, Cocodimonium Hydroxypropyl Hydrolyzed Wheat Protein, Coco-Glucoside, Cocoglucosides Hydroxypropyltrimonium Chloride, Coco-Hydroxysultaine, Coco-Morpholine Oxide, Coconut Acid, Coconut Oil Glycereth-8 Esters, Coco/Oleamidopropyl Betaine, Coco-Sultaine, Coco/Sunfloweramidopropyl Betaine, Cocoylcholine Methosulfate, Cocoyl Glutamic Acid, Cocoyl Hydrolyzed Collagen, Cocoyl Hydrolyzed Keratin, Cocoyl Hydrolyzed Oat Protein, Cocoyl Hydrolyzed Rice Protein, Cocoyl Hydrolyzed Silk, Cocoyl Hydrolyzed Soy Protein, Cocoyl Hydrolyzed Wheat Protein, Cocoyl Sarcosine, Corn Acid, Cottonseed Acid, Cottonseed Oil Glycereth-8 Esters, C10-16 Pareth-1, C10-16 Pareth-2, C11-13 Pareth-6, C11-13 Pareth-9, C11-13 Pareth-10, C11-15 Pareth-30, C11-15 Pareth-40, C12-13 Pareth-1, C12-13 Pareth-23, C12-14 Pareth-5, C12-14 Pareth-9, C13-15 Pareth-21, C14-15 Pareth-8, C20-22 Pareth-30, C20-40 Pareth-40, C20-40 Pareth-95, C22-24 Pareth-33, C30-50 Pareth-40, C9-11 Pareth-6 Carboxylic Acid, C9-11 Pareth-8 Carboxylic Acid, C11-15 Pareth-7 Carboxylic Acid, C12-13 Pareth-5 Carboxylic Acid, C12-13 Pareth-7 Carboxylic Acid, C12-13 Pareth-8 Carboxylic Acid, C12-13 Pareth-12 Carboxylic Acid, C12-15 Pareth-7 Carboxylic Acid, C12-15 Pareth-8 Carboxylic Acid, C12-15 Pareth-12 Carboxylic Acid, C14-15 Pareth-8 Carboxylic Acid, C6-10 Pareth-4 Phosphate, C12-13 Pareth-2 Phosphate, C12-13 Pareth-10 Phosphate, C12-15 Pareth-6 Phosphate, C12-15 Pareth-8 Phosphate, C12-15 Pareth-10 Phosphate, C12-16 Pareth-6 Phosphate, C4-18 Perfluoroalkylethyl Thiohydroxypropyltrimonium Chloride, Cupuassuamidopropyl Betaine, DEA-C12-13 Alkyl Sulfate, DEA-C12-15 Alkyl Sulfate, DEA-Ceteareth-2 Phosphate, DEA-Cetyl Sulfate, DEA-Cocoamphodipropionate, DEA-C12-13 Pareth-3 Sulfate, DEA-Cyclocarboxypropyloleate, DEA-Dodecylbenzenesulfonate, DEA-Isostearate, DEA-Laureth Sulfate, DEA-Lauryl Sulfate, DEA-Linoleate, DEA-Methyl Myristate Sulfonate, DEA-Myreth Sulfate, DEA-Myristate, DEA-Myristyl Sulfate, DEA-Oleth-5 Phosphate, DEA-Oleth-20 Phosphate, DEA PG-Oleate, Deceth-7 Carboxylic Acid, Deceth-7 Glucoside, Deceth-9 Phosphate, Decylamine Oxide, Decyl Betaine, Decyl Glucoside, Decyltetradeceth-30, Decyltetradecylamine Oxide, Diammonium Lauramido-MEA Sulfosuccinate, Diammonium Lauryl Sulfosuccinate, Diammonium Oleamido PEG-2 Sulfosuccinate, Dibutoxymethane, Di-CI 2-15 Pareth-2 Phosphate, Di-CI 2-15 Pareth-4 Phosphate, Di-CI 2-15 Pareth-6 Phosphate, Di-C12-15 Pareth-8 Phosphate, Di-CI 2-15 Pareth-10 Phosphate, Didodecyl Butanetetracarboxylate, Diethylamine Laureth Sulfate, Diethylhexyl Sodium Sulfosuccinate, Dihydroxyethyl C8-10 Alkoxypropylamine Oxide, Dihydroxyethyl C9-11 Alkoxypropylamine Oxide, Dihydroxyethyl C12-15 Alkoxypropylamine Oxide, Dihydroxyethyl Cocamine Oxide, Dihydroxyethyl Lauramine Oxide, Dihydroxyethyl Stearamine Oxide, Dihydroxyethyl Tallowamine Oxide, Dimethicone PEG-7 Phosphate, Dimethicone PEG-10 Phosphate, Dimethicone PEG/PPG-7/4 Phosphate, Dimethicone PEG/PPG-12/4 Phosphate, Dimethicone/Polyglycerin-3 Crosspolymer, Dimethicone Propyl PG-Betaine, Dimyristyl Phosphate, Dioleoylamidoethyl Hydroxyethylmonium Methosulfate, DIPA-Hydrogenated Cocoate, DIPA-Lanolate, DIPA-Myristate, Dipotassium Capryloyl Glutamate, Dipotassium Lauryl Sulfosuccinate, Dipotassium Undecylenoyl Glutamate, Disodium Babassuamido MEA-Sulfosuccinate, Disodium Caproamphodiacetate, Disodium Caproamphodipropionate, Disodium Capryloamphodiacetate, Disodium Capryloamphodipropionate, Disodium Capryloyl Glutamate, Disodium Cetearyl Sulfosuccinate, Disodium Cetyl Phenyl Ether Disulfonate, Disodium Cetyl Sulfosuccinate, Disodium Cocamido MEA-Sulfosuccinate, Disodium Cocamido MIPA PEG-4 Sulfosuccinate, Disodium Cocamido MIPA-Sulfosuccinate, Disodium Cocamido PEG-3 Sulfosuccinate, Disodium Coceth-3 Sulfosuccinate, Disodium Cocoamphocarboxyethylhydroxypropylsulfonate, Disodium Cocoamphodiacetate, Disodium Cocoamphodipropionate, Disodium Coco-Glucoside Sulfosuccinate, Disodium Coco-Sulfosuccinate, Disodium Cocoyl Butyl Gluceth-10 Sulfosuccinate, Disodium Cocoyl Glutamate, Disodium C12-14 Pareth-1 Sulfosuccinate, Disodium C12-14 Pareth-2 Sulfosuccinate, Disodium C12-15 Pareth Sulfosuccinate, Disodium C12-14 Sec-Pareth-3 Sulfosuccinate, Disodium C12-14 Sec-Pareth-5 Sulfosuccinate, Disodium C12-14 Sec-Pareth-7 Sulfosuccinate, Disodium C12-14 Sec-Pareth-9 Sulfosuccinate, Disodium C12-14 Sec-Pareth-12 Sulfosuccinate, Disodium Deceth-5 Sulfosuccinate, Disodium Deceth-6 Sulfosuccinate, Disodium Decyl Phenyl Ether Disulfonate, Disodium Dihydroxyethyl Sulfosuccinylundecylenate, Disodium Ethylene Dicocamide PEG-15 Disulfate, Disodium Hydrogenated Cottonseed Glyceride Sulfosuccinate, Disodium Hydrogenated Tallow Glutamate, Disodium Hydroxydecyl Sorbitol Citrate, Disodium Isodecyl Sulfosuccinate, Disodium Isostearamido MEA-Sulfosuccinate, Disodium Isostearamido MIPA-Sulfosuccinate, Disodium Isostearoamphodiacetate, Disodium Isostearoamphodipropionate, Disodium Isostearyl Sulfosuccinate, Disodium Laneth-5 Sulfosuccinate, Disodium Lauramido MEA-Sulfosuccinate, Disodium Lauramido MIPA Glycol Sulfosuccinate, Disodium Lauramido PEG-2 Sulfosuccinate, Disodium Lauramido PEG-5 Sulfosuccinate, Disodium Laureth-5 Carboxyamphodiacetate, Disodium Laureth-7 Citrate, Disodium Laureth Sulfosuccinate, Disodium Laureth-6 Sulfosuccinate, Disodium Laureth-9 Sulfosuccinate, Disodium Laureth-12 Sulfosuccinate, Disodium Lauriminobishydroxypropylsulfonate, Disodium Lauriminodiacetate, Disodium Lauriminodipropionate, Disodium Lauriminodipropionate Tocopheryl Phosphates, Disodium Lauroamphodiacetate, Disodium Lauroamphodipropionate, Disodium N-Lauroyl Aspartate, Disodium Lauroyl Glutamate, Disodium Lauryl Phenyl Ether Disulfonate, Disodium Lauryl Sulfosuccinate, Disodium Myristamido MEA-Sulfosuccinate, Disodium Nonoxynol-10 Sulfosuccinate, Disodium Oleamido MEA-Sulfosuccinate, Disodium Oleamido MIPA-Sulfosuccinate, Disodium Oleamido PEG-2 Sulfosuccinate, Disodium Oleoamphodipropionate, Disodium Oleth-3 Sulfosuccinate, Disodium Oleyl Phosphate, Disodium Oleyl Sulfosuccinate, Disodium Palmitamido PEG-2 Sulfosuccinate, Disodium Palmitoleamido PEG-2 Sulfosuccinate, Disodium PEG-4 Cocamido MIPA-Sulfosuccinate, Disodium PEG-12 Dimethicone Sulfosuccinate, Disodium PEG-8 Palm Glycerides Sulfosuccinate, Disodium PPG-2-Isodeceth-7 Carboxyamphodiacetate, Disodium Ricinoleamido MEA-Sulfosuccinate, Disodium Sitostereth-14 Sulfosuccinate, Disodium Soyamphodiacetate, Disodium Stearamido MEA-Sulfosuccinate, Disodium Steariminodipropionate, Disodium Stearoamphodiacetate, Disodium Stearoyl Glutamate, Disodium Stearyl Sulfosuccinamate, Disodium Stearyl Sulfosuccinate, Disodium 2-Sulfolaurate, Disodium 2-Sulfopalmitate, Disodium Tallamido MEA-Sulfosuccinate, Disodium Tallowamido MEA-Sulfosuccinate, Disodium Tallowamphodiacetate, Disodium Tallowiminodipropionate, Disodium Tallow Sulfosuccinamate, Disodium Tridecylsulfosuccinate, Disodium Undecylenamido MEA-Sulfosuccinate, Disodium Undecylenamido PEG-2 Sulfosuccinate, Disodium Undecylenoyl Glutamate, Disodium Wheat Germamido MEA-Sulfosuccinate, Disodium Wheat Germamido PEG-2 Sulfosuccinate, Disodium Wheatgermamphodiacetate, Di-TEA-Cocamide Diacetate, Di-TEA-Oleamido PEG-2 Sulfosuccinate, Di-TEA-Palmitoyl Aspartate, Ditridecyl Sodium Sulfosuccinate, Dodecylbenzene Sulfonic Acid, Erucamidopropyl Hydroxysultaine, Ethylhexeth-3 Carboxylic Acid, Ethyl PEG-15 Cocamine Sulfate, Glyceryl Capryl Ether, Hexyldecanoic Acid, Hydrogenated Coconut Acid, Hydrogenated Laneth-25, Hydrogenated Menhaden Acid, Hydrogenated Palm Acid, Hydrogenated Palm Kernel Amine Oxide, Hydrogenated Tallow Acid, Hydrogenated Tallowamine Oxide, Hydrogenated Tallow Betaine, Hydrogenated Talloweth-25, Hydrogenated Tallowoyl Glutamic Acid, Hydrolyzed Candida Bombicola Extract, Hydroxyceteth-60, Hydroxyethyl Acetomonium PG-Dimethicone, Hydroxyethylbutylamine Laureth Sulfate, Hydroxyethyl Carboxymethyl Cocamidopropylamine, Hydroxyethyl Hydroxypropyl C12-15 Alkoxypropylamine Oxide, Hydroxylauryl/Hydroxymyristyl Betaine, Hydroxystearic Acid, Hydroxysuccinimidyl C10-40 Isoalkyl Acidate, Hydroxysuccinimidyl C21-22 Isoalkyl Acidate, Hydroxysultaines, IPDI/PEG-15 Soyamine Oxide Copolymer, IPDI/PEG-15 Soyethonium Ethosulfate Copolymer, IPDI/PEG-15 Soy Glycinate Copolymer, Isoceteth-30, Isolaureth-4 Phosphate, Isopolyglyceryl-3 Dimethicone, Isopolyglyceryl-3 Dimethiconol, Isopropanolamine Lanolate, Isopropylamine Dodecylbenzenesulfonate, Isostearamidopropylamine Oxide, Isostearamidopropyl Betaine, Isostearamidopropyl Morpholine Oxide, Isosteareth-8, Isosteareth-16, Isosteareth-22, Isosteareth-25, Isosteareth-50, Isostearic Acid, Isostearoyl Hydrolyzed Collagen, Jojoba Oil PEG-150 Esters, Jojoba Wax PEG-80 Esters, Jojoba Wax PEG-120 Esters, Laneth-20, Laneth-25, Laneth-40, Laneth-50, Laneth-60, Laneth-75, Lanolin Acid, Lauramidopropylamine Oxide, Lauramidopropyl Betaine, Lauramidopropyl Hydroxysultaine, Lauramine Oxide, Lauraminopropionic Acid, Laurdimoniumhydroxypropyl Decylglucosides Chloride, Laurdimoniumhydroxypropyl Laurylglucosides Chloride, Laureth-16, Laureth-20, Laureth-21, Laureth-23, Laureth-25, Laureth-30, Laureth-38, Laureth-40, Laureth-3 Carboxylic Acid, Laureth-4 Carboxylic Acid, Laureth-5 Carboxylic Acid, Laureth-6 Carboxylic Acid, Laureth-8 Carboxylic Acid, Laureth-10 Carboxylic Acid, Laureth-11 Carboxylic Acid, Laureth-12 Carboxylic Acid, Laureth-13 Carboxylic Acid, Laureth-14 Carboxylic Acid, Laureth-17 Carboxylic Acid, Laureth-6 Citrate, Laureth-7 Citrate, Laureth-1 Phosphate, Laureth-2 Phosphate, Laureth-3 Phosphate, Laureth-4 Phosphate, Laureth-7 Phosphate, Laureth-8 Phosphate, Laureth-7 Tartrate, Laurie Acid, Laurimino Bispropanediol, Lauriminodipropionic Acid, Lauroamphodipropionic Acid, Lauroyl Beta-Alanine, Lauroyl Collagen Amino Acids, Lauroyl Ethyltrimonium Methosulfate, Lauroyl Hydrolyzed Collagen, Lauroyl Hydrolyzed Elastin, Lauroyl Methyl Glucamide, Lauroyl Sarcosine, Lauroyl Silk Amino Acids, Lauryl Betaine, Lauryl Dimethicone/Polyglycerin-3 Crosspolymer, Lauryldimoniumhydroxypropyl Cocoglucosides Chloride, Lauryl Glucoside, Laurylglucosides Hydroxypropyltrimonium Chloride, Lauryl Glycol Hydroxypropyl Ether, Lauryl Hydroxysultaine, Lauryl Malamide, Lauryl Methylglucamide, Lauryl/Myristyl Glycol Hydroxypropyl Ether, Lauryl/Myristyl Wheat Bran/Straw Glycosides, Lauryl Polyglyceryl-3 Polydimethylsiloxyethyl Dimethicone, Lauryl Pyrrolidone, Lauryl Sultaine, Linoleic Acid, Linolenic Acid, Linseed Acid, Lysine Cocoate, Macadamia Seed Oil Glycereth-8 Esters, Magnesium Coceth Sulfate, Magnesium Coco-Sulfate, Magnesium Isododecylbenzenesulfonate, Magnesium Laureth-11 Carboxylate, Magnesium Laureth Sulfate, Magnesium Laureth-5 Sulfate, Magnesium Laureth-8 Sulfate, Magnesium Laureth-16 Sulfate, Magnesium Laureth-3 Sulfosuccinate, Magnesium Lauryl Hydroxypropyl Sulfonate, Magnesium Lauryl Sulfate, Magnesium Methyl Cocoyl Taurate, Magnesium Myreth Sulfate, Magnesium Oleth Sulfate, Magnesium/TEA-Coco-Sulfate, Manicouagan Clay, MEA-Cocoate, MEA-Laureth-6 Carboxylate, MEA-Laureth Sulfate, MEA-Lauryl Sulfate, MEA PPG-6 Laureth-7 Carboxylate, MEA-PPG-8-Steareth-7 Carboxylate, MEA-Undecylenate, Meroxapol 108, Meroxapol 174, Meroxapol 178, Meroxapol 254, Meroxapol 255, Meroxapol 258, Meroxapol 314, Methoxy PEG-450 Amidoglutaroyl Succinimide, Methoxy PEG-450 Amido Hydroxysuccinimidyl Succinamate, Methoxy PEG-450 Maleimide, Methyl Morpholine Oxide, Milkamidopropyl Amine Oxide, Milkamidopropyl Betaine, Minkamidopropylamine Oxide, Minkamidopropyl Betaine, MIPA C12-15 Pareth Sulfate, MIPA-Dodecylbenzenesulfonate, MIPA-Laureth Sulfate, MIPA-Lauryl Sulfate, Mixed Isopropanolamines Lanolate, Mixed Isopropanolamines Lauryl Sulfate, Mixed Isopropanolamines Myristate, Morpholine Oleate, Morpholine Stearate, Myreth-3 Carboxylic Acid, Myreth-5 Carboxylic Acid, Myristalkonium Chloride, Myristamidopropylamine Oxide, Myristamidopropyl Betaine, Myristamidopropyl Dimethylamine Phosphate, Myristamidopropyl Hydroxysultaine, Myristamidopropyl PG-Dimonium Chloride Phosphate, Myristamine Oxide, Myristaminopropionic Acid, Myristic Acid, Myristoyl Ethyltrimonium Methosulfate, Myristoyl Glutamic Acid, Myristoyl Hydrolyzed Collagen, Myristoyl Sarcosine, Myristyl Betaine, Myristyl/Cetyl Amine Oxide, Myristyldimoniumhydroxypropyl Cocoglucosides Chloride, Myristyl Glucoside, Myristyl Phosphate, Nonoxynol-20, Nonoxynol-23, Nonoxynol-25, Nonoxynol-30, Nonoxynol-35, Nonoxynol-40, Nonoxynol-44, Nonoxynol-50, Nonoxynol-100, Nonoxynol-120, Nonoxynol-5 Carboxylic Acid, Nonoxynol-8 Carboxylic Acid, Nonoxynol-10 Carboxylic Acid, Nonoxynol-3 Phosphate, Nonoxynol-4 Phosphate, Nonoxynol-6 Phosphate, Nonoxynol-9 Phosphate, Nonoxynol-10 Phosphate, Nonyl Nonoxynol-30, Nonyl Nonoxynol-49, Nonyl Nonoxynol-100, Nonyl Nonoxynol-150, Nonyl Nonoxynol-7 Phosphate, Nonyl Nonoxynol-8 Phosphate, Nonyl Nonoxynol-9 Phosphate, Nonyl Nonoxynol-10 Phosphate, Nonyl Nonoxynol-11 Phosphate, Nonyl Nonoxynol-15 Phosphate, Nonyl Nonoxynol-24 Phosphate, Oatamidopropyl Betaine, Octoxynol-16, Octoxynol-25, Octoxynol-30, Octoxynol-33, Octoxynol-40, Octoxynol-70, Octoxynol-20 Carboxylic Acid, Octyldodeceth-20, Octyldodeceth-25, Octyldodeceth-30, Oleamidopropylamine Oxide, Oleamidopropyl Betaine, Oleamidopropyl Hydroxysultaine, Oleamine Oxide, Oleic Acid, Oleoyl Hydrolyzed Collagen, Oleoyl Sarcosine, Oleth-20, Oleth-23, Oleth-24, Oleth-25, Oleth-30, Oleth-35, Oleth-40, Oleth-44, Oleth-50, Oleth-3 Carboxylic Acid, Oleth-6 Carboxylic Acid, Oleth-10 Carboxylic Acid, Oleyl Betaine, Olivamidopropylamine Oxide, Olivamidopropyl Betaine, Olive Acid, Olivoyl Hydrolyzed Wheat Protein, Ophiopogon Extract Stearate, Ozonized Oleth-10, Ozonized PEG-10 Oleate, Ozonized PEG-14 Oleate, Ozonized Polysorbate 80, Palm Acid, Palmamidopropyl Betaine, Palmeth-2 Phosphate, Palm itamidopropylamine Oxide, Palm itamidopropyl Betaine, Palmitamine Oxide, Palmitic Acid, Palmitoyl Collagen Amino Acids, Palmitoyl Glycine, Palmitoyl Hydrolyzed Collagen, Palmitoyl Hydrolyzed Milk Protein, Palmitoyl Hydrolyzed Wheat Protein, Palmitoyl Keratin Amino Acids, Palmitoyl Oligopeptide, Palmitoyl Silk Amino Acids, Palm Kernel Acid, Palm Kernelamidopropyl Betaine, Peach Kernel Oil Glycereth-8 Esters, Peanut Acid, PEG-10 Castor Oil, PEG-40 Castor Oil, PEG-44 Castor Oil, PEG-50 Castor Oil, PEG-54 Castor Oil, PEG-55 Castor Oil, PEG-60 Castor Oil, PEG-80 Castor Oil, PEG-100 Castor Oil, PEG-200 Castor Oil, PEG-11 Cocamide, PEG-6 Cocamide Phosphate, PEG-4 Cocamine, PEG-8 Cocamine, PEG-12 Cocamine, PEG-150 Dibehenate, PEG-90 Diisostearate, PEG-75 Dilaurate, PEG-150 Dilaurate, PEG-75 Dioleate, PEG-150 Dioleate, PEG-75 Distearate, PEG-120 Distearate, PEG-150 Distearate, PEG-175 Distearate, PEG-190 Distearate, PEG-250 Distearate, PEG-30 Glyceryl Cocoate, PEG-40 Glyceryl Cocoate, PEG-78 Glyceryl Cocoate, PEG-80 Glyceryl Cocoate, PEG-30 Glyceryl Isostearate, PEG-40 Glyceryl Isostearate, PEG-50 Glyceryl Isostearate, PEG-60 Glyceryl Isostearate, PEG-90 Glyceryl Isostearate, PEG-23 Glyceryl Laurate, PEG-30 Glyceryl Laurate, PEG-25 Glyceryl Oleate, PEG-30 Glyceryl Oleate, PEG-30 Glyceryl Soyate, PEG-25 Glyceryl Stearate, PEG-30 Glyceryl Stearate, PEG-40 Glyceryl Stearate, PEG-120 Glyceryl Stearate, PEG-200 Glyceryl Stearate, PEG-28 Glyceryl Tallowate, PEG-80 Glyceryl Tallowate, PEG-82 Glyceryl Tallowate, PEG-130 Glyceryl Tallowate, PEG-200 Glyceryl Tallowate, PEG-45 Hydrogenated Castor Oil, PEG-50 Hydrogenated Castor Oil, PEG-54 Hydrogenated Castor Oil, PEG-55 Hydrogenated Castor Oil, PEG-60 Hydrogenated Castor Oil, PEG-80 Hydrogenated Castor Oil, PEG-100 Hydrogenated Castor Oil, PEG-200 Hydrogenated Castor Oil, PEG-30 Hydrogenated Lanolin, PEG-70 Hydrogenated Lanolin, PEG-50 Hydrogenated Palmamide, PEG-2 Isostearate, PEG-3 Isostearate, PEG-4 Isostearate, PEG-6 Isostearate, PEG-8 Isostearate, PEG-10 Isostearate, PEG-12 Isostearate, PEG-20 Isostearate, PEG-30 Isostearate, PEG-40 Isostearate, PEG-26 Jojoba Acid, PEG-40 Jojoba Acid, PEG-15 Jojoba Alcohol, PEG-26 Jojoba Alcohol, PEG-40 Jojoba Alcohol, PEG-35 Lanolin, PEG-40 Lanolin, PEG-50 Lanolin, PEG-55 Lanolin, PEG-60 Lanolin, PEG-70 Lanolin, PEG-75 Lanolin, PEG-85 Lanolin, PEG-100 Lanolin, PEG-150 Lanolin, PEG-75 Lanolin Oil, PEG-2 Lauramide, PEG-3 Lauramine Oxide, PEG-20 Laurate, PEG-32 Laurate, PEG-75 Laurate, PEG-150 Laurate, PEG-70 Mango Glycerides, PEG-20 Mannitan Laurate, PEG-8 Methyl Ether Dimethicone, PEG-120 Methyl Glucose Dioleate, PEG-80 Methyl Glucose Laurate, PEG-120 Methyl Glucose Trioleate, PEG-4 Montanate, PEG-30 Oleamine, PEG-20 Oleate, PEG-23 Oleate, PEG-32 Oleate, PEG-36 Oleate, PEG-75 Oleate, PEG-150 Oleate, PEG-20 Palmitate, PEG-150 Polyglyceryl-2 Tristearate, PEG/PPG-28/21 Acetate Dimethicone, PEG/PPG-24/18 Butyl Ether Dimethicone, PEG/PPG-3/17 Copolymer, PEG/PPG-5/35 Copolymer, PEG/PPG-8/55 Copolymer, PEG/PPG-10/30 Copolymer, PEG/PPG-10/65 Copolymer, PEG/PPG-12/35 Copolymer, PEG/PPG-16/17 Copolymer, PEG/PPG-20/9 Copolymer, PEG/PPG-20/20 Copolymer, PEG/PPG-20/60 Copolymer, PEG/PPG-20/65 Copolymer, PEG/PPG-22/25 Copolymer, PEG/PPG-28/30 Copolymer, PEG/PPG-30-35 Copolymer, PEG/PPG-30/55 Copolymer, PEG/PPG-35/40 Copolymer, PEG/PPG-50/40 Copolymer, PEG/PPG-150/35 Copolymer, PEG/PPG-160/30 Copolymer, PEG/PPG-190/60 Copolymer, PEG/PPG-200/40 Copolymer, PEG/PPG-300/55 Copolymer, PEG/PPG-20/22 Methyl Ether Dimethicone, PEG-26-PPG-30 Phosphate, PEG/PPG-4/2 Propylheptyl Ether, PEG/PPG-6/2 Propylheptyl Ether, PEG-7/PPG-2 Propylheptyl Ether, PEG/PPG-8/2 Propylheptyl Ether, PEG/PPG-10/2 Propylheptyl Ether, PEG/PPG-14/2 Propylheptyl Ether, PEG/PPG-40/2 Propylheptyl Ether, PEG/PPG-10/2 Ricinoleate, PEG/PPG-32/3 Ricinoleate, PEG-55 Propylene Glycol Oleate, PEG-25 Propylene Glycol Stearate, PEG-75 Propylene Glycol Stearate, PEG-120 Propylene Glycol Stearate, PEG-5 Rapeseed Sterol, PEG-10 Rapeseed Sterol, PEG-40 Ricinoleamide, PEG-75 Shea Butter Glycerides, PEG-75 Shorea Butter Glycerides, PEG-20 Sorbitan Cocoate, PEG-20 Sorbitan Isostearate, PEG-40 Sorbitan Lanolate, PEG-75 Sorbitan Lanolate, PEG-10 Sorbitan Laurate, PEG-40 Sorbitan Laurate, PEG-44 Sorbitan Laurate, PEG-75 Sorbitan Laurate, PEG-80 Sorbitan Laurate, PEG-20 Sorbitan Oleate, PEG-80 Sorbitan Palmitate, PEG-40 Sorbitan Stearate, PEG-60 Sorbitan Stearate, PEG-160 Sorbitan Triisostearate, PEG-40 Soy Sterol, PEG-2 Stearamide Carboxylic Acid, PEG-9 Stearamide Carboxylic Acid, PEG-20 Stearate, PEG-23 Stearate, PEG-25 Stearate, PEG-30 Stearate, PEG-32 Stearate, PEG-35 Stearate, PEG-36 Stearate, PEG-40 Stearate, PEG-45 Stearate, PEG-50 Stearate, PEG-55 Stearate, PEG-75 Stearate, PEG-90 Stearate, PEG-100 Stearate, PEG-120 Stearate, PEG-150 Stearate, PEG-45 Stearate Phosphate, PEG-20 Tallate, PEG-50 Tallow Amide, PEG-2 Tallowamide DEA, PEG-20 Tallowate, PEG-66 Trihydroxystearin, PEG-200 Trihydroxystearin, PEG-60 Tsubakiate Glycerides, Pelargonic Acid, Pentadoxynol-200, Pheneth-6 Phosphate, Poloxamer 105, Poloxamer 108, Poloxamer 182, Poloxamer 183, Poloxamer 184, Poloxamer 188, Poloxamer 217, Poloxamer 234, Poloxamer 235, Poloxamer 237, Poloxamer 238, Poloxamer 288, Poloxamer 334, Poloxamer 335, Poloxamer 338, Poloxamine 908, Poloxamine 1508, Polydimethylsiloxy PEG/PPG-24/19 Butyl Ether Silsesquioxane, Polydimethylsiloxy PPG-13 Butyl Ether Silsesquioxane, Polyglyceryl-6 Caprate, Polyglyceryl-10 Dilaurate, Polyglyceryl-20 Heptacaprylate, Polyglyceryl-20 Hexacaprylate, Polyglyceryl-2 Lauryl Ether, Polyglyceryl-10 Lauryl Ether, Polyglyceryl-20 Octaisononanoate, Polyglyceryl-6 Pentacaprylate, Polyglyceryl-10 Pentacaprylate, Polyglyceryl-3 Polydimethylsiloxyethyl Dimethicone, Polyglyceryl-6 Tetracaprylate, Polyglyceryl-10 Tetralaurate, Polyglyceryl-6 Tricaprylate, Polyglyceryl-10 Trilaurate, Polyquaternium-77, Polyquaternium-78, Polyquaternium-79, Polyquaternium-80, Polyquaternium-81, Polyquaternium-82, Pomaderris Kumerahou Flower/Leaf Extract, Poria Cocos Extract, Potassium Abietoyl Hydrolyzed Collagen, Potassium Babassuate, Potassium Behenate, Potassium C9-15 Alkyl Phosphate, Potassium C11-15 Alkyl Phosphate, Potassium C12-13 Alkyl Phosphate, Potassium C12-14 Alkyl Phosphate, Potassium Caprate, Potassium Capryloyl Glutamate, Potassium Capryloyl Hydrolyzed Rice Protein, Potassium Castorate, Potassium Cocoate, Potassium Cocoyl Glutamate, Potassium Cocoyl Glycinate, Potassium Cocoyl Hydrolyzed Casein, Potassium Cocoyl Hydrolyzed Collagen, Potassium Cocoyl Hydrolyzed Corn Protein, Potassium Cocoyl Hydrolyzed Keratin, Potassium Cocoyl Hydrolyzed Oat Protein, Potassium Cocoyl Hydrolyzed Potato Protein, Potassium Cocoyl Hydrolyzed Rice Bran Protein, Potassium Cocoyl Hydrolyzed Rice Protein, Potassium Cocoyl Hydrolyzed Silk, Potassium Cocoyl Hydrolyzed Soy Protein, Potassium Cocoyl Hydrolyzed Wheat Protein, Potassium Cocoyl Hydrolyzed Yeast Protein, Potassium Cocoyl PCA, Potassium Cocoyl Sarcosinate, Potassium Cocoyl Taurate, Potassium Cornate, Potassium Cyclocarboxypropyloleate, Potassium Dihydroxyethyl Cocamine Oxide Phosphate, Potassium Dimethicone PEG-7 Phosphate, Potassium Dodecylbenzenesulfonate, Potassium Hempseedate, Potassium Hydrogenated Cocoate, Potassium Hydrogenated Palmate, Potassium Hydrogenated Tallowate, Potassium Hydroxystearate, Potassium Isostearate, Potassium Lanolate, Potassium Laurate, Potassium Laureth-3 Carboxylate, Potassium Laureth-4 Carboxylate, Potassium Laureth-5 Carboxylate, Potassium Laureth-6 Carboxylate, Potassium Laureth-10 Carboxylate, Potassium Laureth Phosphate, Potassium Lauroyl Collagen Amino Acids, Potassium Lauroyl Glutamate, Potassium Lauroyl Hydrolyzed Collagen, Potassium Lauroyl Hydrolyzed Pea Protein, Potassium Lauroyl Hydrolyzed Soy Protein, Potassium Lauroyl PCA, Potassium Lauroyl Pea Amino Acids, Potassium Lauroyl Sarcosinate, Potassium Lauroyl Silk Amino Acids, Potassium Lauroyl Wheat Amino Acids, Potassium Lauryl Phosphate, Potassium Lauryl Sulfate, Potassium Linoleate, Potassium Metaphosphate, Potassium Methyl Cocoyl Taurate, Potassium Myristate, Potassium Myristoyl Glutamate, Potassium Myristoyl Hydrolyzed Collagen, Potassium Octoxynol-12 Phosphate, Potassium Oleate, Potassium Oleoyl Hydrolyzed Collagen, Potassium Olivate, Potassium Olivoyl Hydrolyzed Oat Protein, Potassium Olivoyl Hydrolyzed Wheat Protein, Potassium Olivoyl/Lauroyl Wheat Amino Acids, Potassium Olivoyl PCA, Potassium Palmate, Potassium Palmitate, Potassium Palmitoyl Hydrolyzed Corn Protein, Potassium Palmitoyl Hydrolyzed Oat Protein, Potassium Palmitoyl Hydrolyzed Rice Protein, Potassium Palmitoyl Hydrolyzed Sweet Almond Protein, Potassium Palmitoyl Hydrolyzed Wheat Protein, Potassium Palm Kernelate, Potassium Peanutate, Potassium Rapeseedate, Potassium Ricinoleate, Potassium Safflowerate, Potassium Soyate, Potassium Stearate, Potassium Stearoyl Hydrolyzed Collagen, Potassium Tallate, Potassium Tallowate, Potassium Taurate, Potassium Taurine Laurate, Potassium Trideceth-3 Carboxylate, Potassium Trideceth-4 Carboxylate, Potassium Trideceth-7 Carboxylate, Potassium Trideceth-15 Carboxylate, Potassium Trideceth-19 Carboxylate, Potassium Trideceth-6 Phosphate, Potassium Trideceth-7 Phosphate, Potassium Tsubakiate, Potassium Undecylenate, Potassium Undecylenoyl Hydrolyzed Collagen, Potassium Undecylenoyl Hydrolyzed Rice Protein, PPG-30-Buteth-30, PPG-36-Buteth-36, PPG-38-Buteth-37, PPG-30-Capryleth-4 Phosphate, PPG-10 Cetyl Ether Phosphate, PPG-2 C9-11 Pareth-8, PPG-1-Deceth-5, PPG-3-Deceth-2 Carboxylic Acid, PPG-30 Ethylhexeth-4 Phosphate, PPG-20-Glycereth-30, PPG-2 Hydroxyethyl Coco/Isostearamide, PPG-2-Isodeceth-8, PPG-2-Isodeceth-10, PPG-2-Isodeceth-18, PPG-2-Isodeceth-25, PPG-4-Isodeceth-10, Propyltrimonium Hydrolyzed Collagen, Quaternium-24, Quaternium-52, Quaternium-87, Rapeseed Acid, Rice Bran Acid, Rice Oil Glycereth-8 Esters, Ricinoleamidopropyl Betaine, Ricinoleic Acid, Ricinoleth-40, Safflower Acid, Sapindus Oahuensis Fruit Extract, Saponaria Officinalis Root Powder, Saponins, Sekken-K, Sekken-Na/K, Sekken Soji, Sekken Soji-K, Sesame Oil Glycereth-8 Esters, Sesamidopropylamine Oxide, Sesamidopropyl Betaine, Shea Butteramidopropyl Betaine, Shea Butter Glycereth-8 Esters, Sodium Arachidate, Sodium Arganampohoacetate, Sodium Astrocaryum Murumuruate, Sodium Avocadoate, Sodium Babassuamphoacetate, Sodium Babassuate, Sodium Babassu Sulfate, Sodium Behenate, Sodium Bisglycol Ricinosulfosuccinate, Sodium Bis-Hydroxyethylglycinate Coco-Glucosides Crosspolymer, Sodium Bis-Hydroxyethylglycinate Lauryl-Glucosides Crosspolymer, Sodium Borageamidopropyl PG-Dimonium Chloride Phosphate, Sodium Butoxynol-12 Sulfate, Sodium Butylglucosides Hydroxypropyl Phosphate, Sodium C13-17 Alkane Sulfonate, Sodium C14-18 Alkane Sulfonate, Sodium C12-15 Alkoxypropyl Iminodipropionate, Sodium C10-16 Alkyl Sulfate, Sodium C11-15 Alkyl Sulfate, Sodium C12-13 Alkyl Sulfate, Sodium C12-15 Alkyl Sulfate, Sodium C12-18 Alkyl Sulfate, Sodium C16-20 Alkyl Sulfate, Sodium C9-22 Alkyl Sec Sulfonate, Sodium C14-17 Alkyl Sec Sulfonate, Sodium Caprate, Sodium Caproamphoacetate, Sodium Caproamphohydroxypropylsulfonate, Sodium Caproamphopropionate, Sodium Caproyl Methyltaurate, Sodium Caprylate, Sodium Capryleth-2 Carboxylate, Sodium Capryleth-9 Carboxylate, Sodium Capryloamphoacetate, Sodium Capryloamphohydroxypropylsulfonate, Sodium Capryloamphopropionate, Sodium Capryloyl Glutamate, Sodium Capryloyl Hydrolyzed Wheat Protein, Sodium Caprylyl PG-Sulfonate, Sodium Caprylyl Sulfonate, Sodium Castorate, Sodium Ceteareth-13 Carboxylate, Sodium Cetearyl Sulfate, Sodium Ceteth-13 Carboxylate, Sodium Cetyl Sulfate, Sodium Cocamidopropyl PG-Dimonium Chloride Phosphate, Sodium Cocaminopropionate, Sodium Coceth Sulfate, Sodium Coceth-30 Sulfate, Sodium Cocoabutteramphoacetate, Sodium Cocoa Butterate, Sodium Cocoamphoacetate, Sodium Cocoamphohydroxypropylsulfonate, Sodium Cocoamphopropionate, Sodium Cocoate, Sodium Coco/Babassu/Andiroba Sulfate, Sodium Coco/Babassu Sulfate, Sodium Cocoglucosides Hydroxypropyl Phosphate, Sodium Cocoglucosides Hydroxypropylsulfonate, Sodium Coco-Glucoside Tartrate, Sodium Cocoglyceryl Ether Sulfonate, Sodium Coco/Hydrogenated Tallow Sulfate, Sodium Cocoiminodiacetate, Sodium Cocomonoglyceride Sulfate, Sodium Cocomonoglyceride Sulfonate, Sodium Coco PG-Dimonium Chloride Phosphate, Sodium Coco-Sulfate, Sodium Coco Sulfoacetate, Sodium Cocoyl Alaninate, Sodium Cocoyl Amino Acids, Sodium Cocoyl Collagen Amino Acids, Sodium Cocoyl Glutamate, Sodium Cocoyl Glutaminate, Sodium Cocoyl Glycinate, Sodium Cocoyl/Hydrogenated Tallow Glutamate, Sodium Cocoyl Hydrolyzed Collagen, Sodium Cocoyl Hydrolyzed Keratin, Sodium Cocoyl Hydrolyzed Rice Protein, Sodium Cocoyl Hydrolyzed Silk, Sodium Cocoyl Hydrolyzed Soy Protein, Sodium Cocoyl Hydrolyzed Sweet Almond Protein, Sodium Cocoyl Hydrolyzed Wheat Protein, Sodium Cocoyl Hydrolyzed Wheat Protein Glutamate, Sodium Cocoyl Isethionate, Sodium Cocoyl Methylaminopropionate, Sodium Cocoyl Oat Amino Acids, Sodium Cocoyl/Palmoyl/Sunfloweroyl Glutamate, Sodium Cocoyl Proline, Sodium Cocoyl Sarcosinate, Sodium Cocoyl Taurate, Sodium Cocoyl Threoninate, Sodium Cocoyl Wheat Amino Acids, Sodium C12-14 Olefin Sulfonate, Sodium C14-16 Olefin Sulfonate, Sodium C14-18 Olefin Sulfonate, Sodium C16-18 Olefin Sulfonate, Sodium Cornamphopropionate, Sodium Cottonseedamphoacetate, Sodium C13-15 Pareth-8 Butyl Phosphate, Sodium C9-11 Pareth-6 Carboxylate, Sodium C11-15 Pareth-7 Carboxylate, Sodium C12-13 Pareth-5 Carboxylate, Sodium C12-13 Pareth-8 Carboxylate, Sodium C12-13 Pareth-12 Carboxylate, Sodium C12-15 Pareth-6 Carboxylate, Sodium C12-15 Pareth-7 Carboxylate, Sodium C12-15 Pareth-8 Carboxylate, Sodium C14-15 Pareth-8 Carboxylate, Sodium C12-14 Sec-Pareth-8 Carboxylate, Sodium C14-15 Pareth-PG Sulfonate, Sodium C12-13 Pareth-2 Phosphate, Sodium C13-15 Pareth-8 Phosphate, Sodium C9-15 Pareth-3 Sulfate, Sodium C10-15 Pareth Sulfate, Sodium C10-16 Pareth-2 Sulfate, Sodium C12-13 Pareth Sulfate, Sodium C12-15 Pareth Sulfate, Sodium C12-15 Pareth-3 Sulfate, Sodium C13-15 Pareth-3 Sulfate, Sodium C12-14 Sec-Pareth-3 Sulfate, Sodium C12-15 Pareth-3 Sulfonate, Sodium C12-15 Pareth-7 Sulfonate, Sodium C12-15 Pareth-15 Sulfonate, Sodium Deceth-2 Carboxylate, Sodium Deceth Sulfate, Sodium Decylbenzenesulfonate, Sodium Decylglucosides Hydroxypropyl Phosphate, Sodium Decylglucosides Hydroxypropylsulfonate, Sodium Dilaureth-7 Citrate, Sodium Dilaureth-10 Phosphate, Sodium Dilinoleamidopropyl PG-Dimonium Chloride Phosphate, Sodium Dilinoleate, Sodium Dioleth-8 Phosphate, Sodium Dodecylbenzenesulfonate, Sodium Ethyl 2-Sulfolaurate, Sodium Glyceryl Oleate Phosphate, Sodium Grapeseedamidopropyl PG-Dimonium Chloride Phosphate, Sodium Grapeseedamphoacetate, Sodium Grapeseedate, Sodium Hempseedamphoacetate, Sodium Hexeth-4 Carboxylate, Sodium Hydrogenated Cocoate, Sodium Hydrogenated Cocoyl Methyl Isethionate, Sodium Hydrogenated Palmate, Sodium Hydrogenated Tallowate, Sodium Hydrogenated Tallowoyl Glutamate, Sodium Hydroxylauryldimonium Ethyl Phosphate, Sodium Hydroxypropyl Palm Kernelate Sulfonate, Sodium Hydroxypropylphosphate Decylglucoside Crosspolymer, Sodium Hydroxypropylphosphate Laurylglucoside Crosspolymer, Sodium Hydroxypropylsulfonate Cocoglucoside Crosspolymer, Sodium Hydroxypropylsulfonate Decylglucoside Crosspolymer, Sodium Hydroxypropylsulfonate Laurylglucoside Crosspolymer, Sodium Hydroxystearate, Sodium Isostearate, Sodium Isosteareth-6 Carboxylate, Sodium Isosteareth-11 Carboxylate, Sodium Isostearoamphoacetate, Sodium Isostearoamphopropionate, Sodium N-Isostearoyl Methyltaurate, Sodium Laneth Sulfate, Sodium Lanolate, Sodium Lardate, Sodium Lauramido Diacetate, Sodium Lauraminopropionate, Sodium Laurate, Sodium Laureth-3 Carboxylate, Sodium Laureth-4 Carboxylate, Sodium Laureth-5 Carboxylate, Sodium Laureth-6 Carboxylate, Sodium Laureth-8 Carboxylate, Sodium Laureth-11 Carboxylate, Sodium Laureth-12 Carboxylate, Sodium Laureth-13 Carboxylate, Sodium Laureth-14 Carboxylate, Sodium Laureth-16 Carboxylate, Sodium Laureth-17 Carboxylate, Sodium Laureth Sulfate, Sodium Laureth-5 Sulfate, Sodium Laureth-7 Sulfate, Sodium Laureth-8 Sulfate, Sodium Laureth-12 Sulfate, Sodium Laureth-40 Sulfate, Sodium Laureth-7 Tartrate, Sodium Lauriminodipropionate, Sodium Lauroamphoacetate, Sodium Lauroamphohydroxypropylsulfonate, Sodium Lauroampho PG-Acetate Phosphate, Sodium Lauroamphopropionate, Sodium Lauroyl Aspartate, Sodium Lauroyl Collagen Amino Acids, Sodium Lauroyl Glycine Propionate, Sodium Lauroyl Hydrolyzed Collagen, Sodium Lauroyl Hydrolyzed Silk, Sodium Lauroyl Hydroxypropyl Sulfonate, Sodium Lauroyl Isethionate, Sodium Lauroyl Methylaminopropionate, Sodium Lauroyl Methyl Isethionate, Sodium Lauroyl Millet Amino Acids, Sodium Lauroyl/Myristoyl Aspartate, Sodium Lauroyl Oat Amino Acids, Sodium Lauroyl Sarcosinate, Sodium Lauroyl Silk Amino Acids, Sodium Lauroyl Taurate, Sodium Lauroyl Wheat Amino Acids, Sodium Lauryl Diethylenediaminoglycinate, Sodium Lauryl Glucose Carboxylate, Sodium Laurylglucosides Hydroxypropyl Phosphate, Sodium Laurylglucosides Hydroxypropylsulfonate, Sodium Lauryl Glycol Carboxylate, Sodium Lauryl Hydroxyacetamide Sulfate, Sodium Lauryl Phosphate, Sodium Lauryl Sulfate, Sodium Lauryl Sulfoacetate, Sodium Linoleate, Sodium Macadamiaseedate, Sodium Mangoamphoacetate, Sodium Mangoseedate, Sodium/MEA Laureth-2 Sulfosuccinate, Sodium Methoxy PPG-2 Acetate, Sodium Methyl Cocoyl Taurate, Sodium Methyl Lauroyl Taurate, Sodium Methyl Myristoyl Taurate, Sodium Methyl Oleoyl Taurate, Sodium Methyl Palmitoyl Taurate, Sodium Methyl Stearoyl Taurate, Sodium Methyl 2-Sulfolaurate, Sodium Methyl 2-Sulfopalmitate, Sodium Methyltaurate Isopalmitamide, Sodium Methyltaurine Cocoyl Methyltaurate, Sodium Myreth Sulfate, Sodium Myristate, Sodium Myristoamphoacetate, Sodium Myristoyl Glutamate, Sodium Myristoyl Hydrolyzed Collagen, Sodium Myristoyl Isethionate, Sodium Myristoyl Sarcosinate, Sodium Myristyl Sulfate, Sodium Nonoxynol-6 Phosphate, Sodium Nonoxynol-9 Phosphate, Sodium Nonoxynol-1 Sulfate, Sodium Nonoxynol-3 Sulfate, Sodium Nonoxynol-4 Sulfate, Sodium Nonoxynol-6 Sulfate, Sodium Nonoxynol-8 Sulfate, Sodium Nonoxynol-10 Sulfate, Sodium Nonoxynol-25 Sulfate, Sodium Octoxynol-2 Ethane Sulfonate, Sodium Octoxynol-2 Sulfate, Sodium Octoxynol-6 Sulfate, Sodium Octoxynol-9 Sulfate, Sodium Oleate, Sodium Oleoamphoacetate, Sodium Oleoamphohydroxypropylsulfonate, Sodium Oleoamphopropionate, Sodium Oleoyl Hydrolyzed Collagen, Sodium Oleoyl Isethionate, Sodium Oleth Sulfate, Sodium Oleyl Methyl Isethionate, Sodium Oleyl Sulfate, Sodium Olivamphoacetate, Sodium Olivate, Sodium Olivoyl Glutamate, Sodium Palmamphoacetate, Sodium Palmate, Sodium Palm Glyceride Sulfonate, Sodium Palmitate, Sodium Palmitoyl Hydrolyzed Collagen, Sodium Palmitoyl Hydrolyzed Wheat Protein, Sodium Palmitoyl Sarcosinate, Sodium Palm Kernelate, Sodium Palm Kerneloyl Isethionate, Sodium Palmoyl Glutamate, Sodium Passiflora Edulis Seedate, Sodium Peanutamphoacetate, Sodium Peanutate, Sodium PEG-6 Cocamide Carboxylate, Sodium PEG-8 Cocamide Carboxylate, Sodium PEG-4 Cocamide Sulfate, Sodium PEG-3 Lauramide Carboxylate, Sodium PEG-4 Lauramide Carboxylate, Sodium PEG-8 Palm Glycerides Carboxylate, Sodium Pentaerythrityl Hydroxypropyl Iminodiacetate Dendrimer, Sodium Propoxy PPG-2 Acetate, Sodium Rapeseedate, Sodium Ricebranamphoacetate, Sodium Ricinoleate, Sodium Ricinoleoamphoacetate, Sodium Rose Hipsamphoacetate, Sodium Rosinate, Sodium Safflowerate, Sodium Saffloweroyl Hydrolyzed Soy Protein, Sodium Sesameseedate, Sodium Sesamphoacetate, Sodium Sheabutteramphoacetate, Sodium Soyate, Sodium Soy Hydrolyzed Collagen, Sodium Stearate, Sodium Stearoamphoacetate, Sodium Stearoamphohydroxypropylsulfonate, Sodium Stearoamphopropionate, Sodium Stearoyl Casein, Sodium Stearoyl Glutamate, Sodium Stearoyl Hyaluronate, Sodium Stearoyl Hydrolyzed Collagen, Sodium Stearoyl Hydrolyzed Corn Protein, Sodium Stearoyl Hydrolyzed Silk, Sodium Stearoyl Hydrolyzed Soy Protein, Sodium Stearoyl Hydrolyzed Wheat Protein, Sodium Stearoyl Lactalbumin, Sodium Stearoyl Methyl Isethionate, Sodium Stearoyl Oat Protein, Sodium Stearoyl Pea Protein, Sodium Stearoyl Soy Protein, Sodium Stearyl Dimethyl Glycine, Sodium Stearyl Sulfate, Sodium Sunflowerseedamphoacetate, Sodium Surfactin, Sodium Sweetalmondamphoacetate, Sodium Sweet Almondate, Sodium Tallamphopropionate, Sodium Tallate, Sodium Tallowamphoacetate, Sodium Tallowate, Sodium Tallow Sulfate, Sodium Tamanuseedate, Sodium Taurate, Sodium Taurine Cocoyl Methyltaurate, Sodium Taurine Laurate, Sodium/TEA-Lauroyl Collagen Amino Acids, Sodium/TEA-Lauroyl Hydrolyzed Collagen, Sodium/TEA-Lauroyl Hydrolyzed Keratin, Sodium/TEA-Lauroyl Keratin Amino Acids, Sodium/TEA-Undecylenoyl Collagen Amino Acids, Sodium/TEA-Undecylenoyl Hydrolyzed Collagen, Sodium/TEA-Undecylenoyl Hydrolyzed Corn Protein, Sodium/TEA-Undecylenoyl Hydrolyzed Soy Protein, Sodium/TEA-Undecylenoyl Hydrolyzed Wheat Protein, Sodium Theobroma Grandiflorum Seedate, Sodium Trideceth-3 Carboxylate, Sodium Trideceth-4 Carboxylate, Sodium Trideceth-6 Carboxylate, Sodium Trideceth-7 Carboxylate, Sodium Trideceth-8 Carboxylate, Sodium Trideceth-12 Carboxylate, Sodium Trideceth-15 Carboxylate, Sodium Trideceth-19 Carboxylate, Sodium Trideceth Sulfate, Sodium Tridecylbenzenesulfonate, Sodium Tridecyl Sulfate, Sodium Trimethylolpropane Hydroxypropyl Iminodiacetate Dendrimer, Sodium Undeceth-5 Carboxylate, Sodium Undecylenate, Sodium Undecylenoamphoacetate, Sodium Undecylenoamphopropionate, Sodium Undecylenoyl Glutamate, Sodium Wheat Germamphoacetate, Sorbeth-160 Tristearate, Soy Acid, Soyamidopropylamine Oxide, Soyamidopropyl Betaine, Soybean Oil Glycereth-8 Esters, Stearamidopropylamine Oxide, Stearamidopropyl Betaine, Stearamine Oxide, Steareth-15, Steareth-16, Steareth-20, Steareth-21, Steareth-25, Steareth-27, Steareth-30, Steareth-40, Steareth-50, Steareth-80, Steareth-100, Steareth-2 Phosphate, Steareth-3 Phosphate, Stearic Acid, Stearoxypropyltrimonium Chloride, Stearoyl Glutamic Acid, Stearoyl Sarcosine, Stearyl Betaine, Stearyldimoniumhydroxypropyl Butylglucosides Chloride, Stearyldimoniumhydroxypropyl Decylglucosides Chloride, Stearyldimoniumhydroxypropyl Laurylglucosides Chloride, Sulfated Castor Oil, Sulfated Coconut Oil, Sulfated Glyceryl Oleate, Sulfated Olive Oil, Sulfated Peanut Oil, Sunfloweramide MEA, Sunflower Seed Acid, Sunflowerseedamidopropyl Hydroxyethyldimonium Chloride, Sunflower Seed Oil Glycereth-8 Esters, Tall Oil Acid, Tallow Acid, Tallowamidopropylamine Oxide, Tallowamidopropyl Betaine, Tallowamidopropyl Hydroxysultaine, Tallowamine Oxide, Tallow Betaine, Tallow Dihydroxyethyl Betaine, Tallowoyl Ethyl Glucoside, TEA-Abietoyl Hydrolyzed Collagen, TEA-C12-14 Alkyl Phosphate, TEA-C10-15 Alkyl Sulfate, TEA-C11-15 Alkyl Sulfate, TEA-C12-13 Alkyl Sulfate, TEA-C12-14 Alkyl Sulfate, TEA-C12-15 Alkyl Sulfate, TEA C14-17 Alkyl Sec Sulfonate, TEA-Canolate, TEA-Cocamide Diacetate, TEA-Cocoate, TEA-Coco-Sulfate, TEA-Cocoyl Alaninate, TEA-Cocoyl Glutamate, TEA-Cocoyl Glutaminate, TEA-Cocoyl Glycinate, TEA-Cocoyl Hydrolyzed Collagen, TEA-Cocoyl Hydrolyzed Soy Protein, TEA-Cocoyl Sarcosinate, TEA-Dimethicone PEG-7 Phosphate, TEA-Dodecylbenzenesulfonate, TEA-Hydrogenated Cocoate, TEA-Hydrogenated Tallowoyl Glutamate, TEA-Isostearate, TEA-Isostearoyl Hydrolyzed Collagen, TEA-Lauraminopropionate, TEA-Laurate, TEA-Laurate/Myristate, TEA-Laureth Sulfate, TEA-Lauroyl Collagen Amino Acids, TEA-Lauroyl Glutamate, TEA-Lauroyl Hydrolyzed Collagen, TEA-Lauroyl Keratin Amino Acids, TEA-Lauroyl Methylaminopropionate, TEA-Lauroyl/Myristoyl Aspartate, TEA-Lauroyl Sarcosinate, TEA-Lauryl Phosphate, TEA-Lauryl Sulfate, TEA-Myristaminopropionate, TEA-Myristate, TEA-Myristoyl Hydrolyzed Collagen, TEA-Oleate, TEA-Oleoyl Hydrolyzed Collagen, TEA-Oleoyl Sarcosinate, TEA-Oleyl Sulfate, TEA-Palmitate, TEA-Palm Kernel Sarcosinate, TEA-PEG-3 Cocamide Sulfate, TEA-Rosinate, TEA-Stearate, TEA-Tallate, TEA-T ridecylbenzenesulfonate, TEA-Undecylenate, TEA-Undecylenoyl Hydrolyzed Collagen, Tetramethyl Decynediol, Tetrasodium Dicarboxyethyl Stearyl Sulfosuccinamate, TIPA-Laureth Sulfate, TIPA-Lauryl Sulfate, TIPA-Myristate, TIPA-Stearate, Tocopheryl Phosphate, Trehalose Undecylenoate, TM-C12-15 Pareth-2 Phosphate, TM-C12-15 Pareth-6 Phosphate, TM-C12-15 Pareth-8 Phosphate, TM-C12-15 Pareth-10 Phosphate, Trideceth-20, Trideceth-50, Trideceth-3 Carboxylic Acid, Trideceth-4 Carboxylic Acid, Trideceth-7 Carboxylic Acid, Trideceth-8 Carboxylic Acid, Trideceth-15 Carboxylic Acid, Trideceth-19 Carboxylic Acid, Trideceth-10 Phosphate, Tridecylbenzenesulfonic Acid, Trilaureth-9 Citrate, Trimethylolpropane Hydroxypropyl Bis-Hydroxyethylamine Dendrimer, Trisodium Lauroampho PG-Acetate Chloride Phosphate, Undecanoic Acid, Undeceth-5 Carboxylic Acid, Undecylenamidopropylamine Oxide, Undecylenamidopropyl Betaine, Undecylenic Acid, Undecylenoyl Collagen Amino Acids, Undecylenoyl Glycine, Undecylenoyl Hydrolyzed Collagen, Undecylenoyl Wheat Amino Acids, Undecyl Glucoside, Wheat Germ Acid, Wheat Germamidopropylamine Oxide, Wheat Germamidopropyl Betaine, Yucca Schidigera Leaf/Root/Stem Extract, Yucca Schidigera Stem Extract, Zinc Coceth Sulfatea and Zinc Coco-Sulfate.

Antimicrobials for product protection: For preservative purposes, the cosmetic or pharmaceutical composition according to the present invention preferably includes one or more further antimicrobials which are different from the antimicrobial component (b) of the cosmetic or pharmaceutical composition according to the present invention. Said antimicrobials are suitable or customary in cosmetic or pharmaceutical composition and are valuable in ensuring preservation of the ready-for-use formulations. These antimicrobials act primarily against microorganisms, in particular bacteria, yeast and/or fungi. Specified microorganisms are aerobic mesophilic bacteria or yeast or fungi undesirable in a cosmetic or pharmaceutical product and recognized as a pathogen species that may be harmful for human health or as an indication of hygienic failure in the manufacturing process. Microorganisms considered as specified microorganisms are of the genus Pseudomonas (bacterium), Staphylococcus (bacterium), Escherichia (bacterium), Candida (yeast) and Aspergillus (fungi), and combinations thereof. More preferably, the microorganisms are selected from the group consisting of Pseudomonas aeruginosa, Staphylococcus aureus, Escherichia coli, Candida albicans and Aspergillus brasiliensis, and combinations thereof. By suppressing or inhibiting the microorganisms' growth, the degradation and deterioration of the cosmetic or pharmaceutical preparation, in particular during storage, which sometimes goes along with impairment of the sensory quality of the products, can be prevented or at least minimized.

The composition according to the first or second aspect of the present invention may encompass at least one further antimicrobial for product protection selected from the group consisting of Butylparaben, 2-Benzylheptanol, 4-Hydroxyacetophenone, Alkyl (C 12-22) Trimethyl Ammonium Bromide and Chloride, Ascorbic Acid and salts thereof, Benzalkonium Bromide, Benzalkonium Chloride, Benzalkonium Saccharinate, Benzethonium Chloride, Camphor, Cetylpyridinium Chloride, Chlorhexidine, Chlorhexidine Diacetate, Chlorhexidine Digluconate, Chlorhexidine Dihydrochloride, Chlorocresol, Chloroxylenol, Chlorphenesin, Cyclohexylglycerin, Dehydroacetic Acid, Diazolidinyl Urea, Disodium EDTA, DMDM Hydantoin, Glycerin, Glyceryl Caprate, Hexamidine, Hexamidine Diisethionate, Hexylglycerin, Hydroxyethoxyphenyl Butanol, Hydroxyethoxyphenyl Butanone, Iodopropynyl Butylcarbamate, Isobutylparaben, Itaconic Acid and salts thereof, Lactic Acid, Methyl Salicylate, Methylchloroisothiazolinone, Methylisothiazolinone, Methylparaben and its salts, Phenethyl Alcohol, Polyarginine, Polylysine, Potassium Sorbate, Propylparaben, Silver Chloride, Silver Citrate, Sodium Benzoate, Sodium Ethylparaben, Sodium Levulinate or Levulinic Acid and esters and/or ketals thereof, Sodium Propylparaben, Sorbic Acid, Sorbitan Caprylate, TEA-Salicylate, Tetrasodium Glutamate Diacetate, Triclocarban, Trisodium Ethylenediamine Disuccinate, Zinc Citrate, Trisodium Dicarboxymethyl Alaninate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-pentanediol, 2,3-hexanediol, 2,3-heptanediol, 2,3-octanediol, 2,3-nonanediol, and mixtures of two or more the aforesaid compounds.

In a preferred variant, the further antimicrobial for product protection is selected from the group consisting of Butylparaben, Chlorhexidine, Chlorhexidine Digluconate, Chlorphenesin, Diazolidinyl Urea, Disodium EDTA, DMDM Hydantoin, Ethylparaben, Glyceryl Caprate, Glyceryl Laurate, Hydroxyethoxyphenyl Butanol, Hydroxyethoxyphenyl Butanone, Iodopropynyl Butylcarbamate, Isobutylparaben, Itaconic Acid, Methylchloroisothiazolinone, Methylisothiazolinone, Methylparaben, Phenethyl Alcohol, Potassium Sorbate, Propylparaben, Sodium Benzoate, Sodium Ethylparaben, Sodium Propylparaben, Sorbitan Caprylate, TEA-Salicylate, Tetrasodium Glutamate Diacetate, Trisodium Ethylenediamine Disuccinate, Dehydroacetic Acid, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more the aforesaid compounds.

In a particularly preferred variant, the further antimicrobial for product protection is selected from the group consisting of further antimicrobial for product protection is selected from the group consisting of Butylparaben, Disodium EDTA, DMDM Hydantoin, Ethylparaben, Hydroxyethoxyphenyl Butanol, Hydroxyethoxyphenyl Butanone, Itaconic Acid, Methylchloroisothiazolinone, Methylisothiazolinone, Methylparaben, Methylparaben, Potassium Sorbate, Propylparaben, Sodium Benzoate, Sodium Propylparaben, Dehydroacetic Acid, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more the aforesaid compounds.

The aforesaid antimicrobial for product protection can be used either as a single component or in combination with one or more antimicrobial for product protection as specified above. In order to enhance the antimicrobial effect and to cover a broader range of antimicrobial effect, a combination of two or more preservatives is preferably used.

Green and synthetic polymers: The cosmetic or pharmaceutical composition according to the present invention preferably includes one or more green or synthetic polymers. Suitable cationic polymers are, for example, cationic cellulose derivatives such as, for example, the quaternized hydroxyethyl cellulose obtainable from Amerchol under the name of Polymer JR 400@, cationic starch, copolymers of diallyl ammonium salts and acrylamides, quaternized vinyl pyrrolidone/vinyl imidazole polymers such as, for example, Luviquat® (BASF), condensation products of polyglycols and amines, quaternized collagen polypeptides such as, for example, Lauryldimonium Hydroxypropyl Hydrolyzed Collagen (Lamequat® L, GrOnau), quaternized wheat poly-peptides, polyethyleneimine, cationic silicone polymers such as, for example, amodimethicone, copolymers of adipic acid and dimethylaminohydroxypropyl diethylenetriamine (Cartaretine®, Sandoz), copolymers of acrylic acid with dimethyl diallyl ammonium chloride (Merquat® 550, Chemviron), polyaminopolyamides and crosslinked water-soluble polymers thereof, cationic chitin derivatives such as, for example, quaternized chitosan, optionally in microcrystalline distribution, condensation products of dihaloalkyls, for example dibromobutane, with bis-dialkylamines, for example bis-dimethylamino-1,3-propane, cationic guar gum such as, for example, Jaguar®CBS, Jaguar®C-17, Jaguar®C-16 of Celanese, quaternized ammonium salt polymers such as, for example, Mirapol®A-15, Mirapol®AD-1, Mirapol®AZ-1 of Miranol and the various polyquaternium types (for example 6, 7, 32 or 37) which can be found in the market under the tradenames Rheocare® CC or Ultragel® 300. Suitable anionic, zwitterionic, amphoteric and nonionic polymers are, for example, vinyl acetate/crotonic acid copolymers, vinyl pyrrolidone/vinyl acrylate copolymers, vinyl acetate/butyl maleate/isobornyl acrylate copolymers, methyl vinylether/maleic anhydride copolymers and esters thereof, uncrosslinked and polyol-crosslinked polyacrylic acids, acrylamidopropyl trimethylammonium chloride/acrylate copolymers, octylacryl-amide/methyl methacrylate/tert.-butylaminoethyl methacrylate/2-hydroxypropyl methacrylate copolymers, polyvinyl pyrrolidone, vinyl pyrrolidone/vinyl acetate copolymers, vinyl pyrrolidone/dimethylaminoethyl methacrylate/vinyl caprolactam terpolymers and optionally derivatized cellulose ethers and silicones. In a preferred variant, the cosmetic or pharmaceutical composition according to the present invention preferably includes an uncrosslinked or polyol-crosslinked polyacrylic acid as polymer component.

Thickening agents and/or rheology additives: The cosmetic or pharmaceutical composition according to the present invention preferably includes one or more thickening agents and/or rheology additives. Suitable thickeners are polymeric thickeners, such as Aerosil® types (hydrophilic silicas), polysaccharides, more especially xanthan gum, guar-guar, agar-agar, alginates and tyloses, carboxymethyl cellulose and hydroxyethyl cellulose, also relatively high molecular weight polyethylene glycol monoesters and diesters of fatty acids, polyacrylates (for example Carbopols® [Goodrich] or Synthalens® [Sigma]), polyacrylamides, polyvinyl alcohol and polyvinyl pyrrolidone, surfactants such as, for example, ethoxylated fatty acid glycerides, esters of fatty acids with polyols, for example pentaerythritol or trimethylol propane, narrow-range fatty alcohol ethoxylates and electrolytes, such as sodium chloride and ammonium chloride.

Perfume oils and/or fragrances: The cosmetic or pharmaceutical composition according to the present invention preferably includes one or more perfume oils and/or fragrances. Suitable perfume oils are mixtures of natural and synthetic perfumes. Natural perfumes include the extracts of blossoms (lily, lavender, rose, jasmine, neroli, ylang-ylang), stems and leaves (geranium, patchouli, petitgrain), fruits (anise, coriander, caraway, juniper), fruit peel (bergamot, lemon, orange), roots (nutmeg, angelica, celery, cardamom, costus, iris, calmus), woods (pinewood, sandalwood, guaiac wood, cedarwood, rosewood), herbs and grasses (tarragon, lemon grass, sage, thyme), needles and branches (spruce, fir, pine, dwarf pine), resins and balsams (galbanum, elemi, benzoin, myrrh, olibanum, opoponax). Animal raw materials, for example civet and beaver, may also be used. Typical synthetic perfume compounds are products of the ester, ether, aldehyde, ketone, alcohol and hydrocarbon type. Examples of perfume compounds of the ester type are benzyl acetate, phenoxyethyl isobutyrate, p-tert.butyl cyclohexylacetate, linalyl acetate, dimethyl benzyl carbinyl acetate, phenyl ethyl acetate, linalyl benzoate, benzyl formate, ethylmethyl phenyl glycinate, allyl cyclohexyl propionate, styrallyl propionate and benzyl salicylate. Ethers include, for example, benzyl ethyl ether while aldehydes include, for example, the linear alkanals containing 8 to 18 carbon atoms, citral, citronellal, citronellyloxyacetaldehyde, cyclamen aldehyde, hydroxycitronellal, lilial and bourgeonal. Examples of suitable ketones are the ionones, beta-isomethylionone and methyl cedryl ketone. Suitable alcohols are anethol, citronellol, eugenol, isoeugenol, geraniol, linalool, phenylethyl alcohol and terpineol. The hydrocarbons mainly include the terpenes and balsams. However, it is preferred to use mixtures of different perfume compounds which, together, produce an agreeable perfume. Other suitable perfume oils are essential oils of relatively low volatility which are mostly used as aroma components. Examples are sage oil, camomile oil, clove oil, melissa oil, mint oil, cinnamon leaf oil, lime-blossom oil, juniper berry oil, vetiver oil, olibanum oil, galbanum oil, ladanum oil and lavendin oil. The following are preferably used either individually or in the form of mixtures: bergamot oil, dihydromyrcenol, lilial, lyral, citronellol, phenylethyl alcohol, hexylcinnamaldehyde, geraniol, benzyl acetone, cyclamen aldehyde, linalool, Boisambrene Forte, Ambroxan, indole, hedione, sandelice, citrus oil, mandarin oil, orange oil, allylamyl glycolate, cyclovertal, lavendin oil, clary oil, damascone, geranium oil bourbon, cyclohexyl salicylate, Vertofix Coeur, Iso-E-Super, Fixolide NP, evernyl, iraldein gamma, phenylacetic acid, geranyl acetate, benzyl acetate, rose oxide, romillat, irotyl and floramat.

Anti-cellulite agent: The cosmetic or pharmaceutical composition according to the present invention preferably includes one or more anti-cellulite agents. Anti-cellulite agents and lipolytic agents are preferably selected from the group consisting of beta-adrenergic receptor agonists such as synephrine and its derivatives, and cyclohexyl carbamates. Agents enhancing or boosting the activity of anti-cellulite agents, in particular agents which stimulate and/or depolarise C nerve fibres, are preferably selected from the group consisting of capsaicin and derivatives thereof, vanillyl-nonylamid and derivatives thereof, L-carnitine, coenzym A, isoflavonoides, soy extracts, ananas extract and conjugated linoleic acid.

Fat enhancing agents: The cosmetic or pharmaceutical composition according to the present invention preferably includes one or more fat enhancing agents and/or adipogenic agents as well as agents enhancing or boosting the activity of fat enhancing agents. A fat enhancing agent is for example hydroxymethoxyphenyl propylmethylmethoxybenzofuran (trade name: Sym3D®).

Superfatting agents and/or consistency factors: The cosmetic or pharmaceutical composition according to the present invention preferably includes one or more superfatting agents and/or consistency factors. Superfatting agents may be selected from such substances as, for example, lanolin and lecithin and also polyethoxylated or acylated lanolin and lecithin derivatives, polyol fatty acid esters, monoglycerides and fatty acid alkanolamides, the fatty acid alkanolamides also serving as foam stabilizers. The consistency factors mainly used are fatty alcohols or hydroxyfatty alcohols containing 12 to 22 and preferably 16 to 18 carbon atoms and also partial glycerides, fatty acids or hydroxyfatty acids. A combination of these substances with alkyl oligoglucosides and/or fatty acid N-methyl glucamides of the same chain length and/or polyglycerol poly-12-hydroxystearates is preferably used.

Pearlizing waxes: The cosmetic or pharmaceutical composition according to the present invention preferably includes one or more pearlizing waxes. Suitable pearlizing waxes are, for example, alkylene glycol esters, especially ethylene glycol distearate; fatty acid alkanolamides, especially cocofatty acid diethanolamide; partial glycerides, especially stearic acid monoglyceride; esters of polybasic, optionally hydroxysubstituted carboxylic acids with fatty alcohols containing 6 to 22 carbon atoms, especially long-chain esters of tartaric acid; fatty compounds, such as for example fatty alcohols, fatty ketones, fatty aldehydes, fatty ethers and fatty carbonates which contain in all at least 24 carbon atoms, especially laurone and distearylether; fatty acids, such as stearic acid, hydroxystearic acid or behenic acid, ring opening products of olefin epoxides containing 12 to 22 carbon atoms with fatty alcohols containing 12 to 22 carbon atoms and/or polyols containing 2 to 15 carbon atoms and 2 to 10 hydroxyl groups, and mixtures thereof.

Silicones: In order to impart a silky, spreadable, and luxurious texture and to make skin look and feel smoother, and additionally to improve processability (antifoaming) the cosmetic or pharmaceutical composition according to the present invention preferably includes one or more silicone(s) or silicone derative(s). Suitable silicones can be chosen from the group consisting of Acefylline Methylsilanol Mannuronate, Acetylmethionyl Methylsilanol Elastinate Acrylates/Behenyl, Acrylate/Dimethicone Methacrylate Copolymer, Acrylates/Behenyl Methacrylate/Dimethicone Methacrylate Copolymer, Acrylates/Bis-Hydroxypropyl Dimethicone Crosspolymer, Acrylates/Dimethicone Copolymer, Acrylates/Dimethicone Methacrylate/Ethylhexyl Acrylate Copolymer, Acrylates/Dimethiconol Acrylate Copolymer, Acrylates/Ethylhexyl Acrylate/Dimethicone Methacrylate Copolymer, Acrylates/Octylacrylamide/Diphenyl Amodimethicone Copolymer, Acrylates/Polytrimethylsiloxymethacrylate Copolymer, Acrylates/Propyl Trimethicone Methacrylate Copolymer, Acrylates/Stearyl Acrylate/Dimethicone Methacrylate Copolymer, Acrylates/Tridecyl Acrylate/Triethoxysilylpropyl Methacrylate/Dimethicone Methacrylate Copolymer, Acrylates/Trifluoropropylmethacrylate/Polytrimethyl Siloxymethacrylate Copolymer, Amino Bispropyl Dimethicone, Aminoethylaminopropyl Dimethicone, Aminopropyl Dimethicone, Aminopropyl Phenyl Trimethicone, Aminopropyl Triethoxysilane, Ammonium Dimethicone PEG-7 Sulfate, Amodimethicone, Amodimethicone Hydroxystearate, Amodimethicone/Silsesquioxane Copolymer, Ascorbyl Carboxydecyl Trisiloxane, Ascorbyl Methylsilanol Pectinate, Behenoxy Dimethicone, Behentrimonium Dimethicone PEG-8 Phthalate, Behenyl Dimethicone, Bisamino PEG/PPG-41/3 Aminoethyl PG-Propyl Dimethicone, Bis-Aminopropyl/Ethoxy Aminopropyl Dimethicone, Bis(Butylbenzoate) Diaminotriazine Aminopropyltrisiloxane, Bis-Butyldimethicone Polyglyceryl-3, Bis-Butyloxyamodimethicone/PEG-60 Copolymer, Bis(C13-15 Alkoxy) Hydroxybutamidoamodimethicone, Bis(C13-15 Alkoxy) PG-Amodimethicone, Bis-(C1-8 Alkyl Lauroyl Lysine Decylcarboxamide) Dimethicone, Bis-Cetyl Cetyl Dimethicone, Bis-Cetyl/PEG-8 Cetyl PEG-8 Dimethicone, Bis-Diphenylethyl Disiloxane, Bis-Ethyl Ethyl Methicone, Bis-Gluconamidoethylaminopropyl Dimethicone, Bis-Hydrogen Dimethicone, Bis-Hydroxyethoxypropyl Dimethicone Bis-Hydroxylauryl, Dimethicone/IPDI Copolymer, Bis-Hydroxy/Methoxy Amodimethicone, Bis-Hydroxypropyl Dimethicone Behenate, Bis-Hydroxypropyl Dimethicone/SMDI Copolymer, Bis-Isobutyl PEG-14/Amodimethicone Copolymer, Bis-Isobutyl PEG-15/Amodimethicone Copolymer, Bis-Isobutyl PEG/PPG-20/35/Amodimethicone Copolymer, Bis-Isobutyl PEG/PPG-10/7/Dimethicone Copolymer, Bis-Isobutyl PEG-24/PPG-7/Dimethicone Copolymer, Bis-PEG-1 Dimethicone, Bis-PEG-4 Dimethicone, Bis-PEG-8 Dimethicone, Bis-PEG-12 Dimethicone,Bis-PEG-20 Dimethicone, Bis-PEG-12 Dimethicone Beeswax, Bis-PEG-12 Dimethicone Candelillate, Bis-PEG-15 Dimethicone/IPDI Copolymer, Bis-PEG-15 Methyl Ether Dimethicone, Bis-PEG-18 Methyl Ether Dimethyl Silane, Bis-PEG/PPG-14/14 Dimethicone, Bis-PEG/PPG-15/5 Dimethicone, Bis-PEG/PPG-18/6 Dimethicone, Bis-PEG/PPG-20/20 Dimethicone, Bis-PEG/PPG-16/16 PEG/PPG-16/16 Dimethicone, Bis-PEG/PPG-20/5 PEG/PPG-20/5 Dimethicone, Bisphenylhexamethicone, Bis-Phenylpropyl Dimethicone, Bispolyethylene Dimethicone, Bis-(Polyglyceryl-3 Oxyphenylpropyl) Dimethicone, Bis-(Polyglyceryl-7 Oxyphenylpropyl) Dimethicone, Bis-PPG-15 Dimethicone/IPDI Copolymer, Bis(PPG-7 Undeceneth-21) Dimethicone, Bis-Stearyl Dimethicone, Bis-Trimethoxysilylethyl Tetramethyldisiloxyethyl Dimethicone, Bis-Vinyldimethicone, Bis-Vinyl Dimethicone/Dimethicone Copolymer, Borage Seed Oil PEG-7 Dimethicone Esters, Butyl Acrylate/C6-14 Perfluoroalkylethyl Acrylate/Mercaptopropyl Dimethicone Copolymer, Butyl Acrylate/Hydroxypropyl Dimethicone Acrylate Copolymer, Butyl Dimethicone Acrylate/Cyclohexylmethacrylate/Ethylhexyl Acrylate Copolymer, Butyldimethicone Methacrylate/Methyl Methacrylate Crosspolymer, t-Butyl Dimethyl Silyl Grape Seed Extract, Butyl Polydimethylsiloxyl Ethylene/Propylene/Vinylnorbornene Copolymer, C6-8 Alkyl C3-6 Alkyl Glucoside Dimethicone, C20-24 Alkyl Dimethicone, C24-28 Alkyl Dimethicone, C26-28 Alkyl Dimethicone, C30-45 Alkyl Dimethicone, C30-60 Alkyl Dimethicone, C32 Alkyl Dimethicone, C30-45 Alkyl Dimethicone/Polycyclohexene Oxide Crosspolymer, C26-28 Alkyldimethylsilyl Polypropylsilsesquioxane, C30-45 Alkyldimethylsilyl Polypropylsilsesquioxane, C20-24 Alkyl Methicone, C24-28 Alkyl Methicone, C26-28 Alkyl Methicone, C30-45 Alkyl Methicone, C20-28 Alkyl Perfluorodecylethoxy Dimethicone, C26-54 Alkyl Tetradecyl Dimethicone, Capryl Dimethicone, Caprylyl Dimethicone Ethoxy Glucoside, Caprylyl Methicone, Caprylyl Trimethicone, Carboxydecyl Trisiloxane, Castor Oil Bis-Hydroxypropyl Dimethicone Esters Cerotyl Dimethicone, Cetearyl Dimethicone Crosspolymer, Cetearyl Dimethicone/Vinyl Dimethicone Crosspolymer, Cetearyl Methicone, Cetrimonium Carboxydecyl PEG-8 Dimethicone, Cetrimonium Dimethicone PEG-7 Phthalate, Cetyl Behenyl Dimethicone, Cetyl Dimethicone, Cetyl Dimethicone/Bis-Vinyldimethicone Crosspolymer, Cetyl Hexacosyl Dimethicone, Cetyloxy Dimethicone, Cetyl PEG-8 Dimethicone, Cetyl PEG/PPG-15/15 Butyl Ether Dimethicone, Cetyl PEG/PPG-7/3 Dimethicone, Cetyl PEG/PPG-10/1 Dimethicone, Cetyl Triethylmonium Dimethicone PEG-8 Phthalate, Cetyl Triethylmonium Dimethicone PEG-8 Succinate, Copper Acetyl Tyrosinate Methylsilanol, Copper PCA Methylsilanol, C4-14 Perfluoroalkylethoxy Dimethicone, Cycloethoxymethicone, Cycloheptasiloxane, Cyclohexasiloxane, Cyclomethicone, Cyclopentasiloxane, Cyclophenylmethicone, Cyclotetrasiloxane, mCyclovinylmethicone, Cystine Bis-PG-Propyl Silanetriol, DEA PG-Propyl PEG/PPG-18/21 Dimethicone, Diisostearoyl Trimethylolpropane Siloxy Silicate, Dilauroyl Trimethylolpropane Siloxy Silicate, Dilinoleamidopropyl Dimethylamine Dimethicone PEG-7 Phosphate, Dimethicone, Dimethicone Crosspolymer, Dimethicone Crosspolymer-3, Dimethicone/Divinyldimethicone/Silsesquioxane Crosspolymer, Dimethicone Ethoxy Glucoside, Dimethicone Hydroxypropyl Trimonium Chloride, Dimethicone/Mercaptopropyl Methicone Copolymer, Dimethicone PEG-15 Acetate Dimethicone PEG-8 Adipate, Dimethicone PEG-7 Avocadoate, Dimethicone PEG-8 Avocadoate, Dimethicone PEG-8 Beeswax, Dimethicone PEG-8 Benzoate, Dimethicone PEG-8 Borageate, Dimethicone PEG-7 Cocoate, Dimethicone/PEG-10 Crosspolymer, Dimethicone/PEG-10/15 Crosspolymer, Dimethicone/PEG-15 Crosspolymer, Dimethicone PEG-7 Isostearate, Dimethicone PEG-8 Isostearate, Dimethicone PEG-7 Lactate, Dimethicone PEG-8 Lanolate, Dimethicone PEG-8 Laurate, Dimethicone PEG-8 Meadowfoamate, Dimethicone PEG-7 Octyldodecyl Citrate, Dimethicone PEG-7 Olivate, Dimethicone PEG-8 Olivate, Dimethicone PEG-7 Phosphate, Dimethicone PEG-8 Phosphate, Dimethicone PEG-10 Phosphate, Dimethicone PEG-7 Phthalate, Dimethicone PEG-8 Phthalate, Dimethicone PEG-8 Polyacrylate, Dimethicone PEG/PPG-20/23 Benzoate, Dimethicone PEG/PPG-7/4 Phosphate, Dimethicone PEG/PPG-12/4 Phosphate, Dimethicone PEG-7 Succinate, Dimethicone PEG-8 Succinate, Dimethicone PEG-7 Sulfate, Dimethicone PEG-7 Undecylenate, Dimethicone PG-Diethylmonium Chloride, Dimethicone/Phenyl Vinyl Dimethicone Crosspolymer, Dimethicone/Polyglycerin-3 Crosspolymer, Dimethicone/PPG-20 Crosspolymer, Dimethicone Propylethylenediamine Behenate, Dimethicone Propyl PG-Betaine, Dimethicone/Silsesquioxane Copolymer, Dimethicone Silylate, Dimethicone/Vinyl Dimethicone Crosspolymer, Dimethicone/Vinyltrimethylsiloxysilicate Crosspolymer, Dimethiconol, Dimethiconol Arginine, Dimethiconol Beeswax, Dimethiconol Behenate, Dimethiconol Borageate, Dimethiconol Candelillate, Dimethiconol Carnaubate, Dimethiconol Cysteine, Dimethiconol Dhupa Butterate, Dimethiconol Fluoroalcohol Dilinoleic Acid, Dimethiconol Hydroxystearate, Dimethiconol Illipe Butterate, Dimethiconol/IPDI Copolymer, Dimethiconol Isostearate, Dimethiconol Kokum Butterate, Dimethiconol Lactate, Dimethiconol Meadowfoamate, Dimethiconol Methionine, Dimethiconol/Methylsilanol/Silicate Crosspolymer, Dimethiconol Mohwa Butterate, Dimethiconol Panthenol, Dimethiconol Sal Butterate, Dimethiconol/Silica Crosspolymer, Dimethiconol/Silsesquioxane Copolymer, Dimethiconol Stearate, Dimethiconol/Stearyl, Methicone/Phenyl Trimethicone Copolymer, Dimethoxysilyl Ethylenediaminopropyl Dimethicone, Dimethylaminopropylamido PCA Dimethicone, Dimethyl Oxobenzo Dioxasilane, Dimethylsilanol Hyaluronate, Dioleyl Tocopheryl Methylsilanol, Diphenyl Amodimethicone, Diphenyl Dimethicone, Diphenyl Dimethicone Crosspolymer Diphenyl Dimethicone?/inyl Diphenyl Dimethicone/Silsesquioxane Crosspolymer, Diphenylethyl Benzyloxy Dilsiloxane, Diphenylisopropyl Dimethicone, Diphenylsiloxy Phenyl/Propyl Trimethicone, Diphenylsiloxy Phenyl Trimethicone Disiloxane, Disodium Amodimethicone Disuccinamide, Disodium PEG-12 Dimethicone Sulfosuccinate, Disodium PEG-8 Lauryl Dimethicone Sulfosuccinate, Divinyldimethicone/Dimethicone Copolymer, Divinyldimethicone/Dimethicone Crosspolymer, Drometrizole Trisiloxane, Ethylhexyl Acrylate/VP/Dimethicone Methacrylate Copolymer, Ethyl Methicone, Ethyl Trisiloxane, Fluoro C2-8 Alkyldimethicone, Gluconamidopropyl Aminopropyl Dimethicone, 4-(2-Beta-Glucopyranosiloxy) Propoxy-2-Hydroxybenzophenone, Glyceryl Undecyl Dimethicone, Glycidoxy Dimethicone, Hexadecyl Methicone, Hexyl Dimethicone, Hexyl Methicone, Hexyltrimethoxysilane, Hydrogen Dimethicone, Hydrogen Dimethicone/Octyl Silsesquioxane Copolymer, Hydrolyzed Collagen PG-Propyl Dimethiconol, Hydrolyzed Collagen PG-Propyl Methylsilanediol, Hydrolyzed Collagen PG-Propyl Silanetriol, Hydrolyzed Keratin PG-Propyl Methylsilanediol, Hydrolyzed Sesame Protein PG-Propyl Methylsilanediol, Hydrolyzed Silk PG-Propyl Methylsilanediol, Hydrolyzed Silk PG-Propyl Methylsilanediol Crosspolymer, Hydrolyzed Soy Protein/Dimethicone PEG-7 Acetate, Hydrolyzed Soy Protein PG-Propyl Methylsilanediol, Hydrolyzed Vegetable Protein PG-Propyl Silanetriol, Hydrolyzed Wheat Protein/Cystine Bis-PG-Propyl Silanetriol Copolymer, Hydrolyzed Wheat Protein/Dimethicone PEG-7 Acetate, Hydrolyzed Wheat Protein/Dimethicone PEG-7 Phosphate Copolymer, Hydrolyzed Wheat Protein PG-Propyl Methylsilanediol, Hydrolyzed Wheat Protein PG-Propyl Silanetriol, Hydroxyethyl Acetomonium PG-Dimethicone, Hydroxypropyldimethicone, Hydroxypropyl Dimethicone Behenate, Hydroxypropyl Dimethicone Isostearate, Hydroxypropyl Dimethicone Stearate, Isobutylmethacrylate/Bis-Hydroxypropyl Dimethicone Acrylate Copolymer, Isobutylmethacrylate/Trifluoroethylmethacrylate/Bis-Hydroxypropyl Dimethicone Acrylate Copolymer, Isopentyl Trimethoxycinnamate Trisiloxane, Isopolyglyceryl-3 Dimethicone, Isopolyglyceryl-3 Dimethiconol, Isopropyl Titanium Triisostearate/Triethoxysilylethyl, Polydimethylsiloxyethyl Dimethicone Crosspolymer, Isostearyl Carboxydecyl PEG-8 Dimethicone, Lactoyl Methylsilanol Elastinate, Lauryl Dimethicone, Lauryl Dimethicone PEG-15 Crosspolymer, Lauryl Dimethicone PEG-10 Phosphate, Lauryl Dimethicone/Polyglycerin-3 Crosspolymer, Lauryl Methicone, Lauryl PEG-8 Dimethicone, Lauryl PEG-10 Methyl Ether Dimethicone, Lauryl PEG-9 Polydimethylsiloxyethyl Dimethicone, Lauryl PEG/PPG-18/18 Methicone, Lauryl Phenylisopropyl Methicone, Lauryl Phenylpropyl Methicone, Lauryl Polydimethylsiloxyethyl Dimethicone/Bis-Vinyldimethicone Crosspolymer, Lauryl Polyglyceryl-3 Polydimethylsiloxyethyl Dimethicone, Lauryl Trimethicone, Linoleamidopropyl PG-Dimonium Chloride Phosphate Dimethicone, Methacryloyl Propyltrimethoxysilane, Methicone, Methoxy Amodimethicone/Silsesquioxane Copolymer, Methoxycinnamidopropyl Polysilsesquioxane, Methoxycinnamoylpropyl Silsesquioxane Silicate, Methoxy PEG-13 Ethyl Polysilsesquioxane, Methoxy PEG/PPG-7/3 Aminopropyl Dimethicone, Methoxy PEG/PPG-25/4 Dimethicone, Methoxy PEG-10 Propyltrimethoxysilane, Methyleugenyl PEG-8 Dimethicone, Methylpolysiloxane Emulsion, Methylsilanol Acetylmethionate, Methylsilanol Acetyltyrosine, Methylsilanol Ascorbate, Methylsilanol Carboxymethyl Theophylline, Methylsilanol Carboxymethyl Theophylline Alginate, Methylsilanol Elastinate, Methylsilanol Glycyrrhizinate, Methylsilanol Hydroxyproline, Methylsilanol Hydroxyproline Aspartate, Methylsilanol Mannuronate, Methylsilanol PCA, Methylsilanol PEG-7 Glyceryl Cocoate, Methylsilanol/Silicate Crosspolymer, Methylsilanol Spirulinate, Methylsilanol Tri-PEG-8 Glyceryl Cocoate, Methyl Trimethicone, Methyltrimethoxysilane, Myristylamidopropyl Dimethylamine Dimethicone PEG-7 Phosphate, Myristyl Methicone, Myristyl Trisiloxane, Nylon-611/Dimethicone Copolymer, PCA Dimethicone, PEG-7 Amodimethicone, PEG-8 Amodimethicone, PEG-8 Cetyl Dimethicone, PEG-3 Dimethicone, PEG-6 Dimethicone, PEG-7 Dimethicone, PEG-8 Dimethicone, PEG-9 Dimethicone, PEG-10 Dimethicone, PEG-12 Dimethicone, PEG-14 Dimethicone, PEG-17 Dimethicone, PEG-10 Dimethicone Crosspolymer, PEG-12 Dimethicone Crosspolymer, PEG-8 Dimethicone Dimer Dilinoleate, PEG-8 Dimethicone/Dimer Dilinoleic Acid Copolymer, PEG-10 Dimethicone/Vinyl Dimethicone Crosspolymer, PEG-8 Distearmonium Chloride PG-Dimethicone, PEG-10/Lauryl Dimethicone Crosspolymer, PEG-15/Lauryl Dimethicone Crosspolymer, PEG-15/Lauryl Polydimethylsiloxyethyl Dimethicone Crosspolymer, PEG-8 Methicone, PEG-6 Methicone Acetate, PEG-6 Methyl Ether Dimethicone, PEG-7 Methyl Ether Dimethicone, PEG-8 Methyl Ether Dimethicone, PEG-9 Methyl Ether Dimethicone, PEG-10 Methyl Ether Dimethicone, PEG-11 Methyl Ether Dimethicone, PEG-32 Methyl Ether Dimethicone, PEG-8 Methyl Ether Triethoxysilane, PEG-10 Nonafluorohexyl Dimethicone Copolymer, PEG-4 PEG-12 Dimethicone, PEG-8 PG-Coco-Glucoside Dimethicone, PEG-9 Polydimethylsiloxyethyl Dimethicone, PEG/PPG-20/22 Butyl Ether Dimethicone, PEG/PPG-22/22 Butyl Ether Dimethicone, PEG/PPG-23/23 Butyl Ether Dimethicone, PEG/PPG-24/18 Butyl Ether Dimethicone, PEG/PPG-27/9 Butyl Ether Dimethicone, PEG/PPG-3/10 Dimethicone, PEG/PPG-4/12 Dimethicone, PEG/PPG-6/4 Dimethicone, PEG/PPG-6/11 Dimethicone, PEG/PPG-8/14 Dimethicone, PEG/PPG-8/26 Dimethicone, PEG/PPG-10/2 Dimethicone, PEG/PPG-12/16 Dimethicone, PEG/PPG-12/18 Dimethicone, PEG/PPG-14/4 Dimethicone, PEG/PPG-15/5 Dimethicone, PEG/PPG-15/15 Dimethicone, PEG/PPG-16/2 Dimethicone, PEG/PPG-16/8 Dimethicone, PEG/PPG-17/18 Dimethicone, PEG/PPG-18/6 Dimethicone, PEG/PPG-18/12 Dimethicone, PEG/PPG-18/18 Dimethicone, PEG/PPG-19/19 Dimethicone, PEG/PPG-20/6 Dimethicone, PEG/PPG-20/15 Dimethicone, PEG/PPG-20/20 Dimethicone, PEG/PPG-20/23 Dimethicone, PEG/PPG-20/29 Dimethicone, PEG/PPG-22/23 Dimethicone, PEG/PPG-22/24 Dimethicone, PEG/PPG-23/6 Dimethicone, PEG/PPG-25/25 Dimethicone, PEG/PPG-27/27 Dimethicone, PEG/PPG-30/10 Dimethicone, PEG/PPG-25/25 Dimethicone/Acrylates Copolymer, PEG/PPG-20/22 Methyl Ether Dimethicone, PEG/PPG-24/24 Methyl Ether Glycidoxy Dimethicone, PEG/PPG-10/3 Oleyl Ether Dimethicone, PEG/PPG-5/3 Trisiloxane, PEG-4 Trifluoropropyl Dimethicone Copolymer, PEG-8 Trifluoropropyl Dimethicone Copolymer, PEG-10 Trifluoropropyl Dimethicone Copolymer, PEG-8 Trisiloxane, Perfluorocaprylyl riethoxysilylethyl Methicone, Perfluorononyl Dimethicone, Perfluorononyl Dimethicone/Methicone/Amodimethicone Crosspolymer, Perfluorononylethyl Carboxydecyl Behenyl Dimethicone, Perfluorononylethyl Carboxydecyl Hexacosyl Dimethicone, Perfluorononylethyl Carboxydecyl Lauryl/Behenyl Dimethicone, Perfluorononylethyl Carboxydecyl Lauryl Dimethicone, Perfluorononylethyl Carboxydecyl PEG-8 Dimethicone, Perfluorononylethyl Carboxydecyl PEG-10 Dimethicone, Perfluorononylethyl Dimethicone/Methicone Copolymer, Perfluorononylethyl PEG-8 Dimethicone, Perfluorononylethyl Stearyl Dimethicone, Perfluorooctylethyl/Diphenyl Dimethicone Copolymer, Perfluorooctylethyl Triethoxysilane, Perfluorooctylethyl Trimethoxysilane, Perfluorooctylethyl Trisiloxane, Perfluorooctyl Triethoxysilane, PG-Amodimethicone, Phenethyl Dimethicone, Phenethyl Disiloxane, Phenyl Dimethicone, Phenylisopropyl Dimethicone, Phenyl Methicone, Phenyl Methiconol, Phenylpropyldimethylsiloxysilicate, Phenylpropyl Ethyl Methicone, Phenyl Propyl Trimethicone, Phenyl Propyl Trimethicone/Diphenylmethicone, Phenyl Trimethicone, Platinum Divinyldisiloxane, Polyacrylate-6, Polydiethylsiloxane, Polydimethylsiloxyethyl Dimethicone/Bis-Vinyldimethicone Crosspolymer, Polydimethylsiloxyethyl Dimethicone/Methicone Copolymer, Polydimethylsiloxy PEG/PPG-24/19 Butyl Ether Silsesquioxane, Polydimethylsiloxy PPG-13 Butyl Ether Silsesquioxane, Polyglyceryl-3 Disiloxane Dimethicone, Polyglyceryl-3/Lauryl Polydimethylsiloxyethyl Dimethicone Crosspolymer, Polyglyceryl-3 Polydimethylsiloxyethyl Dimethicone, Poly(Glycol Adipate)/Bis-Hydroxyethoxypropyl Dimethicone Copolymer, Polymethylsilsesquioxane, Polymethylsilsesquioxane/Trimethylsiloxysilicate, Polyphenylsilsesquioxane, Polypropylsilsesquioxane, Polysilicone-1, Polysilicone-2, Polysilicone-3, Polysilicone-4, Polysilicone-5, Polysilicone-6, Polysilicone-7, Polysilicone-8, Polysilicone-9, Polysilicone-10, Polysilicone-11, Polysilicone-12, Polysilicone-13, Polysilicone-14, Polysilicone-15, Polysilicone-16, Polysilicone-17, Polysilicone-18, Polysilicone-19, Polysilicone-20, Polysilicone-21, Polysilicone-18 Cetyl Phosphate, Polysilicone-1 Crosspolymer, Polysilicone-18 Stearate, Polyurethane-10, Potassium Dimethicone PEG-7 Panthenyl Phosphate, Potassium Dimethicone PEG-7 Phosphate, PPG-12 Butyl Ether Dimethicone, PPG-2 Dimethicone, PPG-12 Dimethicone, PPG-27 Dimethicone, PPG-4 Oleth-10 Dimethicone, Propoxytetramethyl Piperidinyl Dimethicone, Propyl Trimethicone, Quaternium-80, Retinoxytrimethylsilane, Silanediol Salicylate, Silanetriol, Silanetriol Arginate, Silanetriol Glutamate, Silanetriol Lysinate, Silanetriol Melaninate, Silanetriol Trehalose Ether, Silica, Silica Dimethicone Silylate, Silica Dimethyl Silylate, Silica Silylate, Silicon Carbide, Silicone Quaternium-1, Silicone Quaternium-2, Silicone Quaternium-2 Panthenol Succinate, Silicone Quaternium-3, Silicone Quaternium-4, Silicone Quaternium-5, Silicone Quaternium-6, Silicone Quaternium-7, Silicone Quaternium-8, Silicone Quaternium-9, Silicone Quaternium-10, Silicone Quaternium-11, Silicone Quaternium-12, Silicone Quaternium-15, SiliconeQuaternium-16, Silicone Quaternium-16/Glycidoxy Dimethicone Crosspolymer, Silicone Quaternium-17, Silicone Quaternium-18, Silicone Quaternium-19, Silicone Quaternium-20, Silicone Quaternium-21, Silicone Quaternium-22, Silicone Quaternium-24, Silicone Quaternium-25, Siloxanetriol Alginate, Siloxanetriol Phytate, Simethicone, Sodium Carboxydecyl PEG-8 Dimethicone, Sodium Dimethicone PEG-7 Acetyl Methyltaurate, Sodium Hyaluronate Dimethylsilanol, Sodium Lactate Methylsilanol, Sodium Mannuronate Methylsilanol, Sodium PCA Methylsilanol, Sodium PG-Propyldimethicone Thiosulfate Copolymer, Sodium PG-Propyl Thiosulfate Dimethicone, Sodium Propoxyhydroxypropyl Thiosulfate Silica, Sorbityl Silanediol, Soy Triethoxysilylpropyldimonium Chloride, Stearalkonium Dimethicone PEG-8 Phthalate, Stearamidopropyl Dimethicone, Steardimonium Hydroxypropyl Panthenyl PEG-7 Dimethicone Phosphate Chloride, Steardimonium Hydroxypropyl PEG-7 Dimethicone Phosphate Chloride, Stearoxy Dimethicone, Stearoxymethicone/Dimethicone Copolymer, Stearoxytrimethylsilane, Stearyl Aminopropyl Methicone, Stearyl Dimethicone, Stearyl/Lauryl Methacrylate Crosspolymer, Stearyl Methicone, Stearyl Triethoxysilanek, Stearyl Trimethicone, Styrene/Acrylates/Dimethicone Acrylate Crosspolymer, Styrene/Acrylates/Dimethicone Copolymer, TEA-Dimethicone PEG-7 Phosphate, Tetrabutoxypropyl Trisiloxane, Tetramethyl Hexaphenyl Tetrasiloxane, Tetramethyl Tetraphenyl Trisiloxane, Tocopheryloxypropyl Trisiloxane, Trideceth-9 PG-Amodimethicone, Triethoxycaprylylsilane, Triethoxysilylethyl Dimethicone/Methicone Copolymer, Triethoxysilylethyl Polydimethylsiloxyethyl Dimethicone, Triethoxysilylethyl Polydimethylsiloxyethyl Hexyl Dimethicone, Triethoxysilylpropylcarbamoyl Ethoxypropyl Butyl Dimethicone, Trifluoromethyl C1-4 Alkyl Dimethicone, Trifluoropropyl Cyclopentasiloxane, Trifluoropropyl Cyclotetrasiloxane, Trifluoropropyl Dimethicone, Trifluoropropyl Dimethicone/PEG-10 Crosspolymer, Trifluoropropyl Dimethicone/Trifluoropropyl Divinyldimethicone Crosspolymer, Trifluoropropyl Dimethicone/Vinyl Trifluoropropyl, Dimethicone/Silsesquioxane Crosspolymer, Trifluoropropyl Dimethiconol, Trifluoropropyldimethyl/trimethylsiloxysilicate, Trifluoropropyl Methicone, Trimethoxycaprylylsilane, Trimethoxysilyl Dimethicone, Trimethyl Pentaphenyl Trisiloxane, Trimethylsiloxyamodimethicone, Trimethylsiloxyphenyl Dimethicone, Trimethylsiloxysilicate, Trimethylsiloxysilicate/Dimethicone Crosspolymer, Trimethylsiloxysilicate/Dimethiconol Crosspolymer, Trimethylsiloxysilylcarbamoyl Pullulan, Trimethylsilyl Hydrolyzed Conchiolin Protein PG-Propyl Methylsilanediol Crosspolymer, Trimethylsilyl Hydrolyzed Silk PG-Propyl Methylsilanediol Crosspolymer, Trimethylsilyl Hydrolyzed Wheat Protein PG-Propyl Methylsilanediol Crosspolymer, Trimethylsilyl Pullulan, Trimethylsilyl Trimethylsiloxy Glycolate, Trimethylsilyl Trimethylsiloxy Lactate, Trimethylsilyl Trimethylsiloxy Salicylate, Triphenyl Trimethicone, Trisiloxane, Tris-Tributoxysiloxymethylsilane, Undecylcrylene Dimethicone, Vinyl Dimethicone, Vinyl Dimethicone/Lauryl Dimethicone Crosspolymer, Vinyl Dimethicone/Methicone Silsesquioxane Crosspolymer, Vinyldimethyl/Trimethylsiloxysilicate Stearyl Dimethicone Crosspolymer, VP/Dimethiconylacrylate/Polycarbamyl/Polyglycol Ester, Zinc Carboxydecyl Trisiloxane, Zinc Dimethicone PEG-8 Succinate, and mixtures thereof.

More preferably the silicones to be contained in the cosmetic or pharmaceutical composition according to the invention are Dimethicone, Cyclomethicone, Cyclopentasiloxane, Cyclotetrasiloxane, Phenyl Trimethicone, and Cyclohexasiloxane.

Waxes and/or stabilizers: Besides natural oils used, one or more waxes may also be present in the cosmetic or pharmaceutical composition according to the present invention, more especially natural waxes such as, for example, candelilla wax, carnauba wax, Japan wax, espartograss wax, cork wax, guaruma wax, rice oil wax, sugar cane wax, ouricury wax, montan wax, beeswax, shellac wax, spermaceti, lanolin (wool wax), uropygial fat, ceresine, ozocerite (earth wax), petrolatum, paraffin waxes and microwaxes; chemically modified waxes (hard waxes) such as, for example, montan ester waxes, sasol waxes, hydrogenated jojoba waxes and synthetic waxes such as, for example, polyalkylene waxes and polyethylene glycol waxes. Metal salts of fatty acids such as, for example, magnesium, aluminium and/or zinc stearate or ricinoleate may be used as stabilizers.

Apart from the abovementioned liquid UV filters, the cosmetic or pharmaceutical composition as defined herein, is advantageously combined with at least one further primary sun protection factor and/or with at least one further secondary sun protection factor, in order to increase the SPF, i.e. to obtain a high SPF and to cover a broad UVA and UVB range.

Primary sun protection factors: Primary sun protection factors in the context of the invention are, for example, organic substances (light filters) which are liquid or crystalline at room temperature and which are capable of absorbing ultraviolet radiation and of releasing the energy absorbed in the form of longer-wave radiation, for example heat.

The cosmetic or pharmaceutical composition according to the invention advantageously contains at least one UV-A filter and/or at least one UV-B filter and/or a broadband filter and/or at least one inorganic pigment. Compositions according to the invention preferably contain at least one UV-B filter or a broadband filter, more particularly preferably at least one UV-A filter and at least one UV-B filter. Preferred cosmetic compositions, preferably topical compositions according to the present invention comprise one, two, three or more sun protection factors selected from the group consisting of 4-aminobenzoic acid and derivatives, salicylic acid derivatives, benzophenone derivatives, dibenzoylmethane derivatives, diphenyl acrylates, 3-imidazol-4-yl acrylic acid and esters thereof, benzofuran derivatives, benzylidene malonate derivatives, polymeric UV absorbers containing one or more organosilicon radicals, cinnamic acid derivatives, camphor derivatives, trianilino-s-triazine derivatives, 2-hydroxyphenylbenzotriazole derivatives, phenylbenzimidazole sulfonic acid derivatives and salts thereof, anthranilic acid menthyl esters, benzotriazole derivatives and indole derivatives.

In addition, a combination with active ingredients which penetrate into the skin and protect the skin cells from inside against sunlight-induced damage and reduce the level of cutaneous matrix metalloproteases is advantageously. Preferred respective ingredients are so called arylhydrocarbon receptor antagonists. Preferred is 2-benzylidene-5,6-dimethoxy-3,3-dimethylindan-1-one.

The UV filters cited below which can be used within the context of the present invention are preferred but naturally are not limiting.

UV filters which are preferably used are selected from the group consisting of
p-aminobenzoic acid
p-aminobenzoic acid ethyl ester (25 mol) ethoxylated (INCI name: PEG-25 PABA)
p-dimethylaminobenzoic acid-2-ethylhexyl ester
p-aminobenzoic acid ethyl ester (2 mol)N-propoxylated
p-aminobenzoic acid glycerol ester
salicylic acid homomenthyl ester (homosalates) (Neo Heliopan®HMS)
salicylic acid-2-ethylhexyl ester (Neo Heliopan®OS)
triethanolamine salicylate
4-isopropyl benzyl salicylate
anthranilic acid menthyl ester (Neo Heliopan®MA)
diisopropyl cinnamic acid ethyl ester
p-methoxycinnamic acid-2-ethylhexyl ester (Neo Heliopan®AV)
diisopropyl cinnamic acid methyl ester
p-methoxycinnamic acid isoamyl ester (Neo Heliopan®E 1000)
p-methoxycinnamic acid diethanolamine salt
p-methoxycinnamic acid isopropyl ester
2-phenylbenzimidazole sulfonic acid and salts (Neo Heliopan®Hydro)
3-(4′-trimethylammonium) benzylidene bornan-2-one methyl sulfate
beta-imidazole-4(5)-acrylic acid (urocanic acid)
3-(4′-sulfo)benzylidene bornan-2-one and salts
3-(4′-methyl benzylidene)-D,L-camphor (Neo Heliopan®MBC)
3-benzylidene-D,L-camphor
N-[(2 and 4)-[2-(oxoborn-3-ylidene) methyl]benzyl] acrylamide polymer
4,4′-[(6-[4-(1,1-dimethyl)aminocarbonyl) phenylamino]-1,3,5-triazine-2,4-diyl)diimino]-bis-(benzoic acid-2-ethylhexyl ester) (Uvasorb®HEB)
benzylidene malonate polysiloxane (Parsol®SLX)
glyceryl ethylhexanoate dimethoxycinnamate
dipropylene glycol salicylate
tris(2-ethylhexyl)-4,4′,4″-(1,3,5-triazine-2,4,6-triyltriimino)tribenzoate (=2,4,6-trianilino-(p-carbo-2′-ethylhexyl-1′-oxy)-1,3,5-triazine) (Uvinul®T150).

In a preferred variant the cosmetic or pharmaceutical composition according to the present invention comprises a combination with one or more broadband filters which are selected from the group consisting of
2-ethylhexyl-2-cyano-3,3-diphenyl acrylate (Neo Heliopan©303)
ethyl-2-cyano-3,3′-diphenyl acrylate
2-hydroxy-4-methoxybenzophenone (Neo Heliopan®BB)
2-hydroxy-4-methoxybenzophenone-5-sulfonic acid
dihydroxy-4-methoxybenzophenone
2,4-dihydroxybenzophenone
tetrahydroxybenzophenone
2,2′-dihydroxy-4,4′-dimethoxybenzophenone
2-hydroxy-4-n-octoxybenzophenone
2-hydroxy-4-methoxy-4′-methyl benzophenone
sodium hydroxymethoxybenzophenone sulfonate
disodium-2,2′-dihydroxy-4,4′-dimethoxy-5,5′-disulfobenzophenone
phenol, 2-(2H-benzotriazol-2-yl)-4-methyl-6-(2-methyl-3(1,3,3,3-tetramethyl-1-(trimethylsilyl)oxy)disiloxyanyl) propyl) (Mexoryl®XL)
2,2′-methylene bis-(6-(2H-benzotriazol-2-yl)-4-1,1,3,3-tetramethylbutyl) phenol) (Tinosorb®M)
2,4-bis-[4-(2-ethylhexyloxy)-2-hydroxyphenyl]-1,3,5-triazine
2,4-bis-[{(4-(2-ethylhexyloxy)-2-hydroxy}phenyl]-6-(4-methoxyphenyl)-1,3,5-triazine (Tinosorb®S)
2,4-bis-[{(4-(3-sulfonato)-2-hydroxypropyloxy)-2-hydroxy}phenyl]-6-(4-methoxyphenyl)-1,3,5-triazine sodium salt
2,4-bis-[{(3-(2-propyloxy)-2-hydroxypropyloxy)-2-hydroxy}phenyl]-6-(4-methoxyphenyl)-1,3,5-triazine
2,4-bis-[{4-(2-ethylhexyloxy)-2-hydroxy}phenyl]-6-[4-(2-methoxyethyl carbonyl) phenylamino]-1,3,5-triazine
2,4-bis-[{4-(3-(2-propyloxy)-2-hydroxypropyloxy)-2-hydroxy}phenyl]-6-[4-(2-ethylcarboxyl) phenylamino]-1,3,5-triazine
2,4-bis-[{4-(2-ethylhexyloxy)-2-hydroxy}phenyl]-6-(1-methylpyrrol-2-yl)-1,3,5-triazine
2,4-bis-[{4-tris-(trimethylsiloxysilylpropyloxy)-2-hydroxy}phenyl]-6-(4-methoxyphenyl)-1,3,5-triazine
2,4-bis-[{4-(2″-methylpropenyloxy)-2-hydroxy}phenyl]-6-(4-methoxyphenyl)-1,3,5-triazine
2,4-bis-[{4-(1′,1′,1′,3′,5′,5′,5′-heptamethylsiloxy-2″-methylpropyloxy)-2-hydroxy}phenyl]-6-(4-methoxyphenyl)-1,3,5-triazine.

In a preferred variant the cosmetic or pharmaceutical composition according to the present invention comprises a combination with one or more UV-A filters which are selected from the group consisting of
4-isopropyl dibenzoyl methane
terephthalylidene dibornane sulfonic acid and salts (Mexoryl®SX)
4-t-butyl-4′-methoxydibenzoyl methane (avobenzone)/(Neo Heliopan®357)
phenylene bis-benzimidazyl tetrasulfonic acid disodium salt (Neo Heliopan®AP)
2,2′-(1,4-phenylene)-bis-(1H-benzimidazole-4,6-disulfonic acid), monosodium salt
2-(4-diethylamino-2-hydroxybenzoyl) benzoic acid hexyl ester (Uvinul® A Plus)
indanylidene compounds.

In a more preferred variant, the cosmetic or pharmaceutical composition according to the present invention comprises a combination with one or more UV filters which are selected from the group consisting of:
p-aminobenzoic acid
3-(4′-trimethylammonium) benzylidene bornan-2-one methyl sulfate
salicylic acid homomenthyl ester (Neo Heliopan®HMS)
2-hydroxy-4-methoxybenzophenone (Neo Heliopan®BB)
2-phenylbenzimidazole sulfonic acid (Neo Heliopan®Hydro)
terephthalylidene dibornane sulfonic acid and salts (Mexoryl®SX)
4-tert-butyl-4′-methoxydibenzoyl methane (Neo Heliopan®357)
3-(4′-sulfo)benzylidene bornan-2-one and salts
2-ethylhexyl-2-cyano-3,3-diphenyl acrylate (Neo Heliopan©303)
N-[(2 and 4)-[2-(oxoborn-3-ylidene) methyl]benzyl] acrylamide polymer
p-methoxycinnamic acid-2-ethylhexyl ester (Neo Heliopan®AV)
p-aminobenzoic acid ethyl ester (25 mol) ethoxylated (INCI name: PEG-25 PABA)
p-methoxycinnamic acid isoamyl ester (Neo Heliopan®E1000)
2,4,6-trianilino-(p-carbo-2′-ethylhexyl-1′-oxy)-1,3,5-triazine (Uvinul®T150)
phenol, 2-(2H-benzotriazol-2-yl)-4-methyl-6-(2-methyl-3(1,3,3,3-tetramethyl-1-(trimethylsilyl)oxy)disiloxyanyl) propyl) (Mexoryl®XL)
4,4′-[(6-[4-(1,1-dimethyl)aminocarbonyl) phenylamino]-1,3,5-triazine-2,4-diyl)diimino]-bis-(benzoic acid-2-ethylhexyl ester) (Uvasorb HEB)
3-(4′-methyl benzylidene)-D,L-camphor (Neo Heliopan®MBC)
3-benzylidene camphor
salicylic acid-2-ethylhexyl ester (Neo Heliopan®OS)
4-dimethylaminobenzoic acid-2-ethylhexyl ester (Padimate O)
hydroxy-4-methoxybenzophenone-5-sulfonic acid and Na salt
2,2′-methylene bis-(6-(2H-benzotriazol-2-yl)-4-1,1,3,3-tetramethylbutyl) phenol) (Tinosorb®M)
phenylene bis-benzimidazyl tetrasulfonic acid disodium salt (Neo Heliopan®AP)
2,4-bis-[{(4-(2-ethylhexyloxy)-2-hydroxy}phenyl]-6-(4-methoxyphenyl)-1,3,5-triazine (Tinosorb®S)
benzylidene malonate polysiloxane (Parsol®SLX)
menthyl anthranilate (Neo Heliopan®MA)
2-(4-diethylamino-2-hydroxybenzoyl) benzoic acid hexyl ester (Uvinul® A Plus)
indanylidene compounds.

In a further preferred variant, the cosmetic or pharmaceutical composition according to the invention contains a total amount of sunscreen agents, i.e. in particular UV filters and/or inorganic pigments (UV filtering pigments) so that the composition according to the invention has a light protection factor of greater than or equal to 2 (preferably greater than or equal to 5). Such compositions according to the invention are particularly suitable for protecting the skin and hair.

Secondary sun protection factors: Besides the groups of primary sun protection factors mentioned above, secondary sun protection factors of the antioxidant type may also be advantageously used in the cosmetic or pharmaceutical composition according to the present invention. Secondary sun protection factors of the antioxidant type interrupt the photochemical reaction chain which is initiated when UV rays penetrate into the skin. Typical examples are amino acids (for example glycine, histidine, tyrosine, tryptophane) and derivatives thereof, imidazoles (for example urocanic acid) and derivatives thereof, peptides, such as D,L-carnosine, D-carnosine, L-carnosine and derivatives thereof (for example anserine), carotinoids, carotenes (for example alpha-carotene, beta-carotene, lycopene) and derivatives thereof, chlorogenic acid and derivatives thereof, liponic acid and derivatives thereof (for example dihydroliponic acid), aurothioglucose, propylthiouracil and other thiols (for example thioredoxine, glutathione, cysteine, cystine, cystamine and glycosyl, N-acetyl, methyl, ethyl, propyl, amyl, butyl and lauryl, palmitoyl, oleyl, alpha-linoleyl, cholesteryl and glyceryl esters thereof) and their salts, dilaurylthiodipropionate, distearyl-thiodipropionate, thiodipropionic acid and derivatives thereof (esters, ethers, peptides, lipids, nucleotides, nucleosides and salts) and sulfoximine compounds (for example butionine sulfoximines, homocysteine sulfoximine, butionine sulfones, penta-, hexa- and hepta-thionine sulfoximine) in very small compatible dosages, also (metal) chelators (for example alpha-hydroxyfatty acids, palmitic acid, phytic acid, lactoferrine), alpha-hydroxy acids (for example citric acid, lactic acid, malic acid), humic acid, bile acid, bile extracts, bilirubin, biliverdin, EDTA, EGTA and derivatives thereof, unsaturated fatty acids and derivatives thereof (for example linoleic acid, oleic acid), folic acid and derivatives thereof, ubiquinone and ubiquinol and derivatives thereof, vitamin C and derivatives thereof (for example ascorbyl palmitate, Mg ascorbyl phosphate, ascorbyl acetate), tocopherols and derivatives (for example vitamin E acetate), vitamin A and derivatives (vitamin A palmitate) and coniferyl benzoate of benzoin resin, rutinic acid and derivatives thereof, glycosyl rutin, ferulic acid, furfurylidene glucitol, carnosine, butyl hydroxytoluene, butyl hydroxyanisole, nordihydroguaiac resin acid, nordihydroguaiaretic acid, trihydroxybutyrophenone, uric acid and derivatives thereof, mannose and derivatives thereof, superoxide dismutase, titanium dioxide (for example dispersions in ethanol), zinc and derivatives thereof (for example ZnO, ZnSO₄), selenium and derivatives thereof (for example selenium methionine), stilbenes and derivatives thereof (for example stilbene oxide, trans-stilbene oxide) and derivatives of these active substances suitable for the purposes of the invention (salts, esters, ethers, sugars, nucleotides, nucleosides, peptides and lipids).

Advantageous inorganic secondary light protection pigments are finely dispersed metal oxides and metal salts. The total quantity of inorganic pigments, in particular hydrophobic inorganic micro-pigments in the finished cosmetic preparation according to the present invention is advantageously from 0.1 to 30% by weight, preferably 0.5 to 10.0% by weight, in each case based on the total weight of the preparation.

Also preferred are particulate UV filters or inorganic pigments, which can optionally be hydrophobed, can be used, such as the oxides of titanium (TiO₂), zinc (ZnO), iron (Fe₂O₃), zirconium (ZrO₂), silicon (SiO₂), manganese (e.g. MnO), aluminium (Al₂O₃), cerium (e.g. Ce₂O₃), and/or mixtures thereof.

Actives modulating skin and/or hair pigmentation: The cosmetic or pharmaceutical composition according to the present invention may include active ingredients for skin and/or hair lightening. Preferred active ingredients for skin and/or hair lightening are selected from the group consisting of kojic acid (5-hydroxy-2-hydroxymethyl-4-pyranone), kojic acid derivatives, preferably kojic acid dipalmitate, arbutin, ascorbic acid, ascorbic acid derivatives, preferably magnesium ascorbyl phosphate, hydroquinone, hydroquinone derivatives, resorcinol, resorcinol derivatives, preferably 4-alkylresorcinols and 4-(1-phenylethyl)1,3-dihydroxybenzene (phenylethyl resorcinol), cyclohexylcarbamates, sulfur-containing molecules, preferably glutathione or cysteine, alpha-hydroxy acids (preferably citric acid, lactic acid, malic acid), salts and esters thereof, N-acetyl tyrosine and derivatives, undecenoyl phenylalanine, gluconic acid, chromone derivatives, preferably aloesin, flavonoids, 1-aminoethyl phosphinic acid, thiourea derivatives, ellagic acid, nicotinamide (niacinamide), zinc salts, preferably zinc chloride or zinc gluconate, thujaplicin and derivatives, triterpenes, preferably maslinic acid, sterols, preferably ergosterol, benzofuranones, preferably senkyunolide, vinyl guiacol, ethyl guiacol, dionic acids, preferably octodecene dionic acid and/or azelaic acid, inhibitors of nitrogen oxide synthesis, preferably L-nitroarginine and derivatives thereof, 2,7-dinitroindazole or thiocitrulline, metal chelators (preferably alpha-hydroxy fatty acids, phytic acid, humic acid, bile acid, bile extracts, EDTA, EGTA and derivatives thereof), retinoids, soy milk and extract, serine protease inhibitors or lipoic acid or other synthetic or natural active ingredients for skin and hair lightening, the latter preferably used in the form of an extract from plants, preferably bearberry extract, rice extract, papaya extract, turmeric extract, mulberry extract, bengkoang extract, nutgrass extract, liquorice root extract or constituents concentrated or isolated therefrom, preferably glabridin or licochalcone A, artocarpus extract, extract of rumex and ramulus species, extracts of pine species (pinus), extracts of vitis species or stilbene derivatives isolated or concentrated therefrom, saxifrage extract, scutelleria extract, grape extract and/or microalgae extract, in particular Tetraselmis suecica extract.

Preferred skin lighteners are kojic acid and phenylethyl resorcinol as tyrosinase inhibitors, beta- and alpha-arbutin, hydroquinone, nicotinamide, dioic acid, Mg ascorbyl phosphate and vitamin C and its derivatives, mulberry extract, Bengkoang extract, papaya extract, turmeric extract, nutgrass extract, licorice extract (containing glycyrrhizin), alpha-hydroxy-acids, 4-alkylresorcinols, 4-hydroxyanisole. These skin lighteners are preferred due to their very good activity, in particular in combination with sclareolide according to the present invention. In addition, said preferred skin lighteners are readily available.

Advantageous skin and hair tanning active ingredients in this respect are substrates or substrate analogues of tyrosinase such as L-tyrosine, N-acetyl tyrosine, L-DOPA or L-dihydroxyphenylalanine, xanthine alkaloids such as caffeine, theobromine and theophylline and derivatives thereof, proopiomelanocortin peptides such as ACTH, alpha-MSH, peptide analogues thereof and other substances which bind to the melanocortin receptor, peptides such as Val-Gly-Val-Ala-Pro-Gly, Lys-Ile-Gly-Arg-Lys or Leu-Ile-Gly-Lys, purines, pyrimidines, folic acid, copper salts such as copper gluconate, chloride or pyrrolidonate, 1,3,4-oxadiazole-2-thiols such as 5-pyrazin-2-yl-1,3,4-oxadiazole-2-thiol, curcumin, zinc diglycinate (Zn(Gly)2), manganese(II) bicarbonate complexes (“pseudocat-alases”), tetrasubstituted cyclohexene derivatives, isoprenoids, melanin derivatives such as Melasyn-100 and MelanZe, diacyl glycerols, aliphatic or cyclic diols, psoralens, prostaglandins and analogues thereof, activators of adenylate cyclase and compounds which activate the transfer of melanosomes to keratinocytes such as serine proteases or agonists of the PAR-2 receptor, extracts of plants and plant parts of the chrysanthemum species, san-guisorba species, walnut extracts, urucum extracts, rhubarb extracts, microalgae extracts, in particular Isochrysis galbana, trehalose, erythru-lose and dihydroxyacetone. Flavonoids which bring about skin and hair tinting or brown-ing (e.g. quercetin, rhamnetin, kaempferol, fisetin, genistein, daidzein, chrysin and api-genin, epicatechin, diosmin and diosmetin, morin, quercitrin, naringenin, hesperidin, phloridzin and phloretin) can also be used.

Hair growth activators or inhibitors: Cosmetic or pharmaceutical compositions according to the present invention may also comprise one or more hair growth activators, i.e., agents to stimulate hair growth. Hair growth activators are preferably selected from the group consisting of pyrimidine derivatives such as 2,4-diaminopyrimidine-3-oxide (Aminexil), 2,4-diamino-6-piperidinopyrimidine-3-oxide (Minoxidil) and derivatives thereof, 6-amino-1,2-dihydro-1-hydroxy-2-imino-4-piperidinopyrimidine and its derivatives, xanthine alkaloids such as caffeine, theobromine and theophylline and derivatives thereof, quercetin and derivatives, dihydroquercetin (taxifolin) and derivatives, potassium channel openers, antiandrogenic agents, synthetic or natural 5-reductase inhibitors, nicotinic acid esters such as tocopheryl nicotinate, benzyl nicotinate and C1-C6 alkyl nicotinate, proteins such as for example the tripeptide Lys-Pro-Val, diphencypren, hormones, finasteride, dutasteride, flutamide, bicalutamide, pregnane derivatives, progesterone and its derivatives, cyproterone acetate, spironolactone and other diuretics, calcineurin inhibitors such as FK506 (Tacrolimus, Fujimycin) and its derivatives, Cyclosporin A and derivatives thereof, zinc and zinc salts, polyphenols, procyanidins, proanthocyanidins, phytosterols such as for example beta-sitosterol, biotin, eugenol, (±)-beta-citronellol, panthenol, glycogen for example from mussels, extracts from microorganisms, algae, plants and plant parts of for example the genera dandelion (Leontodon or Taraxacum), Orthosiphon, Vitex, Coffea, Paullinia, Theobroma, Asiasarum, Cucurbita or Styphnolobium, Serenoa repens (saw palmetto), Sophora flavescens, Pygeum africanum, Panicum miliaceum, Cimicifuga racemosa, Glycine max, Eugenia caryophyllata, Cotinus coggygria, Hibiscus rosa-sinensis, Camellia sinensis, Ilex paraguariensis, Isochrysis galbana, licorice, grape, apple, barley or hops and/or hydrolysates from rice or wheat.

Alternatively, the cosmetic or pharmaceutical composition according to the present invention may include one or more hair growth inhibitors (as described above), i.e., agents to reduce or prevent hair growth. Hair growth inhibitors are preferably selected from the group consisting of activin, activin derivatives or activin agonists, ornithine decarboxylase inhibitors such as alpha-difluoromethylornithine or pentacyclic triterpenes like for example ursolic acid, betulin, betulinic acid, oleanolic acid and derivatives thereof, 5alpha-reductase inhibitors, androgen receptor antagonists, S-adenosylmethionine decarboxylase inhibitors, gamma-glutamyl transpeptidase inhibitors, transglutaminase inhibitors, soybean-derived serine protease inhibitors, extracts from microorganisms, algae, different microalgae or plants and plant parts of for example the families Leguminosae, Solanaceae, Graminae, Asclepiadaceae or Cucurbitaceae, the genera Chondrus, Gloiopeltis, Ceramium, Durvillea, Glycine max, Sanguisorba officinalis, Calendula officinalis, Hamamelis virginiana, Arnica montana, Salix alba, Hypericum perforatum or Gymnema sylvestre.

Enzyme inhibitors: The cosmetic or pharmaceutical composition according to the present invention may comprise one or more enzyme inhibitors. Suitable enzyme inhibitors are, for example, esterase inhibitors. These are preferably trialkyl citrates, such as trimethyl citrate, tripropyl citrate, triisopropyl citrate, tributyl citrate and, in particular, triethyl citrate (Hydagen CAT). The substances inhibit enzyme activity, thereby reducing the formation of odour. Other substances which are suitable esterase inhibitors are sterol sulfates or phosphates, such as, for example, lanosterol, cholesterol, campesterol, stigmasterol and sitosterol sulfate or phosphate, dicarboxylic acids and esters thereof, such as, for example, glutaric acid, monoethyl glutarate, diethyl glutarate, adipic acid, monoethyl adipate, diethyl adipate, malonic acid and diethyl malonate, hydroxycarboxylic acids and esters thereof, such as, for example, citric acid, malic acid, tartaric acid or diethyl tartrate, and zinc glycinate.

Film formers: The cosmetic or pharmaceutical composition according to the present invention may include one or more film formers. Standard film formers are preferably chitosan, microcrystalline chitosan, quaternized chitosan, polyvinyl pyrrolidone, vinyl pyrrolidone/vinyl acetate copolymers, polymers of the acrylic acid series, quaternary cellulose derivatives, collagen, hyaluronic acid and salts thereof and similar compounds.

Carriers and hydrotropes: The cosmetic or pharmaceutical composition according to the present invention may comprise a carrier or a mixture of different carriers. Preferred cosmetics carrier materials are solid or liquid at 25° C. and 1013 mbar (including highly viscous substances) as for example glycerol, 1,2-propylene glycol, 1,2-butylene glycol, 1,3-propylene glycol, 1,3-butylene glycol, ethanol, water, and mixtures of two or more of said liquid carrier materials with water. Optionally, these preparations according to the invention may be produced using preservatives or solubilizers. Other preferred liquid carrier substances, which may be a component of a preparation according to the invention are selected from the group consisting of oils such as vegetable oil, neutral oil and mineral oil.

Preferred solid carrier materials, which may be a component of the cosmetic or pharmaceutical composition according to the invention are hydrocolloids, such as starches, degraded starches, chemically or physically modified starches, dextrins, (powdery) maltodextrins (preferably with a dextrose equivalent value of 5 to 25, preferably of 10-20), lactose, silicon dioxide, glucose, modified celluloses, gum arabic, ghatti gum, traganth, karaya, carrageenan, pullulan, curdlan, xanthan gum, gellan gum, guar flour, carob bean flour, alginates, agar, pectin, inulin, and mixtures of two or more of these solids, in particular maltodextrins (preferably with a dextrose equivalent value of 15-20), lactose, silicon dioxide and/or glucose.

In addition, hydrotropes, for example ethanol, isopropyl alcohol or polyols, may be used to improve flow behaviour. Suitable polyols preferably contain 2 to 15 carbon atoms and at least two hydroxyl groups. The polyols may contain other functional groups, more especially amino groups, or may be modified with nitrogen. Typical examples are
glycerol;
alkylene glycols such as, for example, ethylene glycol, diethylene glycol, propylene glycol, butylene glycol, hexylene glycol and polyethylene glycols with an average molecular weight of 100 to 1000 Dalton;
technical oligoglycerol mixtures with a degree of self-condensation of 1.5 to 10, such as for example technical diglycerol mixtures with a diglycerol content of 40 to 50% by weight;
methylol compounds such as, in particular, trimethylol ethane, trimethylol propane, trimethylol butane, pentaerythritol and dipentaerythritol;
lower alkyl glucosides, particularly those containing 1 to 8 carbon atoms in the alkyl group, for example methyl and butyl glucoside;
sugar alcohols containing 5 to 12 carbon atoms, for example sorbitol or mannitol,
sugars containing 5 to 12 carbon atoms, for example glucose or sucrose;
amino sugars, for example glucamine;
dialcoholamines, such as diethanolamine or 2-aminopropane-1,3-diol.

Dyes: The cosmetic or pharmaceutical composition according to the present invention may comprise one or more dyes. Suitable dyes are any of the substances suitable and approved for cosmetic purposes. Examples include cochineal red A (CI. 16255), patent blue V (CI. 42051), indigotin (CI. 73015), chlorophyllin (CI. 75810), quinoline yellow (CI. 47005), titanium dioxide (CI. 77891), indanthrene blue RS (CI. 69800) and madder lake (CI. 58000). Luminol may also be present as a luminescent dye. Advantageous coloured pigments are for example titanium dioxide, mica, iron oxides (e.g. Fe₂O₃ Fe₃O₄, FeO(OH)) and/or tin oxide. Advantageous dyes are for example carmine, Berlin blue, chromium oxide green, ultramarine blue and/or manganese violet.

In addition to the above-described substances, further ingredients commonly used in the cosmetic or pharmaceutical industry, which are suitable or customary in the compositions of the present invention, can be used.

The 1,2-nonanediol or the 2,3-nonanediol or the mixture comprising 1,2-nonanediol and 2,3-nonanediol is present in the cosmetic or pharmaceutical composition according to the first or second aspect of the present invention, in an amount of 0.001 to 15.0% by weight, based on the total weight of the composition. In a preferred variant, the cosmetic or pharmaceutical composition comprises the 1,2-nonanediol or the 2,3-nonanediol or the mixture comprising 1,2-nonanediol and 2,3-nonanediol in an amount of 0.005 to 10.0% by weight, based on the total weight of the composition. In a particular preferred variant, the 1,2-nonanediol or 2,3-nonanediol or the mixture comprising 1,2-nonanediol and 2,3-nonanediol is advantageously used in the cosmetic or pharmaceutical composition in an amount of at 0.01 to 5.0% by weight, based on the total weight of the composition. In a most preferred variant, the 1,2-nonanediol or 2,3-nonanediol or the mixture comprising 1,2-nonanediol and 2,3-nonanediol is advantageously used in the cosmetic or pharmaceutical composition in an amount of 0.02 to 3.0% by weight, based on the total weight of the composition.

For the nonanediol mixture, the above amounts relate on the total content of the alkanediolsalkanediols1,2-nonanediol and 2,3-nonanediol in the mixture, i.e., the amount is the sum of the content of the alkanediolsalkanediols1,2-nonanediol and 2,3-nonanediol in the mixture.

The at least one linear alkanediol or the mixture comprising at least one first linear alkanediol and one or more second linear alkanediols according to the second aspect of the present invention is present in the cosmetic or pharmaceutical composition in an amount of 0.001 to 15.0% by weight, based on the total weight of the composition. In a preferred variant, the cosmetic or pharmaceutical composition comprises the at least one linear alkanediol or the mixture comprising at least one first linear alkanediol and one or more second linear alkanediols in an amount of 0.005 to 10.0% by weight, based on the total weight of the composition. In a particular preferred variant, the at least one linear alkanediol or the mixture comprising at least one first linear alkanediol and one or more second linear alkanediols is advantageously used in the cosmetic or pharmaceutical composition in an amount of at 0.01 to 5.0% by weight, based on the total weight of the composition. In a most preferred variant, the at least one linear alkanediol or the mixture comprising at least one first linear alkanediol and one or more second linear alkanediols is advantageously used in the cosmetic or pharmaceutical composition in an amount of at 0.02 to 3.0% by weight, based on the total weight of the composition.

For the alkane mixture, the above amounts relate to the total content of the first linear alkanediol and the second linear alkanediol in the mixture, i.e. the amount is the sum of the content of the first linear alkanediol and the second linear alkanediol in the mixture.

In a particular preferred variant according to the first aspect of the present invention, the cosmetic or pharmaceutical composition comprises the 2,3-nonanediol or the 2,3-nonanediol or the mixture comprising 1,2-nonanediol and 2,3-nonanediol in an amount of 0.001 to 15.0% by weight, preferred in an amount of 0.005 to 10.0% by weight, more preferred in an amount of 0.01 to 5.0% by weight and most preferred in an amount of 0.02 to 3.0% by weight.

Even more preferred, the cosmetic or pharmaceutical composition comprises the 2,3-nonanediol or the 2,3-nonanediol of the mixture comprising 1,2-nonanediol and 2,3-nonanediol in an amount of 0.001 to 0.5% weight, preferably in an amount of 0.005 to 0.1% by weight and most preferably in an amount of 0.01 to 0.075% by weight, based on the total weight of the composition.

In a particular preferred variant according to the first aspect of the present invention, the cosmetic or pharmaceutical composition comprises the 2,3-alkanediol as linear alkanediol or of the mixture comprising at least one first linear alkanediol and one or more second linear alkanediol in an amount of 0.001 to 15.0% by weight, preferably in an amount of 0.005 to 10.0% by weight, more preferred in an amount of 0.01 to 5.0% by weight and most preferred in an amount of 0.02 to 3.0% by weight.

Even more preferred, the cosmetic or pharmaceutical composition comprises the 2,3-alkanediol as linear alkanediol or of the mixture comprising at least one first linear alkanediol and one or more second linear alkanediol in an amount of 0.001 to 0.5% weight, preferably in an amount of 0.005 to 0.1% by weight and most preferably in an amount of 0.01 to 0.075% by weight, based on the total weight of the composition.

The antimicrobial component is present in the cosmetic or pharmaceutical composition according to the present invention, in an amount of 0.001 to 15.0% by weight, based on the total weight of the composition. In a preferred variant, the cosmetic or pharmaceutical composition comprises the antimicrobial component in an amount of 0.005 to 10.0% by weight, based on the total weight of the composition. In a particular preferred variant, the antimicrobial component is advantageously used in the cosmetic or pharmaceutical composition in an amount of at 0.01 to 5.0% by weight, based on the total weight of the composition.

As it is demonstrated in the following examples the presence of a 1,2-alkanediol, in particular 1,2-heptanediol, 1,2-octanediol, 1,2-nonanediol and 1,2-undecanediol, considerably enhances the antimicrobial effect of an antimicrobial component containing cosmetic or pharmaceutical composition upon topical application on skin. Surprisingly, the cosmetic or pharmaceutical composition as disclosed herein, comprising a combination of a 1,2-alkanediol with an antimicrobial component showed excellent antimicrobial activity against well-known pathogens on the skin as described herein. Surprisingly, the combination of an antimicrobial component with an 1,2-alkanediol, in particular 1,2-heptanediol, 1,2-nonanediol and 1,2-undecanediol, even leads to a synergistic increase in antimicrobial activity of said antimicrobial component, against a plurality of microorganisms of the mammalian natural microbiota on the skin or scalp or mucosa. Especially, 1,2-nonanediol has a strong antimicrobial efficacy when used in combination with different antimicrobial components resulting in Synergy Indices <1.0.

A synergistic effect is an effect achieved when two different chemical substances or biological structures interact resulting in an overall effect that is greater than the sum of individual effects of any of them.

Antimicrobial effect in the context of the present invention means that the microorganisms' growth is reduced or even inhibited, so that the number of living microorganisms on the skin or scalp stagnates or is reduced.

A synergistic antimicrobial effect is achieved for a combination of a 2,3-alkanediol, in particular 2,3-heptanediol, 2,3-nonanediol and 2,3-undecanediol, with an antimicrobial component.

Due to the synergistic antimicrobial effect resulting from a combination of a 1,2-alkanediol or a 2,3-alkanediol and an antimicrobial component, the amount of the antimicrobial component in a cosmetic or pharmaceutical composition can be reduced compared to a composition including an antimicrobial component without the addition of a 1,2-alkanediol or 2,3-alkanediol.

Also a mixtures comprising a 1,2-alkanediol and a 2,3-alkanediol considerably enhances the antimicrobial effect of an antimicrobial component containing cosmetic or pharmaceutical composition upon topical application on skin. Surprisingly, the cosmetic or pharmaceutical composition as disclosed herein, comprising a mixture comprising a 1,2-alkanediol and a 2,3-alkanediol when used with an antimicrobial component showed excellent antimicrobial activity against well-known pathogens on the skin as described herein. Surprisingly, the combination of an antimicrobial component (Benzyl Alcohol, o-Cymen-5-ol and Caprylhydroxamic acid) with a mixture of 1,2-heptanediol and 2,3-heptanediol or a mixture of 1,2-nonanediol and 2,3-nonanediol considerably inhibits microorganisms' growth and resulting in synergy indices of <1.0.

Hence, the use of 1,2-nonanediol or 2,3-nonanediol or a mixture comprising 1,2-nonanediol and 2,3-nonanediol or a linear alkanediol or a mixture of a first linear alkanediol with a second linear alkanediol according to the present invention allows for the use of less antimicrobial component in a cosmetic or pharmaceutical composition. In other words: the range of efficiency of the antimicrobial component is broadened, either by achieving a higher performance of an antimicrobial component with the same amounts, or, vice versa, by achieving the same performance with lower amounts of an antimicrobial component, so that the overall content of the antimicrobial component in the composition can be reduced.

In addition to the above described synergistically antimicrobial effect, the use of a 1,2-alkanediol, in particular 1,2-heptanediol, 1,2-nononediol and 1,2-undecanediol, has surprisingly a modulatory effect on the sebum production of sebaceous glands. By the use of a 1,2-alkanediol the sebum production of the sebaceous glands could be balanced and/or a sebum overproduction can be reduced effectively.

Even a stronger sebum reducing effect is achieved for a 2,3-alkanediol, in particular 2,3-heptanediol, 2,3-nonanediol and 2,3-undecanediol: 1,2-heptanediol shows a sebum reduction of 20% whereas 2,3-heptanediol shows a sebum reduction of 30%; 1,2-nonanediol reduces sebum secretion by 28% whereas the 2,3-nonanediol reduces sebum secretion by 40%. The best sebum reduction was observed for 1,2-undecanediol with 45%, whereas the 2,3-undecanediol leads to a sebum reduction of more than 50%. Hence, the 2,3-alkanediol substances have a distinguished effect on sebum reduction compared to the corresponding 1,2-alkanediol substances.

Furthermore, the 2,3-alkanediols have the benefit that they have an antimicrobial efficacy on the one hand and an enhanced sebum reducing effect compared to the respective 1,2-alkanediols. Additionally, the 2,3-alkanediols have the advantage that they are liquid, and, thus, can be easier incorporated into semi-finished products or final products and furnished in higher concentrations in the final product. Hence, a good compromise can be achieved when the 2,3-alkanediol is combined in such a way as to maintain the antimicrobial and sebum reducing effect, while improving processability in formulations.

Also combinations of a 1,2-alkanediol and a 2,3-alkanediol, where the 2,3-alkanediol represents the antimicrobial component as defined and described above, result in a synergistic antimicrobial effect against microorganisms of the natural microbiota of mammalian skin as it is shown in the following examples.

Since several microbial species on the skin or on the scalp are lipid dependent and feed on the human sebum, such as Corynebacterium, Propionibacterium, the sebum reduction can support the reduction of the number of microorganism or microorganisms' growth on the skin or scalp. In this way, sebum reduction and microorganism reduction indirectly support dandruff or acne reduction.

This sebum balancing or reducing effect was surprising, since no simple linear correlation between molecule length and activity of the 1,2-alkanediols or the 2,3-alkanediols can be established.

The cosmetic or pharmaceutical, preferably dermatological, composition according to the first or second aspect of the present invention is intended for topical applications. The term “topical” is understood to mean external applications on a mammal's skin, which are in particular for the treatment, protection, care and cleansing of the skin, scalp, eyelashes, eyebrows, nails, mucous membranes and hair. The mammal is preferably a human.

A preferred variant relates to a cosmetic product for topical application on a mammal's skin or mucosa. The mammal is preferably a human.

For topical application, the cosmetic products are either rinse off or leave on preparations.

The cosmetic or pharmaceutical, in particular dermatological, composition according to the first or second aspect of the present invention can be present in different forms of, e.g., in the form of a dispersion, in the form of a liquid surfactant formulation, in the form of a solid surfactant formulation, or in the form of an aqueous, aqueous/alcoholic, in particular aqueous/ethanolic, or an aqueous/glycolic based solution.

The cosmetic or pharmaceutical, preferably dermatological, composition according to the first or second aspect of the present invention is preferably a dispersion. The term “dispersion” in the context of the present invention means, that the cosmetic or pharmaceutical composition is a disperse two-phase system consisting of colloidal particles (disperse phase) and a medium in which they are suspended (disperse medium). Both phases are not miscible with each other, only with an emulsifier. Such dispersions, for example emulsions, comprise the at least oil component preferably in an amount of 1% by weight, more preferably in an amount of 3% by weight.

If the cosmetic or pharmaceutical composition according to the present invention is an emulsion, the oil component is present in the cosmetic or pharmaceutical composition in an amount of 0.01 to 50.0% by weight, based on the total weight of the composition. In a preferred variant, the cosmetic or pharmaceutical composition comprises the oil component in an amount of 1.0 to 40.0% by weight, based on the total weight of the composition. In a particular preferred variant, the oil component is advantageously used in the cosmetic or pharmaceutical composition in an amount of at 3.0 to 30% by weight, based on the total weight of the composition.

Preferably, the cosmetic or pharmaceutical composition according to the first or second aspect of the present invention takes various forms such as an emulsion, in particular a O/W emulsion, a W/O emulsion, a multiple emulsion, a hydro dispersion gel, a balm, a multiple emulsion of the water-in-oil type (W/0/WO) or of the oil-in-water type (O/W/O), PIT emulsion, Pickering emulsion, a micro-emulsion, a liquid, a lotion, a suspension, a milk, an ointment, a paste, a gel, a cream based, an oil based or a liposome-based formulation.

In a further alternative, the cosmetic or pharmaceutical composition according to the present invention is a liquid surfactant formulation.

Such liquid surfactant formulations include for example shampoo, shower gel, micellar water, liquid soap, cleansing preparations.

If the cosmetic or pharmaceutical composition according to the present invention is a liquid surfactant formulation, the surfactant component is present in the cosmetic or pharmaceutical composition in an amount of 1 to 40% by weight, preferably in an amount of 3 to 30% by weight, more preferably in an amount of 5 to 25% by weight, based on the total weight of the composition.

In a further alternative, the cosmetic or pharmaceutical composition according to the present invention is a solid surfactant formulation.

Such solid surfactant formulations include for example solid shampoos, solid body wash, bar soaps, etc.

Alternatively, the cosmetic or pharmaceutical composition as disclosed herein is an aqueous or aqueous/alcoholic or aqueous/glycolic based solution. Said solution is a homogeneous one phase system of water and additional components. The aqueous/alcoholic or aqueous/glycolic based solution comprises an aliphatic alcohol or a glycol in an amount of 0.1 to ≤50% by weight, preferably in an amount of 0.5 to 30% by weight, more preferably in an amount of 2 to 20% by weight, based on the total weight of the solution. The aliphatic alcohol is preferably selected from the group consisting of ethanol, isopropanol, n-propanol. The glycol is preferably selected from the group consisting of glycerin, propylene glycol, butylene glycol or dipropylene glycol. Preferably, the overall water content in the final solutions can be 50% by weight, more preferably 70% by weight, even more preferably 80% by weight, and in special cases particularly preferably even 90% by weight, based on the total weight of the solution. New applications such as those including solutions for water wipes have high water content.

Such aqueous or aqueous/alcoholic or aqueous/glycolic based solutions include for example deo/antiperspirant preparations, after shave, cleansing preparations, or anti-acne preparations. In a preferred variant, the inventive composition can be used for wet-wipe applications; they may then most preferably contain 95% by weight water, or even 98% by weight water.

In a preferred variant, the cosmetic or pharmaceutical composition according to the present invention is in the form of an emulsion as defined herein, advantageously in the form of an oil-in-water (O/W) emulsion comprising an oily phase dispersed in an aqueous phase in the presence of an O/W emulsifier.

In another preferred variant, the cosmetic or pharmaceutical composition according to the present invention is in the form of an aqueous or aqueous/alcoholic, preferably aqueous/ethanolic, or aqueous/glycolic based solution. Typically, this could be glycerin in water compositions.

The above formulations or compositions are prepared according to usual and known methods.

Due to the enhanced antimicrobial and sebum balancing or reducing properties, the composition according to the present invention are beneficial for cosmetic, non-therapeutic or therapeutic use.

Thus, in a further aspect, the composition according to the first or second aspect of the present invention is a preparation for cosmetic use and/or non-therapeutic use for personal care, skin protection, skin care, scalp protection, scalp care, hair care, nail care, or in the prevention and/or treatment of skin conditions, intolerant or sensitive skin, skin irritation, skin reddening, pruritus (itching), loss of skin volume, loss of skin elasticity, skin dryness, flaking, greasiness; or for animal care.

In a preferred variant, the cosmetic product as disclosed herein is a serum, scrub, lotion, soap (bar soap, liquid soap, syndet), ointment, wax, powder, solution, emulsion, paste, suspension, tablet, gel, stick, aerosol, most preferably taking the form of an aerosol-based deo spray, deo pump spray, deo stick, deo roll on, deo cream, deo wipes, deo crystals, Pickering emulsions, hydro dispersion gels, skin balms, shampoo, shower gel, foam bath, micellar water, facial cleansing solutions, cleansing wipes, intimate spray, intimate cream, intimate wash lotion, intimate wipes, aerosol-based foot spray, foot pump spray, foot bath, foot balm, mouth wash concentrate, mouth wash ready to use, and tooth paste, etc.

Alternatively, the composition according to the present invention is a preparation for medical use.

The pharmaceutical, in particular dermatological, composition according to the present invention is preferably a preparation in the prevention and/or treatment of a condition of the skin, scalp or mucosa.

Preferably, the pharmaceutical composition according to the first or second aspect of the present invention is used in the prevention and/or treatment of dermatological or keratological diseases, in particular dermatological or keratological diseases caused by microorganisms or associated with a sebum overproduction or severe dandruff, wherein the dermatological or keratological diseases are selected from the group consisting of eczema, psoriasis, seborrhea, dermatitis, erythema, pruritus (itching), inflammation, irritation, fibrosis, lichen planus, pityriasis rosea, pityriasis versicolor, autoimmune bullous diseases, urticaria, angioedema, allergic skin reactions, wound healing and tissue regeneration.

In order to be used, the cosmetic or pharmaceutical, in particular dermatological, composition according to the first or second aspect of the present invention is applied to the skin, hair, scalp and/or nails in an adequate amount in such manner as is customary with cosmetics and dermatological products.

Surprisingly, the use of a 1,2-alkanediol or a 2,3-alkanediol as described herein in combination with an antimicrobial component results in an enhanced, i.e., synergistically, antimicrobial effect inhibiting microorganisms' growth on a mammal's skin or mucosa. Additionally, the application of a 1,2-alkanediol or of a 2,3-alkanediol has a favourable, i.e., suppressing, effect on the sebum production of sebaceous glands by suppressing and, thus, reducing sebum production or has a favourable impact on dandruff or acne.

Thus, the present invention relates in a further aspect to the use of 1,2-nonanediol or 2,3-nonanediol or a mixture comprising 1,2-nonanediol and 2,3-nonanediol or at least one linear alkanediol or a mixture comprising at least one first linear alkanediol and one or more second linear alkanediols as defined herein as cosmetic or pharmaceutical.

Due to its distinguished antimicrobial and sebum reducing effect 2,3-alkanediols selected from the group consisting of 2,3-pentanediol, 2,3-hexanediol, 2,3-heptanediol, 2,3-nonanediol, 2,3-undecanediol, and 2,3-tridecanediol are most preferred in the use as cosmetic or pharmaceutical.

Alternatively, the present invention relates to the use of 1,2-nonanediol or 2,3-nonanediol or a mixture comprising 1,2-nonanediol and 2,3-nonanediol or at least one linear alkanediol or a mixture comprising at least one first linear alkanediol and one or more second linear alkanediols as defined herein for reducing or inhibiting the microbial growth on skin or scalp. The microorganisms are preferably bacteria and/or fungi of the natural microbiota of mammalian skin or mucosa. Preferably, the use is directed against the growth of microorganism from the genus Staphylococcus, Corynebacterium, Anaerococcus, Finegoldia, Moraxella, Porphyromonas, Fusobacterium, Aspergillus, Candida, Malassezia, Escherichia, Peptoniphilus, Streptococcus, Lactobacillus, Gardnerella, Fannyhessea, Epidermophyton, Trichophyton, Cutibacterium, and combinations thereof.

Alternatively, the present invention relates to the use of 1,2-nonanediol or 2,3-nonanediol or a mixture comprising 1,2-nonanediol and 2,3-nonanediol or at least one linear alkanediol or a mixture comprising at least one first linear alkanediol and one or more second linear alkanediols as defined herein for balancing sebum production or reducing sebum overproduction on skin or scalp and/or for suppressing dandruff on skin, scalp or nails.

Due to the synergistic antimicrobial effect, the present invention relates additionally to the use of 1,2-nonanediol or 2,3-nonanediol or a mixture comprising 1,2-nonanediol and 2,3-nonanediol or at least one linear alkanediol or a mixture comprising at least one first linear alkanediol and one or more second linear alkanediols as defined herein to reduce the amount of an antimicrobial component in a cosmetic or pharmaceutical composition, i.e., as antimicrobial component replacer.

In the use according to the present invention and described herein, the linear 1,2-alkanediol is preferably selected from the group consisting of 1,2-heptanediol, 1,2-nonanediol, 1,2-undecanediol, and 1,2-tridecanediol or the linear 2,3-alkanediol is preferably selected from the group consisting of 2,3-pentanediol, 2,3-hexanediol, 2,3-heptanediol, 2,3-nonanediol, 2,3-undecanediol, and 2,3-tridecanediol or the mixture comprising a linear 1,2-alkanediol and a linear 2,3-alkanediol is preferably selected from the group consisting of a mixture including 1,2-pentanediol and 2,3-pentanediol; a mixture including 1,2-hexanediol and 2,3-hexaneidol; a mixture including 1,2-heptanediol and 2,3-heptanediol; a mixture including 1,2-octanediol and 2,3-octanediol; a mixture including 1,2-nonanediol and 2,3-nonanediol; a mixture including 1,2-undecanediol and 2,3-undecanediol; and a mixture including 1,2-tridecanediol and 2,3-tridecanediol.

In the use according to the present invention, 1,2-nonanediol, 1,2-undecanediol, 2,3-nonanediol and/or 2,3-undecanediol are most preferred.

Due to the above described beneficial effect of the 1,2-alkanediols or 2,3-alkanediols or mixtures thereof as defined herein, the present invention relates in another aspect to personal hygiene products, a deodorant, an anti-perspirant, an anti-acne product, or an anti-dandruff product comprising a linear 1,2-alkanediol selected from the group consisting of 1,2-heptanediol, 1,2-nonanediol, 1,2-undecanediol, and 1,2-tridecanediol or comprising a linear 2,3-alkanediol selected from the group consisting of 2,3-pentanediol, 2,3-hexanediol, 2,3-heptanediol, 2,3-octanediol, 2,3-nonanediol, 2,3-undecanediol, and 2,3-tridecanediol or comprising a mixture of a linear 1,2-alkanediol with a linear 2,3-alkanediol selected from the group consisting of a mixture including 1,2-pentanediol and 2,3-pentanediol; a mixture including 1,2-hexanedial and 2,3-hexanediol; a mixture including 1,2-heptanediol and 2,3-heptanediol; a mixture including 1,2-octanediol and 2,3-octanediol; a mixture including 1,2-nonanediol and 2,3-nonanediol; a mixture including 1,2-undecanediol and 2,3-undecanediol; and a mixture including 1,2-tridecanediol and 2,3-tridecanediol.

The cosmetic or pharmaceutical products show excellent antimicrobial activity against the microorganism growth by inhibition or reduction of the number of living microorganism of the skin or scalp.

Additionally, the cosmetic or pharmaceutical products show a positive impact on sebum reduction, i.e., balancing sebum production and/or reducing sebum production, and suppression of dandruff.

In order to optimize their range of efficacy, the aforementioned products are combined with further active agents as specified in detail with regard to the cosmetic or pharmaceutical composition according to the first or second aspect of the present invention above. These agents can in some instances provide one or more than one additional benefit. Preferably, these active agents are preferably selected from the group agents consisting of sebum reducing agents, anti-acne agents, anti-dandruff agents, anti-inflammatory agents, anti-itch agents, anti-Malassezia agents, cleansing agents, odour absorbers and/or antiperspirant active agents, lenitive substances, moisturising and/or moisture-retaining substances, physiological cooling agents, Tetraselmis suecica extract, and mixtures of two or more of afore specified agents.

Personal hygiene products mean consumer products used in personal hygiene, i.e. a non-medicinal consumable product that is intended to be used in the topical care and grooming of the body or hair and is rubbed, poured, sprinkled, or sprayed on, introduced into, or otherwise applied, to a body, human or animal, for cleansing, beautifying, promoting attractiveness, or altering the appearance without affecting the body's structure or functions, and includes products specifically for use in such activities as cleansing, toning, moisturizing, hydrating, exfoliating, conditioning, anointing, massaging, coloring/decorating, soothing, deodorizing, perfuming, and styling.

Preferably, the personal hygiene product is selected from the group consisting of body (including foot) deodorant care products (for example deo spray aerosol based and deo pump spray, deo stick, deo roll on, deo cream, deo wipes, deo crystals, water free deo products), skin care (for example o/w emulsions, w/o emulsions, multiple emulsions, Pickering emulsions, hydro dispersion gels, balms, water free products) and cleansing products (for example shampoo, shower gel, foam bath, micellar water, facial cleansing solutions, water free cleansing products, cleansing wipes), intimate care and cleansing products (for example spray, cream, lotion, fluid, surfactant based rinse off preparations, wipes), foot care and cleansing products (for example aerosol spray, pump spray, foot bath, o/w or w/o emulsion, balm), and oral care products (for example mouth wash concentrate, mouth wash ready to use, tooth paste). That is, the cosmetic product is primarily intended to be applied under the armpit, to the face, to the feet, to the intimate region or to the oral mucosa for the primary purposes of providing a hygienic and “healthy” feeling. It is advantageous to formulate the cosmetic product as disclosed herein in a way that enables users to incorporate the desired effects, in particular the desired antimicrobial effect or the desired sebum suppressing effect, into their daily hygienic routine. This is achieved by, for example, providing the cosmetic product in the form of the above-mentioned (daily) care products.

In a final aspect, the present invention relates to a method for reducing the sebum overproduction on skin or scalp and/or for suppressing dandruff on skin, scalp or nails, comprising the following steps:
(a) providing of at least one linear 1,2-alkanediol selected from the group consisting of 1,2-heptanediol, 1,2-nonanediol, 1,2-undecanediol, and 1,2-tridecanediol or at least one linear 2,3-alkanediol selected from the group consisting of 2,3-pentanediol, 2,3-hexanediol, 2,3-heptanediol, 2,3-nonanediol, 2,3-undecanediol, and 2,3-tridecanediol or of a mixture of a linear 1,2-alkanediol with a linear 2,3-alkanediol selected from the group consisting of a mixture including 1,2-pentanediol and 2,3-pentanediol; a mixture including 1,2-hexanediol and 2,3-hexanediol; a mixture including 1,2-heptanediol and 2,3-heptanediol; a mixture including 1,2-octanediol and 2,3-octanediol; a mixture including 1,2-nonanediol and 2,3-nonanediol; a mixture including 1,2-undecanediol and 2,3-undecanediol; and a mixture including 1,2-tridecanediol and 2,3-tridecanediol;
(b) preparing of a cosmetic or pharmaceutical preparation to be topically applied and incorporating the alkanediol or the alkanediol mixture from step (a) into the cosmetic or pharmaceutical preparation; and
(c) applying the cosmetic or pharmaceutical preparation from step (b) to relevant skin or scalp areas;
wherein the amount of the alkanediol is sufficient to reduce the sebum overproduction or dandruff on skin, scalp or nails.

The present invention shall now be described in detail with reference to the following examples, which are merely illustrative of the present invention, such that the content of the present invention is not limited by or to the following examples.

### EXAMPLES

### Example 1: Evaluation of Product Inherent Smell

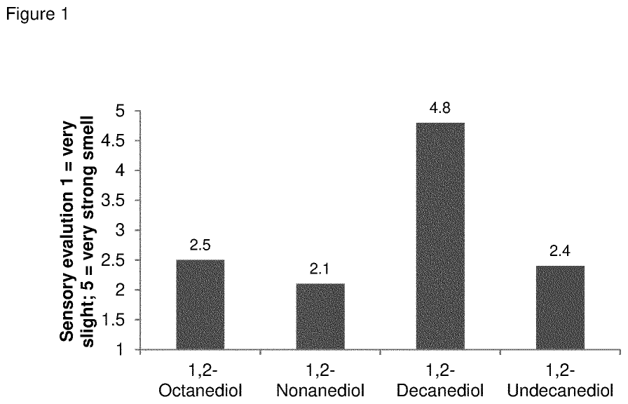

The product inherent smell of 1,2-octanediol, 1,2-nonanediol, 1,2-decanediol and 1,2-undecanediol was sensory evaluated by 10 persons and rated on a scale from 1 (very light smell) to 5 (very strong smell). 1,2-nonanediol resulted in the lowest smell, while 1,2-decanediol had the strongest. The results of the sensory evaluation are shown in FIG. 1.

### Example 2: Antimicrobial Activity: Minimum Inhibitory Concentration (MIC)

The Minimum Inhibitory Concentration (MIC) test is a test on growth inhibition. The estimation of MICs is executed in 96 well plates. Through the comparison of bacterial growth with positive and negative controls via optical density (OD), different concentrations of given test substances are evaluated.

Bacteria were cultivated under adjusted conditions based on information of the German Collection of Microorganisms and Cell Cultures (DSMZ) and stored in 50% glycerol prior to use. Afterwards, the following method was applied: Growth medium (according to microorganism) was added to each well of the microplate. Then, the test-substances were added in different concentrations, positive controls (water controls only with medium) and negative controls (benchmarks according to organism). Finally, the microorganisms were added in a concentration of 10⁶ CFU/ml (CFU=colony forming units). The incubation of the microplates was carried out at appropriate conditions.

According to the resulting growth, substance concentrations are labelled either as inhibiting or not and MIC is defined as the lowest concentration where a complete growth inhibition is visible. Experiments at concentration limits (highest concentration labelled as growth and lowest concentration labelled as inhibiting) are performed at least twice.

TABLE 1
Antimicrobial properties of 1,2-heptanediol, 1,2-nonanediol, and
1,2-undecanediol in comparison to 1,2-decanediol against microorganisms
naturally related to the human skin

| | Staphylococcus | Candida | Escherichia | Staphylococcus | Corynebacterium | Propionibacterium |
|---|---|---|---|---|---|---|
| | aureus | albicans | coli | hominis | jeikeium | acnes |
| 1,2- | 10000 | 5000 | 5000 | 10000 | 7500 | > 5000 |
| Heptanediol | | | | | | |
| 1,2- | 1000 | 1000 | 500 | 1000 | 2500 | 1000 |
| Nonanediol | | | | | | |
| 1,2- | 250 | 250 | 250 | 500 | 2500 | 500 |
| Decanediol | | | | | | |
| 1,2- | 125 | 64 | 250 | 125 | 250 | 250 |
| Undecanediol | | | | | | |

Minimum Inhibitory Concentration (MIC) are given in ppm. “>5000” indicates that 5000 ppm was tested and no growth inhibition found, higher values were not investigated.

As it is obvious from Table 1, 1,2-undecanediol has the lowest MIC concentration against all tested microorganisms; this means that less amounts of 1,2-undecanediol are required for inhibiting the microorganisms' growth. Thus, the antimicrobial performance of 1,2-undecanediol is better than 1,2-decanediol according to the prior art. In addition, 1,2-nonanediol has similar MIC values as 1,2-decanediol, which on the other hand, however, has an unpleasant inherent smell.

TABLE 2
Antimicrobial properties of 2,3-heptanediol, 2,3-octanediol, 2,3-
nonanediol and 2,3-undecanediol in comparison to 1,2-decanediol against
microorganisms naturally related to the human skin

| | Staphylococcus | Candida | Escherichia | Staphylococcus | Corynebacterium | Propionibacterium |
|---|---|---|---|---|---|---|
| | aureus | albicans | coli | hominis | jeikeium | acnes |
| 1,2- | 250 | 250 | 250 | 500 | 2500 | 500 |
| Decanediol | | | | | | |
| 2,3- | 20000 | > 5000 | >5000 | > 5000 | > 5000 | > 5000 |
| Heptanediol | | | | | | |
| 2,3- | 5000 | 5000 | 2500 | > 5000 | 5000 | > 5000 |
| Octanediol | | | | | | |
| 2,3- | 2500 | 2500 | 1000 | 5000 | 2500 | 2500 |
| Nonanediol | | | | | | |
| 2,3- | 250 | 250 | 500 | 250 | 250 | 250 |
| Undecanedi | | | | | | |
| ol | | | | | | |

Minimum Inhibitory Concentration (MIC) are given in ppm. “>5000” indicates that 5000 ppm was tested and no growth inhibition found, higher values were not investigated.

As it is obvious from Table 2, 2,3-undecanediol has the lowest MIC values against all tested microorganisms. This means that less amounts of 2,3-undecanediol are required for inhibiting microorganisms' growth. In addition, 2,3-undecanediol has similar MIC values as 1,2-decanediol, which on the other hand, however, has an unpleasant inherent smell. The antimicrobial performance of 2,3-undecanediol against C. jeikeium is even better than that of 1,2-decanediol.

### Example 3: Synergistic Activity

The synergistic activity of 1,2-nonanediol in combination with different antimicrobial compounds was determined by measuring the Minimum Inhibitory Concentrations (MIC) according to the method as described in Example 2.

To verify that the tested combinations act synergistically against microorganisms of the natural microbiota of mammalian skin, a corresponding experiment was conducted with Staphylococcus aureus, a well-known pathogen on the skin. The synergistic activities of 1,2-nonanediol and different antimicrobial compounds in a mixture of 20% and 80%, 80% and 20%, or 50% and 50% were determined by measuring the MIC of the mixture as indicated in methods section.

In order to calculate the synergy index with MIC values, the Kull equation (I) was used as follows:

SI=(MICₘᵢₓₜᵤᵣₑ×P_{A})/MIC_{A}+(MICₘᵢₓₜᵤᵣₑ×P_{B})/MIC_{B} (I)
wherein
SI is the Synergy Index according to Kull
MIC_{A} is the MIC value for Substance A
MIC_{B} is the MIC value for Substance B
MICₘᵢₓₜᵤᵣₑ is the MIC value for the mixture of substances A and B
P_{A} is the proportion of the substance A in the mixture
P_{B} is the proportion of the substance B in the mixture

TABLE 3
MIC values and synergy index of 1,2-nonanediol in combination
with different antimicrobials against Staphylococcus aureus

| | | | | MIC | MIC | MIC | |
|---|---|---|---|---|---|---|---|
| | | Portion | Portion | Component | Component | Mixture | |
| | | Component | Component | A | B | in | Synergy |
| Component A | Component B | A | B | in ppm | in ppm | ppm | Index |
| 1,2- | Ethylhexylglycerin | 0.2 | 0.8 | 1000 | 2000 | 1000 | 0.6 |
| Nonanediol | | | | | | | |
| 1,2- | Ethylhexylglycerin | 0.8 | 0.2 | 1000 | 2000 | 1000 | 0.9 |
| Nonanediol | | | | | | | |
| 1,2- | Glyceryl caprylate | 0.2 | 0.8 | 1000 | 2500 | 1000 | 0.52 |
| Nonanediol | | | | | | | |
| 1,2- | Glyceryl caprylate | 0.8 | 0.2 | 1000 | 2500 | 1000 | 0.88 |
| Nonanediol | | | | | | | |
| 1,2- | Phenoxyethanol | 0.2 | 0.8 | 1000 | 5000 | 2500 | 0.9 |
| Nonanediol | | | | | | | |
| 1,2- | Phenoxyethanol | 0.8 | 0.2 | 1000 | 5000 | 1000 | 0.84 |
| Nonanediol | | | | | | | |
| 1,2- | Caprylhydroxamic | 0.5 | 0.5 | 1000 | 1000 | 250 | 0.25 |
| Nonanediol | acid | | | | | | |
| 1,2- | Climbazole | 0.5 | 0.5 | 1000 | 250 | 250 | 0.63 |
| Nonanediol | | | | | | | |
| 1,2- | Benzyl Alcohol | 0.5 | 0.5 | 1000 | 5000 | 1000 | 0.6 |
| Nonanediol | | | | | | | |
| 1,2- | o-cymen-5-ol | 0.5 | 0.5 | 1000 | 250 | 250 | 0.63 |
| Nonanediol | | | | | | | |
| 1,2- | Propanediol | 0.5 | 0.5 | 1000 | 500 | 500 | 0.75 |
| Nonanediol | Caprylate | | | | | | |

For all tested antimicrobial components, synergistic effects regarding the antimicrobial efficacy were found with SI values below 1. For example, the MIC values of 1,2-nonanediol and ethylhexylglycerin are 1000 ppm and 2000 ppm, respectively. For a mixture of 20%:80% of 1,2-nonanediol and ethylhexylglycerin a MIC value of 1000 was determined, resulting in a Synergy Index of 0.6. Even the increase of the 1,2-nonanediol concentration from 20% to 80% and the reduction of the ethylhexylglycerin from 80% to 20% in the mixture resulted in a synergistic effect. This means that with the addition of a 1,2-alkanediol, preferably 1,2-nonanediol, the concentration of the antimicrobial component can be reduced while the same antimicrobial effect can be obtained. Hence, the use of 1,2-nonanediol allows for the use of less antimicrobial component in a cosmetic or pharmaceutical composition for achieving the same antimicrobial effect compared to a cosmetic or pharmaceutical composition comprising the same antimicrobial component but without the use of 1,2-nonanediol.

The synergistic activity of mixtures including a 1,2-alkanediol and a 2,3-alkanediol in combination with different antimicrobial compounds was determined by measuring the Minimum Inhibitory Concentrations (MIC) according to the method as described in Example 2.

To verify that the tested combinations act synergistically against microorganisms of the natural microbiota of mammalian skin, corresponding experiments were conducted with Staphylococcus aureus, a well-known pathogen on the skin. The synergistic activities of different alkanediol mixtures including a 1,2-alkanediol and a 2,3-alkanediol and different antimicrobial compounds in ratios as specified in the following Table 4 were determined by measuring the MIC of the mixture as indicated in methods section.

In order to calculate the synergy index with MIC values, the Kull equation (I) was used as described above

TABLE 4
MIC values and synergy index of different alkanediol mixtures
including a 1,2-alkanediol and 2,3-alkanediol in combination with different
antimicrobials against Staphylococcus aureus

| | | | | | | | MIC | | | |
|---|---|---|---|---|---|---|---|---|---|---|
| | | | | | | MIC | Component | MIC | MIC | |
| | | | Portion | Portion | Portion | Component | B | Component | Mixture | |
| Component | Component | Component | Component | Component | Component | A in | in | C in | in | Synergy |
| A | B | C | A | B | C | ppm | ppm | ppm | ppm | Index |
| 1,2- | Benzyl | 2,3- | 0.5 | 0.45 | 0.05 | 7500 | 5000 | 20000 | 5000 | 0.80 |
| Heptanediol | Alcohol | Heptanediol | | | | | | | | |
| 1,2- | o-Cymen- | 2,3- | 0.5 | 0.45 | 0.05 | 7500 | 250 | 20000 | 500 | 0.93 |
| Heptanediol | 5-ol | Heptanediol | | | | | | | | |
| 1,2- | Capryl- | 2,3-Nonanediol | 0.5 | 0.45 | 0.05 | 1000 | 1000 | 2500 | 500 | 0.49 |
| Nonanediol | hydroxamic | | | | | | | | | |
| | acid | | | | | | | | | |
| 1,2- | Benzyl | 2,3-Nonanediol | 0.5 | 0.45 | 0.05 | 1000 | 5000 | 2500 | 1000 | 0.61 |
| Nonanediol | Alcohol | | | | | | | | | |
| 1,2- | o-Cymen- | 2,3-Nonanediol | 0.5 | 0.45 | 0.05 | 1000 | 250 | 2500 | 250 | 0.58 |
| Nonanediol | 5-ol | | | | | | | | | |

For all tested antimicrobial components, synergistic effects regarding the antimicrobial efficacy were found with SI values below 1. For example, the MIC values of a mixture comprising 1,2-nonanediol and 2,3-nonanediol and o-Cymen-5-ol are 1000 ppm, 2500 ppm and 250 ppm, respectively. For a mixture of 1,2-nonanediol (0.45%) and 2,3-nonanediol (5%) and o-Cymen-5-ol (50%) a MIC value of 250 was determined, resulting in a Synergy Index of 0.58. Hence, the simultaneously use of a 1,2-alkanediol and a 2,3-alkanediol allows for the use of less antimicrobial component in a cosmetic or pharmaceutical composition for achieving the same antimicrobial effect compared to a cosmetic or pharmaceutical composition comprising the same antimicrobial component but without the use of the alkanediol mixture.

The synergistic activity of mixtures including a 1,2-alkanediol and a 2,3-alkanediol, wherein the 2,3-alkanediol serves as the antimicrobial component (b), was determined by measuring the Minimum Inhibitory Concentrations (MIC) according to the method as described in Example 2 and 3.

To verify that the tested combinations act synergistically against microorganisms a corresponding experiment was conducted with Aspergillus brasiliensis. The synergistic activities of different alkanediol mixtures including a 1,2-alkanediol and a 2,3-alkanediol corresponding to the antimicrobial component (b) in ratios as specified in the following Table 5 were determined by measuring the MIC of the mixture as indicated in methods section.

In order to calculate the synergy index with MIC values, the Kull equation (I) was used as described in Example 3

TABLE 5
MIC values and synergy index of different alkanediol mixtures including
a 1,2-alkanediol and a 2,3-alkanediol as antimicrobial component against
Aspergillus brasiliensis

| | | | | MIC | MIC | | |
|---|---|---|---|---|---|---|---|
| | | Portion | Portion | Component | Component | MIC | |
| Component | Component | Component | Component | A in | B in | in ppm | Synergy |
| A | B | A | B | ppm | ppm | Mixture | Index |
| 1,2- | 2,3- | 0.9 | 0.1 | 7500 | 20000 | 7500 | 0.94 |
| Hexandiol | Hexandiol | | | | | | |
| 1,2- | 2,3- | | | 5000 | 7500 | 2500 | 0.49 |
| Heptandiol | Heptandiol | | | | | | |
| 1,2- | 2,3- | | | 2500 | 2500 | 500 | 0.20 |
| Octandiol | Octandiol | | | | | | |
| 1,2- | 2,3- | | | 500 | 1000 | 250 | 0.48 |
| Nonandiol | Nonandiol | | | | | | |

In addition, a synergistic antimicrobial efficacy could be observed for a composition according to present invention being an alkanediol mixture including 1,2-heptanediol and 2,3-heptanediol (90:10) against Pseudomonas aeruginosa. The Synergy Index for said alkanediol mixture was 0.50.

### Example 4: Sebum Control

Additional tests were performed with human sebaceous glands in order to verify the modulatory activity of different 1,2-alkanediols on sebum secretion. Several microbial species on the skin are lipid dependent and feed on the human sebum (e.g., Corynebacterium, Propionibacterium). Accordingly, sebum reduction can indirectly support the reduction of microorganisms on the skin and in this way also indirectly support e.g., dandruff or acne reduction.

For these experiments sebaceous glands had been micro-dissected and cultured with medium. The medium without any supplement served as untreated control. The medium with 5 μM Capsaicin served as positive control. Additionally, the culture medium was supplemented with different concentrations of alkanediols. After six days the sebaceous glands were collected and lipids and proteins quantified. The total lipid amount being representative of sebum production was obtained by normalizing the quantified lipids upon the quantified proteins (i.e., mg of lipids/mg of proteins).

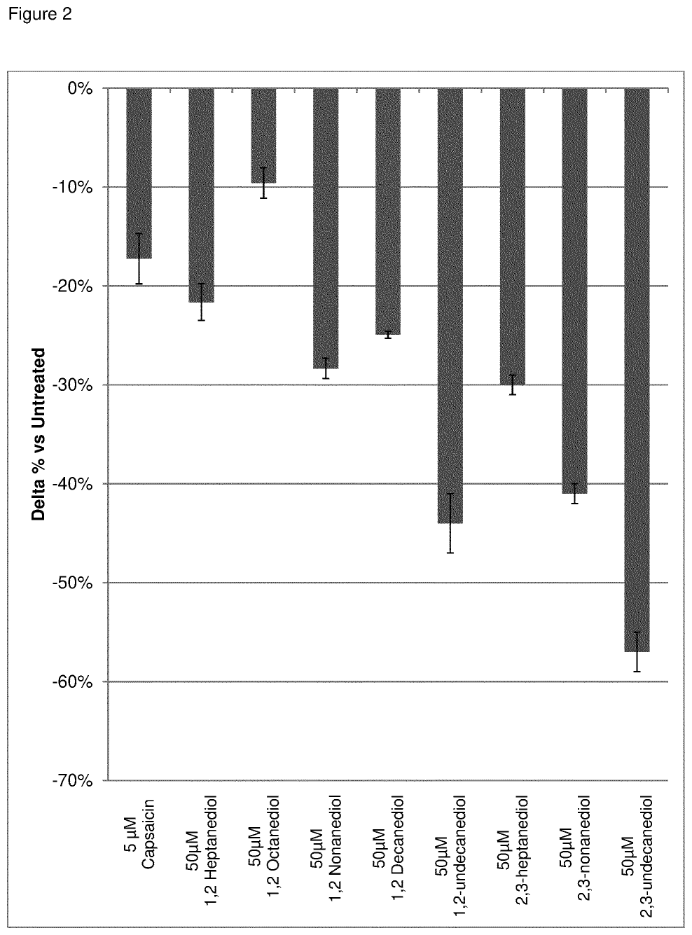

The results are shown in FIG. 2.

As shown in the FIG. 2, all tested 1,2-alkanediols showed sebum reduction compared to an untreated control. This demonstrates the sebum reduction activity of 1,2-alkanediols. Even a stronger sebum reducing effect is achieved for 2,3-alkanediols, in particular 2,3-heptanediol, 2,3-nonanediol and 2,3-undecanediol: 1,2-heptanediol shows a sebum reduction of 22% whereas 2,3-heptanediol shows a sebum reduction of 30%; 1,2-nonanediol reduces sebum secretion by 28% whereas the 2,3-nonanediol reduces sebum secretion by 41%. The best sebum reduction was observed for 1,2-undecanediol with 44%, whereas the 2,3-undecanediol leads to a sebum reduction of more than 50%. Hence, the 2,3-alkanediol substances have a distinguished effect on sebum reduction compared to the corresponding 1,2-alkanediol substances. Nevertheless, no linear correlation between molecule length and activity was found. The best performance of the 1,2-alkanediols had 1,2-undecanediol and the best performance of the 2,3-alkanediols had 2,3-nonanediol and 2,3-undecanediol.

All results shown in this figure resulted from a single experimental run with human sebaceous glands derived from the same human donor.

For 1,2-nonanediol additionally the concentration dependency was investigated and experimentally confirmed.

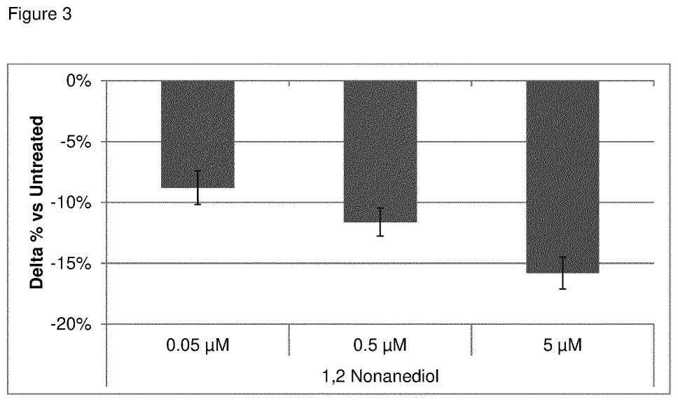

The results are shown in FIG. 3.

All results shown in this figure resulted from a single experimental run with human sebaceous glands derived from the same human donor being different from the experimental results shown above.

Based on these results, it is assumed that 1,2-alkanediols, especially 1,2-nonanediol, have microbiol balancing or reducing properties indirectly via sebum reduction.

### Example 5: Solubility of Alkanediols in Water

For the determination of the maximum water solubility of an alkanediol, water and different concentrations of the respective alkanediol were blended by stirring. The assessment of solubility was carried out at ambient temperature after 24 h storage at 5° C. The determination of whether the alkanediol has dissolved in water is based entirely on visual observation. An alkanediol compound has dissolved if the mixture is clear and shows no signs of cloudiness or precipitation.

TABLE 6

| Tradename/ | | Maximum solubility |
|---|---|---|
| Chemical name | INCI | in water (% by weight) |
| Hydrolite ® 5 green | Pentylene Glycol | 50 |
| Hydrolite ® 6 | Hexanediol | 50 |
| 1,2-heptanediol | Heptanediol | 2.5 |
| Hydrolite ® 8 | Caprylyl Glycol | 0.5 |
| SymDiol ® 68 | Hexanediol, Caprylyl | 1.0 |
| | Glycol | |
| 1,2-nonanediol | Nonanediol | 0.1 |
| SymClariol ® | Decylene Glycol | — |
| 1,2-undecanediol | Undecanediol | — |
| 2,3-heptanediol | — | 3.6 |
| 2,3-octanediol | — | 1.5 |
| 2,3-nonanediol | — | 0.3 |
| 2,3-undecanediol | — | — |

### Example 6: Formulation Examples

The following formulations according to the present invention were prepared:
Deodorant Spray
Deodorant Spray with AcH
Deodorant Roll-on
Cool Relief Deo Plus
Sport Fit Deo
Antiperspirant/deodorant roll-on
Deodorant formulation in the form of a roll-on-gel
Clear deo anti-perspirant roll-on
Deodorant Aerosol
Deodorant Stick
Antiperspirant Stick
Intimate Sensi-Gel
Mild feminine wash
Mosquito & tick repellent Spray
Insect Repellent Spray, aqueous/ethanolic
Insect Repellent solution for wipes
Anti-Sweat Sunscreen Lotion with repellent
Syndet antimicrobial soap bar
Syndet soap bar
Ice Crystal Soap
Antimicrobial toilet soap bar
Acne Scar Remover
Balancing Face Toner
Biotic Acne Control Gel
SunCare Foam For Acne Prone Skin, exp. SPF 30
Cool Sebum Control Cleanser

In the following formulations the following five perfume oils PO1, PO2, PO3, PO4, or PO5 were each used as fragrance.

TABLE 7
Composition of perfume oil 1; PO1 (amounts in ‰ by weight)

| Ingredients | Amount |
|---|---|
| ALDEHYDE C14 SO-CALLED | 2 |
| ALLYL AMYL GLYCOLATE 10% DPG | 5 |
| ANISIC ALDEHYDE PURE | 5 |
| APPLE OLIFFAC TYPE | 10 |
| BENZYLACETATE | 50 |
| BERGAMOT IDENTOIL ® COLOURLESS | 15 |
| CANTHOXAL | 5 |
| CETALOX 10% IPM | 3 |
| CITRONELLOL 950 | 40 |
| DAMASCENONE TOTAL 1% DPG | 5 |
| DAMASCONE ALPHA 10% DPG | 5 |
| DAMASCONE DELTA 10% DPG | 2 |
| DIMETHYL BENZYL CARBINYL BUTYRATE | 2 |
| DIPROPYLENE GLYCOL | 178 |
| EBANOL | 2 |
| ETHYL DECADIENOATE TRANS CIS-2,4 10% IPM | 2 |
| FLOROSA | 5 |
| FRAMBINON ® 10% DPG | 7 |
| GALAXOLIDE 50% IN IPM | 100 |
| GALBEX TYPE BASE | 1 |
| GERANYL ACETATE PURE | 2 |
| HEDIONE | 30 |
| HELIOTROPIN | 10 |
| HEXENYL ACETATE CIS-3 10% DPG | 1 |
| HEXENYL SALICYLATE CIS-3 | 5 |
| HEXYL CINNAMIC ALDEHYDE ALPHA | 70 |
| HEXYL SALICYLATE | 50 |
| HYDROXY CITRONELLAL | 10 |
| ISO E SUPER | 15 |
| ISORALDEINE 70 | 20 |
| LEAFOVERT ® | 1 |
| LILIAL | 60 |
| LINALOOL | 60 |
| LINALYL ACETATE | 20 |
| LYRAL | 7 |
| MANZANATE | 2 |
| PHENOXANOL | 7 |
| PHENYLETHYL ALCOHOL | 120 |
| SANDAL MYSORE CORE | 2 |
| SANDRANOL ® | 7 |
| STYRALYL ACETATE | 3 |
| TAGETES RCO 10% TEC | 2 |
| TERPINEOL PURE | 20 |
| TETRAHYDROGERANIOL 10% DPG | 5 |
| TONALIDE | 7 |
| VERTOCITRAL 10% DPG | 5 |
| VERTOFIX | 15 |
| Total: | 1000 |

TABLE 8
Composition of perfume oil 2; PO2 (amounts in ‰ by weight)

| Ingredients | Amount |
|---|---|
| Acetophenone, 10% in DPG | 10 |
| n-Undecanal | 5 |
| Aldehyde C14, so-called (peach aldehyde) | 15 |
| Allylamyl glycolate, 10% in DPG | 20 |
| Amyl salicylate | 25 |
| Benzyl acetate | 60 |
| Citronellol | 80 |
| d-Limonene | 50 |
| Decenol trans-9 | 15 |
| Dihydromyrcenol | 50 |
| Dimethylbenzylcarbinyl acetate | 30 |
| Diphenyloxide | 5 |
| Eucalyptol | 10 |
| Geraniol | 40 |
| Nerol | 20 |
| Geranium oil | 15 |
| Hexenol cis-3, 10% in DPG | 5 |
| Hexenyl salicylate cis-3 | 20 |
| Indole, 10% in DPG | 10 |
| Alpha-ionone | 15 |
| Beta-ionone | 5 |
| Lilial ® (2-methyl-3-(4-tert-butyl-phenyl)propanal) | 60 |
| Linalool | 40 |
| Methylphenyl acetate | 10 |
| Phenylethyl alcohol | 275 |
| Styrolyl acetate | 20 |
| Terpineol | 30 |
| Tetrahydrolinalool | 50 |
| Cinnamyl alcohol | 10 |
| Total: | 1000 |

TABLE 9
Composition of perfume oil 3; PO3 (amounts in ‰ by weight)

| Ingredients | Amount |
|---|---|
| Benzyl acetate | 60 |
| Citronellyl acetate | 60 |
| Cyclamenaldehyde (2-methyl-3-(4-isopropylphenyl)propanal | 20 |
| Dipropylene glycol (DPG) | 60 |
| Ethyllinalool | 40 |
| Florol (2-isobutyl-4-methyltetrahydro-2H-pyran-4-ol) | 30 |
| Globanone ® [(E/Z)-8-cyclohexadecen-1-one] | 180 |
| Hedione ® (methyldihydrojasmonate) | 140 |
| Hexenyl salicylate, cis-3 | 10 |
| Vertocitral (2,4-dimethyl-3-cyclohexenecarboxaldehyde) | 5 |
| Hydratropaldehyde, 10% in DPG | 5 |
| Isodamascone (1-(2,4,4-trimethyl-2-cyclohexen-1-yl)-2- | 5 |
| Isomuscone (cyclohexadecanone) | 40 |
| Jacinthaflor (2-methyl-4-phenyl-1,3-dioxolane) | 10 |
| Cis-jasmone, 10% in DPG | 20 |
| Linalool | 50 |
| Linalyl acetate | 30 |
| Methyl benzoate, 10% in DPG | 25 |
| para-Methyl cresol, 10% in DPG | 10 |
| Nerol | 20 |
| Phenylpropylaldehyde | 5 |
| 2-Phenylethyl alcohol | 82 |
| Tetrahydrogeraniol | 13 |
| 2,2-Dimethyl-3-cyclohexyl-1-propanol | 80 |
| Total: | 1000 |

TABLE 10
Composition of perfume oil 4; PO4 (amounts in ‰ by weight)

| Ingredients | Amount |
|---|---|
| AMBRETTOLIDE (MACRO) | 10 |
| AMBROXIDE 10% in IPM | 10 |
| BENZYL ACETATE | 20 |
| BENZYL SALICYLATE | 15 |
| BERGAMOT OIL. bergapten-free | 60 |
| CALONE ® 1951 10% in DPG | 15 |
| COUMARIN | 5 |
| CYCLOGALBANATE ® 10% in DPG | 10 |
| ALPHA -DAMASCONE 1% in DPG | 20 |
| DIHYDROMYRCENOL | 10 |
| ETHYL LINALOOL | 75 |
| ETHYL LINALYLACETATE | 50 |
| ETHYL MALTOL 1% in DEP | 10 |
| ETHYLENE BRASSYLATE (MACRO) | 80 |
| FLOROSA | 40 |
| GERANYLACETATE | 10 |
| HEDIONE ® HC/30 | 35 |
| HEDIONE ® | 210 |
| HELIONAL ® | 15 |
| HELVETOLIDE ® (ALICYC) | 30 |
| HEXENYLSALICYLATE CIS-3 | 20 |
| ISO E SUPER ® | 40 |
| LEAFOVERT ® 10% in DEP | 10 |
| LILIAL ® | 80 |
| LYRAL ® | 20 |
| MANDARIN OIL | 10 |
| STYRALYL ACETATE | 5 |
| SYMROSE ® | 15 |
| VANILLIN 10% in DEP | 20 |
| DIPROPYLENE GLYCOL (DPG) | 50 |
| TOTAL: | 1000 |

TABLE 11
Composition of perfume oil 5; PO5 (amounts in ‰ by weight)

| Ingredients | Amount |
|---|---|
| AMAROCITE ® | 10 |
| AMBROCENIDE ® 10% in DPG | 5 |
| AMBROXIDE | 15 |
| AURELIONE ® (7/8-Cyclohexadecenone) (MACRO) | 70 |
| BERGAMOT OIL, bergapten-free | 90 |
| CALONE ® 1951 10% in DPG | 20 |
| CARAWAY OIL | 10 |
| CITRAL | 20 |
| COUMARIN | 10 |
| ALPHA-DAMASCONE 1% in DPG | 15 |
| DIHYDROMYRCENOL | 70 |
| ESTRAGON OIL | 10 |
| ETHYL LINALOOL | 100 |
| ETHYL LINALYLACETATE | 90 |
| EUGENOL | 10 |
| EVERNYL ® | 5 |
| FRUCTATE ® | 5 |
| GERANIUM OIL | 5 |
| HEDIONE ® HC/30 | 100 |
| HELIONAL ® | 10 |
| INDOLE 10% in DPG | 5 |
| ISO E SUPER ® | 100 |
| KEPHALIS ® | 5 |
| LAVENDER OIL | 40 |
| CITRUS OIL | 80 |
| LILIAL ® | 30 |
| MANDARIN OIL | 20 |
| MUSCENONE (MACRO) | 5 |
| SANDRANOL ® | 10 |
| VANILLIN 10% in DPG | 5 |
| DIPROPYLENE GLYCOL | 30 |
| TOTA: | 1000 |

Cosmetic formulations (compositions)—amounts are indicated as % by weight for all formulations.

TABLE 12
Deodorant Spray

| Ingredients | INCI | Amount |
|---|---|---|
| Aqua/Water | Aqua | ad 100 |
| Volarest ™ FL | Acrylates/Beheneth-25 | 1.2 |
| Dracorin ® GOC | Glyceryl oleate citrate; | 2.0 |
| | Caprylic/Capric triglyceride | |
| SymDeo ® Plus | Lauryl alcohol, Phenoxyethanol; | 0.5 |
| | 2-Benzylheptanol; Decylene | |
| | Glycol | |
| SymOcide ® PS | Phenoxyethanol; Decylene | 0.8 |
| | Glycol; 1,2-Hexanediol | |
| Dow Corning ® 245 Fluid | Cyclomethicone | 5.0 |
| Isopropyl Myristate | Isopropyl myristate | 2.0 |
| Farnesol | Farnesol | 0.25 |
| Ethylhexylglycerin | Ethylhexylglycerin | 0.2 |
| SymDeo ® B125 | 2-Methyl 5-Cycloheylpentanol | 0.15 |
| Tego ® Cosmo P 813MB | Polyglyceryl-3 Caprylate | 0.25 |
| Perfume oil PO1, PO2, PO3, | Perfume | 0.8 |
| PO4, or PO5 | | |
| Frescolat ® ML nat | Menthyl Lactate | 0.2 |
| Menthol | Menthol | 0.1 |
| Sodium Hydroxide, 10% | Aqua; Sodium hydroxide | 0.6 |
| 1,2-heptanediol | 1,2-heptanediol | 0.5 |
| 2,3-heptanediol | 2,3-heptanediol | 0.02 |
| 1,2-nonanediol | 1,2-nonanediol | 0.2 |
| 2,3-nonanediol | 2,3-nonanediol | 0.01 |

TABLE 13
Deodorant Spray with AcH

| Ingredients | INCI | Amount |
|---|---|---|
| Triethylcitrate | Triethylcitrate | 3.0 |
| Menthol | Menthol | 0.1 |
| Solubilizer ® | PEG-40 Hydrogenated Castor | 1.0 |
| | Oil, Trideceth-9, Propylene | |
| | Glycol, Aqua | |
| Ethanol 96% | Ethanol | 30.0 |
| 1,2-nonanediol | 1,2-nonanediol | 0.5 |
| 2,3-nonanediol | 2,3-nonanediol | 0.05 |
| 2,3-octanediol | 2,3-octanediol | 0.1 |
| Ethylhexylglycerin | Ethylhexylglycerin | 0.3 |
| SymDeo ® B125 | 2-Methyl 5-Cycloheylpentanol | 0.25 |
| SymClariol ® | Decylene Glycol | 0.25 |
| Frescolat ® ML | Menthyl Lactate | 0.6 |
| Tego ® Cosmo P 813MB | Polyglyceryl-3 Caprylate | 0.5 |
| Locron ® L (50%) | Aqua, Aluminum Chlorohydrate | 25.0 |
| Perfume oil PO1, PO2, PO3, | Perfume | 0.5 |
| PO4, or PO5 | | |
| Water, demin. | Aqua | ad 100 |

TABLE 14
Deodorant Roll-on

| Ingredients | INCI | Amount |
|---|---|---|
| Aqua/Water | Aqua | ad 100 |
| Hydrolite ® CG | Caprylyl Glycol | 0.3 |
| 1,2-heptanediol | 1,2-heptanediol | 1.0 |
| 2,3-heptanediol | 2,3-heptanediol | 0.05 |
| 1,2-nonanediol | 1,2-nonanediol | 0.1 |
| 2,3-nonanediol | 2,3-nonanediol | 0.05 |
| Menthol | Menthol | 0.1 |
| Frescolat ® ML | Menthyl Lactate | 1.0 |
| SymSave ® H | Hydroxyacetophenone | 0.5 |
| EDTA NA2 | Disodium EDTA | 0.1 |
| Dracorin ® GOC | Glyceryl oleate citrate; | 4.0 |
| | Caprylic/Capric triglyceride | |
| Dragoxat ® 89 | Ethylhexyl isononanoate | 5.0 |
| SymMollient ® S | Cetearyl Nonanoate | 1.0 |
| Carbopol ® Ultrez 20 | Acrylates/C10-30 Alkyl | 0.2 |
| Polymer | acrylate crosspolymer | |
| Pemulen ™ TR-2 Polymeric | Acrylates/C10-30 Alkyl | 0.2 |
| Emulsifier | acrylate crosspolymer | |
| Farnesol | Farnesol | 0.3 |
| SymGuard ® CD | Phenylpropanol, o-Cymen-5-ol, | 0.3 |
| | Decylene Glycol | |
| Perfume oil PO1, PO2, PO3, | Perfume | 0.5 |
| PO4, or PO5 | | |
| Sodium Hydroxide, | Aqua; Sodium hydroxide | 0.2 |
| 50% solution | | |

TABLE 15
Cool Relief Deo Plus

| Ingredients | INCI | Amount |
|---|---|---|
| Aqua/Water | Aqua | ad 100 |
| Veegum ® HS Granules | Magnesium Aluminium | 1.0 |
| | Silicate | |
| Glycerin | Glycerin | 5.0 |
| EDTA NA2 | Disodium EDTA | 0.1 |
| Dracorin ® 100 SEP | Glyceryl Stearate, PEG-100 | 6.0 |
| | Stearate | |
| Lanette 16 | Cetyl alcohol | 2.0 |
| Eumulgin ® B2 | Ceteareth-20 | 6.0 |
| Eutanol ® G | Octyldodecanol | 8.0 |
| Dow Cornng FZ-3196 | Caprylyl Methicone | 4.0 |
| Volatile | | |
| Tween 20 | Polysorbate20 | 0.3 |
| SymDeo ® B125 | 2-Methyl 5-Cyclohexylpentanol | 0.3 |
| Dragosantol ® 100 | Bisabolol | 0.05 |
| Aqua/Water | Aqua | 15.0 |
| Locron ® P | Aluminium Chlorohydrate | 15.0 |
| 1,2-nonanediol/2,3-nonanediol | 1,2-nonanediol/2,3-nonanediol | 1.0 |
| (95:5 w/w %) | | |
| SymOcide ® PS | Phenoxyethanol, Decylene | 1.0 |
| | Glycol, 1,2-Hexanediol | |
| Frescolat ® ML | Menthyl Lactate | 0.5 |
| Vitacel ® CS020 | Cellulose | 1.0 |
| Extrapone ® Rose | Aqua, Glycerin, Xanthan Gum, | 1.0 |
| Quartz GW | Quartz Powder | |
| Perfume oil PO1, PO2, PO3, | Perfume | 1.0 |
| PO4, or PO5 | | |
| Sodium hydroxide | Aqua, Sodium hydroxide | 0.5 |
| 10% solution | | |

TABLE 16
Sport Fit Deo

| Ingredients | INCI | Amount |
|---|---|---|
| SymLite ® G8 | Glyceryl caprylate | 0.15 |
| Menthol | Menthol | 0.05 |
| Ethanol | Alcohol denat | ad 100 |
| SymClariol ® | Decylene Glycol | 0.25 |
| 1,2-heptanediol, | 1,2-heptanediol, 2,3-heptanediol | 0.5 |
| 2,3-heptanediol | | |
| (95:5 w/w %) | | |
| SymDeo ® B125 | 2-Methyl 5-Cyclohexylpentanol | 0.25 |
| Frescolat ® ML | Menthyl Lactate | 0.5 |
| Extrapone ® Ginseng | Propylene Glycol, Aqua, Panax | 0.1 |
| | ginseng root extract, Lactic acid | |
| Extrapone ® Peppermint | Aqua, Propylene Glycol, Mentha | 0.1 |
| | Piperita Leaf extract | |
| Ethylhexyglycerin | Ethylhexylglycerin | 0.2 |
| Perfume oil PO1, PO2, PO3, | Perfume | 1.0 |
| PO4, or PO5 | | |

TABLE 17
Antiperspirant/deodorant roll-on

| Ingredients | INCI | Amount |
|---|---|---|
| SymLite ® G8 | Glyceryl caprylate | 0.15 |
| Dragosantol ® 100 | Bisabolol | 0.1 |
| 1,2-heptanediol | 1,2-heptanediol | 0.1 |
| 2,3-heptanediol | 2,3-heptanediol | 0.005 |
| 1,2-nonanediol | 1,2-nonanediol | 0.15 |
| 2,3-nonanediol | 2,3-nonanediol | 0.01 |
| Ethanol 96% | Ethanol | 30.0 |
| Farnesol | Farnesol | 0.5 |
| Perfume oil PO1, PO2, PO3, | Perfume | 1.5 |
| PO4, or PO5 | | |
| Frescolat ®ML cryst. | Menthyl Lactate | 0.2 |
| Irgasan ® DP 300 | Triclosan | 0.3 |
| Natrosol ® 250 HHR | Hydroxyethyl-cellulose | 0.3 |
| Solubilizer 611674 | PEG-40 Hydrogenated Castor | 2.0 |
| | Oil, Trideceth-9, Water (Aqua) | |
| SymDeo ® B125 | 2-Methyl 5-Cyclohexylpentanol | 0.5 |
| Water (demineralized) | Water (Aqua) | ad 100 |
| Zirkonal L 450 | Aluminium Zirconium | 37.0 |
| | Pentachloro-hydrate | |
| | (40% aqueous solution) | |

TABLE 18
Deodorant formulation in the form of a roll-on gel

| Ingredients | INCI | Amount |
|---|---|---|
| 1,3-butylene glycol | 1,3-butylene glycol | 2.0 |
| Cremophor ® CO 40 | PEG-40-hydrogenated | 2.0 |
| Surfactant | castor oil | |
| Hydroxyethylcellulose | Hydroxyethylcellulose | 0.5 |
| Perfume oil PO1, PO2, PO3, | Perfume | 0.3 |
| PO4, or PO5 | | |
| 1,3-propanediol | 1,3 Propanediol | 0.5 |
| SymGuard ® CD | 3-Phenylpropanol, | 0.4 |
| | o-cymen-3-ol, | |
| | Decylene glycol | |
| Ethylhexylglycerin | Ethylhexylglycerin | 0.1 |
| 1,2-heptanediol | 1,2-heptanediol | 0.5 |
| 1,2-nonanediol, 2,3-nonanediol | 1,2-nonanediol, | 0.5 |
| (95:5 w/w %) | 2,3-nonanediol | |
| SymLite ® G8 | Glyceryl Caprylate | 0.15 |
| Frescolat ® ML cryst. | Menthyl Lactate | 0.4 |
| Water | Aqua | ad 100 |

TABLE 19
Clear deo anti-perspirant roll-on

| Ingredients | INCI | Amount |
|---|---|---|
| Methocel ™ E4M Premium | Hydroxypropyl | 0.5 |
| | Methylcellulose | |
| Water | Water (Aqua) | ad 100 |
| Neo-PCL Water Soluble N | Trideceth-9, PEG-5 | 1.0 |
| | Ethylhexanoate, Water (Aqua) | |
| Solubilizer | PEG-40 Hydrogenated Castor | 3.0 |
| | Oil, Trideceth-9, Propylene | |
| | Glycol, Water (Aqua) | |
| Deolite | Dimethyl Phenylpropanol, | 0.5 |
| | Pentylene Glycol | |
| Locron ® LW | Aluminium Chlorohydrate | 25.0 |
| Aloe Vera Gel Concentrate 10/1 | Aloe Barbadensis Leaf Juice | 1.0 |
| 1,2-Propylene Glycol 99 P GC | Propylene Glycol | 4.0 |
| Ethanol 96% | Alcohol Denat. | 30.0 |
| Frescolat ® ML | Menthyl Lactate | 1.5 |
| Perfume oil PO1, PO2, PO3, | Perfume | 1.0 |
| PO4, or PO5 | | |
| 1,2-undecanediol, 2,3- | 1,2-undecanediol, 2,3- | 0.1 |
| undecanediol (w/w % 95:5) | undecanediol | |
| 1,2-heptanediol, 2,3-heptanediol | 1,2-heptanediol, | 1.0 |
| (w/w % 95:5) | 2,3-heptanediol | |

TABLE 20
Deodorant Aerosol

| Ingredients | INCI | Amount |
|---|---|---|
| Belsil ® DM 0.65 | Disiloxane | ad 100 |
| Isoadipate | Diisopropyl adipate | 5.0 |
| Tegosoft ® TN | C12-15 Alkyl benzoate | 10.0 |
| Vitamin E Acetate | Tocopheryl acetate | 0.5 |
| Farnesol | Farnesol | 0.3 |
| Ethylhexylglycerin | Ethylhexylglycerin | 0.2 |
| 2,3-heptanediol | 2,3-heptanediol | 0.1 |
| 1,2-nonanediol | 1,2-nonanediol | 0.2 |
| SymDeo ® MPP | Dimethyl Phenylbutanol | 0.2 |
| Frescolat ® ML nat | Menthyl Lactate | 0.25 |
| Perfume oil PO1, PO2, PO3, | Perfume | 0.6 |
| PO4, or PO5 | | |

TABLE 21
Deodorant stick

| Ingredients | INCI | Amount |
|---|---|---|
| Sodium stearate | Sodium stearate | 8.0 |
| PPG-3 Myristyl ether | PPG-3 Myristyl ether | 70.0 |
| 1,2-propylene glycol | 1,2-propylene glycol | 10.0 |
| 1,1-dimethyl-3- | 1,1-dimethyl-3- | 0.2 |
| phenylpropanol | phenylpropanol | |
| 2-butyloctanoic acid | 2-butyloctanoic acid | 0.2 |
| Perfume oil PO1, PO2, PO3, | Perfume | 0.6 |
| PO4, or PO5 | | |
| Water | Aqua | ad 100 |
| Menthol | Menthol | 0.01 |
| SymDeo ® Plus | Lauryl Alcohol, | 0.5 |
| | Phenoxyethanol, | |
| | 2-Benzylheptanol, Decylene | |
| | Glycol | |
| 3-((−)-Menthoxy)propane- | 3-((−)-Menthoxy)propane- | 0.1 |
| 1,2-diol | 1,2-diol | |
| N-Ethyl-p-menthane-3- | Ethyl Menthane Carboxamide | 0.3 |
| carboxamide (Cooling Agent | | |
| WS-3) | | |
| SymLite ® G8 | Glyceryl Caprylate | 0.15 |
| 1-2-nonanediol | 1,2-nonanediol | 0.2 |
| 1,2-heptanediol | 1,2-heptanediol | 0.50 |

TABLE 22
Antiperspirant stick

| Ingredients | INCI | Amount |
|---|---|---|
| PCL-Liquid ® 100 | Cetearyl ethylhexanonate | ad 100 |
| Silicone Fluid 345 | Cyclomethicone | 10.0 |
| Crodacol ™ C90 | Cetyl Alcohol | 8.0 |
| Syncrowax ™ HGCL | C18-36 Triglyceride | 8.0 |
| Crodamol ™ PTC | Pentaerythritol | 5.0 |
| | Tetracaprylate/Caprate | |
| Syncrowax ™ HRC | Tribehenin | 4.0 |
| Brij ™ O5 | Oleth-5 | 1.0 |
| Titanium Dioxide | Titanium Dioxide | 1.0 |
| Menthol | Menthol | 0.05 |
| Frescolat ® ML Nat | Menthyl Lactate | 0.75 |
| Rezal 36GP | Aluminium Tetrachlorohydrex | 20.0 |
| | GLY | |
| Dry-Flo ® C | Aluminium Starch Octenyl | 22.5 |
| | Succinate | |
| N-Ethyl-p-menthane-3- | Ethyl Menthane Carboxamide | 0.5 |
| carboxamide (Cooling Agent | | |
| WS-3) | | |
| Frescolat ® MGA | Menthone Glycerin Acetal | 0.6 |
| Preservative | Phenoxyethanol | 0.8 |
| Perfume oil PO1, PO2, PO3, | Perfume | 0.6 |
| PO4, or PO5 | | |
| 1,2-heptanediol, 2,3-heptanediol | 1,2-heptanediol, | 0.5 |
| (95:5 w/w %) | 2,3-heptanediol | |
| 2,3-nonanediol | 2,3-nonanediol | 0.05 |

TABLE 23
Intimate Sensi-Gel

| Ingredients | INCI | Amount |
|---|---|---|
| Aqua/Water | Aqua | ad 100 |
| Sodium Benzoate | Sodium benzoate | 0.3 |
| Keltrol ® CG-T | Xanthan Gum | 0.2 |
| Cyamopsis Tetragonolobus | Cyamopsis Tetragonolobus | 0.1 |
| Gum | Gum | |
| Glycerin | Glycerin | 4.0 |
| Tego ® Betain F50 | Cocamidopropyl betaine | 14.0 |
| Solubilizer ® | PEG-40 Hydrogenated castor | 3.0 |
| | oil, Trideceth-9, Propylene | |
| | Glycol, Aqua | |
| 1,2-heptanediol | 1,2-heptanediol | 0.5 |
| Hydrolite ® 6 | 1,2 Hexanediol | 1.0 |
| SymGuard ® CD | Phenylpropanol, o-cymen-5-ol, | 0.3 |
| | Decylene Glycol | |
| SymRelief ® 100 | Bisabolol, Zingiber | 0.1 |
| | officinale root extract | |
| Perfume oil PO1, PO2, PO3, | Perfume | 0.2 |
| PO4, or PO5 | | |
| Frescolat ® X-cool | Menthyl ethylamido oxalate | 0.4 |
| Frescolat ® Plus | Menthol, Menthyl Lactate | 0.3 |
| Plantacare 2000 UP | Decyl Glucoside | 2.0 |
| Lactic acid 90% Nat. | Lactic Acid, Aqua | 0.3 |

TABLE 24
Mild feminine wash

| Ingredients | INCI | Amount |
|---|---|---|
| Aqua/Water | Aqua | ad 100 |
| SymSave ® H | Hydroxyacetophenone | 0.5 |
| Keltrol CG-T | Xanthan Gum | 0.5 |
| Glycerin | Glycerin | 2.0 |
| Propylene Glycol | Propylene Glycol | 3.0 |
| 1,2-undecanediol | 1,2-undecanediol | 0.2 |
| 2,3-heptanediol | 2,3-heptanediol | 0.05 |
| Tego Betain F50 | Cocamidopropyl betaine | 14.0 |
| 1,2-nonanediol | 1,2-nonanediol | 0.2 |
| SymGuard ® CD | Phenylpropanol, o-cymen-5-ol, | 0.3 |
| Frescolat ® ML nat | Menthyl Lactate | 0.2 |
| Plantacare ® 2000 UP | Decyl Glucoside | 2.0 |
| Extrapone ® Witch | Glycerin, Aqua, Hamamelis | 1.0 |
| Hazel GW | Virginiana Bark/-Leaf/Twig | |
| | Extract | |
| Solubilizer | PEG-40 Hydrogenated castor | 1.5 |
| | oil, Trideceth-9, Propylene | |
| Perfume oil PO1, PO2, PO3, | Perfume | 0.2 |
| PO4, or PO5 | | |
| SymCalmin ® | Pentylene Glycol, Butylene | 1.0 |
| | Blycol, Hydroxyphenyl | |
| Lactic acid 90% Nat. | Lactic Acid, Aqua | 0.32 |

TABLE 25
Mosquito & tick repellent Spray

| Ingredients | INCI | Amount |
|---|---|---|
| Ethanol | Ethanol | ad 100 |
| DEET | Diethyl Toluamide | 20.0 |
| Citriodiol | Cyclized Eucalyptus Citriodora | 3.0 |
| Perfume Oil PO1, PO2, PO3, | Perfume | 0.2 |
| PO4, or PO5 | | |
| 1,2-heptanediol | 1,2-heptanediol | 1.5 |
| 2,3-heptanediol | 2,3-heptanediol | 0.02 |
| 1,2-octanediol | Caprylyl Glycol | 0.2 |
| 2,3-octanediol | 2,3-octanediol | 0.01 |

TABLE 26
Insect Repellent Spray, aqueous/ethanolic

| Ingredients | INCI | Amount |
|---|---|---|
| Ethanol | Ethanol | 60.0 |
| Water | Water (Aqua) | 20.0 |
| Perfume Oil PO1, PO2, PO3, | Perfume | 0.3 |
| PO4 or PO5 | | |
| Icaridin | Hydroxyethyl Isobutyl | 20.0 |
| Eucalyptus Oil | Eucalyptus Oil | 0.3 |
| SymSol PF-3 | Water (Aqua). Sodium Lauryl | 1.5 |
| | Sulfoacetate. Pentylene Glycol. | |
| | Sodium Oleoyl Sarcosinate. | |
| 1,2-nonanediol | 1,2-nonanediol | 0.3 |
| 2,3-nonanediol | 2,3-nonanediol | 0.01 |
| 1,2-octanediol | Caprylyl Glycol | 0.3 |
| 2,3-octanediol | 2,3-octanediol | 0.05 |

TABLE 27
Insect Repellent Solution for Wipes

| Ingredients | INCI | Amount |
|---|---|---|
| Arlamol E | PPG-15 Stearylether | 3.0 |
| Polyethylenglycol 400 | PEG-8 | 5.0 |
| Ethanol | Ethanol | 35.0 |
| Glycerin | Glycerin | 4.0 |
| IR3535 | Etyhl | 20.0 |
| Citronellol | Citronellol | 0.5 |
| Perfume Oil PO1, PO2, PO3, | Perfume | 0.3 |
| PO4, or PO5 | | |
| Solubilizer | Trideceth-9 PEG 40, | 2.5 |
| | Hydrogenated Castor Oil | |
| Water | Water (Aqua) | ad 100 |
| 1,2-nonanediol | 1,2-nonanediol | 0.2 |
| 2,3-nonanediol | 2,3-nonanediol | 0.01 |
| 1,2-octanediol | Caprylyl Glycol | 0.3 |
| 2,3-octanediol | 2,3-octanediol | 0.05 |

TABLE 28
Anti-Sweat Sunscreen Lotion with repellent

| Ingredients | INCI | Amount |
|---|---|---|
| Aqua/Water | Aqua | ad 100 |
| 1,2-heptanediol | 1,2-heptanediol | 1.0 |
| 2,3-heptanediol | 2,3-heptanediol | 0.01 |
| 1,2-octanediol | Caprylyl Glycol | 0.3 |
| 2,3-octanediol | 2,3-octanediol | 0.05 |
| Citric Acid 30% Sol, | Aqua, Citric acid | 0.6 |
| Aristoflex ® Velvet | Polyacrylate crosspolymer 11 | 0.6 |
| Glycerin 99.5% | Glycerin, Aqua | 2.0 |
| SymSol ® PF-3 | Aqua, Pentylene glycol, Sodium lauryl sulfoacetate, | 1.0 |
| | Sodium oleoyl sarcosinate, Sodium chloride, | |
| | Sodium oleate | |
| SymSave ® H | Hydroxyacetophenone | 0.3 |
| Neo Heliopan ® 357 | Butyl methoxydibenzoylmethane | 3.0 |
| Neo Heliopan ® 303 | Octocrylene | 10.0 |
| Neo Heliopan ® OS | Ethylhexyl salicylate | 5.0 |
| Neo Heliopan ® HMS | Homosalate | 10.0 |
| Neo Heliopan ® E1000 | Isoamyl p-methoxycinnamate | 2.0 |
| Neo Heliopan ® BMT | Bis-ethylhexyloxyphenol methoxyphenyl triazine | 3.0 |
| DEET | Diethyl Toluamide | 5.0 |
| Cpoconut Oil | Coconut Oil | 2.0 |
| Dragoxat ® 89 | Ethylhexyl isononanoate | 6.0 |
| SymMollient ® S | Cetearyl nonanoate | 1.0 |
| EDTA ® BD | Disodium edta | 0.1 |
| SymDecanox ™ HA | Caprylic/capric triglyceride | 1.0 |
| | Hydroxymethoxyphenyl decanone | |
| Frescolat ® Plus | Menthol, Menthyl lactate | 0.3 |
| Citriodiol | Cyclized Eucalyptus Citriodora Leaf/Twig Oil | 3.0 |
| N-Ethyl-p-menthane- | Ethyl Menthane Carboxamide | 0.2 |
| 3-carboxamide | | |
| (Cooling Agent WS-3) | | |
| SymDeo ® Plus | Lauryl alcohol, Phenoxyethanol, 2-benzylheptanol, | 1.0 |
| | Decylene glycol | |
| Hydroviton ® PLUS | Aqua, Pentylene glycol, Glycerin, Fructose, Urea, | 4.0 |
| 2290 | Citric acid, Sodium hydroxide, Maltose, Sodium pca, | |
| | Sodium chloride, Sodium lactate, Trehalose, | |
| | Allantoin, Sodium hyaluronate, Glucose | |
| Sensocel ® 10 | Cellulose | 2.0 |
| Perfume Oil PO1, | Perfume | 0.5 |
| PO2, PO3, PO4, or | | |
| PO5 | | |

TABLE 29
Syndet antimicrobial soap bar

| Ingredients | INCI | Amount |
|---|---|---|
| Zetesap 813 A | Disodium Lauryl Sulfosuccinate, | ad 100 |
| | Sodium Lauryl Sulfate, Corn | |
| | Starch, Cetearyl Alcohol, | |
| | Paraffin, Titanium Dioxide | |
| Amphotensid GB 2009 | Disodium Cocoamphodiacetate | 6.0 |
| Allantoin | Allantoin | 1.0 |
| Perfume oil PO1; PO2; PO3; | Perfume | 1.0 |
| PO4 or PO5 | | |
| SymOcide C | o-cymen-5-ol | 0.1 |
| 1,2-undecanediol/2,3- | 1,2-undecanediol, 2,3- | 0.2 |
| undecanediol (95:5) | undecanediol | |
| 1,2-nonanediol/2,3-nonanediol | 1,2-nonanediol, 2,3-nonanediol | 0.5 |
| (98:2 w/w %) | | |

TABLE 30
Syndet soap bar

| Ingredients | INCI | Amount |
|---|---|---|
| Fenopon AC-78 | Sodium Cocoyl Isethionate | 20.0 |
| Natriumlaurylsulfoacetate | Sodium Lauryl Sulfoacetate | 16.0 |
| Paraffin | Paraffin | 19.0 |
| Wax. microcrystalline | Microcrystalline Wax | 1.0 |
| Corn Starch | Corn Starch | 8.0 |
| Coconut acid | Coconut acid | 2.0 |
| Lauric acid diethanol amide | Lauramide DEA | 2.0 |
| Dextrin | Dextrin | 21.0 |
| Lactic acid. 88% | Lactic Acid | 1.0 |
| SymGuard CD | 3-Phenylpropanol. o-cymen-5- | 0.3 |
| | ol. Decylene glycol | |
| Thymol | Thymol | 0.05 |
| Water | Water | ad 100 |
| Perfume oil PO1; PO2; PO3; | Perfume | 1.0 |
| PO4 or PO5 | | |
| 1,2-heptanediol/ | 1,2-heptanediol/ | 3.0 |
| 2,3-heptanediol | 2,3-heptanediol | |
| blend (95:5 w/w %) | blend (95:5 w/w %) | |

TABLE 31
Antimicrobial toilet soap bar

| Ingredients | Amount |
|---|---|
| Sodium soap from tallow | 60.0 |
| Sodium soap from palm oil | 27.0 |
| Glycerol | 2.0 |
| Sodium Chloride | 0.5 |
| 1-Hydroxyethane-1.1-diphosphonic acid. tetrasodium salt | 0.3 |
| Alpha-Tocopherol | 0.1 |
| Pigment Yellow 1 | 0.02 |
| Water | ad 100 |
| Perfume oil PO1; PO2; PO3; PO4 or PO5 | 3.0 |
| Farnesol | 0.2 |
| 2,3-heptanediol | 1.0 |
| 2,3-octanediol | 1.0 |

TABLE 32
Ice Crystal Soap

| Ingredients | INCI | Amount |
|---|---|---|
| Crystal SLEA/SLS Free | Aqua, Glycerin, Sorbitol, Sodium | ad 100 |
| | stearate, Sodium Laurate, | |
| | propylene Glycol, Sodium | |
| | Oleate, Sodium Myristate, | |
| | Sodium chloride, Glyceryl | |
| | Laurate, Cocaidopropyl Betaine, | |
| | Cocos Nucifera oil, Sodium | |
| | Thiosulfate, Sodium Citrate, | |
| | Citric Acid, Tetrasodium | |
| | Iminodisuccinate, Tetrasodium | |
| | Etidronate | |
| Frescolat MGA | Menthone Glycerin Acetale | 2.0 |
| 1,2-heptanediol | 1,2-heptanediol | 0.5 |
| 2,3-nonanediol | 2,3-nonanediol | 0.05 |
| Menthol | Menthol | 0.05 |
| Extrapone Glacier | Glycerin, Aqua | 1.0 |
| Water GW | | |

TABLE 33
Acne Scar Remover

| Ingredients | INCI | Amount |
|---|---|---|
| Aqua/Water | Aqua | ad 100 |
| 1,2-heptanediol | 1,2-heptanediol | 1.0 |
| 2,3-heptanediol | 2,3-heptanediol | 0.01 |
| 1,2-octanediol | Caprylyl Glycol | 0.3 |
| 2,3-octanediol | 2,3-octanediol | 0.05 |
| 1,2-undecanediol | 1,2-undecanediol | 0.1 |
| Glycerin | Glycerin | 2.0 |
| Edeta ® BD | Disodium Edta | 0.1 |
| Potassium Sorbate | Potassium Sorbate | 0.1 |
| Hydrolite ® 5 green | Pentylene Glycol | 2.0 |
| SymSave ® H | Hydroxyacetophenone | 0.5 |
| SymDiol ® 68 | 1,2-Hexanediol, Caprylyl Glycol | 0.5 |
| Extrapone ® Cucumber CL | Aqua, Propylene Glycol, | 1.0 |
| | Cucumis Sativus (Cucumber) | |
| | Juice | |
| SymGlucan ® | Aqua, Glycerin, 1,2-Hexanediol, | 5.0 |
| | Caprlyl Glycol, Beta-Glucan | |
| Dracorin ® GOC | Glyceryl Oleate Citrate, | 3.0 |
| | Caprylic/Capric Triglyceride | |
| Dragoxat ® 89 | Ethylhexyl Isononanoate | 3.0 |
| PCL-Liquid ® 100 | Cetearyl Ethylhexanoate | 2.0 |
| Isoadipate | Diisopropyl Adipate | 1.0 |
| Xiameter ® PMX-200 | Dimethicone | 0.5 |
| Silicone Fluid 100 cs | | |
| Frescolat ® ML | Methyl Lactate | 0.5 |
| SymClariol ® | Decylene Glycol | 0.5 |
| SymBright ® 2036 | Sclareolide | 0.2 |
| Sepimax ZEN | Polyacrylate Crosspolmer-6 | 1.5 |
| Orgasol 2002 DU NAT COS | Nylon-12 | 2.0 |
| Perfume oil PO1, PO2, PO3, | Perfume | 0.3 |
| PO4, or PO5 | | |

TABLE 34
Balancing Face Toner

| Ingredients | INCI | Amount |
|---|---|---|
| Aqua/Water | Aqua | Ad 100 |
| 2,3-heptanediol | 2,3-heptanediol | 0.5 |
| 2,3-octanediol | 2,3-octanediol | 0.5 |
| Glycerin | Glycerin | 3.00 |
| Edta Na2 | Disodium Edta | 0.10 |
| SymSave ® H | Hydroxyacetophenone | 0.50 |
| Dow Corning 2501 Wax | Bis-Peg-18 Methyl Ether | 3.00 |
| | Dimethyl Silane | |
| Solubilizer | Peg-40 Hydrogenated Castor | 2.00 |
| | Oil, Trideceth-9, Propylene | |
| | Glycol, Aqua | |
| Frescolat ® ML nat. | Menthyl Lactate | 0.30 |
| Hydrolite ® 5 | Pentylene Glycol | 5.00 |
| Perfume oil PO1, PO2, PO3, | Perfume | 0.10 |
| PO4, or PO5 | | |
| SymControl ® Care | Aqua, Glycerin, Tetraselmis | 2.00 |
| | Suecica Extract, Sodium | |
| | Benzoate, Lactic Acid, | |
| | Potassium Sorbate | |
| Actipone ® Ginger | Aqua, Butylene Glycol, Zingiber | 0.20 |
| | Officinale Root Extract, Peg-40 | |
| | Hydrogenated Castor Oil, | |
| | Trideceth-9 | |
| Sodium Hydroxide 10% | Aqua | 0.01 |
| solution | Sodium Hydroxide | |

TABLE 35
Biotic Acne Control Gel

| Ingredients | INCI | Amount |
|---|---|---|
| Aqua/Water | Aqua | Ad 100 |
| 1,2-nonanediol/2,3 | 1,2-nonanediol, 2,3-nonanediol | 0.2 |
| nonanediol blend | | |
| (95:5 w/w %) | | |
| SymSave ® H | Hydroxyacetophenone | 0.50 |
| Carbopol ® Ultrez 21 | Acrylates/C10-30 Alkyl Acrylate | 0.50 |
| Polymer | Crosspolymer | |
| Solubilizer | Peg-40 Hydrogenated Castor | 2.50 |
| | Oil, Trideceth-9, Propylene | |
| | Glycol, Aqua | |
| SymClariol ® | Decylene Glycol | 0.50 |
| SymMollient ® W/S | Trideceth-9, Peg-5 | 1.00 |
| | Isononanoate, Aqua | |
| Hydrolite ® 5 green | Pentylene Glycol | 5.00 |
| Dragosantol | Bisabolol | 0.05 |
| Farnesol | Farnesol | 0.10 |
| Ethanol | Alcohol, Aqua | 5.00 |
| Flowerpone ® Lavender | Aqua, Peg-40 Hydrogenated | 0.50 |
| | Castor Oil, Trideceth-9, | |
| | Pentylene Glycol, Lavandula | |
| | Angustifolia Flower Extract, | |
| | Propylene Glycol, Bisabolol | |
| SymReboot ™ L19 | Maltodextrin, Lactobacillus | 0.50 |
| | Ferment | |
| Sodium Hydroxide 10% | Aqua, Sodium Hydroxide | 1.10 |
| solution | | |
| Color | | 0.20 |

TABLE 36
SunCare Foam For Acne Prone Skin, exp. SPF 30

| Ingredients | INCI | Amount |
|---|---|---|
| Neo Heliopan ® OS | Ethylhexyl Salicylate | 5.0 |
| Neo Heliopan ® 357 | Butyl Methoxydibenzoylmethane | 5.0 |
| Neo Heliopan ® HMS | Homosalate | 10.0 |
| Neo Heliopan ® E1000 | Isoamyl P-Methoxycinnamate | 2.0 |
| Cetiol ® AB | C12-15 Alkyl Benzoate | 5.0 |
| Corapan ® TQ | Diethylhexyl 2,6-Naphthalate | 3.0 |
| Dragoxat ® 89 | Ethylhexyl Isononanoate | 1.0 |
| Isoadipate | Diisopropyl Adipate | 3.0 |
| Dracorin ® GOC | Glyceryl Oleate Citrate, | 3.0 |
| | Caprylic/Capric Triglyceride | |
| SymDecanox ™ HA | Caprylic/Capric Triglyceride, | 1.0 |
| | Hydroxymethoxyphenyl | |
| | Decanone | |
| Edeta ® BD | Disodium Edta | 0.1 |
| Keltrol ® CG-SFT | Xanthan Gum | 0.2 |
| Aqua/Water | Aqua | ad 100 |
| 1,2-heptanediol | 1,2-heptanediol | 0.5 |
| 2,3-octanediol | 2,3-octanediol | 0.1 |
| Neo Heliopan ® Hydro | Phenylbenzimidazole Sulfonic | 3.0 |
| | Acid | |
| Biotive ® L-Arginine | Arginine | 1.0 |
| Sodium Hydroxide 10% | Aqua, Sodium Hydroxide, | 2.0 |
| solution | | |
| SymSol ® PF-3 | Aqua, Pentylene Glycol, Sodium | 3.0 |
| | Lauryl Sulfoacetate, Sodium | |
| | Oleoyl Sarcosinate, | |
| | Sodium Chloride, Sodium | |
| | Oleate | |
| SymDiol ® 68 | 1,2-Hexanediol, Caprylyl Glycol | 0.3 |
| SymSave ® H | Hydroxyacetophenone | 0.5 |
| Perfume oil PO1, PO2, | Perfume | 0.5 |
| PO3, PO4, or PO5 | | |
| SymClariol ® | Decylene Glycol | 0.5 |
| SymGlucan ® | Aqua, Glycerin, 1,2-Hexanediol, | 2.0 |
| | Caprylyl Glycol, Beta-Glucan | |

TABLE 37
Cool Sebum Control Cleanser

| Ingredients | INCI | Amount |
|---|---|---|
| Proteol ™ APL | Sodium Cocoyl Apple Amino | 5.00 |
| | Acids | |
| Aqua/Water | Aqua | ad 100 |
| 1,2-heptanediol | 1,2-heptanediol | 0.5 |
| 2,3-nonanediol | 2,3-nonanediol | 0.01 |
| 1,2-nonanediol | 1,2-nonanediol | 0.3 |
| Neo-PCL Water Soluble N | Trideceth-9, Peg-5 | 15.00 |
| | Ethylhexanoate, Aqua | |
| Xiameter ® XM | Peg-12 Dimethicone | 5.00 |
| OFX-0193 Fluid | | |
| Glycerin | Glycerin | 8.00 |
| Perfume oil PO1, PO2, | Perfume | 0.50 |
| PO3, PO4, or PO5 | | |
| Hydrolite ® 5 | Pentylene Glycol | 3.00 |
| SymTriol ® | Caprylyl Glycol, 1,2-Hexanediol, | 0.50 |
| | Methylbenzyl Alcohol | |
| SymClariol ® | Decylene Glycol | 0.50 |
| Frescolat ® ML Cryst | Menthyl Lactate | 1.00 |
| Actipone ® Camu | Aqua, Glycerin, Myrciaria Dubia | 0.50 |
| Camu GW | Fruit Juice | |
| SymRelief ® S | Bisabolol, | 0.05 |
| | Hydroxymethoxyphenyl | |
| | Decanone | |
| SymRepair ® 100 | Hexyldecanol, Bisabolol, | 1.00 |
| | Cetylhydroxyproline | |
| | Palmitamide | |
| | Stearic Acid, Brassica | |
| | Campestris (Rapeseed) Sterols | |

1. A cosmetic or pharmaceutical composition comprising
(a1) 1,2-nonanediol;
(b1) at least one antimicrobial component; and
(c2) optionally at least one cosmetically or pharmaceutically active substance and/or additive;
or
(a2) 2,3-nonanediol;
(b2) at least one antimicrobial component; and
(c2) optionally at least one cosmetically or pharmaceutically active substance and/or additive;
or
(a2) a mixture comprising 1,2-nonanediol and 2,3-nonanediol;
(b2) at least one antimicrobial component; and
(c2) optionally at least one cosmetically or pharmaceutically active substance and/or additive.

2. A cosmetic or pharmaceutical composition, comprising
(a) at least one linear alkanediol having a carbon chain of 5 to 14 carbon atoms or a mixture comprising at least one first linear alkanediol having a carbon chain of 5 to 14 carbon atoms and one or more second linear alkanediols having a carbon chain of 5 to 14 carbon atoms which is different from the first linear alkanediol;
(b) at least one antimicrobial component; and
(c) optionally at least one cosmetically or pharmaceutically active substance and/or additive.

3. A cosmetic or pharmaceutical composition according to claim 2, wherein the at least one linear alkanediol or the first linear alkanediol is selected from the group consisting of pentanediol, hexanediol, heptanediol, octanediol, nonanediol, decanediol, undecanediol, dodecanediol, and tridecanediol.

4. (canceled)

5. A cosmetic or pharmaceutical composition according to claim 2, wherein the second linear alkanediol is selected from the group consisting of pentanediol, hexanediol, heptanediol, octanediol, nonanediol, decanediol, undecanediol, dodecanediol, and tridecanediol.

6. A cosmetic or pharmaceutical composition according to claim 2, wherein the at least one linear alkanediol, the first linear alkanediol or the second linear alkanediol is a (x,x+1) constitutional isomer, wherein x stands for the number of the carbon atom in the alkanediol chain, to which the OH groups of the alkanediol are chemically bonded.

7. A cosmetic or pharmaceutical composition according to claim 2, wherein the at least one linear alkanediol or the first linear alkanediol, is selected from the group consisting of
1,2-pentanediol, 2,3-pentanediol, 1,2-hexanediol, 2,3-hexanediol,
1,2-heptanediol, 2,3-heptanediol, 1,2-octanediol, 2,3-octanediol,
1,2-nonanediol, 2,3-nonanediol, 1,2-decanediol, 2,3-decanediol,
1,2-undecanediol, 2,3-undecanediol, 1,2-dodecanediol, 2,3-dodecanediol,
1,2-tridecanediol, 2,3-tridecanediol, and mixtures thereof; and/or
wherein the second linear alkanediol is selected from the group consisting of
1,2-pentanediol, 2,3-pentanediol, 1,2-hexanediol, 2,3-hexanediol,
1,2-heptanediol, 2,3-heptanediol, 1,2-octanediol, 2,3-octanediol,
1,2-nonanediol, 2,3-nonanediol, 1,2-decanediol, 2,3-decanediol,
1,2-undecanediol, 2,3-undecanediol, 1,2-dodecanediol, 2,3-dodecanediol,
1,2-tridecanediol, 2,3-tridecanediol, and mixtures thereof.

8. A cosmetic or pharmaceutical composition according to claim 7, wherein the mixture comprising at least one first linear alkanediol and one or more second linear alkanediols is selected from the group consisting of
a mixture comprising 1,2-hexanediol and 2,3-hexanediol;
a mixture comprising 1,2-heptanediol and 2,3-heptanediol;
a mixture comprising 1,2-octanediol and 2,3-octanediol;
a mixture comprising 1,2-nonanediol and 2,3-nonanediol;
a mixture comprising 1,2-decanediol and 2,3-decanediol;
a mixture comprising 1,2-undecanediol and 2,3-undecanediol;
a mixture comprising 1,2-dodecanediol and 2,3-dodecanediol;
a mixture comprising 1,2-tridecanediol and 2,3-tridecanediol
a mixture comprising 1,2-hexanediol and 2,3-octanediol;
a mixture comprising 1,2-octanediol and 2,3-hexanediol;
a mixture comprising 1,2-octanediol and 2,3-heptanediol
a mixture comprising 1,2-pentanediol and 2,3-hexanediol;
a mixture comprising 1,2-pentanediol and 1,2-heptanediol;
a mixture comprising 1,2-pentanediol and 2,3-heptanediol;
a mixture comprising 1,2-pentanediol and 2,3-octanediol;
a mixture comprising 1,2-pentanediol and 1,2-nonanediol;
a mixture comprising 1,2-pentanediol and 2,3-nonanediol
a mixture comprising 1,2-heptanediol and 1,2-octanediol;
a mixture comprising 1,2-heptanediol and 2,3-octanediol;
a mixture comprising 1,2-heptanediol and 1,2-nonanediol; and
a mixture comprising 1,2-heptanediol and 2,3-nonanediol.

9-11. (canceled)

12. A cosmetic or pharmaceutical composition according to claim 7, wherein the at least one linear alkanediol, the first linear alkanediol, and/or the second linear alkanediol is selected from the group consisting of 2,3-pentanediol, 2,3-hexanediol, 2,3-heptanediol, 2,3-octanediol, 2,3-nonanediol, 2,3-decanediol, 2,3-undecanediol, 2,3-dodecanediol, 2,3-tridecanediol, and mixtures thereof.

13. A cosmetic or pharmaceutical composition according to claim 2,
wherein the mixture comprising at least one first linear alkanediol and one or more second linear alkanediols comprises the first linear alkanediol and the second alkanediol in a ratio in a range of 50:50 to 99.9:0.1.

14. A cosmetic or pharmaceutical composition according claim 2, comprising no 1,2-decanediol.

15. A cosmetic or pharmaceutical composition according to claim 2, wherein the at least one antimicrobial component (b) is selected from the group consisting of Phenoxyethanol, Farnesol, Bisabolol, Ethylhexylglycerin, Triethyl Citrate, Hydroxyacetophenone, Benzyl Alcohol, 2-Methyl 5-cyclohexylpentanol, Benzoic Acid, Sorbic Acid, Dehydroacetic Acid, Sorbitan Caprylate, Caprylhydroxamic Acid, Dimethyl phenylbutanol, Dimethyl phenylpropanol, Levulinic Acid and esters and/or ketals thereof, Anisic Acid, 2-Benzylheptanol, Polyglyceryl-3 Caprylate, Polyglyceryl-10 Laurate, Glyceryl Caprate, Polyglyceryl-2 Caprate, Zinc Ricinoleate, Glyceryl Laurate, Glyceryl Caprylate, Xylityl Sesquicaprylate, Phenylpropanol, Lauryl Alcohol, o-Cymen-5-ol, Octenidine HCl, Undecylenoyl Glycine, Sodium Caproyl/Lauroyl Lactylate, Ethyl Lauroyl Arginate Laurate, PCA Ethyl Cocoyl Arginate, Salvia Officinalis (Sage) Oil, Tropolone, Oiroctone Olamine, Triclosan, Triclocarban, Chloroxylenol, Chlorocresol, Benzalkonium Chloride, Benzalkonium Bromide, Benzalkonium Saccharinate, Benzethonium Chloride, Chlorphenesin, Alkyl (C12-22) Trimethyl Ammonium Bromide and Chloride, Zinc Pyrithione, Climbazole, Chlorhexidine, Chlorhexidine Diacetate, Chlorhexidine Digluconate, Chlorhexidine Dihydrochloride, Hexamidine, Hexamidine Diisethionate, Silver Chloride, Silver Citrate, Hexylglycerin, Cyclohexylglycerin, Glycerin, Tetraselmis suecica extract, Mannitol, Sorbitol, Lactic Acid, Sodium Lauroyl Lactylate, Sodium Caproyl Lactylate, Salicylic Acid and its salts, Capryloyl Glycine, Ethyl Lauroyl Arginate HCl, Thymol, Jasmol, Zinc Citrate, Cetylpyridinium Chloride, Menthol, Eucalyptol, Methyl Salicylate, Ethanol, Camphor, Polyarginine, Polylysine, Gluconic Acid and salts thereof, Ascorbic Acid and salts thereof, Benzyl Salicylate, Disodium EDTA, Methylparaben and its salts, Sodium Benzoate, Potassium Sorbate, Propylparaben, Methylchloroisothiazolinone, Methylisothiazolinone, Butylparaben, Ethylparaben, Sodium Propylparaben, DMDM Hydantoin, Hydroxyethoxyphenyl Butanone, Hydroxyethoxyphenyl Butanol, Itaconic Acid and salts thereof, Propanediol Caprylate, 4-Hydroxyacetophenone, Iodopropynyl Butylcarbamate, Isobutylparaben, Sodium Ethylparaben, Diazolidinyl Urea, Glyceryl Caprylate, Sodium Phytate or Phytic acid, Sodium Levulinate or Levulinic Acid and esters and/or ketals thereof, TEA-Salicylate, Phenethyl Alcohol, Tetrasodium Glutamate Diacetate, Trisodium Ethylenediamine Disuccinate, Sodium Lactate, 4-Hydroxybenzoic acid and its salts and esters, thyme oil, eugenol, oil of cloves, mint oil, DMC, antibacterial fragrances, Frambinon, Xylityl caprylate, Benzoic acid 3-hyroxypropylester, Anisic acid 3-hydroxypropylester, 3-Hydroxypropyl 2-hydroxybenzoate, 1,3-propanediol, 1,2-pentanediol, 1,2-hexanediol, 1,2-heptanediol, 1,2-octanediol, 1,2-nonanediol, 1,2-decanediol, 2,3-pentanediol, 2,3-hexanediol, 2,3-heptanediol, 2,3-octanediol, 2,3-nonanediol, and mixtures of two or more the aforesaid antimicrobial components.

16. A cosmetic or pharmaceutical composition according to claim 2, wherein the at least one cosmetically or pharmaceutically active substance and/or additive is selected from the group consisting of agents against ageing of the skin, antioxidants, chelating agents, emulsifiers, surfactants, antimicrobials for product protection, green and synthetic polymers, rheology additives, oils, fragrances or perfume oils, polyols, and mixtures of two or more of the aforementioned substances.

17. A cosmetic or pharmaceutical composition according to claim 2, further comprising sebum reducing agents, anti-acne agents, anti-dandruff agents, anti-inflammatory and/or redness and/or itch ameliorating agents, odour absorbers and/or antiperspirant active agents, lenitive substances, moisturising and/or moisture-retaining substances, physiological cooling agents, Tetraselmis suecica extract, or mixtures of two or more of aforementioned agents or substances.

18. A cosmetic or pharmaceutical composition according to claim 2, comprising the at least one linear alkanediol or the mixture comprising at least one first linear alkanediol and one or more second linear alkanediols in an amount of 0.001 to 15.0% by weight based on the total weight of the composition.

19. A cosmetic or pharmaceutical composition according to claim 1, comprising
the 2,3-nonanediol or the 2,3-nonanediol of the mixture comprising 1,2-nonanediol
and 2,3-nonanediol in an amount of 0.001 to 15.0% by weight based on the total weight of the composition.

20. A cosmetic or pharmaceutical composition according to claim 2, comprising the at least one antimicrobial component in an amount of 0.001 to 15.0% by weight based on the total weight of the composition.

21. A cosmetic or pharmaceutical composition according to claim 2, wherein
the cosmetic or pharmaceutical composition is a dispersion comprising an oil component in an amount of ≥1% by weight, and wherein the cosmetic or pharmaceutical composition is in the form of an emulsion, a liquid, a lotion, as suspension, a milk, an ointment, a paste, a gel, a cream based formulation, an oil based formulation, or a liposome-based formulation; or
the cosmetic or pharmaceutical composition is a liquid surfactant formulation; or
the cosmetic or pharmaceutical composition is a solid surfactant formulation; or
the cosmetic or pharmaceutical composition is an aqueous or aqueous/alcoholic or aqueous/glycolic based solution, comprising the alcohol or glycol in an amount of 0.1% to ≤50% by weight, based on the total weight of the solution.

22-23. (canceled)

24. A method of preventing and/or treating a dermatological or keratological disease in a patient in need thereof, comprising administering a therapeutically effective amount of a pharmaceutical composition of claim 2.

25. A method of reducing or inhibiting microbial activity on skin or scalp comprising applying the cosmetic or pharmaceutical composition of claim 2 to the relevant areas of the skin or scalp.

26-28. (canceled)

29. A method for reducing the sebum overproduction on skin or scalp and/or for suppressing dandruff on skin, scalp or nails, comprising the following steps:
(a). providing at least one linear 1,2-alkanediol selected from the group consisting of 1,2-heptanediol, 1,2-nonanediol, 1,2-undecanediol, and 1,2-tridecanediol or at least one linear 2,3-alkanediol selected from the group consisting of 2,3-pentanediol, 2,3-hexanediol, 2,3-heptanediol, 2,3-octanediol, 2,3-nonanediol, 2,3-decanediol, 2,3-undecanediol, 2,3-dodecanediol and 2,3-tridecanediol or a mixture comprising a linear 1,2-alkanediol and a linear 2,3-alkanediol selected from the group consisting of a mixture comprising 1,2-pentanediol and 2,3-pentanediol; a mixture comprising 1,2-hexanediol and 2,3-hexanediol; a mixture comprising 1,2-heptanediol and 2,3-heptanediol; a mixture comprising 1,2-octanediol and 2,3-octanediol; a mixture comprising 1,2-nonanediol and 2,3-nonanediol; a mixture comprising 1,2-decanediol and 2,3-decanediol; a mixture comprising 1,2-undecanediol and 2,3-undecanediol; a mixture comprising 1,2-dodecanediol and 2,3-decanediol; and a mixture comprising 1,2-tridecanediol and 2,3-tridecanediol;
(b) preparing a cosmetic or pharmaceutical preparation to be topically applied by incorporating the alkanediol or the alkanediol mixture from step (a) into the cosmetic or pharmaceutical preparation; and
(c) applying the cosmetic or pharmaceutical preparation from step (b) to relevant skin or scalp areas;
wherein the amount of the alkanediol is sufficient to reduce the sebum overproduction or dandruff on skin, scalp or nails.
